(19) United States
(12) Patent Application Publication
(10) Pub. No.: US 2025/0290072 A1
(43) Pub. Date: Sep. 18, 2025

(54) MODULATION OF SYNGAP1 GENE TRANSCRIPTION USING ANTISENSE OLIGONUCLEOTIDES TARGETING REGULATORY RNAS

(71) Applicant: CAMP4 Therapeutics Corporation, Cambridge, MA (US)

(72) Inventors: Ali Al Abdullatif, Somerville, MA (US); Alfica Sehgal, Belmont, MA (US); Gokul Ramaswami, New York, NY (US); Preeti Kashinath Sathe, Cambridge, MA (US); Yeliz Yuva-Aydemir, Grafton, MA (US); David A. Bumcrot, Belmont, MA (US)

(21) Appl. No.: 19/222,816

(22) Filed: May 29, 2025

Related U.S. Application Data
(63) Continuation of application No. PCT/US2023/082182, filed on Dec. 1, 2023.
(60) Provisional application No. 63/385,695, filed on Dec. 1, 2022.

(51) Int. Cl. C12N 15/113

(52) U.S. Cl. CPC C12N 15/113; C12N 2310/11; C12N 2310/315; C12N 2310/321; C12N 2310/3231; C12N 2310/3341

(57) ABSTRACT

Described herein are methods of modulating SYNGAP1 gene transcription using antisense oligonucleotides (ASOs) targeting regulatory RNAs, such as promoter-associated RNAs, enhancer RNAs, and natural antisense transcripts (NATs). These methods are useful for increasing expression of SYNGAP1 mRNA and protein, thereby treating diseases associated with SYNGAP1 mutations.

### CROSS REFERENCE TO RELATED APPLICATIONS

This application is a continuation of International Application No. PCT/US2023/082182, filed on Dec. 1, 2023, which claims the benefit of U.S. Provisional Application No. 63/385,695, filed Dec. 1, 2022, each of which are hereby incorporated in their entirety by reference.

### SEQUENCE LISTING

The instant application contains a Sequence Listing which is hereby incorporated by reference in its entirety. Said XML copy, created on Jan. 11, 2024, is named CTC-032WO_SL.xml, and is 7,070,103 bytes in size.

### BACKGROUND

Transcription factors bind specific sequences in promoter and enhancer DNA elements to regulate gene transcription. It was recently reported that active promoters and enhancer elements are themselves transcribed, generating noncoding regulatory RNAs (regRNAs) such as promoter-associated RNAs (paRNAs) and enhancer RNAs (eRNAs) (see Sartorelli and Lauberth, Nat. Struct. Mol. Biol. (2020) 27: 521-28). Unlike coding RNAs, regRNAs are transcribed bi-directionally. Various models have been proposed for the functions of regRNAs, including nucleosome remodeling (see Mousavi et al., Mol. Cell (2013) 51(5):606-17), modulation of enhancer-promoter looping (see Lai et al., Nature (2013) 494(7438):497-501), and direct interaction with transcription regulators (see Sigova et al., Science (2015) 350, 978-81).

Approximately 1-2% of all cases of intellectual disabilities are due to mutations in the SYNGAP1 gene. SYNGAP1-related intellectual disability (SYNGAP1-ID) is a neurological disorder characterized by moderate to severe impaired intellectual development with delayed psychomotor development. Mental retardation, autosomal dominant 5 (MRD5), also known as intellectual disability autosomal; dominant 5, is a SYNGAP1-ID that is caused by an autosomal recessive mutation in the SYNGAP1 gene. SYNGAP1-related non-syndromic intellectual disability (NSID) is a result of a heterozygous pathogenic mutation in SYNGAP1 (approximately 89% of cases) or a deletion of 6p21.3 (approximately 11% of cases). SYNGAP1-related NSID presents as moderate to severe cognitive impairment, mild hypotonia, global developmental delay, delayed language development, disordered sleep, oral dyspraxia, inattention, impulsivity, physical aggression, mood swings, sullenness, and rigidity. In addition, 94-98% of cases of MRD5 and SYNGAP1-related NSID also present with epilepsy. There is no cure or treatment for MRD5 or SYNGAP1-related NSID. Patient treatment is limited to treatment of epilepsy and behavioral management. Thus, additional therapeutics are needed.

Gene expression has been generally known as an undruggable biological process. Despite on-going efforts into understanding the biology of gene transcription and regRNAs, clinically suitable methods of modulating gene expression are limited. There remains a need for new and useful methods for treating diseases associated with aberrant gene expression.

### SUMMARY

In one aspect, provided herein are antisense oligonucleotides (ASO) complementary to at least 8 contiguous nucleotides of a regulatory RNA of human SYNGAP1, wherein the regulatory RNA has a nucleotide sequence selected from the group consisting of SEQ ID NOs: 1-7.

In another aspect, provided herein are antisense oligonucleotides (ASO) complementary to at least 8 contiguous nucleotides of a regulatory RNA of human SYNGAP1, wherein the regulatory RNA has a nucleotide sequence selected from the group consisting of SEQ ID NOs: 4, 5, or 6.

In some embodiments, the ASO is complementary to a sequence in the regRNA that is no more than 200 nucleotides from the 3′ end of the regRNA.

In some embodiments, the ASO is complementary to a sequence in the regRNA that is no more than 200 nucleotides from the 5′ end of the regRNA.

In some embodiments, the regRNA is not a polyadenylated RNA.

In some embodiments, the regulatory RNA has a nucleotide sequence of SEQ ID NO: 1, and the ASO comprises a nucleotide sequence selected from the group consisting of SEQ ID NOs: 10-59, 220-250, 261-267, 272-278, 526-528, 542-591, 702-728, 729-735-741, 988-990, and 1004-2961.

In some embodiments, the regulatory RNA has a nucleotide sequence of SEQ ID NO: 2, and the ASO comprises a nucleotide sequence selected from the group consisting of SEQ ID NOs: 60-73.

In some embodiments, the regulatory RNA has a nucleotide sequence of SEQ ID NO: 3, and the ASO comprises a nucleotide sequence selected from the group consisting of SEQ ID NOs: 74-109, 251-260, 268-271, and 279.

In some embodiments, the regulatory RNA has a nucleotide sequence of SEQ ID NO: 4 or 6, and the ASO comprises a nucleotide sequence selected from the group consisting of SEQ ID NOs: 110-219, 280-525, 529-541, 592-701, 742-891, 906-987, 991-1003, and 2962-4852.

In some embodiments, the regulatory RNA has a nucleotide sequence of SEQ ID NO: 5, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961.

In some embodiments, the ASO comprises the nucleotide sequence of at least 8 contiguous nucleotides of chr6:33419695-33419939.

In some embodiments, the ASO comprises the nucleotide sequence of at least 8 contiguous nucleotides of chr6:33453987-33454269.

In some embodiments, the ASO comprises the nucleotide sequence of at least 8 contiguous nucleotides of chr6:33419674-33419940.

In some embodiments, the ASO is no more than 50, 40, 30, 25, 20, 18, or 16 nucleotides in length.

In some embodiments, the ASO comprises a RNA polynucleotide comprising one or more chemical modifications.

In some embodiments, at least 3, 4, or 5 nucleotides at the 5′ end and at least 3, 4, or 5 nucleotides at the 3′ end of the ASO comprise ribonucleotides with one or more chemical modifications.

In some embodiments, the one or more chemical modifications comprise a nucleotide sugar modification comprising one or more of 2′-O—C₁₋₄alkyl such as 2′-O-methyl (2′-OMe), 2′-deoxy (2′-H), 2′-OC₁₋₃alkyl-O—C₁₋₃alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

In some embodiments, the one or more chemical modifications comprise an internucleotide linkage modification comprising one or more of phosphorothioate (“PS” or (P(S))), phosphoramidate (P(NR₁R₂) such as dimethylaminophosphoramidate (P(N(CH₃)₂)), phosphonocarboxylate (P(CH₂)ₙCOOR) such as phosphonoacetate “PACE” (P(CH₂COO⁻)), thiophosphonocarboxylate ((S)P(CH₂)ₙCOOR) such as thiophosphonoacetate “thioPACE” ((S)P(CH₂COO⁻)), alkylphosphonate (P(C₁₋₃alkyl) such as methylphosphonate —P(CH₃), boranophosphonate (P(BH₃)), or phosphorodithioate (P(S)₂).

In some embodiments, the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

In some embodiments, the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

In some embodiments, the ASO further comprises a GalNAc moiety, optionally a GalNAc3 moiety.

In some embodiments, the ASO does not comprise 10 or more contiguous nucleotides of unmodified DNA.

In some embodiments, the ASO does not comprise a deoxyribonucleotide.

In some embodiments, the ASO does not comprise an unmodified ribonucleotide.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

In some embodiments, each ribonucleotide of the ASO is modified by 2′-O-methoxyethyl.

In some embodiments, each nucleotide of the ASO is a ribonucleotide modified by 2′-O-methoxyethyl.

In some embodiments, the ASO comprises 10 or more contiguous nucleotides of unmodified DNA flanked by at least 3 nucleotides of modified ribonucleotides at each of the 5′ end and the 3′ end.

In some embodiments, each cytidine in the ASO is modified by 5-methyl.

In some embodiments, the regRNA is a Natural Antisense Transcript (NAT).

In some embodiments, the regRNA is a paRNA.

In another aspect, provided herein are pharmaceutical compositions comprising an ASO disclosed herein and a pharmaceutically acceptable carrier or excipient carrier.

In another aspect, provided herein are methods of increasing transcription of SYNGAP1 in a human cell, the method comprising contacting the cell with an ASO disclosed herein or a pharmaceutical composition disclosed herein.

In some embodiments, the cell is a neuron.

In some embodiments, the ASO increases the amount of the regulatory RNA in the cell.

In some embodiments, the ASO increases the stability of the regulatory RNA in the cell.

In some embodiments, the method results in increased SYNGAP1 mRNA in the cell.

In some embodiments, the method results in increased SYNGAP1 protein in the cell.

In one aspect, provided herein are methods of treating a disease or disorder, the method comprising administering to a subject in need thereof an effective amount of an ASO disclosed herein or a pharmaceutical composition disclosed herein.

In some embodiments, the disease or disorder is a SYNGAP1-related disease or disorder.

In some embodiments, the SYNGAP1-related disorder is SYNGAP1-related intellectual disability (ID), mental retardation, autosomal dominant 5 (MRD5), or SYNGAP1-related non-syndromic intellectual disability (NSID).

In some embodiments, the disease or disorder is a central nervous system (CNS) disorder or a peripheral nervous system (PNS) disorder.

In some embodiments, the disease or disorder is an affective disorder (e.g., depression), schizophrenia, Alzheimer's disease, Parkinson's disease, Huntington's disease, an autism spectrum disorder (ASD), (e.g., Asperger's syndrome, autistic disorder, Pervasive Developmental Disorder-Not Otherwise Specified (PDD-NOS)), or a CNS or PNS trauma (e.g., brain or spinal cord ischemia or trauma, stroke, or a neurological deficit associated with surgery or anesthesia).

In some embodiments, administration of the ASO modulates SYNGAP1 gene expression in the subject (e.g., in a cell or tissue of the subject) relative to a pre-administration baseline level.

In some embodiments, the ASO increases the amount of the regulatory RNA in a cell of the subject.

In some embodiments, the ASO increases the stability of the regulatory RNA in a cell of the subject.

In some embodiments, administration of the ASO increases SYNGAP1 gene expression in a cell of the subject relative to a pre-administration baseline level.

In some embodiments, the cell is a neuron.

### BRIEF DESCRIPTION OF THE DRAWINGS

FIG. 1A shows an illustrative schematic of eRNA, paRNA, mRNA, and natural antisense transcript (NAT) of a gene on the chromosome. The eRNA, paRNA, and NAT are all non-coding RNAs. The eRNA is transcribed bidirectionally from an enhancer of the gene. The paRNA is transcribed from the promoter of the gene, same as the mRNA, but in the antisense direction. The NAT is transcribed from a downstream promoter of its own in the antisense direction, such that the transcript overlaps at least partially with the mRNA. FIG. 1B shows an illustrative schematic of the interaction of regRNA with enhancer and promoter regions to recruit transcription factors and regulators that modulate gene expression.

FIG. 2 provides exemplary ASO sequences and chemistries targeting human SYNGAP1 regRNAs. Light gray shading indicates 2′-MOE; * indicates 5Me-C; dark gray shading indicates LNA; dark gray line indicates phosphodiester bond (PO); white indicates DNA. FIG. 2 discloses SEQ ID NOS 592-701, 742-851, 544-568, 4853, 4854, 569-594, 4855-4901, 702-719, 4902, 720-728, 4903-4913, 729-735, 4914-4917, 736-738, 4918, 739-741 and 4919, respectively, in order of appearance.

FIG. 3 shows that SYNGAP1 regRNAs RR86 and RR93 were detected in HEK293 and SK-N-AS cells, as well as human brain samples via RNA capture seq and qPCR.

FIGS. 4A and 4B show SYNGAP1 mRNA levels in HEK293 cells (FIG. 4A) and SK-N-AS cells (FIG. 4B) after treatment with the indicated SYNGAP1 regRNA targeting ASOs, or a gapmer non-targeting control (NTC) ASO (CO-1588), a steric NTC ASO (CO-1589), or untreated control (“UTC” or “No ASO”). FIGS. 4C and 4D show a dose dependent increase of SYNGAP1 mRNA levels in HEK293 cells (FIG. 4C) and SK-N-AS cells (FIG. 4D) after treatment with the indicated SYNGAP1 regRNA targeting ASOs, as compared to cells treated with a gapmer NTC ASO (control; CO-1588). FIGS. 4E and 4F show a dose dependent increase of SYNGAP1 mRNA levels in HEK293 cells (FIG. 4E) and SK-N-AS cells (FIG. 4F) after treatment with the indicated ASOs, as compared to cells treated with a gapmer NTC ASO (control; CO-1588).

FIG. 5 shows SYNGAP1 mRNA levels in SK-N-AS cells and HEK293 cells after treatment with the indicated SYNGAP1 regRNA targeting ASOs, or a gapmer non-targeting control (NTC) ASO (CO-1588), a steric NTC ASO (CO-1589), or untreated control (“UTC”).

FIG. 6 shows a dose dependent upregulation of SYNGAP1 mRNA levels in both SK-N-AS and HEK293 cells after treatment with the indicated SYNGAP1 regRNA targeting ASOs, as compared to untreated control (“UTC”) or cells treated with a gapmer NTC ASO (control; CO-1588).

FIG. 7 shows SYNGAP1 mRNA levels in neurons differentiated from human induced pluripotent stem cells after treatment with the indicated concentrations of SYNGAP1 regRNA targeting ASOs or a gapmer NTC ASO (CO-1588; control).

### DETAILED DESCRIPTION

The present disclosure provides antisense oligonucleotides (ASOs) targeting regulatory RNAs, such as promoter-associated RNAs (paRNAs) and enhancer RNAs (eRNAs), and methods using these ASOs to regulate gene expression. These methods are useful for modulating the levels of gene products, for example, modulating expression levels of SYNGAP1, to thereby treat SYNGAP1-related disorder (e.g., diseases associated with SYNGAP1 mutations), such as mental retardation, autosomal dominant 5 (MRD5) and SYNGAP1-related non-syndromic intellectual disability (NSID) or other disease or disorders.

Various aspects of the compositions and methods described in the present application are set forth in the sections below.

### I. Definitions

To facilitate an understanding of the present application, a number of terms and phrases are defined below.

The terms “a” and “an” as used herein mean “one or more” and include the plural unless the context is inappropriate.

As used herein, the terms “SYNGAPl” or “synaptic Ras GTPase activating protein 1” refer to the gene of NCBI Gene ID: 8831 or Hugo Gene Nomenclature Committee (HGNC) ID: 11497 when used in reference to the human gene, or the protein of UniProt Accession No. Q96PVO (human) when used in reference to a human version of the protein, and to the gene of NCBI Gene ID: 240057 when used in reference to the mouse gene, or to the protein of UniProt Accession No. J3QQ18 (mouse), when used in reference to a mouse version of the protein, and related isoforms and orthologs of the foregoing. SYNGAP1 is a protein of the post-synaptic density (PSD) of glutamatergic neurons that interacts with PSD95 and SAP102, and is capable of positively or negatively regulating the density of N-Methyl-D-aspartic acid (NMDA) and α-amino-3-hydroxy-5-methyl-4-isoxazolepropionic acid (AMPA) receptors at the glutamatergic synapses, and also negatively regulates small G protein signaling downstream of glutamate receptor activation (see, e.g., Jeyabalan et al. (2016) Front. Cell Neurosci. 10: 32, incorporated herein by reference). In some embodiments, a SYNGAP1 protein comprises an isoform of SYNGAP1 (e.g., an N-terminus isoform A, B, and C and/or a C-terminus isoform alpha1 (α1), alpha2 (α2), beta (β), or gamma (γ) of human SYNGAP1. In some embodiments, a SYNGAP1 protein comprises an isoform selected from SYNGAP1 Aα1, SYNGAP1 Aα2, SYNGAP1 Aβ, SYNGAP1 Aγ, SYNGAP1 Bα1, SYNGAP1 Bα2, SYNGAP1 Bβ, SYNGAP1 Bγ, SYNGAP1 Cα1, SYNGAP1 Cα2, SYNGAP1 Cβ, SYNGAP1 Cγ, or any combination of the foregoing isoforms.

As used herein, the terms “regulatory RNA” and “regRNA” are used interchangeably to refer to a noncoding RNA transcribed from a regulatory element of a gene (e.g., a protein-coding gene), wherein the gene is not the noncoding RNA itself. Exemplary regulatory elements include but are not limited to promoters, enhancers, super-enhancers, and natural antisense transcripts. A noncoding RNA transcribed from a promoter, in the antisense direction, is also called “promoter RNA” or “paRNA.” A noncoding RNA transcribed from an enhancer or super-enhancer, in either the sense direction or the anti-sense direction, is also called “enhancer RNA” or “eRNA.”

As used herein, the term “nascent RNA” refers to an RNA that is still being transcribed or has just been transcribed by RNA polymerase and remains tethered to the DNA from which it is transcribed. An RNA that has dissociated from the DNA from which it is transcribed is also called an “untethered RNA.”

As used herein, the term “antisense oligonucleotide” or “ASO” refers to a single-stranded oligonucleotide having a nucleotide sequence that hybridizes with a target nucleic acid under suitable conditions or a conjugate comprising such single-stranded oligonucleotide. In some embodiments, the disclosure encompasses pharmaceutically acceptable salts of any of the ASOs described herein. Suitable pharmaceutically acceptable salts include, but are not limited to, sodium, potassium, calcium, and magnesium salts. In some embodiments, the ASOs provided herein are lyophilized and isolated as salts (e.g., sodium salts).

As used herein, in some embodiments, the stability of a regRNA is reversely correlated with the degradation rate of the regRNA. In some embodiments, where an ASO increases the stability of a regRNA, it reduces the degradation rate of the regRNA. In some embodiments, where an ASO decreases the stability of a regRNA, it increases the degradation rate of the regRNA. In some embodiments, the degradation rate of a regRNA can be measured by blocking synthesis of new regRNA and assessing the half-life of the existing regRNA.

As used herein, the terms “subject” and “patient” refer to an organism to be treated by the methods and compositions described herein. Such organisms preferably include, but are not limited to, mammals (e.g., rodents (e.g., mice), primates, simians, equines, bovines, porcines, canines, felines, and the like), and more preferably include humans.

As used herein, the term “effective amount” refers to the amount of a compound (e.g., a compound of the present application) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, PA (1975).

Throughout the description, where compositions are described as having, including, or comprising specific components, or where processes and methods are described as having, including, or comprising specific steps, it is contemplated that, additionally, there are compositions described in the present application that consist essentially of, or consist of, the recited components, and that there are processes and methods according to the present application that consist essentially of, or consist of, the recited processing steps.

As a general matter, compositions specifying a percentage are by weight unless otherwise specified. Further, if a variable is not accompanied by a definition, then the previous definition of the variable controls.

### II. Antisense Oligonucleotides

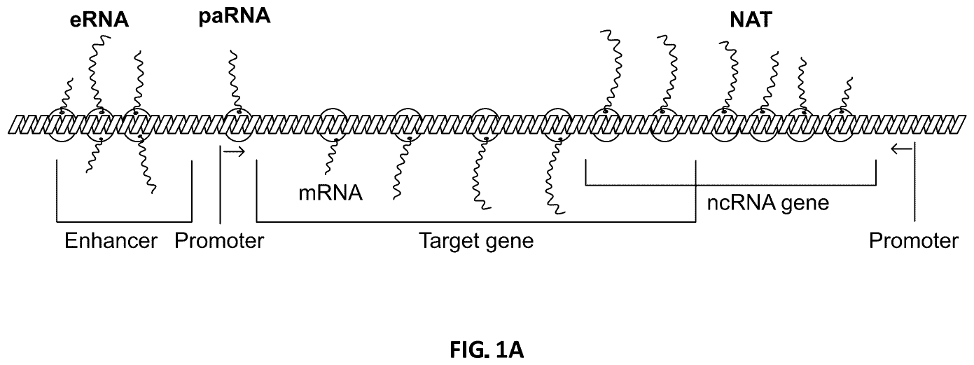

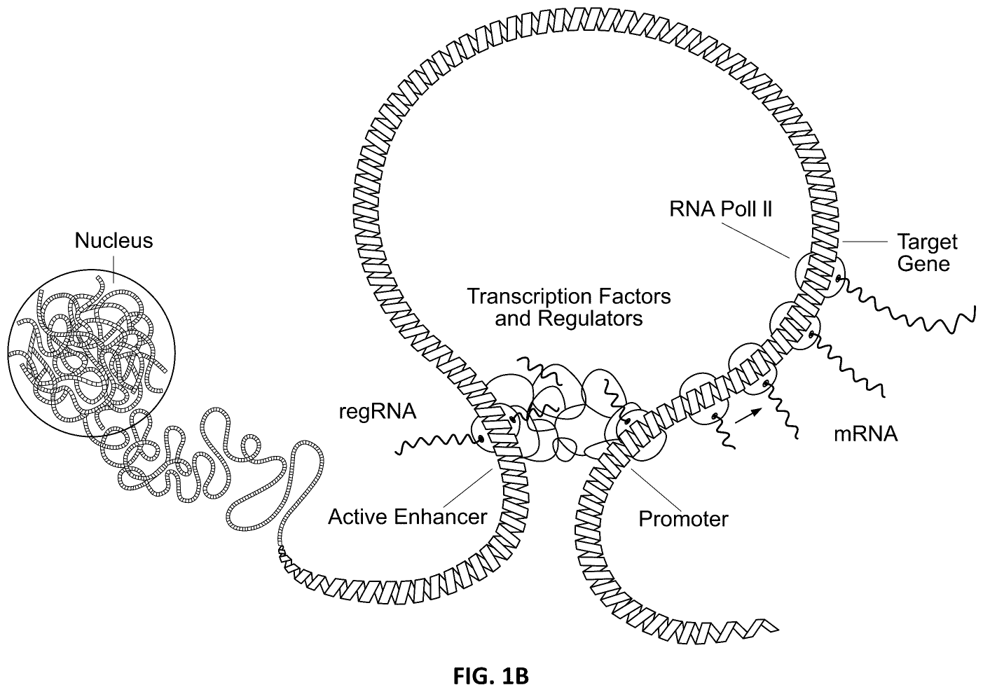

In some embodiments, the antisense oligonucleotides (ASO) disclosed herein hybridize with or target a regRNA (e.g., an eRNA, a paRNA, or a NAT) transcribed from a regulatory element of a SYNGAP1 gene, also referred to herein as a “SYNGAP1 regRNA”. It is understood that NATs, eRNAs, and paRNAs are regRNAs modulating (e.g., facilitating or upregulating) gene expression (FIG. 1). In some embodiments, the SYNGAP1 regRNA is a human SYNGAP1 regRNA. In some embodiments, the SYNGAP1 regRNA is a mouse SYNGAP1 regRNA. In certain embodiments, the SYNGAP1 regRNA is an eRNA. In certain embodiments, the SYNGAP1 regRNA is a paRNA. In certain embodiments, the SYNGAP1 regRNA is a NAT. In certain embodiments, the SYNGAP1 regRNA is not a polyadenylated RNA.

eRNAs can be identified using methods known in the art, such as Assay for Transposase-Accessible Chromatin using sequencing (ATAC-seq), global run-on sequencing, precision run-on sequencing, cap analysis gene expression, and histone modification analysis (see, e.g., Sartorelli & Lauberth, Nat. Struct. Mol. Biol. (2020) 27:521-28; PCT Application Publication No. WO2013/177248). paRNAs are RNAs transcribed from promoters of target genes in the antisense direction (transcripts in the sense direction are mRNAs of the target genes). They can be identified by similar methods, taking into account of their specific location and orientation. The nucleotide sequences of exemplary human and mouse SYNGAP1 regRNAs are provided in Table 1 below. Any of these human and mouse SYNGAP1 regRNAs are contemplated as a target regRNA of an ASO disclosed herein.

TABLE 1
Exemplary regRNAs

| SEQ | | |
|---|---|---|
| ID | | |
| NO | Name | Sequence |
| 1 | RR86_v1 | TAGTAGGAGGGCACAGGCCCAGGGTGTCCTCGCAACGGGCGTCCCGGGGAGCTGCTGAAGT |
| | human | CCTCCCCGCTGCAGAGGACCGAGAGGCAGGGCCCGGGAGAGAACAGCCCGGTCGGAGGGGG |
| | regRNA | TGGGCCGTCAGCCGCCCCGGAAAGCTGCGTTTCCCGGTGATTAAGTGTAGCACCCGCCCCC |
| | | GGTAGGTCCTGAGAGGGGCGAGATTAGTTGGGTACTTACCATGTGGGCCTGGCACTTGCCC |
| | | ACATCACTTTGTGTCTTCCCACCGGCCCTGCGAGGGTGGGGGTTGCCGTCCTCAGTTTCCA |
| | | GGCGGGGGAAACGGGCTGGAAGCGTCACTCAGCTAATAGTAGTGGACCTGGGACTGGAACT |
| | | CAGATTTGCTAAGGAACCAGGCAGGTTCTTTCCTGTTTTCTGCACGGGAGCATTTTAACCA |
| | | GCAATTAAAGAAACCTTATTTCTAGGTAATTTTTAAAAGA |
| 2 | RR87 | GCCGCCCCTCACCTGCCAATGATGCTCTTGATCTGGGAATCCTTCTCTGCCTGCTGCTGCC |
| | human | TTAGCCTCTTCTCACTCTGCTCCAGTCGGGCCTGATACTGCATCAGGATTTTGCTGGTTTG |
| | regRNA | TTCTTCCTGGGACAGCAGCCTCCGCTCATACTCTTCCAGCTTCCGGTTGGACATGTGCAGC |
| | | CGCTCTTTCA |
| 3 | RR88 | GATCCCTGCAAATGTAATCAAACTAAGAAGAGGCCAGCCATGGTATCTCATTCCTGTAATC |
| | human | CCAGCACTTTGGGAGGCCGAGGTGGGTGGATTGCCTGAGGTCAGGAGTTCGAGCCAGCCTG |
| | regRNA | GCCAACATAGTGAAACCCCGTCTCTAATAAAAATACAAAAACAAATTAGCTGGGCATAGTG |
| | | GCACGTGCCTGTAATCCCAGCTACTCAGGAGGCTGAGGCAGGAGAATCACTTGAACCTGGA |
| | | AGGCGGAGGTTGCAGTGAGCTGAGCTCATGCCATTGCACTCCAAGCCTGGGAAACAAGAGC |
| | | GATATTCCATCTCAAAAAAAAGAGGCCATACTGAATTAGAGTGGACCCTTAACCCACCTTA |
| | | TAAAAACTGGTGTCTTTATATGGAGAGGGAGATTTGGAGACACAGAGATACAGGATACAGA |
| | | GACACACAGAAGGAAGCCAGCTACATGAAGATGGAGGCAGAAATTAGACGGATGCAGTTAT |
| | | AAGCCCAGGAATGCCAAGGACTGCTGGCAACCTCCGGAAGCTAGGAAGAGGCAAGGAAGGA |
| | | TTCCCTGCTACAGCATCAAAGAGAGCGTGGCCCTGCTGATACCTTGCTTTTGGACTTCCAG |
| | | CCTCCTGAACTGTGAGAGAATAAATTTCTGTTGTTTTAC |
| 4 | RR93_v1 | AGGAGGAGGAGAGAGGAGGAGAGAGGAGCAGAGAGAAGCAGAGAGGAGGAGAGAGGAGGAG |
| | paRNA | GAGGAGGAGAATAAGAGCCAACGGCAGCAGCGGCAGCCGAGAGAGAGGGGGGGCGGGGGAG |
| | human | CGAGCGAGAGCGAAGAGAGCAGGAGGAGGAGGAGGAGGGAGAGACTCTGCAGCCCCCACCC |
| | regRNA | CTACTCCGGGAGGCCCAGATTGTGAGAGAGAGAGAGACCCCTGACTCAAAAACACACCAGA |
| | | GAGAGACAGAGGTCCTAAAGATGCAGACCCCAGACCCTGAGACAGAGAGACCCTAGACCCA |
| | | CAGAGAAAGATACTTCACCACCCCCCACTGTGGGGTAATTTGAGACACAGGGCCAAAATCT |
| | | AGAGGAAAAGAGACTCCTGATTTGAGGAAAAAACGACCCCAGATCCCCCAGAGAGAGAAGC |
| | | TCAATATCAAAATCTGAAAATCCAGTCTAAAAAGGGTTCTCCATACCTACAGATATCTTAG |
| | | ACTCCAGACCCTGAGATGATGATTTCAGGGACCAGGACCTGAGACCTAGACTCAGAAAAAG |
| | | ATGAGGCCCAGATTCAGAAAGAGCCAGATGTAGAATTAGAGCTTAGAGAACTCAGACCTAG |
| | | AAAGAGACCCCAGACTCAGATCTCAGAGACTGATAACTTAGAGACAGAGACACTCCTGACT |
| | | TAAGGGGAGATAGAGACTTCAGCTGGTCCGGAGATGGACAACTCAGAGACCCAAAATTCAT |
| | | GAAGAAGACTTCGAAAGACCTCAGAGTGCGACCACAGACCCTGAAACATAGAAACCCCCCA |
| | | AACCAGAAGCATAAAAGGAAACTCAATCCTAAGAAAGATCTCCACACAAATACTGAAAGAC |
| | | CCCTC |
| 5 | RR86_v2 | GTAGGTCCTGAGAGGGGCGAGATTAGTTGGGTACTTACCATGTGGGCCTGGCACTTGCCCA |
| | (nucleotides | CATCACTTTGTGTCTTCCCACCGGCCCTGCGAGGGTGGGGGTTGCCGTCCTCAGTTTCCAG |
| | 185-467 of | GCGGGGGAAACGGGCTGGAAGCGTCACTCAGCTAATAGTAGTGGACCTGGGACTGGAACTC |
| | RR86_v1) | AGATTTGCTAAGGAACCAGGCAGGTTCTTTCCTGTTTTCTGCACGGGAGCATTTTAACCAG |
| | human | CAATTAAAGAAACCTTATTTCTAGGTAATTTTTAAAAGA |
| | regRNA | |
| 6 | RR93_v2 | AGAGCGAAGAGAGCAGGAGGAGGAGGAGGAGGGAGAGACTCTGCAGCCCCCACCCCTACTC |
| | human | CGGGAGGCCCAGATTGTGAGAGAGAGAGAGACCCCTGACTCAAAAACACACCAGAGAGAGA |
| | regRNA | CAGAGGTCCTAAAGATGCAGACCCCAGACCCTGAGACAGAGAGACCCTAGACCCACAGAGA |
| | | AAGATACTTCACCACCCCCCACTGTGGGGTAATTTGAGACACAGGGCCAAAATCTAGAGGA |
| | | AAAGAGACTCCTGATTTGAGGAAAAAACGACCCCAGATCCCCCAGAGAGAGAAGCTCAATA |
| | | TCAAAATCTGAAAATCCAGTCTAAAAAGGGTTCTCCATACCTACAGATATCTTAGACTCCA |
| | | GACCCTGAGATGATGATTTCAGGGACCAGGACCTGAGACCTAGACTCAGAAAAAGATGAGG |
| | | CCCAGATTCAGAAAGAGCCAGATGTAGAATTAGAGCTTAGAGAACTCAGACCTAGAAAGAG |
| | | ACCCCAGACTCAGATCTCAGAGACTGATAACTTAGAGACAGAGACACTCCTGACTTAAGGG |
| | | GAGATAGAGACTTCAGCTGGTCCGGAGATGGACAACTCAGAGACCCAAAATTCATGAAGAA |
| | | GACTTCGAAAGACCTCAGAGTGCGACCACAGACCCTGAAACATAGAAACCCCCCAAACCAG |
| | | AAGCATAAAAGGAAACTCAATCCTAAGAAAGATCTCCACACAAATACTGAAAGACCCCTCC |
| | | GAAATCTGTCTCAGAGACAAACTCCAAACTCAAAGACAGAGATCTCAGGGAACCTCCCCTC |
| | | CCCACTTCCCTGCCCTAGAACCTCCGAGAGGTATAACCCTGACGTCAGCCTGGGAAACTCC |
| | | GAGGCATCCCCACCACCAGACCAATGACCTCAGACCTTGAAGGGAGGGGAAA |
| 7 | RR121 | CACCGTACCTCTGAAGGGGGCATAGGACATCGCGGGGATGCTCCCCCGATGGATGGAGGCT |
| | mouse | CGAGACCTGCTCATCAGGCCTGAGGGGGAGGGGGGGGGGGGACGGGGGGAGAAAAAGAAGA |
| | regRNA | GAGAAAGAGGGGGAGAGAAAGGGGGAGAAGGAGGAGGTGGAGGAGGAGGAGGAGGAGGAGA |
| | | GAGGAGGAGAGAGGAGCGGAGAGGAGCAGAGAGGAGGAGAGAGGAGGAGGAGGAGGAGAAT |
| | | AAGAGCCAACGGCAGCAGCGGCAGCAGAGAGAGGGGGGGCGGGGGAAGCGAGCTAGAGAAG |
| | | AGAGAGCAGGAGGAGGAGGGAGAGACTGCAGCCCCCACCCCTACCCAGGGAGACCCGGAAA |
| | | AGGAGAGAACGAGGGACCCCTGACTCATACACACCAGGGAAGTCCCGTAGAAGCTGACCCA |
| | | AGACCCTCAGACAGACCCCAGACCCAGAGAAAGATACTCACCACCTCACCCCCCCCCCACC |
| | | TCCATACTCAACAAATTGGGTCTCACAGATCCAAATCTAGAAGGAAAGAGACCTCTGAGCT |
| | | CAGACCCCACCCCCTAAATTAAAGAAAGGAGTCAGGGACCAGAATATGAGACTCTAGTGTC |
| | | AGCAAGGTTCTCCAGACCCATGTTAATCTTATTCGAACCCTGGACTTGTAGTTTGGGAGAC |
| | | CCAAAACAGAGACCTAGACCCAAAGAAAAAGATGAAGCCAATATTAGAAAGTGCCCCAGGC |
| | | CAGAATTAAATTTTAGAATCTAGCTAGAGGTAAGAAACCCCACATTCGTATCTCAAAGAAA |
| | | CTGCAAACTTGGGGATGAAGGAGGAGACATTCGGCTGACCCAAAGATGAAGATGACCACAT |
| | | AGAGATCTAAAATACATTACATCTGAAAGATGTGAGTTAGTGACCGATTGCGAGGTTCAAT |
| | | TTCCTGAAACAACTCCCCAGGTAACCCAGACCCTAGGAAGGGCCCAAATATTGGACCCCCA |
| | | CCCCCACAGGACCCAAATATTGGACCCCCAACCCCTCACCTACACACACAA |

The present disclosure describes ASOs that may be used to increase expression of the target gene SYNGAP1 (e.g., human SYNGAP1 or murine SYNGAP1). Without wishing to be bound by theory, this increased gene expression may be due to increasing the amount or stability of the targeted SYNGAP1 regRNA, or interference with regRNA-associated repressors that inhibit the expression of the gene to thereby increase SYNGAP1 gene expression. These ASOs are different from the ASOs previously described that were designed to inhibit eRNAs (see, e.g., PCT Application Publication Nos. WO2013/177248 and WO2017/075406). Without wishing to be bound by theory, it is hypothesized that the ASOs' ability to upregulate SYNGAP1 gene expression is attributable to the selection of a target sequence in the regRNA and/or the chemical modifications of the ASOs.

Increased SYNGAP1 gene expression can be at least about 0.1%, 0.5%, 1%, 2%, 3%, 4%, 5%, 6%, 7%, 8%, 9%, 10%, 15%, 20%, 25%, 30%, 35%, 40%4, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 350%, 400%, 450%, 500%, 550%, 600%, 650%, 700%, 750%, 800%, 850%, 900%, 950%, or 1000% or more increase in expression as compared to baseline gene expression, gene expression prior to treatment, or gene expression after treatment with a control ASO. Increased SYNGAP1 gene expression can be at least about 0.1-fold, 0.5-fold, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold or more increase in expression as compared to baseline gene expression, gene expression prior to treatment, or gene expression after treatment with a control ASO.

ASOs that hybridize to (e.g., are complementary to) a portion of any of the regulatory RNAs provided herein (e.g., as described in Table 1 above), are contemplated by the present disclosure. In some embodiments, the regulatory RNA has a nucleotide sequence of any one of SEQ ID NO: 1, 2, 3, 4, 5, 6, and 7.

### Sequences of ASOs

In certain embodiments, an ASO disclosed herein is complementary to a sequence in a SYNGAP1 regRNA (e.g., a SYNGAP1 regRNA provided in Table 1) that is no more than 300, 250, 200, 150, 100, 50, 40, 30, 20, 10, 8, 5, or 1 nucleotide(s) from the 5′ or 3′ end of the SYNGAP1 regRNA. In certain embodiments, the ASO disclosed herein is complementary to a sequence in the SYNGAP1 regRNA that is no more than 300, 250, 200, 150, 100, 50, 40, 30, 20, or 10 nucleotides from the 5′ end of the SYNGAP1 regRNA (i.e., the 5′ most nucleotide of the regRNA sequence forming a duplex with the ASO is no more than 300, 250, 200, 150, 100, 50, 40, 30, 20, 10, 8, 5, or 1 nucleotide(s) from the 5′ end of the SYNGAP1 regRNA). In certain embodiments, the ASO disclosed herein is complementary to a sequence in the SYNGAP1 regRNA that is no more than 300, 250, 200, 150, 100, 50, 40, 30, 20, 10, 8, 5, or 1 nucleotide(s) nucleotides from the 3′ end of the SYNGAP1 regRNA (i.e., the 3′ most nucleotide of the regRNA sequence forming a duplex with the ASO is no more than 300, 250, 200, 150, 100, 50, 40, 30, 20, or 10 nucleotides from the 3′ end of the SYNGAP1 regRNA). In some embodiments, provided herein are ASOs comprising a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of a portion of a SYNGAP1 regRNA provided herein (e.g., a regRNA comprising or consisting a portion of or the full length nucleotide sequence provided in any one of SEQ ID NOs: 1, 2, 3, 4, 5, 6, and 7). In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of a SYNGAP1 regRNA identified herein as RR86_v1 (SEQ ID NO: 1). In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of a 3′ portion of SEQ ID NO: 1 (e.g., nucleotides 185-467, 186-467, 187-467, 188-467, 189-467, 190-467, 191-467, 192-467, 193-467, 194-467, 195-467, 196-467, 197-467, 198-467, 199-467, 200-467, 201-467, 202-467, 203-467, 204-467, 205-467, 210-467, 215-467, or 220-467 of SEQ ID NO: 1). In some embodiments, provided herein are ASOs that do not comprise or consist of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of a 5′ portion of SEQ ID NO: 1 (e.g., nucleotides 1-184, 1-183, 1-182, 1-181, 1-180, 1-179, 1-178, 1-177, 1-176, 1-175, 1-174, 1-173, 1-172, 1-171, 1-170, 1-169, 1-168, 1-167, 1-166, 1-165, 1-164, 1-163, 1-162, or 1-161 of SEQ ID NO: 1).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR87 (SEQ ID NO: 2).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR88 (SEQ ID NO: 3).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR93_v1 (SEQ ID NO: 4).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR86_v2 (SEQ ID NO: 5).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR93_v2 (SEQ ID NO: 6).

In some embodiments, provided herein are ASOs comprising or consisting of a nucleotide sequence that is complementary to at least 8 nucleotides (e.g., 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides) of the SYNGAP1 regRNA identified herein as RR121 (SEQ ID NO: 7).

In certain embodiments, the ASO is no more than 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, or 100 nucleotides in length. In certain embodiments, the ASO is at least 8, 10, 15, 20, 25, 30, 40, 50, 60, 70, 80, 90, or 100 nucleotides in length. In certain embodiments, the ASO is at least 8, 9, 10, 11, 12, 13, 14, 15, 16, 17, 18, 19, or 20 nucleotides in length. In certain embodiments, the ASO is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In certain embodiments, the ASO is designed to lack a stable secondary structure formed within itself or between each other, thereby increasing the amount of the ASO in a single-stranded form ready to hybridize with the SYNGAP1 regRNA. Methods to predict secondary structures are known in the art (see, e.g., Seetin and Mathews, Methods Mol. Biol. (2012) 905:99-122; Zhao et al., PLoS Comput. Biol. (2021) 17(8):e1009291) and web-based programs (e.g., RNAfold) are available to public users.

For example, ASOs have been designed to target a human SYNGAP1 regRNA (e.g., an eRNA, a NAT or a paRNA). The nucleotide sequences of some of these ASOs are provided in Table 2 below. In some embodiments, an ASO of the disclosure comprises or consists of a nucleotide sequence provided in any one of Tables 2-4. In some embodiments, an ASO of the disclosure comprises or consists of a nucleotide sequence and/or a chemistry modification as provided in Table 2. Any chemical modification or combination of chemical modifications described herein can be applied to any ASO sequence provided herein (e.g., in Table 2, 3, or 4). In some embodiments, an ASO comprises or consists of a nucleotide sequence and/or a chemistry modification of any one of SEQ ID NOs: 10-4852. In some embodiments, an ASO comprises or consists of a nucleotide sequence and/or a chemistry modification of any one of SEQ ID NOs: 10-1003, 1004-2961, or 2962-4852.

Additional ASO sequences that target SYNGAP1 regRNAs RR86_v1, RR86_v2, RR93_v1, and RR93_v2 are provided in Tables 3 and 4. In some embodiments, an ASO of the disclosure comprises or consists of a nucleotide sequence selected from any one of the ASOs provided in Tables 2-4. In some embodiments, the ASO comprises or consists of a nucleotide sequence as set for in any one of SEQ ID NOs: 1004-2961 or 2962-4852.

TABLE 2
Exemplary SYNGAP1 regRNA-targeting ASO sequences and descriptions of chemical modifications

| | | | | Sequence |
|---|---|---|---|---|
| | | | | MOE (M); DNA (d); |
| SEQ ID NO | Name | Sequence | SEQ ID NO | LNA (l); 2′ OMethyl (m); PS (=); PO(-); 5-MethylCytosine (5C); Teg-GalNAc [TEG] |
| 10 | CO-7451 | ACTTCAGCAGCTCCCCGGGA | 542 | MA=M5C=MT=MT=M5C=MA=MG=M5C=MA=MG=M5C=MT=M5C=M5C=M5C=M5C=MG=MG=MG=MA |
| 11 | CO-7452 | TGCCTCTCGGTCCTCTGCAG | 543 | MT=MG=M5C=M5C=MT=M5C=MT=M5C=MG=MG=MT=M5C=M5C=MT=M5C=MT=MG=M5C=MA=MG |
| 12 | CO-7426 | ACTTCAGCAGCTCCCCGGGA | 544 | MA=M5C=MT=MT=M5C=dA=dG=d5C=dA=dG=d5C=dT=d5C=d5C=d5C=M5C=MG=MG=MG=MA |
| 13 | CO-7427 | TGCCTCTCGGTCCTCTGCAG | 545 | MT=MG=M5C=M5C=MT=d5C=dT=d5C=dG=dG=dT=d5C=d5C=dT=d5C=MT=MG=M5C=MA=MG |
| 14 | CO-7428 | AATCTCGCCCCTCTCAGGAC | 546 | MA=MA=MT=M5C=MT=d5C=dG=d5C=d5C=d5C=d5C=dT=d5C=dT=d5C=MA=MG=MG=MA=M5C |
| 15 | CO-7429 | CCAACTAATCTCGCCCCTCT | 547 | M5C=M5C=MA=MA=M5C=dT=dA=dA=dT=d5C=dT=d5C=dG=d5C=d5C=M5C=M5C=MT=M5C=MT |
| 16 | CO-7430 | TAAGTACCCAACTAATCTCG | 548 | MT=MA=MA=MG=MT=dA=d5C=d5c=d5C=dA=dA=d5C=dT=dA=dA=MT=M5C=MT=M5C=MG |
| 17 | CO-7431 | CAGGCCCACATGGTAAGTAC | 549 | M5C=MA=MG=MG=M5C=d5C=d5C=dA=d5C=dA=dT=dG=dG=dT=dA=MA=MG=MT=MA=M5C |
| 18 | CO-7432 | GCAAGTGCCAGGCCCACATG | 550 | MG=M5C=MA=MA=MG=dT=dG=d5C=d5C=dA=dG=dG=d5C=d5C=d5C=MA=M5C=MA=MT=MG |
| 19 | CO-7433 | TGGGAAGACACAAAGTGATG | 551 | MT=MG=MG=MG=MA=dA=dG=dA=d5C=dA=d5C=dA=dA=dA=dG=MT=MG=MA=MT=MG |
| 20 | CO-7434 | ACTGAGGACGGCAACCCCCA | 552 | MA=M5C=MT=MG=MA=dG=dG=dA=d5C=dG=dG=d5C=dA=dA=d5C=M5C=M5C=M5C=M5C=MA |
| 21 | CO-7435 | CTGGAAACTGAGGACGGCAA | 553 | M5C=MT=MG=MG=MA=dA=dA=d5C=dT=dG=dA=dG=dG=dA=d5C=MG=MG=M5C=MA=MA |
| 22 | CO-7436= | TTTCCCCCGCCTGGAAACTG | 554 | MT=MT=MT=M5C=M5C=d5C=d5C=d5C=dG=d5C=d5C=dT=dG=dG=dA=MA=MA=M5C=MT=MG |
| 23 | CO-7437 | GTGACGCTTCCAGCCCGTTT | 555 | MG=MT=MG=MA=M5C=dG=d5C=dT=dT=d5C=d5C=dA=dG=d5C=d5C=M5C=MG=MT=MT=MT |
| 24 | CO-7438 | TAGCTGAGTGACGCTTCCAG | 556 | MT=MA=MG=M5C=MT=dG=dA=dG=dT=dG=dA=d5C=dG=d5C=dT=MT=M5C=M5C=MA=MG |
| 25 | CO-7439 | TACTATTAGCTGAGTGACGC | 557 | MT=MA=M5C=MT=MA=dT=dT=dA=dG=d5C=dT=dG=dA=dG=dT=MG=MA=M5C=MG=M5C |
| 26 | CO-7440 | GGTCCACTACTATTAGCTGA | 558 | MG=MG=MT=M5C=M5C=dA=d5C=dT=dA=d5C=dT=dA=dT=dT=dA=MG=M5C=MT=MG=MA |
| 27 | CO-7441 | GTCCCAGGTCCACTACTATT | 559 | MG=MT=M5C=M5C=M5C=dA=dG=dG=dT=d5C=d5C=dA=d5C=dT=dA=M5C=MT=MA=MT=MT |
| 28 | CO-7442 | GTTCCAGTCCCAGGTCCACT | 560 | MG=MT=MT=M5C=M5C=dA=dG=dT=d5C=d5C=d5C=dA=dG=dG=dT=M5C=M5C=MA=M5C=MT |
| 29 | CO-7443 | AATCTGAGTTCCAGTCCCAG | 561 | MA=MA=MT=M5C=MT=dG=dA=dG=dT=dT=d5C=d5C=dA=dG=dT=M5C=M5C=M5C=MA=MG |
| 30 | CO-7444 | CCTTAGCAAATCTGAGTTCC | 562 | M5C=M5C=MT=MT=MA=dG=d5C=dA=dA=dA=dT=d5C=dT=dG=dA=MG=MT=MT=M5C=M5C |
| 31 | CO-7445 | GCCTGGTTCCTTAGCAAATC | 563 | MG=M5C=M5C=MT=MG=dG=dT=dT=d5C=d5C=dT=dT=dA=dG=d5C=MA=MA=MA=MT=M5C |
| 32 | CO-7446 | GTGCAGAAAACAGGAAAGAA | 564 | MG=MT=MG=M5C=MA=dG=dA=dA=dA=dA=d5C=dA=dG=dG=dA=MA=MA=MG=MA=MA |
| 33 | CO-7447 | GCTCCCGTGCAGAAAACAGG | 565 | MG=M5C=MT=M5C=M5C=d5C=dG=dT=dG=d5C=dA=dG=dA=dA=dA=MA=M5C=MA=MG=MG |
| 34 | CO-7448 | TGGTTAAAATGCTCCCGTGC | 566 | MT=MG=MG=MT=MT=dA=dA=dA=dA=dT=dG=d5C=dT=d5C=d5C=M5C=MG=MT=MG=M5C |
| 35 | CO-7449 | AATTGCTGGTTAAAATGCTC | 567 | MA=MA=MT=MT=MG=d5C=dT=dG=dG=dT=dT=dA=dA=dA=dA=MT=MG=M5C=MT=M5C |
| 36 | CO-7450 | AAGGTTTCTTTAATTGCTGG | 568 | MA=MA=MG=MG=MT=dT=dT=d5C=dT=dT=dT=dA=dA=dT=dT=MG=M5C=MT=MG=MG |
| 37 | CO-7453 | AATCTCGCCCCTCTCAGGAC | 569 | MA=MA=MT=M5C=MT=M5C=MG=M5C=M5C=M5C=M5C=MT=M5C=MT=M5C=MA=MG=MG=MA=M5C |
| 38 | CO-7454 | CCAACTAATCTCGCCCCTCT | 570 | M5C=M5C=MA=MA=M5C=MT=MA=MA=MT=M5C=MT=M5C=MG=M5C=M5C=M5C=M5C=MT=M5C=MT |
| 39 | CO-7455 | TAAGTACCCAACTAATCTCG | 571 | MT=MA=MA=MG=MT=MA=M5C=M5C=M5C=MA=MA=M5C=MT=MA=MA=MT=M5C=MT=M5C=MG |
| 40 | CO-7456 | CAGGCCCACATGGTAAGTAC | 572 | M5C=MA=MG=MG=M5C=M5C=M5C=MA=M5C=MA=MT=MG=MG=MT=MA=MA=MG=MT=MA=M5C |
| 41 | CO-7457 | GCAAGTGCCAGGCCCACATG | 573 | MG=M5C=MA=MA=MG=MT=MG=M5C=M5C=MA=MG=MG=M5C=M5C=M5C=MA=M5C=MA=MT=MG |
| 42 | CO-7458 | TGGGAAGACACAAAGTGATG | 574 | MT=MG=MG=MG=MA=MA=MG=MA=M5C=MA=M5C=MA=MA=MA=MG=MT=MG=MA=MT=MG |
| 43 | CO-7459 | ACTGAGGACGGCAACCCCCA | 575 | MA=M5C=MT=MG=MA=MG=MG=MA=M5C=MG=MG=M5C=MA=MA=M5C=M5C=M5C=M5C=M5C=MA |
| 44 | CO-7460 | CTGGAAACTGAGGACGGCAA | 576 | M5C=MT=MG=MG=MA=MA=MA=M5C=MT=MG=MA=MG=MG=MA=M5C=MG=MG=M5C=MA=MA |
| 45 | CO-7461 | TTTCCCCCGCCTGGAAACTG | 577 | MT=MT=MT=M5C=M5C=M5C=M5C=M5C=MG=M5C=M5C=MT=MG=MG=MA=MA=MA=M5C=MT=MG |
| 46 | CO-7462 | GTGACGCTTCCAGCCCGTTT | 578 | MG=MT=MG=MA=M5C=MG=M5C=MT=MT=M5C=M5C=MA=MG=M5C=M5C=M5C=MG=MT=MT=MT |
| 47 | CO-7463 | TAGCTGAGTGACGCTTCCAG | 579 | MT=MA=MG=M5C=MT=MG=MA=MG=MT=MG=MA=M5C=MG=M5C=MT=MT=M5C=M5C=MA=MG |
| 48 | CO-7464 | TACTATTAGCTGAGTGACGC | 580 | MT=MA=M5C=MT=MA=MT=MT=MA=MG=M5C=MT=MG=MA=MG=MT=MG=MA=M5C=MG=M5C |
| 49 | CO-7465 | GGTCCACTACTATTAGCTGA | 581 | MG=MG=MT=M5C=M5C=MA=M5C=MT=MA=M5C=MT=MA=MT=MT=MA=MG=M5C=MT=MG=MA |
| 50 | CO-7466 | GTCCCAGGTCCACTACTATT | 582 | MG=MT=M5C=M5C=M5C=MA=MG=MG=MT=M5C=M5C=MA=M5C=MT=MA=M5C=MT=MA=MT=MT |
| 51 | CO-7467 | GTTCCAGTCCCAGGTCCACT | 583 | MG=MT=MT=M5C=M5C=MA=MG=MT=M5C=M5C=M5C=MA=MG=MG=MT=M5C=M5C=MA=M5C=MT |
| 52 | CO-7468 | AATCTGAGTTCCAGTCCCAG | 584 | MA=MA=MT=M5C=MT=MG=MA=MG=MT=MT=M5C=M5C=MA=MG=MT=M5C=M5C=M5C=MA=MG |
| 53 | CO-7469 | CCTTAGCAAATCTGAGTTCC | 585 | M5C=M5C=MT=MT=MA=MG=M5C=MA=MA=MA=MT=M5C=MT=MG=MA=MG=MT=MT=M5C=M5C |
| 54 | CO-7470 | GCCTGGTTCCTTAGCAAATC | 586 | MG=M5C=M5C=MT=MG=MG=MT=MT=M5C=M5C=MT=MT=MA=MG=M5C=MA=MA=MA=MT=M5C |
| 55 | CO-7471 | GTGCAGAAAACAGGAAAGAA | 587 | MG=MT=MG=M5C=MA=MG=MA=MA=MA=MA=M5C=MA=MG=MG=MA=MA=MA=MG=MA=MA |
| 56 | CO-7472 | GCTCCCGTGCAGAAAACAGG | 588 | MG=M5C=MT=M5C=M5C=M5C=MG=MT=MG=M5C=MA=MG=MA=MA=MA=MA=M5C=MA=MG=MG |
| 57 | CO-7473 | TGGTTAAAATGCTCCCGTGC | 589 | MT=MG=MG=MT=MT=MA=MA=MA=MA=MT=MG=M5C=MT=M5C=M5C=M5C=MG=MT=MG=M5C |
| 58 | CO-7474 | AATTGCTGGTTAAAATGCTC | 590 | MA=MA=MT=MT=MG=M5C=MT=MG=MG=MT=MT=MA=MA=MA=MA=MT=MG=M5C=MT=M5C |
| 59 | CO-7475 | AAGGTTTCTTTAATTGCTGG | 591 | MA=MA=MG=MG=MT=MT=MT=M5C=MT=MT=MT=MA=MA=MT=MT=MG=M5C=MT=MG=MG |
| 60 | CO-7476 | GCAGCAGGCAGAGAAGGATT | | |
| 61 | CO-7477 | GTCCCAGGAAGAACAAACCA | | |
| 62 | CO-7478 | GCTGCTGTCCCAGGAAGAAC | | |
| 63 | CO-7479 | CTGGAAGAGTATGAGCGGAG | | |
| 64 | CO-7480 | CGGAAGCTGGAAGAGTATGA | | |
| 65 | CO-7481 | TCCAACCGGAAGCTGGAAGA | | |
| 66 | CO-7482 | GGCTGCACATGTCCAACCGG | | |
| 67 | CO-7483 | GCAGCAGGCAGAGAAGGATT | | |
| 68 | CO-7484 | GTCCCAGGAAGAACAAACCA | | |
| 69 | CO-7485 | GCTGCTGTCCCAGGAAGAAC | | |
| 70 | CO-7486 | CTGGAAGAGTATGAGCGGAG | | |
| 71 | CO-7487 | CGGAAGCTGGAAGAGTATGA | | |
| 72 | CO-7488 | TCCAACCGGAAGCTGGAAGA | | |
| 73 | CO-7489 | GGCTGCACATGTCCAACCGG | | |
| 74 | CO-7490 | GGAATGAGATACCATGGCTG | | |
| 75 | CO-7491 | TCAGTATGGCCTCTTTTTTT | | |
| 76 | CO-7492 | TCTAATTCAGTATGGCCTCT | | |
| 77 | CO-7493 | GTTAAGGGTCCACTCTAATT | | |
| 78 | CO-7494 | TATAAGGTGGGTTAAGGGTC | | |
| 79 | CO-7495 | GACACCAGTTTTTATAAGGT | | |
| 80 | CO-7496 | CCATATAAAGACACCAGTTT | | |
| 81 | CO-7497 | CTCCCTCTCCATATAAAGAC | | |
| 82 | CO-7498 | TATCTCTGTGTCTCCAAATC | | |
| 83 | CO-7499 | GTATCCTGTATCTCTGTGTC | | |
| 84 | CO-7500 | GTGTGTCTCTGTATCCTGTA | | |
| 85 | CO-7501 | GTAGCTGGCTTCCTTCTGTG | | |
| 86 | CO-7502 | CTTCATGTAGCTGGCTTCCT | | |
| 87 | CO-7503 | CTCCATCTTCATGTAGCTGG | | |
| 88 | CO-7504 | GCATCCGTCTAATTTCTGCC | | |
| 89 | CO-7505 | CTTATAACTGCATCCGTCTA | | |
| 90 | CO-7506 | GCTGTAGCAGGGAATCCTTC | | |
| 91 | CO-7507 | GCCACGCTCTCTTTGATGCT | | |
| 92 | CO-7508 | GGAATGAGATACCATGGCTG | | |
| 93 | CO-7509 | TCAGTATGGCCTCTTTTTTT | | |
| 94 | CO-7510 | TCTAATTCAGTATGGCCTCT | | |
| 95 | CO-7511 | GTTAAGGGTCCACTCTAATT | | |
| 96 | CO-7512 | TATAAGGTGGGTTAAGGGTC | | |
| 97 | CO-7513 | GACACCAGTTTTTATAAGGT | | |
| 98 | CO-7514 | CCATATAAAGACACCAGTTT | | |
| 99 | CO-7515 | CTCCCTCTCCATATAAAGAC | | |
| 100 | CO-7516 | TATCTCTGTGTCTCCAAATC | | |
| 101 | CO-7517 | GTATCCTGTATCTCTGTGTC | | |
| 102 | CO-7518 | GTGTGTCTCTGTATCCTGTA | | |
| 103 | CO-7519 | GTAGCTGGCTTCCTTCTGTG | | |
| 104 | CO-7520 | CTTCATGTAGCTGGCTTCCT | | |
| 105 | CO-7521 | CTCCATCTTCATGTAGCTGG | | |
| 106 | CO-7522 | GCATCCGTCTAATTTCTGCC | | |
| 107 | CO-7523 | CTTATAACTGCATCCGTCTA | | |
| 108 | CO-7524 | GCTGTAGCAGGGAATCCTTC | | |
| 109 | CO-7525 | GCCACGCTCTCTTTGATGCT | | |
| 110 | CO-9338 | CGTTGGCTCTTATTCTCCTC | 592 | M5C=MG=MT=MT=MG=dG=d5C=dT=d5C=dT=dT=dA=dT=dT=d5C=MT=M5C=M5C=MT=M5C |
| 111 | CO-9339 | CTCTCTGGTGTGTTTTTGAG | 593 | M5C=MT=M5C=MT=M5C=dT=dG=dG=dT=dG=dT=dG=dT=dT=dT=MT=MT=MG=MA=MG |
| 112 | CO-9340 | TGCATCTTTAGGACCTCTGT | 594 | MT=MG=M5C=MA=MT=d5C=dT=dT=dT=dA=dG=dG=dA=d5C=d5C=MT=M5C=MT=MG=MT |
| 113 | CO-9341 | GGTGGTGAAGTATCTTTCTC | 595 | MG=MG=MT=MG=MG=dT=dG=dA=dA=dG=dT=dA=dT=d5C=dT=MT=MT=M5C=MT=M5C |
| 114 | CO-9342 | GTCTCAAATTACCCCACAGT | 596 | MG=MT=M5C=MT=M5C=dA=dA=dA=dT=dT=dA=d5C=d5C=d5C=d5C=MA=M5C=MA=MG=MT |
| 115 | CO-9343 | AGATTTTGGCCCTGTGTCTC | 597 | MA=MG=MA=MT=MT=dT=dT=dG=dG=d5C=d5C=d5C=dT=dG=dT=MG=MT=M5C=MT=M5C |
| 116 | CO-9344 | GAGTCTCTTTTCCTCTAGAT | 598 | MG=MA=MG=MT=M5C=dT=d5C=dT=dT=dT=dT=d5C=d5C=dT=d5C=MT=MA=MG=MA=MT |
| 117 | CO-9345 | GGTCGTTTTTTCCTCAAATC | 599 | MG=MG=MT=M5C=MG=dT=dT=dT=dT=dT=dT=d5C=d5C=dT=d5C=MA=MA=MA=MT=M5C |
| 118 | CO-9346 | TGATATTGAGCTTCTCTCTC | 600 | MT=MG=MA=MT=MA=dT=dT=dG=dA=dG=d5C=dT=dT=d5C=dT=M5C=MT=M5C=MT=M5C |
| 119 | CO-9347 | GTAGGTATGGAGAACCCTTT | 601 | MG=MT=MA=MG=MG=dT=dA=dT=dG=dG=dA=dG=dA=dA=d5C=M5C=M5C=MT=MT=MT |
| 120 | CO-9348 | GGAGTCTAAGATATCTGTAG | 602 | MG=MG=MA=MG=MT=d5C=dT=dA=dA=dG=dA=dT=dA=dT=d5C=MT=MG=MT=MA=MG |
| 121 | CO-9349 | GAGTCTAGGTCTCAGGTCCT | 603 | MG=MA=MG=MT=M5C=dT=dA=dG=dG=dT=d5C=dT=d5C=dA=dG=MG=MT=M5C=M5C=MT |
| 122 | CO-9350 | GCTCTAATTCTACATCTGGC | 604 | MG=M5C=MT=M5C=MT=dA=dA=dT=dT=d5C=dT=dA=d5C=dA=dT=M5C=MT=MG=MG=M5C |
| 123 | CO-9351 | CTAGGTCTGAGTTCTCTAAG | 605 | M5C=MT=MA=MG=MG=dT=d5C=dT=dG=dA=dG=dT=dT=d5C=dT=M5C=MT=MA=MA=MG |
| 124 | CO-9352 | GTCTCTAAGTTATCAGTCTC | 606 | MG=MT=M5C=MT=M5C=dT=dA=dA=dG=dT=dT=dA=dT=d5C=dA=MG=MT=M5C=MT=M5C |
| 125 | CO-9353 | TAAGTCAGGAGTGTCTCTGT | 607 | MT=MA=MA=MG=MT=d5C=dA=dG=dG=dA=dG=dT=dG=dT=d5C=MT=M5C=MT=MG=MT |
| 126 | CO-9354 | GTCTCTATCTCCCCTTAAGT | 608 | MG=MT=M5C=MT=M5C=dT=dA=dT=d5C=dT=d5C=d5c=d5C=d5C=dT=MT=MA=MA=MG=MT |
| 127 | CO-9355 | ATCTCCGGACCAGCTGAAGT | 609 | MA=MT=M5C=MT=M5C=d5C=dG=dG=dA=d5C=d5C=dA=dG=d5C=dT=MG=MA=MA=MG=MT |
| 128 | CO-9356 | GTCTCTGAGTTGTCCATCTC | 610 | MG=MT=M5C=MT=M5C=dT=dG=dA=dG=dT=dT=dG=dT=d5C=d5C=MA=MT=M5C=MT=M5C |
| 129 | CO-9357 | TCTTCATGAATTTTGGGTCT | 611 | MT=M5C=MT=MT=M5C=dA=dT=dG=dA=dA=dT=dT=dT=dT=dG=MG=MG=MT=M5C=MT |
| 130 | CO-9358 | CGCACTCTGAGGTCTTTCGA | 612 | M5C=MG=M5C=MA=M5C=dT=d5C=dT=dG=dA=dG=dG=dT=d5C=dT=MT=MT=M5C=MG=MA |
| 131 | CO-9359 | GGGTTTCTATGTTTCAGGGT | 613 | MG=MG=MG=MT=MT=dT=d5C=dT=dA=dT=dG=dT=dT=dT=d5C=MA=MG=MG=MG=MT |
| 132 | CO-9360 | CTTTTATGCTTCTGGTTTGG | 614 | M5C=MT=MT=MT=MT=dA=dT=dG=d5C=dT=dT=d5C=dT=dG=dG=MT=MT=MT=MG=MG |
| 133 | CO-9361 | GTGTGGAGATCTTTCTTAGG | 615 | MG=MT=MG=MT=MG=dG=dA=dG=dA=dT=d5C=dT=dT=dT=d5C=MT=MT=MA=MG=MG |
| 134 | CO-9362 | GGTCTTTCAGTATTTGTGTG | 616 | MG=MG=MT=M5C=MT=dT=dT=d5C=dA=dG=dT=dA=dT=dT=dT=MG=MT=MG=MT=MG |
| 135 | CO-9363 | GTTGGCTCTTATTCTCCTCC | 617 | MG=MT=MT=MG=MG=d5C=dT=d5C=dT=dT=dA=dT=dT=d5C=dT=M5C=M5C=MT=M5C=M5C |
| 136 | CO-9364 | GCTCTCTTCGCTCTCGCTCG | 618 | MG=M5C=MT=M5C=MT=d5C=dT=dT=d5C=dG=d5C=dT=d5C=dT=d5C=MG=M5C=MT=M5C=MG |
| 137 | CO-9365 | CTGCAGAGTCTCTCCCTCCT | 619 | M5C=MT=MG=M5C=MA=dG=dA=dG=dT=d5C=dT=d5C=dT=d5C=d5C=M5C=MT=M5C=M5C=MT |
| 138 | CO-9366 | CTGTCTCTCTCTGGTGTGTT | 620 | M5C=MT=MG=MT=M5C=dT=d5C=dT=d5C=dT=d5C=dT=dG=dG=dT=MG=MT=MG=MT=MT |
| 139 | CO-9367 | GGACCTCTGTCTCTCTCTGG | 621 | MG=MG=MA=M5C=M5C=dT=d5C=dT=dG=dT=d5C=dT=d5C=dT=d5C=MT=M5C=MT=MG=MG |
| 140 | CO-9368 | GTCTAGGGTCTCTCTGTCTC | 622 | MG=MT=M5C=MT=MA=dG=dG=dG=dT=d5C=dT=d5C=dT=d5C=dT=MG=MT=M5C=MT=M5C |
| 141 | CO-9369 | TGTGGGTCTAGGGTCTCTCT | 623 | MT=MG=MT=MG=MG=dG=dT=d5C=dT=dA=dG=dG=dG=dT=d5C=MT=M5C=MT=M5C=MT |
| 142 | CO-9370 | CTTTCTCTGTGGGTCTAGGG | 624 | M5C=MT=MT=MT=M5C=dT=d5C=dT=dG=dT=dG=dG=dG=dT=d5C=MT=MA=MG=MG=MG |
| 143 | CO-9371 | GAAGTATCTTTCTCTGTGGG | 625 | MG=MA=MA=MG=MT=dA=dT=d5C=dT=dT=dT=d5C=dT=d5C=dT=MG=MT=MG=MG=MG |
| 144 | CO-9372 | GGGTGGTGAAGTATCTTTCT | 626 | MG=MG=MG=MT=MG=dG=dT=dG=dA=dA=dG=dT=dA=dT=d5C=MT=MT=MT=M5C=MT |
| 145 | CO-9373 | GGCCCTGTGTCTCAAATTAC | 627 | MG=MG=M5C=M5C=M5C=dT=dG=dT=dG=dT=d5C=dT=d5C=dA=dA=MA=MT=MT=MA=M5C |
| 146 | CO-9374 | GATTTTGGCCCTGTGTCTCA | 628 | MG=MA=MT=MT=MT=dT=dG=dG=d5C=d5C=d5C=dT=dG=dT=dG=MT=M5C=MT=M5C=MA |
| 147 | CO-9375 | CCTCTAGATTTTGGCCCTGT | 629 | M5C=M5C=MT=M5C=MT=dA=dG=dA=dT=dT=dT=dT=dG=dG=d5C=M5C=M5C=MT=MG=MT |
| 148 | CO-9376 | CTGTAGGTATGGAGAACCCT | 630 | M5C=MT=MG=MT=MA=dG=dG=dT=dA=dT=dG=dG=dA=dG=dA=MA=M5C=M5C=M5C=MT |
| 149 | CO-9377 | TCTGGCTCTTTCTGAATCTG | 631 | MT=M5C=MT=MG=MG=d5C=dT=d5C=dT=dT=dT=d5C=dT=dG=dA=MA=MT=M5C=MT=MG |
| 150 | CO-9378 | CTACATCTGGCTCTTTCTGA | 632 | M5C=MT=MA=M5C=MA=dT=d5C=dT=dG=dG=d5C=dT=d5C=dT=dT=MT=M5C=MT=MG=MA |
| 151 | CO-9379 | GGGTCTCTTTCTAGGTCTGA | 633 | MG=MG=MG=MT=M5C=dT=d5C=dT=dT=dT=d5C=dT=dA=dG=dG=MT=M5C=MT=MG=MA |
| 152 | CO-9380 | GTCAGGAGTGTCTCTGTCTC | 634 | MG=MT=M5C=MA=MG=dG=dA=dG=dT=dG=dT=d5C=dT=d5C=dT=MG=MT=M5C=MT=M5C |
| 153 | CO-9381 | GCTGAAGTCTCTATCTCCCC | 635 | MG=M5C=MT=MG=MA=dA=dG=dT=d5C=dT=d5C=dT=dA=dT=d5C=MT=M5C=M5C=M5C=M5C |
| 154 | CO-9382 | TCTCCGGACCAGCTGAAGTC | 636 | MT=M5C=MT=M5C=M5C=dG=dG=dA=d5C=d5C=dA=dG=d5C=dT=dG=MA=MA=MG=MT=M5C |
| 155 | CO-9383 | GGGTCTCTGAGTIGTCCATC | 637 | MG=MG=MG=MT=M5C=dT=d5C=dT=dG=dA=dG=dT=dT=dG=dT=M5C=M5C=MA=MT=M5C |
| 156 | CO-9384 | GGTCGCACTCTGAGGTCTTT | 638 | MG=MG=MT=M5C=MG=d5C=dA=d5C=dT=d5C=dT=dG=dA=dG=dG=MT=M5C=MT=MT=MT |
| 157 | CO-9385 | GGGTCTGTGGTCGCACTCTG | 639 | MG=MG=MG=MT=M5C=dT=dG=dT=dG=dG=dT=d5C=dG=d5C=dA=M5C=MT=M5C=MT=MG |
| 158 | CO-9386 | GTTTCAGGGTCTGTGGTCGC | 640 | MG=MT=MT=MT=M5C=dA=dG=dG=dG=dT=d5C=dT=dG=dT=dG=MG=MT=M5C=MG=M5C |
| 159 | CO-9387 | TCTATGTTTCAGGGTCTGTG | 641 | MT=M5C=MT=MA=MT=dG=dT=dT=dT=d5C=dA=dG=dG=dG=dT=M5C=MT=MG=MT=MG |
| 160 | CO-9388 | TTTTCAGATTTTGATATTGA | 642 | MT=MT=MT=MT=M5C=dA=dG=dA=dT=dT=dT=dT=dG=dA=dT=MA=MT=MT=MG=MA |
| 161 | CO-9389 | CCTGAAATCATCATCTCAGG | 643 | M5C=M5C=MT=MG=MA=dA=dA=dT=d5C=dA=dT=d5C=dA=dT=d5C=MT=M5C=MA=MG=MG |
| 162 | CO-9390 | AGGATTGAGTTTCCTTTTAT | 644 | MA=MG=MG=MA=MT=dT=dG=dA=dG=dT=dT=dT=d5C=d5C=dT=MT=MT=MT=MA=MT |
| 163 | CO-9391 | GAGATCTTTCTTAGGATTGA | 645 | MG=MA=MG=MA=MT=d5C=dT=dT=dT=d5C=dT=dT=dA=dG=dG=MA=MT=MT=MG=MA |
| 164 | CO-9392 | CTCTGAGACAGATTTCGGAG | 646 | M5C=MT=M5C=MT=MG=dA=dG=dA=d5C=dA=dG=dA=dT=dT=dT=M5C=MG=MG=MA=MG |
| 165 | CO-9393 | CGTTGGCTCTTATTCTCCTC | 647 | M5C=MG=MT=MT=MG=MG=M5C=MT=M5C=MT=MT=MA=MT=MT=M5C=MT=M5C=M5C=MT=M5C |
| 166 | CO-9394 | CTCTCTGGTGTGTTTTTGAG | 648 | M5C=MT=M5C=MT=M5C=MT=MG=MG=MT=MG=MT=MG=MT=MT=MT=MT=MT=MG=MA=MG |
| 167 | CO-9395 | TGCATCTTTAGGACCTCTGT | 649 | MT=MG=M5C=MA=MT=M5C=MT=MT=MT=MA=MG=MG=MA=M5C=M5C=MT=M5C=MT=MG=MT |
| 168 | CO-9396 | GGTGGTGAAGTATCTTTCTC | 650 | MG=MG=MT=MG=MG=MT=MG=MA=MA=MG=MT=MA=MT=M5C=MT=MT=MT=M5C=MT=M5C |
| 169 | CO-9397 | GTCTCAAATTACCCCACAGT | 651 | MG=MT=M5C=MT=M5C=MA=MA=MA=MT=MT=MA=M5C=M5C=M5C=M5C=MA=M5C=MA=MG=MT |
| 170 | CO-9398 | AGATTTTGGCCCTGTGTCTC | 652 | MA=MG=MA=MT=MT=MT=MT=MG=MG=M5C=M5C=M5C=MT=MG=MT=MG=MT=M5C=MT=M5C |
| 171 | CO-9399 | GAGTCTCTTTTCCTCTAGAT | 653 | MG=MA=MG=MT=M5C=MT=M5C=MT=MT=MT=MT=M5C=M5C=MT=M5C=MT=MA=MG=MA=MT |
| 172 | CO-9400 | GGTCGTTTTTTCCTCAAATC | 654 | MG=MG=MT=M5C=MG=MT=MT=MT=MT=MT=MT=M5C=M5C=MT=M5C=MA=MA=MA=MT=M5C |
| 173 | CO-9401 | TGATATTGAGCTTCTCTCTC | 655 | MT=MG=MA=MT=MA=MT=MT=MG=MA=MG=M5C=MT=MT=M5C=MT=M5C=MT=M5C=MT=M5C |
| 174 | CO-9402 | GTAGGTATGGAGAACCCTTT | 656 | MG=MT=MA=MG=MG=MT=MA=MT=MG=MG=MA=MG=MA=MA=M5C=M5C=M5C=MT=MT=MT |
| 175 | CO-9403 | GGAGTCTAAGATATCTGTAG | 657 | MG=MG=MA=MG=MT=M5C=MT=MA=MA=MG=MA=MT=MA=MT=M5C=MT=MG=MT=MA=MG |
| 176 | CO-9404 | GAGTCTAGGTCTCAGGTCCT | 658 | MG=MA=MG=MT=M5C=MT=MA=MG=MG=MT=M5C=MT=M5C=MA=MG=MG=MT=M5C=M5C=MT |
| 177 | CO-9405 | GCTCTAATTCTACATCTGGC | 659 | MG=M5C=MT=M5C=MT=MA=MA=MT=MT=M5C=MT=MA=M5C=MA=MT=M5C=MT=MG=MG=M5C |
| 178 | CO-9406 | CTAGGTCTGAGTTCTCTAAG | 660 | M5C=MT=MA=MG=MG=MT=M5C=MT=MG=MA=MG=MT=MT=M5C=MT=M5C=MT=MA=MA=MG |
| 179 | CO-9407 | GTCTCTAAGTTATCAGTCTC | 661 | MG=MT=M5C=MT=M5C=MT=MA=MA=MG=MT=MT=MA=MT=M5C=MA=MG=MT=M5C=MT=M5C |
| 180 | CO-9408 | TAAGTCAGGAGTGTCTCTGT | 662 | MT=MA=MA=MG=MT=M5C=MA=MG=MG=MA=MG=MT=MG=MT=M5C=MT=M5C=MT=MG=MT |
| 181 | CO-9409 | GTCTCTATCTCCCCTTAAGT | 663 | MG=MT=M5C=MT=M5C=MT=MA=MT=M5C=MT=M5C=M5C=M5C=M5C=MT=MT=MA=MA=MG=MT |
| 182 | CO-9410 | ATCTCCGGACCAGCTGAAGT | 664 | MA=MT=M5C=MT=M5C=M5C=MG=MG=MA=M5C=M5C=MA=MG=M5C=MT=MG=MA=MA=MG=MT |
| 183 | CO-9411 | GTCTCTGAGTTGTCCATCTC | 665 | MG=MT=M5C=MT=M5C=MT=MG=MA=MG=MT=MT=MG=MT=M5C=M5C=MA=MT=M5C=MT=M5C |
| 184 | CO-9412 | TCTTCATGAATTTTGGGTCT | 666 | MT=M5C=MT=MT=M5C=MA=MT=MG=MA=MA=MT=MT=MT=MT=MG=MG=MG=MT=M5C=MT |
| 185 | CO-9413 | CGCACTCTGAGGTCTTTCGA | 667 | M5C=MG=M5C=MA=M5C=MT=M5C=MT=MG=MA=MG=MG=MT=M5C=MT=MT=MT=M5C=MG=MA |
| 186 | CO-9414 | GGGTTTCTATGTTTCAGGGT | 668 | MG=MG=MG=MT=MT=MT=M5C=MT=MA=MT=MG=MT=MT=MT=M5C=MA=MG=MG=MG=MT |
| 187 | CO-9415 | CTTTTATGCTTCTGGTTTGG | 669 | M5C=MT=MT=MT=MT=MA=MT=MG=M5C=MT=MT=M5C=MT=MG=MG=MT=MT=MT=MG=MG |
| 188 | CO-9416 | GTGTGGAGATCTTTCTTAGG | 670 | MG=MT=MG=MT=MG=MG=MA=MG=MA=MT=M5C=MT=MT=MT=M5C=MT=MT=MA=MG=MG |
| 189 | CO-9417 | GGTCTTTCAGTATTTGTGTG | 671 | MG=MG=MT=M5C=MT=MT=MT=M5C=MA=MG=MT=MA=MT=MT=MT=MG=MT=MG=MT=MG |
| 190 | CO-9418 | GTTGGCTCTTATTCTCCTCC | 672 | MG=MT=MT=MG=MG=M5C=MT=M5C=MT=MT=MA=MT=MT=M5C=MT=M5C=M5C=MT=M5C=M5C |
| 191 | CO-9419 | GCTCTCTTCGCTCTCGCTCG | 673 | MG=M5C=MT=M5C=MT=M5C=MT=MT=M5C=MG=M5C=MT=M5C=MT=M5C=MG=M5C=MT=M5C=MG |
| 192 | CO-9420 | CTGCAGAGTCTCTCCCTCCT | 674 | M5C=MT=MG=M5C=MA=MG=MA=MG=MT=M5C=MT=M5C=MT=M5C=M5C=M5C=MT=M5C=M5C=MT |
| 193 | CO-9421 | CTGTCTCTCTCTGGTGTGTT | 675 | M5C=MT=MG=MT=M5C=MT=M5C=MT=M5C=MT=M5C=MT=MG=MG=MT=MG=MT=MG=MT=MT |
| 194 | CO-9422 | GGACCTCTGTCTCTCTCTGG | 676 | MG=MG=MA=M5C=M5C=MT=M5C=MT=MG=MT=M5C=MT=M5C=MT=M5C=MT=M5C=MT=MG=MG |
| 195 | CO-9423 | GTCTAGGGTCTCTCTGTCTC | 677 | MG=MT=M5C=MT=MA=MG=MG=MG=MT=M5C=MT=M5C=MT=M5C=MT=MG=MT=M5C=MT=M5C |
| 196 | CO-9424 | TGTGGGTCTAGGGTCTCTCT | 678 | MT=MG=MT=MG=MG=MG=MT=M5C=MT=MA=MG=MG=MG=MT=M5C=MT=M5C=MT=M5C=MT |
| 197 | CO-9425 | CTTTCTCTGTGGGTCTAGGG | 679 | M5C=MT=MT=MT=M5C=MT=M5C=MT=MG=MT=MG=MG=MG=MT=M5C=MT=MA=MG=MG=MG |
| 198 | CO-9426 | GAAGTATCTTTCTCTGTGGG | 680 | MG=MA=MA=MG=MT=MA=MT=M5C=MT=MT=MT=M5C=MT=M5C=MT=MG=MT=MG=MG=MG |
| 199 | CO-9427 | GGGTGGTGAAGTATCTTTCT | 681 | MG=MG=MG=MT=MG=MG=MT=MG=MA=MA=MG=MT=MA=MT=M5C=MT=MT=MT=M5C=MT |
| 200 | CO-9428 | GGCCCTGTGTCTCAAATTAC | 682 | MG=MG=M5C=M5C=M5C=MT=MG=MT=MG=MT=M5C=MT=M5C=MA=MA=MA=MT=MT=MA=M5C |
| 201 | CO-9429 | GATTTTGGCCCTGTGTCTCA | 683 | MG=MA=MT=MT=MT=MT=MG=MG=M5C=M5C=M5C=MT=MG=MT=MG=MT=M5C=MT=M5C=MA |
| 202 | CO-9430 | CCTCTAGATTTTGGCCCTGT | 684 | M5C=M5C=MT=M5C=MT=MA=MG=MA=MT=MT=MT=MT=MG=MG=M5C=M5C=M5C=MT=MG=MT |
| 203 | CO-9431 | CTGTAGGTATGGAGAACCCT | 685 | M5C=MT=MG=MT=MA=MG=MG=MT=MA=MT=MG=MG=MA=MG=MA=MA=M5C=M5C=M5C=MT |
| 204 | CO-9432 | TCTGGCTCTTTCTGAATCTG | 686 | MT=M5C=MT=MG=MG=M5C=MT=M5C=MT=MT=MT=M5C=MT=MG=MA=MA=MT=M5C=MT=MG |
| 205 | CO-9433 | CTACATCTGGCTCTTTCTGA | 687 | M5C=MT=MA=M5C=MA=MT=M5C=MT=MG=MG=M5C=MT=M5C=MT=MT=MT=M5C=MT=MG=MA |
| 206 | CO-9434 | GGGTCTCTTTCTAGGTCTGA | 688 | MG=MG=MG=MT=M5C=MT=M5C=MT=MT=MT=M5C=MT=MA=MG=MG=MT=M5C=MT=MG=MA |
| 207 | CO-9435 | GTCAGGAGTGTCTCTGTCTC | 689 | MG=MT=M5C=MA=MG=MG=MA=MG=MT=MG=MT=M5C=MT=M5C=MT=MG=MT=M5C=MT=M5C |
| 208 | CO-9436 | GCTGAAGTCTCTATCTCCCC | 690 | MG=M5C=MT=MG=MA=MA=MG=MT=M5C=MT=M5C=MT=MA=MT=M5C=MT=M5C=M5C=M5C=M5C |
| 209 | CO-9437 | TCTCCGGACCAGCTGAAGTC | 691 | MT=M5C=MT=M5C=M5C=MG=MG=MA=M5C=M5C=MA=MG=M5C=MT=MG=MA=MA=MG=MT=M5C |
| 210 | CO-9438 | GGGTCTCTGAGTTGTCCATC | 692 | MG=MG=MG=MT=M5C=MT=M5C=MT=MG=MA=MG=MT=MT=MG=MT=M5C=M5C=MA=MT=M5C |
| 211 | CO-9439 | GGTCGCACTCTGAGGTCTTT | 693 | MG=MG=MT=M5C=MG=M5C=MA=M5C=MT=M5C=MT=MG=MA=MG=MG=MT=M5C=MT=MT=MT |
| 212 | CO-9440 | GGGTCTGTGGTCGCACTCTG | 694 | MG=MG=MG=MT=M5C=MT=MG=MT=MG=MG=MT=M5C=MG=M5C=MA=M5C=MT=M5C=MT=MG |
| 213 | CO-9441 | GTTTCAGGGTCTGTGGTCGC | 695 | MG=MT=MT=MT=M5C=MA=MG=MG=MG=MT=M5C=MT=MG=MT=MG=MG=MT=M5C=MG=M5C |
| 214 | CO-9442 | TCTATGTTTCAGGGTCTGTG | 696 | MT=M5C=MT=MA=MT=MG=MT=MT=MT=M5C=MA=MG=MG=MG=MT=M5C=MT=MG=MT=MG |
| 215 | CO-9443 | TTTTCAGATTTTGATATTGA | 697 | MT=MT=MT=MT=M5C=MA=MG=MA=MT=MT=MT=MT=MG=MA=MT=MA=MT=MT=MG=MA |
| 216 | CO-9444 | CCTGAAATCATCATCTCAGG | 698 | M5C=M5C=MT=MG=MA=MA=MA=MT=M5C=MA=MT=M5C=MA=MT=M5C=MT=M5C=MA=MG=MG |
| 217 | CO-9445 | AGGATTGAGTTTCCTTTTAT | 699 | MA=MG=MG=MA=MT=MT=MG=MA=MG=MT=MT=MT=M5C=M5C=MT=MT=MT=MT=MA=MT |
| 218 | CO-9446 | GAGATCTTTCTTAGGATTGA | 700 | MG=MA=MG=MA=MT=M5C=MT=MT=MT=M5C=MT=MT=MA=MG=MG=MA=MT=MT=MG=MA |
| 219 | CO-9447 | CTCTGAGACAGATTTCGGAG | 701 | M5C=MT=M5C=MT=MG=MA=MG=MA=M5C=MA=MG=MA=MT=MT=MT=M5C=MG=MG=MA=MG |
| 220 | CO-10604 | GCAAGTGCCAGGCCCACATG | 702 | MG=M5C=MA=MA=MG=dT=dG=M5C=d5C=dA=MG=dG=d5C=M5C=d5C=MA=M5C=MA=MT=MG |
| 221 | CO-10605 | GCAAGTGCCAGGCCCACATG | 703 | MG=M5C=LA=MA=MG=dT=dG=d5C=d5C=dA=dG=dG=d5C=d5C=d5C=MA=M5C=LA=MT=MG |
| 222 | CO-10606 | GCAAGTGCCAGGCCCACATG | 704 | MG=M5C=LA=MA=LG=dT=dG=d5C=d5C=dA=dG=dG=d5C=d5C=d5C=MA=M5C=MA=MT=MG |
| 223 | CO-10607 | GCAAGTGCCAGGCCCACATG | 705 | MG=L5C=MA=LA=MG=dT=dG=d5C=d5C=dA=dG=dG=d5C=d5C=d5C=MA=L5C=MA=LT=MG |
| 224 | CO-10608 | GCAAGTGCCAGGCCCACATG | 706 | MG=L5C=MA=LA=MG=dT=dG=M5C=d5C=dA=MG=dG=d5C=M5C=d5C=MA=L5C=MA=LT |
| 225 | CO-10609 | GCAAGTGCCAGGCCCACATG | 707 | LG=M5C=LA=MA=MG=dT=dG=d5C=d5C=dA=dG=dG=d5C=d5C=d5C=MA=M5C=LA=MT |
| 226 | CO-10610 | GCAAGTGCCAGGCCCACATG | 708 | MG=M5C=MA-MA-MG=dT=dG=d5C=d5C=dA=dG=dG=d5C=d5C=d5C=MA-M5C-MA=MT=MG |
| 227 | CO-10611 | GCAAGTGCCAGGCCCACATG | 709 | MG=M5C-MA-MA-MG=dT=dG=d5C=d5C=dA=dG=dG=d5C=d5C=d5C=MA-M5C-MA-MT=MG |
| 228 | CO-10612 | GGCAAGTGCCAGGCCCACATGG | 710 | MG=MG=M5C=MA=MA=MG=dT=dG=d5C=d5C=dA=dG=dG=d5C=d5C=d5C=MA=M5C=MA=MT=MG=MG |
| 229 | CO-10613 | AAGTGCCAGGCCCACA | | |
| 230 | CO-10614 | TGGGAAGACACAAAGTGATG | 711 | MT=MG=MG=MG=MA=dA=dG=MA=d5C=dA=M5C=dA=dA=MA=dG=MT=MG=MA=MT=MG |
| 231 | CO-10615 | TGGGAAGACACAAAGTGATG | 712 | MT=MG=LG=MG=MA=dA=dG=dA=d5C=dA=d5C=dA=dA=dA=dG=MT=MG=LA=MT=MG |
| 232 | CO-10616 | TGGGAAGACACAAAGTGATG | 713 | MT=MG=LG=MG=LA=dA=dG=dA=d5C=dA=d5C=dA=dA=dA=dG=MT=MG=MA=MT=MG |
| 233 | CO-10617 | TGGGAAGACACAAAGTGATG | 714 | MT=LG=MG=LG=MA=dA=dG=dA=d5C=dA=d5C=dA=dA=dA=dG=MT=LG=MA=LT=MG |
| 234 | CO-10618 | TGGGAAGACACAAAGTGATG | 715 | MT=LG=MG=LG=MA=dA=dG=MA=d5C=dA=M5C=dA=dA=MA=dG=MT=LG=MA=LT=MG |
| 235 | CO-10619 | TGGGAAGACACAAAGTGATG | 716 | LT=MG=LG=MG=MA=dA=dG=dA=d5C=dA=d5C=dA=dA=dA=dG=MT=MG=LA=MT=LG |
| 236 | CO-10620 | TGGGAAGACACAAAGTGATG | 717 | MT=MG=MG-MG-MA=dA=dG=dA=d5C=dA=d5C=dA=dA=dA=dG=MT-MG-MA=MT=MG |
| 237 | CO-10621 | TGGGAAGACACAAAGTGATG | 718 | MT=MG-MG-MG-MA=dA=dG=dA=d5C=dA=d5C=dA=dA=dA=dG=MT-MG-MA-MT=MG |
| 238 | CO-10622 | GTGGGAAGACACAAAGTGATGT | 719 | MG=MT=MG=MG=MG=MA=dA=dG=dA=d5C=dA=d5C=dA=dA=dA=dG=MT=MG=MA=MT=MG=MT |
| 239 | CO-10623 | GGAAGACACAAAGTGA | | |
| 240 | CO-10624 | GCTCCCGTGCAGAAAACAGG | 720 | MG=M5C=MT=M5C=M5C=d5C=dG=MT=dG=d5C=MA=dG=dA=MA=dA=MA=M5C=MA=MG=MG |
| 241 | CO-10625 | GCTCCCGTGCAGAAAACAGG | 721 | MG=M5C=LT=M5C=M5C=d5C=dG=dT=dG=d5C=dA=dG=dA=dA=dA=MA=M5C=LA=MG=MG |
| 242 | CO-10626 | GCTCCCGTGCAGAAAACAGG | 722 | MG=M5C=LT=M5C=L5C=d5C=dG=dT=dG=d5C=dA=dG=dA=dA=dA=MA=M5C=MA=MG=MG |
| 243 | CO-10627 | GCTCCCGTGCAGAAAACAGG | 723 | MG=L5C=MT=L5C=M5C=d5C=dG=dT=dG=d5C=dA=dG=dA=dA=dA=MA=L5C=MA=LG=MG |
| 244 | CO-10628 | GCTCCCGTGCAGAAAACAGG | 724 | MG=L5C=MT=L5C=M5C=d5C=dG=MT=dG=d5C=MA=dG=dA=MA=dA=MA=L5C=MA=LG=MG |
| 245 | CO-10629 | GCTCCCGTGCAGAAAACAGG | 725 | LG=M5C=LT=M5C=M5C=d5C=dG=dT=dG=d5C=dA=dG=dA=dA=dA=MA=M5C=LA=MG=LG |
| 246 | CO-10630 | GCTCCCGTGCAGAAAACAGG | 726 | MG=M5C=MT-M5C-M5C=d5C=dG=dT=dG=d5C=dA=dG=dA=dA=dA=MA-M5C-MA=MG=MG |
| 247 | CO-10631 | GCTCCCGTGCAGAAAACAGG | 727 | MG=M5C-MT-M5C-M5C=d5C=dG=dT=dG=d5C=dA=dG=dA=dA=dA=MA-M5C-MA-MG=MG |
| 248 | CO-10632 | TGCTCCCGTGCAGAAAACAGGA | 728 | MT=MG=M5C=MT=M5C=M5C=d5C=dG=dT=dG=d5C=dA=dG=dA=dA=dA=MA=M5C=MA=MG=MG=MA |
| 250 | CO-10633 | TCCCGTGCAGAAAACA | | |
| 251 | CO-10634 | TATAAGGTGGGTTAAGGGTC | | |
| 252 | CO-10635 | TATAAGGTGGGTTAAGGGTC | | |
| 253 | CO-10636 | TATAAGGTGGGTTAAGGGTC | | |
| 254 | CO-10637 | TATAAGGTGGGTTAAGGGTC | | |
| 255 | CO-10638 | TATAAGGTGGGTTAAGGGTC | | |
| 256 | CO-10639 | TATAAGGTGGGTTAAGGGTC | | |
| 257 | CO-10640 | TATAAGGTGGGTTAAGGGTC | | |
| 258 | CO-10641 | TATAAGGTGGGTTAAGGGTC | | |
| 259 | CO-10642 | TTATAAGGTGGGTTAAGGGTCC | | |
| 260 | CO-10643 | TAAGGTGGGTTAAGGG | | |
| 261 | CO-10644 | CCAGGCCCACATGGTAAGTA | 729 | M5C=M5C=MA=MG=MG=d5C=d5C=d5C=dA=d5C=dA=dT=dG=dG=dT=MA=MA=MG=MT=MA |
| 262 | CO-10645 | CAAGTGCCAGGCCCACATGG | 730 | M5C=MA=MA=MG=MT=dG=d5C=d5C=dA=dG=dG=d5C=d5C=d5C=dA=M5C=MA=MT=MG=MG |
| 263 | CO-10646 | GGGAAGACACAAAGTGATGT | 731 | MG=MG=MG=MA=MA=dG=dA=d5C=dA=d5C=dA=dA=dA=dG=dT=MG=MA=MT=MG=MT |
| 264 | CO-10647 | CGGTGGGAAGACACAAAGTG | 732 | M5C=MG=MG=MT=MG=dG=dG=dA=dA=dG=dA=d5C=dA=d5C=dA=MA=MA=MG=MT=MG |
| 265 | CO-10648 | CGTGCAGAAAACAGGAAAGA | 733 | M5C=MG=MT=MG=M5C=dA=dG=dA=dA=dA=dA=d5C=dA=dG=dG=MA=MA=MA=MG=MA |
| 266 | CO-10649 | CCCGTGCAGAAAACAGGAAA | 734 | M5C=M5C=M5C=MG=MT=dG=d5C=dA=dG=dA=dA=dA=dA=d5C=dA=MG=MG=MA=MA=MA |
| 267 | CO-10650 | TGCTCCCGTGCAGAAAACAG | 735 | MT=MG=M5C=MT=M5C=d5C=d5C=dG=dT=dG=d5C=dA=dG=dA=dA=MA=MA=M5C=MA=MG |
| 268 | CO-10651 | GGGTTAAGGGTCCACTCTAA | | |
| 269 | CO-10652 | TAAGGTGGGTTAAGGGTCCA | | |
| 270 | CO-10653 | TTTTATAAGGTGGGTTAAGG | | |
| 271 | CO-10654 | CAGTTTTTATAAGGTGGGTT | | |
| 272 | CO-10655 | CAAGTGCCAGGCCCACATGG | 736 | M5C=LA=MA=LG=MT=dG=d5C=d5C=dA=dG=dG=d5C=d5C=d5C=dA=M5C=LA=MT=LG=MG |
| 273 | CO-10656 | GGGAAGACACAAAGTGATGT | 737 | MG=LG=MG=LA=MA=dG=dA=d5C=dA=d5C=dA=dA=dA=dG=dT=MG=LA=MT=LG=MT |
| 274 | CO-10657 | TGCTCCCGTGCAGAAAACAG | 738 | MT=LG=M5C=LT=M5C=d5C=d5C=dG=dT=dG=d5C=dA=dG=dA=dA=MA=LA=M5C=LA=MG |
| 275 | CO-10658 | TAAGGTGGGTTAAGGGTCCA | | |
| 276 | CO-10795 | GCAAGTGCCAGGCCCACATG | 739 | MG=M5C=LA=MA=LG=dT=dG=d5C=d5C=dA=dG=dG=d5C=d5C=d5C=LA=M5C=LA=MT=MG |
| 277 | CO-10796 | TGGGAAGACACAAAGTGATG | 740 | MT=MG=LG=MG=LA=dA=dG=dA=d5C=dA=d5C=dA=dA=dA=dG=LT=MG=LA=MT=MG |
| 278 | CO-10797 | GCTCCCGTGCAGAAAACAGG | 741 | MG=M5C=LT=M5C=L5C=d5C=dG=dT=dG=d5C=dA=dG=dA=dA=dA=LA=M5C=LA=MG=MG |
| 279 | CO-10798 | TATAAGGTGGGTTAAGGGTC | | |
| 280 | CO-11470 | CTCTGGTGTGTTTTTGAGTC | 742 | M5C=MT=M5C=MT=MG=dG=dT=dG=dT=dG=dT=dT=dT=dT=dT=MG=MA=MG=MT=M5C |
| 281 | CO-11471 | TCTCTCTGGTGTGTTTTTGA | 743 | MT=M5C=MT=M5C=MT=d5C=dT=dG=dG=dT=dG=dT=dG=dT=dT=MT=MT=MT=MG=MA |
| 282 | CO-11472 | AGGACCTCTGTCTCTCTCTG | 744 | MA=MG=MG=MA=M5C=d5C=dT=d5C=dT=dG=dT=d5C=dT=d5C=dT=M5C=MT=M5C=MT=MG |
| 283 | CO-11473 | CTTTAGGACCTCTGTCTCTC | 745 | M5C=MT=MT=MT=MA=dG=dG=dA=d5C=d5C=dT=d5C=dT=dG=dT=M5C=MT=M5C=MT=M5C |
| 284 | CO-11474 | CATCTTTAGGACCTCTGTCT | 746 | M5C=MA=MT=M5C=MT=dT=dT=dA=dG=dG=dA=d5C=d5C=dT=d5C=MT=MG=MT=M5C=MT |
| 285 | CO-11475 | AGGGTCTCTCTGTCTCAGGG | 747 | MA=MG=MG=MG=MT=d5C=dT=d5C=dT=d5C=dT=dG=dT=d5C=dT=M5C=MA=MG=MG=MG |
| 286 | CO-11476 | CTAGGGTCTCTCTGTCTCAG | 748 | M5C=MT=MA=MG=MG=dG=dT=d5C=dT=d5C=dT=d5C=dT=dG=dT=M5C=MT=M5C=MA=MG |
| 287 | CO-11477 | GGTCTAGGGTCTCTCTGTCT | 749 | MG=MG=MT=M5C=MT=dA=dG=dG=dG=dT=d5C=dT=d5C=dT=d5C=MT=MG=MT=M5C=MT |
| 288 | CO-11478 | TGGGTCTAGGGTCTCTCTGT | 750 | MT=MG=MG=MG=MT=d5C=dT=dA=dG=dG=dG=dT=d5C=dT=d5C=MT=M5C=MT=MG=MT |
| 289 | CO-11479 | CTGTGGGTCTAGGGTCTCTC | 751 | M5C=MT=MG=MT=MG=dG=dG=dT=d5C=dT=dA=dG=dG=dG=dT=M5C=MT=M5C=MT=M5C |
| 290 | CO-11480 | CTCTGTGGGTCTAGGGTCTC | 752 | M5C=MT=M5C=MT=MG=dT=dG=dG=dG=dT=d5C=dT=dA=dG=dG=MG=MT=M5C=MT=M5C |
| 291 | CO-11481 | TTCTCTGTGGGTCTAGGGTC | 753 | MT=MT=M5C=MT=M5C=dT=dG=dT=dG=dG=dG=dT=d5C=dT=dA=MG=MG=MG=MT=M5C |
| 292 | CO-11482 | ATCTTTCTCTGTGGGTCTAG | 754 | MA=MT=M5C=MT=MT=dT=d5C=dT=d5C=dT=dG=dT=dG=dG=dG=MT=M5C=MT=MA=MG |
| 293 | CO-11483 | AAGTATCTTTCTCTGTGGGT | 755 | MA=MA=MG=MT=MA=dT=d5C=dT=dT=dT=d5C=dT=d5C=dT=dG=MT=MG=MG=MG=MT |
| 294 | CO-11484 | GTGAAGTATCTTTCTCTGTG | 756 | MG=MT=MG=MA=MA=dG=dT=dA=dT=d5C=dT=dT=dT=d5C=dT=M5C=MT=MG=MT=MG |
| 295 | CO-11485 | TGGTGAAGTATCTTTCTCTG | 757 | MT=MG=MG=MT=MG=dA=dA=dG=dT=dA=dT=d5C=dT=dT=dT=M5C=MT=M5C=MT=MG |
| 296 | CO-11486 | CTCAAATTACCCCACAGTGG | 758 | M5C=MT=M5C=MA=MA=dA=dT=dT=dA=d5C=d5C=d5C=d5C=dA=d5C=MA=MG=MT=MG=MG |
| 297 | CO-11487 | GTGTCTCAAATTACCCCACA | 759 | MG=MT=MG=MT=M5C=dT=d5C=dA=dA=dA=dT=dT=dA=d5C=d5C=M5C=M5C=MA=M5C=MA |
| 298 | CO-11488 | CTGTGTCTCAAATTACCCCA | 760 | M5C=MT=MG=MT=MG=dT=d5C=dT=d5C=dA=dA=dA=dT=dT=dA=M5C=M5C=M5C=M5C=MA |
| 299 | CO-11489 | CCCTGTGTCTCAAATTACCC | 761 | M5C=M5C=M5C=MT=MG=dT=dG=dT=d5C=dT=d5C=dA=dA=dA=dT=MT=MA=M5C=M5C=M5C |
| 300 | CO-11490 | TTGGCCCTGTGTCTCAAATT | 762 | MT=MT=MG=MG=M5C=d5C=d5C=dT=dG=dT=dG=dT=d5C=dT=d5C=MA=MA=MA=MT=MT |
| 301 | CO-11491 | TTTTGGCCCTGTGTCTCAAA | 763 | MT=MT=MT=MT=MG=dG=d5C=d5C=d5C=dT=dG=dT=dG=dT=d5C=MT=M5C=MA=MA=MA |
| 302 | CO-11492 | CTAGATTTTGGCCCTGTGTC | 764 | M5C=MT=MA=MG=MA=dT=dT=dT=dT=dG=dG=d5C=d5C=d5C=dT=MG=MT=MG=MT=M5C |
| 303 | CO-11493 | TTCCTCTAGATTTTGGCCCT | 765 | MT=MT=M5C=M5C=MT=d5C=dT=dA=dG=dA=dT=dT=dT=dT=dG=MG=M5C=M5C=M5C=MT |
| 304 | CO-11494 | CTTTTCCTCTAGATTTTGGC | 766 | M5C=MT=MT=MT=MT=d5C=d5C=dT=d5C=dT=dA=dG=dA=dT=dT=MT=MT=MG=MG=M5C |
| 305 | CO-11495 | AGTCTCTTTTCCTCTAGATT | 767 | MA=MG=MT=M5C=MT=d5C=dT=dT=dT=dT=d5C=d5C=dT=d5C=dT=MA=MG=MA=MT=MT |
| 306 | CO-11496 | CAAATCAGGAGTCTCTTTTC | 768 | M5C=MA=MA=MA=MT=d5C=dA=dG=dG=dA=dG=dT=d5C=dT=d5C=MT=MT=MT=MT=M5C |
| 307 | CO-11497 | CCTCAAATCAGGAGTCTCTT | 769 | M5C=M5C=MT=M5C=MA=dA=dA=dT=d5C=dA=dG=dG=dA=dG=dT=M5C=MT=M5C=MT=MT |
| 308 | CO-11498 | CGTTTTTTCCTCAAATCAGG | 770 | M5C=MG=MT=MT=MT=dT=dT=dT=d5C=d5C=dT=d5C=dA=dA=dA=MT=M5C=MA=MG=MG |
| 309 | CO-11499 | GTCGTTTTTTCCTCAAATCA | 771 | MG=MT=M5C=MG=MT=dT=dT=dT=dT=dT=d5C=d5C=dT=d5C=dA=MA=MA=MT=M5C=MA |
| 310 | CO-11500 | GATATTGAGCTTCTCTCTCT | 772 | MG=MA=MT=MA=MT=dT=dG=dA=dG=d5C=dT=dT=d5C=dT=d5C=MT=M5C=MT=M5C=MT |
| 311 | CO-11501 | TTTGATATTGAGCTTCTCTC | 773 | MT=MT=MT=MG=MA=dT=dA=dT=dT=dG=dA=dG=d5C=dT=dT=M5C=MT=M5C=MT=M5C |
| 312 | CO-11502 | GATTTTGATATTGAGCTTCT | 774 | MG=MA=MT=MT=MT=dT=dG=dA=dT=dA=dT=dT=dG=dA=dG=M5C=MT=MT=M5C=MT |
| 313 | CO-11503 | TCAGATTTTGATATTGAGCT | 775 | MT=M5C=MA=MG=MA=dT=dT=dT=dT=dG=dA=dT=dA=dT=dT=MG=MA=MG=M5C=MT |
| 314 | CO-11504 | ACTGGATTTTCAGATTTTGA | 776 | MA=M5C=MT=MG=MG=dA=dT=dT=dT=dT=d5C=dA=dG=dA=dT=MT=MT=MT=MG=MA |
| 315 | CO-11505 | TAGACTGGATTTTCAGATTT | 777 | MT=MA=MG=MA=M5C=dT=dG=dG=dA=dT=dT=dT=dT=d5C=dA=MG=MA=MT=MT=MT |
| 316 | CO-11506 | CTTTTTAGACTGGATTTTCA | 778 | M5C=MT=MT=MT=MT=dT=dA=dG=dA=d5C=dT=dG=dG=dA=dT=MT=MT=MT=M5C=MA |
| 317 | CO-11507 | ACCCTTTTTAGACTGGATTT | 779 | MA=M5C=M5C=M5C=MT=dT=dT=dT=dT=dA=dG=dA=d5C=dT=dG=MG=MA=MT=MT=MT |
| 318 | CO-11508 | AGAACCCTTTTTAGACTGGA | 780 | MA=MG=MA=MA=M5C=d5C=d5C=dT=dT=dT=dT=dT=dA=dG=dA=M5C=MT=MG=MG=MA |
| 319 | CO-11509 | TGGAGAACCCTTTTTAGACT | 781 | MT=MG=MG=MA=MG=dA=dA=d5C=d5C=d5C=dT=dT=dT=dT=dT=MA=MG=MA=M5C=MT |
| 320 | CO-11510 | GTATGGAGAACCCTTTTTAG | 782 | MG=MT=MA=MT=MG=dG=dA=dG=dA=dA=d5C=d5C=d5C=dT=dT=MT=MT=MT=MA=MG |
| 321 | CO-11511 | GATATCTGTAGGTATGGAGA | 783 | MG=MA=MT=MA=MT=d5C=dT=dG=dT=dA=dG=dG=dT=dA=dT=MG=MG=MA=MG=MA |
| 322 | CO-11512 | TAAGATATCTGTAGGTATGG | 784 | MT=MA=MA=MG=MA=dT=dA=dT=d5C=dT=dG=dT=dA=dG=dG=MT=MA=MT=MG=MG |
| 323 | CO-11513 | AGTCTAAGATATCTGTAGGT | 785 | MA=MG=MT=M5C=MT=dA=dA=dG=dA=dT=dA=dT=d5C=dT=dG=MT=MA=MG=MG=MT |
| 324 | CO-11514 | GGACCTCTGTCTCTCTCTGG | 786 | MG=LG=MA=M5C=M5C=dT=d5C=dT=dG=dT=d5C=dT=d5C=dT=d5C=MT=M5C=MT=LG=MG |
| 325 | CO-11515 | GGACCTCTGTCTCTCTCTGG | 787 | MG=MG=MA=L5C=M5C=dT=d5C=dT=dG=dT=d5C=dT=d5C=dT=d5C=MT=L5C=MT=MG=MG |
| 326 | CO-11516 | GGACCTCTGTCTCTCTCTGG | 788 | MG=LG=MA=L5C=M5C=dT=d5C=dT=dG=dT=d5C=dT=d5C=dT=d5C=MT=L5C=MT=LG=MG |
| 327 | CO-11517 | GGACCTCTGTCTCTCTCTGG | 789 | LG=MG=MA=L5C=M5C=dT=d5C=dT=dG=dT=d5C=dT=d5C=dT=d5C=MT=L5C=MT=MG=LG |
| 328 | CO-11518 | GGACCTCTGTCTCTCTCTGG | 790 | MG=MG=MA-M5C-M5C=dT=d5C=dT=dG=dT=d5C=dT=d5C=dT=d5C=MT-M5C-MT=MG=MG |
| 329 | CO-11519 | GGACCTCTGTCTCTCTCTGG | 791 | MG=MG-MA-M5C-M5C=dT=d5C=dT=dG=dT=d5C=dT=d5C=dT=d5C=MT-M5C-MT-MG=MG |
| 330 | CO-11520 | GGACCTCTGTCTCTCTCTGG | 792 | MG-MG-MA-M5CM5C=dT=d5C=dT=dG=dT=d5C=dT=d5C=dT=d5C=MT-M5C-MT-MG-MG |
| 331 | CO-11521 | GGACCTCTGTCTCTCTCTGG | 793 | MG=MG-MA=M5C-M5C=dT=d5C=dT=dG=dT=d5C=dT=d5C=dT=d5C=MT-M5C=MT-MG=MG |
| 332 | CO-11522 | GGACCTCTGTCTCTCTCTGG | 794 | MG-MG=MA-M5C=M5C=dT=d5C=dT=dG=dT=d5C=dT=d5C=dT=d5C=MT=M5C-MT=MG=MG |
| 333 | CO-11523 | GGACCTCTGTCTCTCTCTGG | 795 | MG=MG=MA=M5C=M5C=dT=d5C=MT=dG=dT=M5C=dT=d5C=MT=d5C=MT=M5C=MT=MG=MG |
| 334 | CO-11524 | AGGACCTCTGTCTCTCTCTGGT | 796 | MA=MG=MG=MA=M5C=M5C=dT=d5C=dT=dG=dT=d5C=dT=d5C=dT=d5C=MT=M5C=MT=MG=MG=MT |
| 335 | CO-11525 | AGGACCTCTGTCTCTCTCTGGT | 797 | LA=MG=MG=LA=M5C=M5C=dT=d5C=dT=dG=dT=d5C=dT=d5C=dT=d5C=MT=M5C=LT=MG=MG=LT |
| 336 | CO-11526 | AGGACCTCTGTCTCTCTCTGGT | 798 | MA=MG=LG=MA=L5C=M5C=dT=d5C=dT=dG=dT=d5C=dT=d5C=dT=d5C=MT=L5C=MT=LG=MG=MT |
| 337 | CO-11527 | AGGACCTCTGTCTCTCTCTGGT | 799 | MA-MG=MG-MA=M5C-M5C=dT=d5C=dT=dG=dT=d5C=dT=d5C=dT=d5C=MT-M5C=MT-MG=MG=MT |
| 338 | CO-11528 | AGGACCTCTGTCTCTCTCTGGT | 800 | MA=MG-MG=MA-M5C=M5C=dT=d5C=dT=dG=dT=d5C=dT=d5C=dT=d5C=MT=M5C-MT=MG-MG=MT |
| 339 | CO-11529 | AGGACCTCTGTCTCTCTCTGGT | 801 | MA=MG-MG=MA=M5C-M5C=dT=d5C=dT=dG=dT=d5C=dT=d5C=dT=d5C=MT-M5C=MT=MG-MG=MT |
| 340 | CO-11530 | TAGGACCTCTGTCTCTCTCTGGTG | 802 | MT=MA=MG=MG=MA=M5C=M5C=dT=d5C=dT=dG=dT=d5C=dT=d5C=dT=d5C=MT=M5C=MT=MG=MG=MT=MG |
| 341 | CO-11531 | TAGGACCTCTGTCTCTCTCTGGTG | 803 | LT=MA=MG=LG=MA=M5C=M5C=dT=d5C=dT=dG=dT=d5C=dT=d5C=dT=d5C=MT=M5C=MT=LG=MG=MT=LG |
| 342 | CO-11532 | TAGGACCTCTGTCTCTCTCTGGTG | 804 | MT=MA=LG=MG=MA=L5C=M5C=dT=d5C=dT=dG=dT=d5C=dT=d5C=dT=d5C=MT=L5C=MT=MG=LG=MT=MG |
| 343 | CO-11533 | TAGGACCTCTGTCTCTCTCTGGTBG | 805 | MT=MA-MG=MG-MA=M5C-M5C=dT=d5C=dT=dG=dT=d5C=dT=d5C=dT=d5C=MT-M5C=MT-MG=MG-MT=MG |
| 344 | CO-11534 | TAGGACCTCTGTCTCTCTCTGGTG | 806 | MT-MA=MG-MG=MA-M5C=M5C=dT=d5C=dT=dG=dT=d5C=dT=d5C=dT=d5C=MT=M5C-MT=MG-MG=MT-MG |
| 345 | CO-11535 | TAGGACCTCTGTCTCTCTCTGGTG | 807 | MT=MA-MG=MG-MA=M5C=M5C=dT=d5C=dT=dG=dT=d5C=dT=d5C=dT=d5C=MT=M5C=MT-MG=MG-MT=MG |
| 346 | CO-11536 | GGCCCTGTGTCTCAAATTAC | 808 | MG=LG=M5C=M5C=M5C=dT=dG=dT=dG=dT=d5C=dT=d5C=dA=dA=MA=MT=MT=LA=M5C |
| 347 | CO-11537 | GGCCCTGTGTCTCAAATTAC | 809 | MG=MG=M5C=L5C=M5C=dT=dG=dT=dG=dT=d5C=dT=d5C=dA=dA=MA=LT=MT=MA=M5C |
| 348 | CO-11538 | GGCCCTGTGTCTCAAATTAC | 810 | MG=LG=M5C=L5C=M5C=dT=dG=dT=dG=dT=d5C=dT=d5C=dA=dA=MA=LT=MT=LA=M5C |
| 349 | CO-11539 | GGCCCTGTGTCTCAAATTAC | 811 | LG=MG=M5C=L5C=M5C=dT=dG=dT=dG=dT=d5C=dT=d5C=dA=dA=MA=LT=MT=MA=L5C |
| 350 | CO-11540 | GGCCCTGTGTCTCAAATTAC | 812 | MG=MG=M5C-M5C-M5C=dT=dG=dT=dG=dT=d5C=dT=d5C=dA=dA=MA-MT-MT=MA=M5C |
| 351 | CO-11541 | GGCCCTGTGTCTCAAATTAC | 813 | MG=MG-M5C-M5C-M5C=dT=dG=dT=dG=dT=d5C=dT=d5C=dA=dA=MA-MT-MT-MA=M5C |
| 352 | CO-11542 | GGCCCTGTGTCTCAAATTAC | 814 | MG-MG-M5C-M5C-M5C=dT=dG=dT=dG=dT=d5C=dT=d5C=dA=dA=MA-MT-MT-MA-M5C |
| 353 | CO-11543 | GGCCCTGTGTCTCAAATTAC | 815 | MG=MG-M5C=M5C-M5C=dT=dG=dT=dG=dT=d5C=dT=d5C=dA=dA=MA-MT=MT-MA=M5C |
| 354 | CO-11544 | GGCCCTGTGTCTCAAATTAC | 816 | MG-MG=M5C-M5C=M5C=dT=dG=dT=dG=dT=d5C=dT=d5C=dA=dA=MA=MT-MT=MA-M5C |
| 355 | CO-11545 | GGCCCTGTGTCTCAAATTAC | 817 | MG=MG=M5C=M5C=M5C=dT=dG=MT=dG=dT=M5C=dT=d5C=MA=dA=MA=MT=MT=MA=M5C |
| 356 | CO-11546 | TGGCCCTGTGTCTCAAATTACC | 818 | MT=MG=MG=M5C=M5C=M5C=dT=dG=dT=dG=dT=d5C=dT=d5C=dA=dA=MA=MT=MT=MA=M5C=M5C |
| 357 | CO-11547 | TGGCCCTGTGTCTCAAATTACC | 819 | LT=MG=MG=L5C=M5C=M5C=dT=dG=dT=dG=dT=d5C=dT=d5C=dA=dA=MA=MT=LT=MA=M5C=L5C |
| 358 | CO-11548 | TGGCCCTGTGTCTCAAATTACC | 820 | MT=MG=LG=M5C=L5C=M5C=dT=dG=dT=dG=dT=d5C=dT=d5C=dA=dA=MA=LT=MT=LA=M5C=M5C |
| 359 | CO-11549 | TGGCCCTGTGTCTCAAATTACC | 821 | MT-MG=MG-M5C=M5C-M5C=dT=dG=dT=dG=dT=d5C=dT=d5C=dA=dA=MA-MT=MT-MA=M5C=M5C |
| 360 | CO-11550 | TGGCCCTGTGTCTCAAATTACC | 822 | MT=MG-MG=M5C-M5C=M5C=dT=dG=dT=dG=dT=d5C=dT=d5C=dA=dA=MA=MT-MT=MA-M5C=M5C |
| 361 | CO-11551 | TGGCCCTGTGTCTCAAATTACC | 823 | MT=MG-MG=M5C=M5C-M5C=dT=dG=dT=dG=dT=d5C=dT=d5C=dA=dA=MA-MT=MT=MA-M5C=M5C |
| 362 | CO-11552 | TTGGCCCTGTGTCTCAAATTACCC | 824 | MT=MT=MG=MG=M5C=M5C=M5C=dT=dG=dT=dG=dT=d5C=dT=d5C=dA=dA=MA=MT=MT=MA=M5C=M5C=M5C |
| 363 | CO-11553 | TTGGCCCTGTGTCTCAAATTACCC | 825 | LT=MT=MG=LG=M5C=M5C=M5C=dT=dG=dT=dG=dT=d5C=dT=d5C=dA=dA=MA=MT=MT=LA=M5C=M5C=L5C |
| 364 | CO-11554 | TTGGCCCTGTGTCTCAAATTACCC | 826 | MT=MT=LG=MG=M5C=L5C=M5C=dT=dG=dT=dG=dT=d5C=dT=d5C=dA=dA=MA=LT=MT=MA=L5C=M5C=M5C |
| 365 | CO-11555 | TTGGCCCTGTGTCTCAAATTACCC | 827 | MT=MT-MG=MG-M5C=M5C-M5C=dT=dG=dT=dG=dT=d5C=dT=d5C=dA=dA=MA-MT=MT-MA=M5C-M5C=M5C |
| 366 | CO-11556 | TTGGCCCTGTGTCTCAAATTACCC | 828 | MT-MT=MG-MG=M5C-M5C=M5C=dT=dG=dT=dG=dT=d5C=dT=d5C=dA=dA=MA=MT-MT=MA-M5C=M5C-M5C |
| 367 | CO-11557 | TTGGCCCTGTGTCTCAAATTACCC | 829 | MT=MT-MG=MG-M5C=M5C=M5C=dT=dG=dT=dG=dT=d5C=dT=d5C=dA=dA=MA=MT=MT-MA=M5C- |
| | | | | M5C=M5C |
| 368 | CO-11558 | CTGTAGGTATGGAGAACCCT | 830 | M5C=LT=MG=MT=MA=dG=dG=dT=dA=dT=dG=dG=dA=dG=dA=MA=M5C=M5C=L 5C=MT |
| 369 | CO-11559 | CTGTAGGTATGGAGAACCCT | 831 | M5C=MT=MG=LT=MA=dG=dG=dT=dA=dT=dG=dG=dA=dG=dA=MA=L5C=M5C=M5C=MT |
| 370 | CO-11560 | CTGTAGGTATGGAGAACCCT | 832 | M5C=LT=MG=LT=MA=dG=dG=dT=dA=dT=dG=dG=dA=dG=dA=MA=L5C=M5C=L5C=MT |
| 371 | CO-11561 | CTGTAGGTATGGAGAACCCT | 833 | L5C=MT=MG=LT=MA=dG=dG=dT=dA=dT=dG=dG=dA=dG=dA=MA=L5C=M5C=M5C=LT |
| 372 | CO-11562 | CTGTAGGTATGGAGAACCCT | 834 | M5C=MT=MG-MT-MA=dG=dG=dT=dA=dT=dG=dG=dA=dG=dA=MA-M5C-M5C=M5C=MT |
| 373 | CO-11563 | CTGTAGGTATGGAGAACCCT | 835 | M5C=MT-MG-MT-MA=dG=dG=dT=dA=dT=dG=dG=dA=dG=dA=MA-M5C-M5C-M5C=MT |
| 374 | CO-11564 | CTGTAGGTATGGAGAACCCT | 836 | M5C-MT-MG-MT-MA=dG=dG=dT=dA=dT=dG=dG=dA=dG=dA=MA-M5C-M5C-M5C-MT |
| 375 | CO-11565 | CTGTAGGTATGGAGAACCCT | 837 | M5C=MT-MG=MT-MA=dG=dG=dT=dA=dT=dG=dG=dA=dG=dA=MA-M5C=M5C-M5C=MT |
| 376 | CO-11566 | CTGTAGGTATGGAGAACCCT | 838 | M5C-MT=MG-MT=MA=dG=dG=dT=dA=dT=dG=dG=dA=dG=dA=MA=M5C-M5C=M5C-MT |
| 377 | CO-11567 | CTGTAGGTATGGAGAACCCT | 839 | M5C=MT=MG=MT=MA=dG=dG=MT=dA=dT=MG=dG=dA=MG=dA=MA=M5C=M5C=M5C=MT |
| 378 | CO-11568 | TCTGTAGGTATGGAGAACCCTT | 840 | MT=M5C=MT=MG=MT=MA=dG=dG=dT=dA=dT=dG=dG=dA=dG=dA=MA=M5C=M5C=M5C=MT=MT |
| 379 | CO-11569 | TCTGTAGGTATGGAGAACCCTT | 841 | LT=M5C=MT=LG=MT=MA=dG=dG=dT=dA=dT=dG=dG=dA=dG=dA=MA=M5C=L5C=M5C=MT=LT |
| 380 | CO-11570 | TCTGTAGGTATGGAGAACCCTT | 842 | MT=M5C=LT=MG=LT=MA=dG=dG=dT=dA=dT=dG=dG=dA=dG=dA=MA=L5C=M5C=L5C=MT=MT |
| 381 | CO-11571 | TCTGTAGGTATGGAGAACCCTT | 843 | MT-M5C=MT-MG=MT-MA=dG=dG=dT=dA=dT=dG=dG=dA=dG=dA=MA-M5C=M5C-M5C=MT-MT |
| 382 | CO-11572 | TCTGTAGGTATGGAGAACCCTT | 844 | MT=M5C-MT=MG-MT=MA=dG=dG=dT=dA=dT=dG=dG=dA=dG=dA=MA=M5C-M5C=M5C-MT=MT |
| 383 | CO-11573 | TCTGTAGGTATGGAGAACCCTT | 845 | MT=M5C-MT=MG=MT-MA=dG=dG=dT=dA=dT=dG=dG=dA=dG=dA=MA-M5C=M5C=M5C-MT=MT |
| 384 | CO-11574 | ATCTGTAGGTATGGAGAACCCTTT | 846 | MA=MT=M5C=MT=MG=MT=MA=dG=dG=dT=dA=dT=dG=dG=dA=dG=dA=MA=M5C=M5C=M5C=MT=MT=MT |
| 385 | CO-11575 | ATCTGTAGGTATGGAGAACCCTTT | 847 | LA=MT=M5C=LT=MG=MT=MA=dG=dG=dT=dA=dT=dG=dG=dA=dG=dA=MA=M5C=M5C=L5C=MT=MT=LT |
| 386 | CO-11576 | ATCTGTAGGTATGGAGAACCCTTT | 848 | MA=MT=L5C=MT=MG=LT=MA=dG=dG=dT=dA=dT=dG=dG=dA=dG=dA=MA=L5C=M5C=M5C=LT=MT=MT |
| 387 | CO-11577 | ATCTGTAGGTATGGAGAACCCTTT | 849 | MA=MT-M5C=MT-MG=MT-MA=dG=dG=dT=dA=dT=dG=dG=dA=dG=dA=MA-M5C=M5C-M5C=MT-MT=MT |
| 388 | CO-11578 | ATCTGTAGGTATGGAGAACCCTTT | 850 | MA-MT=M5C-MT=MG-MT=MA=dG=dG=dT=dA=dT=dG=dG=dA=dG=dA=MA=M5C-M5C=M5C-MT=MT-MT |
| 389 | CO-11579 | ATCTGTAGGTATGGAGAACCCTTT | 851 | MA=MT-M5C=MT-MG=MT=MA=dG=dG=dT=dA=dT=dG=dG=dA=dG=dA=MA=M5C=M5C-M5C=MT-MT=MT |
| 390 | CO-12331 | CTTTCTCTGTGGGTCTAGGG | 852 | M5C=MT=MT=LT=M5C=dT=d5C=dT=dG=dT=dG=dG=dG=dT=d5C=MT=LA=MG=MG=MG |
| 391 | CO-12332 | CTTTCTCTGTGGGTCTAGGG | 853 | M5C=MT=LT=MT=M5C=dT=d5C=dT=dG=dT=dG=dG=dG=dT=d5C=MT=MA=LG=MG=MG |
| 392 | CO-12333 | CTTTCTCTGTGGGTCTAGGG | 854 | M5C=LT=MT=MT=M5C=dT=d5C=dT=dG=dT=dG=dG=dG=dT=d5C=MT=MA=MG=LG=MG |
| 393 | CO-12334 | CTTTCTCTGTGGGTCTAGGG | 855 | M5C=LT=MT=LT=M5C=dT=d5C=dT=dG=dT=dG=dG=dG=dT=d5C=MT=LA=MG=LG=MG |
| 394 | CO-12335 | CTTTCTCTGTGGGTCTAGGG | 856 | L5C=MT=MT=LT=M5C=dT=d5C=dT=dG=dT=dG=dG=dG=dT=d5C=MT=LA=MG=MG=LG |
| 395 | CO-12336 | CTTTCTCTGTGGGTCTAGGG | 857 | L5C=MT=LT=MT=M5C=dT=d5C=dT=dG=dT=dG=dG=dG=dT=d5C=MT=MA=LG=MG=LG |
| 396 | CO-12337 | CTTTCTCTGTGGGTCTAGGG | 858 | M5C=MT=LT=MT=L5C=dT=d5C=dT=dG=dT=dG=dG=dG=dT=d5C=LT=MA=LG=MG=MG |
| 397 | CO-12338 | CTTTCTCTGTGGGTCTAGGG | 859 | M5C=MT=MT=MT=M5C=dT=d5C=MT=dG=dT=MG=dG=dG=MT=d5C=MT=MA=MG=MG=MG |
| 398 | CO-12339 | CTTTCTCTGTGGGTCTAGGG | 860 | M5C=LT=MT=LT=M5C=dT=d5C=MT=dG=dT=MG=dG=dG=MT=d5C=MT=LA=MG=LG=MG |
| 399 | CO-12340 | CTTTCTCTGTGGGTCTAGGG | 861 | M5C=MT=MT-MT-M5C=dT=d5C=dT=dG=dT=dG=dG=dG=dT=d5C=MT-MA-MG=MG=MG |
| 400 | CO-12341 | CTTTCTCTGTGGGTCTAGGG | 862 | M5C=MT-MT-MT-M5C=dT=d5C=dT=dG=dT=dG=dG=dG=dT=d5C=MT-MA-MG-MG=MG |
| 401 | CO-12342 | CTTTCTCTGTGGGTCTAGGG | 863 | M5C-MT-MT-MT-M5C=dT=d5C=dT=dG=dT=dG=dG=dG=dT=d5C=MT-MA-MG-MG-MG |
| 402 | CO-12343 | CTTTCTCTGTGGGTCTAGGG | 864 | M5C=MT-MT=MT-M5C=dT=d5C=dT=dG=dT=dG=dG=dG=dT=d5C=MT-MA=MG-MG=MG |
| 403 | CO-12344 | CTTTCTCTGTGGGTCTAGGG | 865 | M5C-MT=MT-MT=M5C=dT=d5C=dT=dG=dT=dG=dG=dG=dT=d5C=MT=MA-MG=MG-MG |
| 404 | CO-12345 | TCTTTCTCTGTGGGTCTAGGGT | 866 | MT=M5C=MT=MT=MT=M5C=dT=d5C=dT=dG=dT=dG=dG=dG=dT=d5C=MT=MA=MG=MG=MG=MT |
| 405 | CO-12346 | TCTTTCTCTGTGGGTCTAGGGT | 867 | LT=M5C=MT=LT=MT=M5C=dT=d5C=dT=dG=dT=dG=dG=dG=dT=d5C=MT=MA=LG=MG=MG=MT |
| 406 | CO-12347 | TCTTTCTCTGTGGGTCTAGGGT | 868 | MT=M5C=LT=MT=LT=M5C=dT=d5C=dT=dG=dT=dG=dG=dG=dT=d5C=MT=LA=MG=LG=MG=MT |
| 407 | CO-12348 | TCTTTCTCTGTGGGTCTAGGGT | 869 | MT-M5C=MT-MT=MT-M5C=dT=d5C=dT=dG=dT=dG=dG=dG=dT=d5C=MT-MA=MG-MG=MG-MT |
| 408 | CO-12349 | TCTTTCTCTGTGGGTCTAGGGT | 870 | MT=M5C-MT=MT-MT=M5C=dT=d5C=dT=dG=dT=dG=dG=dG=dT=d5C=MT=MA-MG=MG-MG=MT |
| 409 | CO-12350 | TCTTTCTCTGTGGGTCTAGGGT | 871 | MT=M5C-MT=MT=MT-M5C=dT=d5C=dT=dG=dT=dG=dG=dG=dT=d5C=MT-MA=MG=MG-MG=MT |
| 410 | CO-12351 | ATCTTTCTCTGTGGGTCTAGGGTC | 872 | MA=MT=M5C=MT=MT=MT=M5C=dT=d5C=dT=dG=dT=dG=dG=dG=dT=d5C=MT=MA=MG=MG=MG=MT=M5C |
| 411 | CO-12352 | ATCTTTCTCTGTGGGTCTAGGGTC | 873 | LA=MT=M5C=LT=MT=MT=M5C=dT=d5C=dT=dG=dT=dG=dG=dG=dT=d5C=MT=MA=MG=LG=MG=MT=L5C |
| 412 | CO-12353 | ATCTTTCTCTGTGGGTCTAGGGTC | 874 | MA=MT=L5C=MT=MT=LT=M5C=dT=d5C=dT=dG=dT=dG=dG=dG=dT=d5C=MT=LA=MG=MG=LG=MT=M5C |
| 413 | CO-12354 | ATCTTTCTCTGTGGGTCTAGGGTC | 875 | MA=MT-M5C=MT-MT=MT-M5C=dT=d5C=dT=dG=dT=dG=dG=dG=dT=d5C=MT-MA=MG-MG=MG-MT=M5C |
| 414 | CO-12355 | ATCTTTCTCTGTGGGTCTAGGGTC | 876 | MA-MT=M5C-MT=MT-MT=M5C=dT=d5C=dT=dG=dT=dG=dG=dG=dT=d5C=MT=MA-MG=MG-MG=MT-M5C |
| 415 | CO-12356 | ATCTTTCTCTGTGGGTCTAGGGTC | 877 | MA=MT-M5C=MT-MT=MT=M5C=dT=d5C=dT=dG=dT=dG=dG=dG=dT=d5C=MT=MA=MG-MG=MG-MT=M5C |
| 416 | CO-15399 | GGACCTCTGTCTCTCTCTGG | 878 | MG=MG=MA=M5C=M5C=dT=d5C=dT=dG=dT=d5C=dT=d5C=dT=d5C=MT=M5C=MT=MG=MG=dSp=dSp= |
| | | | | dSp=dSp=dSp=dSp=dSp=dSp=dSp=dSp=dSp=dSp=dSp=dSp=dSp=dSp=dSp=dSp=dSp=dSp= |
| 417 | CO-15400 | CTGTAGGTATGGAGAACCCT | 879 | M5C=MT=MG=MT=MA=dG=dG=dT=dA=dT=dG=dG=dA=dG=dA=MA=M5C=M5C=M5C=MT=dSp=dSp=dSp= |
| | | | | dSp=dSp=dSp=dSp=dSp=dSp=dSp=dSp=dSp=dSp=dSp=dSp=dsp=dSp=dSp=dSp=dSp |
| 418 | CO-15523 | CTGTAGGTATGGAGAACCCT | 880 | M5C=MT=MG=MT=LA=dG=dG=dT=dA=dT=dG=dG=dA=dG=dA=LA=M5C=M5C=M5C=MT |
| 419 | CO-15524 | CTGTAGGTATGGAGAACCCT | 881 | M5C=MT=LG=MT=MA=dG=dG=dT=dA=dT=dG=dG=dA=dG=dA=MA=M5C=L5C=M5C=MT |
| 420 | CO-15525 | CTGTAGGTATGGAGAACCCT | 882 | L5C=MT=MG=MT=MA=dG=dG=dT=dA=dT=dG=dG=dA=dG=dA=MA=M5C=M5C=M5C=LT |
| 421 | CO-15526 | CTGTAGGTATGGAGAACCCT | 883 | M5C=MT=LG=MT=LA=dG=dG=dT=dA=dT=dG=dG=dA=dG=dA=LA=M5C=L5C=M5C=MT |
| 422 | CO-15527 | CTGTAGGTATGGAGAACCCT | 884 | L5C=MT=LG=MT=MA=dG=dG=dT=dA=dT=dG=dG=dA=dG=dA=MA=M5C=L5C=M5C=LT |
| 423 | CO-15528 | CTGTAGGTATGGAGAACCCT | 885 | L5C=MT=LG=MT=LA=dG=dG=dT=dA=dT=dG=dG=dA=dG=dA=LA=M5C=L5C=M5C=LT |
| 424 | CO-15529 | GGACCTCTGTCTCTCTCTGG | 886 | MG=MG=MA=M5C=L5C=dT=d5C=dT=dG=dT=d5C=dT=d5C=dT=d5C=LT=M5C=MT=MG=MG |
| 425 | CO-15530 | GGACCTCTGTCTCTCTCTGG | 887 | MG=MG=LA=M5C=M5C=dT=d5C=dT=dG=dT=d5C=dT=d5C=dT=d5C=MT=M5C=LT=MG=MG |
| 426 | CO-15531 | GGACCTCTGTCTCTCTCTGG | 888 | LG=MG=MA=M5C=M5C=dT=d5C=dT=dG=dT=d5C=dT=d5C=dT=d5C=MT=M5C=MT=MG=LG |
| 427 | CO-15532 | GGACCTCTGTCTCTCTCTGG | 889 | MG=MG=LA=M5C=L5C=dT=d5C=dT=dG=dT=d5C=dT=d5C=dT=d5C=LT=M5C=LT=MG=MG |
| 428 | CO-15533 | GGACCTCTGTCTCTCTCTGG | 890 | LG=MG=LA=M5C=M5C=dT=d5C=dT=dG=dT=d5C=dT=d5C=dT=d5C=MT=M5C=LT=MG=LG |
| 429 | CO-15534 | GGACCTCTGTCTCTCTCTGG | 891 | LG=MG=LA=M5C=L5C=dT=d5C=dT=dG=dT=d5C=dT=d5C=dT=d5C=LT=M5C=LT=MG=LG |
| 430 | CO-16535 | GGCTCTTATTCTCCTCCTCC | 892 | MG=MG=M5C=MT=M5C=MT=MT=MA=MT=MT=M5C=MT=M5C=M5C=MT=M5C=M5C=MT=M5C=M5C |
| 431 | CO-16536 | TTGGCTCTTATTCTCCTCCT | 893 | MT=MT=MG=MG=M5C=MT=M5C=MT=MT=MA=MT=MT=M5C=MT=M5C=M5C=MT=M5C=M5C=MT |
| 432 | CO-16537 | CCGTTGGCTCTTATTCTCCT | 894 | M5C=M5C=MG=MT=MT=MG=MG=M5C=MT=M5C=MT=MT=MA=MT=MT=M5C=MT=M5C=M5C=MT |
| 433 | CO-16538 | TGCCGTTGGCTCTTATTCTC | 895 | MT=MG=M5C=M5C=MG=MT=MT=MG=MG=M5C=MT=M5C=MT=MT=MA=MT=MT=M5C=MT=M5C |
| 434 | CO-16539 | GCTGCCGTTGGCTCTTATTC | 896 | MG=M5C=MT=MG=M5C=M5C=MG=MT=MT=MG=MG=M5C=MT=M5C=MT=MT=MA=MT=MT=M5C |
| 435 | CO-16540 | CTGCTGCCGTTGGCTCTTAT | 897 | M5C=MT=MG=M5C=MT=MG=M5C=M5C=MG=MT=MT=MG=MG=M5C=MT=M5C=MT=MT=MA=MT |
| 436 | CO-16541 | CGCTGCTGCCGTTGGCTCTT | 898 | M5C=MG=M5C=MT=MG=M5C=MT=MG=M5C=M5C=MG=MT=MT=MG=MG=M5C=MT=M5C=MT=MT |
| 437 | CO-16542 | GGCTCTTATTCTCCTCCTCC | 899 | MG=MG=M5C=MT=M5C=dT=dT=dA=dT=dT=d5C=dT=d5C=d5C=dT=M5C=M5C=MT=M5C=M5C |
| 438 | CO-16543 | TTGGCTCTTATTCTCCTCCT | 900 | MT=MT=MG=MG=M5C=dT=d5C=dT=dT=dA=dT=dT=d5C=dT=d5C=M5C=MT=M5C=M5C=MT |
| 439 | CO-16544 | CCGTTGGCTCTTATTCTCCT | 901 | M5C=M5C=MG=MT=MT=dG=dG=d5C=dT=d5C=dT=dT=dA=dT=dT=M5C=MT=M5C=M5C=MT |
| 440 | CO-16545 | TGCCGTTGGCTCTTATTCTC | 902 | MT=MG=M5C=M5C=MG=dT=dT=dG=dG=d5C=dT=d5C=dT=dT=dA=MT=MT=M5C=MT=M5C |
| 441 | CO-16546 | GCTGCCGTTGGCTCTTATTC | 903 | MG=M5C=MT=MG=M5C=d5C=dG=dT=dT=dG=dG=d5C=dT=d5C=dT=MT=MA=MT=MT=M5C |
| 442 | CO-16547 | CTGCTGCCGTTGGCTCTTAT | 904 | M5C=MT=MG=M5C=MT=dG=d5C=d5C=dG=dT=dT=dG=dG=d5C=dT=M5C=MT=MT=MA=MT |
| 443 | CO-16548 | CGCTGCTGCCGTTGGCTCTT | 905 | M5C=MG=M5C=MT=MG=d5C=dT=dG=d5C=d5C=dG=dT=dT=dG=dG=M5C=MT=M5C=MT=MT |
| 444 | CO-17739 | CTGGTGTGTTTTTGAGTCAG | 906 | M5C=MT=MG=MG=MT=dG=dT=dG=dT=dT=dT=dT=dT=dG=dA=MG=MT=M5C=MA=MG |
| 445 | CO-17740 | TCTGGTGTGTTTTTGAGTCA | 907 | MT=M5C=MT=MG=MG=dT=dG=dT=dG=dT=dT=dT=dT=dT=dG=MA=MG=MT=M5C=MA |
| 446 | CO-17741 | TCTCTGGTGTGTTTTTGAGT | 908 | MT=M5C=MT=M5C=MT=dG=dG=dT=dG=dT=dG=dT=dT=dT=dT=MT=MG=MA=MG=MT |
| 447 | CO-17742 | ATCTTTAGGACCTCTGTCTC | 909 | MA=MT=M5C=MT=MT=dT=dA=dG=dG=dA=d5C=d5C=dT=d5C=dT=MG=MT=M5C=MT=M5C |
| 448 | CO-17743 | GGTCTGCATCTTTAGGACCT | 910 | MG=MG=MT=M5C=MT=dG=d5C=dA=dT=d5C=dT=dT=dT=dA=dG=MG=MA=M5C=M5C=MT |
| 449 | CO-17744 | GGGTCTGCATCTTTAGGACC | 911 | MG=MG=MG=MT=M5C=dT=dG=d5C=dA=dT=d5C=dT=dT=dT=dA=MG=MG=MA=M5C=M5C |
| 450 | CO-17745 | TCTAGGGTCTCTCTGTCTCA | 912 | MT=M5C=MT=MA=MG=dG=dG=dT=d5C=dT=d5C=dT=d5C=dT=dG=MT=M5C=MT=M5C=MA |
| 451 | CO-17746 | GGGTCTAGGGTCTCTCTGTC | 913 | MG=MG=MG=MT=M5C=dT=dA=dG=dG=dG=dT=d5C=dT=d5C=dT=M5C=MT=MG=MT=M5C |
| 452 | CO-17747 | GTGGGTCTAGGGTCTCTCTG | 914 | MG=MT=MG=MG=MG=dT=d5C=dT=dA=dG=dG=dG=dT=d5C=dT=M5C=MT=M5C=MT=MG |
| 453 | CO-17748 | TCTGTGGGTCTAGGGTCTCT | 915 | MT=M5C=MT=MG=MT=dG=dG=dG=dT=d5C=dT=dA=dG=dG=dG=MT=M5C=MT=M5C=MT |
| 454 | CO-17749 | TCTCTGTGGGTCTAGGGTCT | 916 | MT=M5C=MT=M5C=MT=dG=dT=dG=dG=dG=dT=d5C=dT=dA=dG=MG=MG=MT=M5C=MT |
| 455 | CO-17750 | TTTCTCTGTGGGTCTAGGGT | 917 | MT=MT=MT=M5C=MT=d5C=dT=dG=dT=dG=dG=dG=dT=d5C=dT=MA=MG=MG=MG=MT |
| 456 | CO-17751 | TGAAGTATCTTTCTCTGTGG | 918 | MT=MG=MA=MA=MG=dT=dA=dT=d5C=dT=dT=dT=d5C=dT=d5C=MT=MG=MT=MG=MG |
| 457 | CO-17752 | GGTGAAGTATCTTTCTCTGT | 919 | MG=MG=MT=MG=MA=dA=dG=dT=dA=dT=d5C=dT=dT=dT=d5C=MT=M5C=MT=MG=MT |
| 458 | CO-17753 | GTGGTGAAGTATCTTTCTCT | 920 | MG=MT=MG=MG=MT=dG=dA=dA=dG=dT=dA=dT=d5C=dT=dT=MT=M5C=MT=M5C=MT |
| 459 | CO-17754 | TCAAATTACCCCACAGTGGG | 921 | MT=M5C=MA=MA=MA=dT=dT=dA=d5C=d5C=d5C=d5C=dA=d5C=dA=MG=MT=MG=MG=MG |
| 460 | CO-17755 | TCTCAAATTACCCCACAGTG | 922 | MT=M5C=MT=M5C=MA=dA=dA=dT=dT=dA=d5C=d5C=d5C=d5C=dA=M5C=MA=MG=MT=MG |
| 461 | CO-17756 | TGTCTCAAATTACCCCACAG | 923 | MT=MG=MT=M5C=MT=d5C=dA=dA=dA=dT=dT=dA=d5C=d5C=d5C=M5C=MA=M5C=MA=MG |
| 462 | CO-17757 | TGTGTCTCAAATTACCCCAC | 924 | MT=MG=MT=MG=MT=d5C=dT=d5C=dA=dA=dA=dT=dT=dA=d5C=M5C=M5C=M5C=MA=M5C |
| 463 | CO-17758 | CCTGTGTCTCAAATTACCCC | 925 | M5C=M5C=MT=MG=MT=dG=dT=d5C=dT=d5C=dA=dA=dA=dT=dT=MA=M5C=M5C=M5C=M5C |
| 464 | CO-17759 | GCCCTGTGTCTCAAATTACC | 926 | MG=M5C=M5C=M5C=MT=dG=dT=dG=dT=d5C=dT=d5C=dA=dA=dA=MT=MT=MA=M5C=M5C |
| 465 | CO-17760 | TTTGGCCCTGTGTCTCAAAT | 927 | MT=MT=MT=MG=MG=d5C=d5C=d5C=dT=dG=dT=dG=dT=d5C=dT=M5C=MA=MA=MA=MT |
| 466 | CO-17761 | ATTTTGGCCCTGTGTCTCAA | 928 | MA=MT=MT=MT=MT=dG=dG=d5C=d5C=d5C=dT=dG=dT=dG=dT=M5C=MT=M5C=MA=MA |
| 467 | CO-17762 | TCTAGATTTTGGCCCTGTGT | 929 | MT=M5C=MT=MA=MG=dA=dT=dT=dT=dT=dG=dG=d5C=d5C=d5C=MT=MG=MT=MG=MT |
| 468 | CO-17763 | CTCTAGATTTTGGCCCTGTG | 930 | M5C=MT=M5C=MT=MA=dG=dA=dT=dT=dT=dT=dG=dG=d5C=d5C=M5C=MT=MG=MT=MG |
| 469 | CO-17764 | TCCTCTAGATTTTGGCCCTG | 931 | MT=M5C=M5C=MT=M5C=dT=dA=dG=dA=dT=dT=dT=dT=dG=dG=M5C=M5C=M5C=MT=MG |
| 470 | CO-17765 | TTTTCCTCTAGATTTTGGCC | 932 | MT=MT=MT=MT=M5C=d5C=dT=d5C=dT=dA=dG=dA=dT=dT=dT=MT=MG=MG=M5C=M5C |
| 471 | CO-17766 | TTTTAGACTGGATTTTCAGA | 933 | MT=MT=MT=MT=MA=dG=dA=d5C=dT=dG=dG=dA=dT=dT=dT=MT=M5C=MA=MG=MA |
| 472 | CO-17767 | CCCTTTTTAGACTGGATTTT | 934 | M5C=M5C=M5C=MT=MT=dT=dT=dT=dA=dG=dA=d5C=dT=dG=dG=MA=MT=MT=MT=MT |
| 473 | CO-17768 | AACCCTTTTTAGACTGGATT | 935 | MA=MA=M5C=M5C=M5C=dT=dT=dT=dT=dT=dA=dG=dA=d5C=dT=MG=MG=MA=MT=MT |
| 474 | CO-17769 | GAACCCTTTTTAGACTGGAT | 936 | MG=MA=MA=M5C=M5C=d5C=dT=dT=dT=dT=dT=dA=dG=dA=d5C=MT=MG=MG=MA=MT |
| 475 | CO-17770 | GAGAACCCTTTTTAGACTGG | 937 | MG=MA=MG=MA=MA=d5C=d5C=d5C=dT=dT=dT=dT=dT=dA=dG=MA=M5C=MT=MG=MG |
| 476 | CO-17771 | GGAGAACCCTTTTTAGACTG | 938 | MG=MG=MA=MG=MA=dA=d5C=d5C=d5C=dT=dT=dT=dT=dT=dA=MG=MA=M5C=MT=MG |
| 477 | CO-17772 | ATGGAGAACCCTTTTTAGAC | 939 | MA=MT=MG=MG=MA=dG=dA=dA=d5C=d5C=d5C=dT=dT=dT=dT=MT=MA=MG=MA=M5C |
| 478 | CO-17773 | TATGGAGAACCCTTTTTAGA | 940 | MT=MA=MT=MG=MG=dA=dG=dA=dA=d5C=d5C=d5C=dT=dT=dT=MT=MT=MA=MG=MA |
| 479 | CO-17774 | GGTATGGAGAACCCTTTTTA | 941 | MG=MG=MT=MA=MT=dG=dG=dA=dG=dA=dA=d5C=d5C=d5C=dT=MT=MT=MT=MT=MA |
| 480 | CO-17775 | AGGTATGGAGAACCCTTTTT | 942 | MA=MG=MG=MT=MA=dT=dG=dG=dA=dG=dA=dA=d5C=d5C=d5C=MT=MT=MT=MT=MT |
| 481 | CO-17776 | TGTAGGTATGGAGAACCCTT | 943 | MT=MG=MT=MA=MG=dG=dT=dA=dT=dG=dG=dA=dG=dA=dA=M5C=M5C=M5C=MT=MT |
| 482 | CO-17777 | ATATCTGTAGGTATGGAGAA | 944 | MA=MT=MA=MT=M5C=dT=dG=dT=dA=dG=dG=dT=dA=dT=dG=MG=MA=MG=MA=MA |
| 483 | CO-17778 | AGATATCTGTAGGTATGGAG | 945 | MA=MG=MA=MT=MA=dT=d5C=dT=dG=dT=dA=dG=dG=dT=dA=MT=MG=MG=MA=MG |
| 484 | CO-17779 | AAGATATCTGTAGGTATGGA | 946 | MA=MA=MG=MA=MT=dA=dT=d5C=dT=dG=dT=dA=dG=dG=dT=MA=MT=MG=MG=MA |
| 485 | CO-17780 | CTAAGATATCTGTAGGTATG | 947 | M5C=MT=MA=MA=MG=dA=dT=dA=dT=d5C=dT=dG=dT=dA=dG=MG=MT=MA=MT=MG |
| 486 | CO-17781 | TCTAAGATATCTGTAGGTAT | 948 | MT=M5C=MT=MA=MA=dG=dA=dT=dA=dT=d5C=dT=dG=dT=dA=MG=MG=MT=MA=MT |
| 487 | CO-17782 | GTCTAAGATATCTGTAGGTA | 949 | MG=MT=M5C=MT=MA=dA=dG=dA=dT=dA=dT=d5C=dT=dG=dT=MA=MG=MG=MT=MA |
| 488 | CO-17783 | GAGTCTAAGATATCTGTAGG | 950 | MG=MA=MG=MT=M5C=dT=dA=dA=dG=dA=dT=dA=dT=d5C=dT=MG=MT=MA=MG=MG |
| 489 | CO-17784 | TGGAGTCTAAGATATCTGTA | 951 | MT=MG=MG=MA=MG=dT=d5C=dT=dA=dA=dG=dA=dT=dA=dT=M5C=MT=MG=MT=MA |
| 490 | CO-17785 | CTGGAGTCTAAGATATCTGT | 952 | M5C=MT=MG=MG=MA=dG=dT=d5C=dT=dA=dA=dG=dA=dT=dA=MT=M5C=MT=MG=MT |
| 491 | CO-17786 | TCTGGAGTCTAAGATATCTG | 953 | MT=M5C=MT=MG=MG=dA=dG=dT=d5C=dT=dA=dA=dG=dA=dT=MA=MT=M5C=MT=MG |
| 492 | CO-17787 | GTCTGGAGTCTAAGATATCT | 954 | MG=MT=M5C=MT=MG=dG=dA=dG=dT=d5C=dT=dA=dA=dG=dA=MT=MA=MT=M5C=MT |
| 493 | CO-17788 | GGTCTGGAGTCTAAGATATC | 955 | MG=MG=MT=M5C=MT=dG=dG=dA=dG=dT=d5C=dT=dA=dA=dG=MA=MT=MA=MT=M5C |
| 494 | CO-17789 | GGGTCTGGAGTCTAAGATAT | 956 | MG=MG=MG=MT=M5C=dT=dG=dG=dA=dG=dT=d5C=dT=dA=dA=MG=MA=MT=MA=MT |
| 495 | CO-18344 | CTTTCTCTGTGGGTCTAGGG | 957 | M5C=MT=MT=LT=M5C=dT=d5C=dT=dG=dT=dG=dG=dG=dT=d5C=MT=MA=MG=MG=MG |
| 496 | CO-18345 | CTTTCTCTGTGGGTCTAGGG | 958 | M5C=MT=MT=MT=M5C=dT=d5C=dT=dG=dT=dG=dG=dG=dT=d5C=MT=LA=MG=MG=MG |
| 497 | CO-18346 | CTTTCTCTGTGGGTCTAGGG | 959 | M5C=MT=MT=MT=L5C=dT=d5C=dT=dG=dT=dG=dG=dG=dT=d5C=LT=MA=MG=MG=MG |
| 498 | CO-18347 | CTTTCTCTGTGGGTCTAGGG | 960 | M5C=MT=MT=MT=L5C=dT=d5C=dT=dG=dT=dG=dG=dG=dT=d5C=MT=MA=MG=MG=MG |
| 499 | CO-18348 | CTTTCTCTGTGGGTCTAGGG | 961 | M5C=MT=MT=MT=M5C=dT=d5C=dT=dG=dT=dG=dG=dG=dT=d5C=LT=MA=MG=MG=MG |
| 500 | CO-18349 | CTTTCTCTGTGGGTCTAGGG | 962 | M5C=MT=MT=LT=L5C=dT=d5C=dT=dG=dT=dG=dG=dG=dT=d5C=MT=MA=MG=MG=MG |
| 501 | CO-18350 | CTTTCTCTGTGGGTCTAGGG | 963 | M5C=MT=MT=MT=M5C=dT=d5C=dT=dG=dT=dG=dG=dG=dT=d5C=LT=LA=MG=MG=MG |
| 502 | CO-18351 | CTTTCTCTGTGGGTCTAG | 964 | M5C=MT=MT=MT=M5C=dT=d5C=dT=dG=dT=dG=dG=dG=dT=d5C=MT=MA=MG |
| 503 | CO-18352 | CTTTCTCTGTGGGTCTAG | 965 | M5C=MT=MT=LT=M5C=dT=d5C=dT=dG=dT=dG=dG=dG=dT=d5C=MT=LA=MG |
| 504 | CO-18353 | CTTTCTCTGTGGGTCTAG | 966 | M5C=MT=MT=LT=M5C=dT=d5C=dT=dG=dT=dG=dG=dG=dT=d5C=MT=MA=MG |
| 505 | CO-18354 | CTTTCTCTGTGGGTCTAG | 967 | M5C=MT=MT=MT=M5C=dT=d5C=dT=dG=dT=dG=dG=dG=dT=d5C=MT=LA=MG |
| 506 | CO-18355 | CTTTCTCTGTGGGTCTAG | 968 | M5C=MT=MT=MT=L5C=dT=d5C=dT=dG=dT=dG=dG=dG=dT=d5C=LT=MA=MG |
| 507 | CO-18356 | CTTTCTCTGTGGGTCTAG | 969 | M5C=MT=MT=MT=L5C=dT=d5C=dT=dG=dT=dG=dG=dG=dT=d5C=MT=MA=MG |
| 508 | CO-18357 | CTTTCTCTGTGGGTCTAG | 970 | M5C=MT=MT=MT=M5C=dT=d5C=dT=dG=dT=dG=dG=dG=dT=d5C=LT=MA=MG |
| 509 | CO-18358 | CTTTCTCTGTGGGTCTAG | 971 | M5C=MT=MT=LT=L5C=dT=d5C=dT=dG=dT=dG=dG=dG=dT=d5C=MT=MA=MG |
| 510 | CO-18359 | CTTTCTCTGTGGGTCTAG | 972 | M5C=MT=MT=MT=M5C=dT=d5C=dT=dG=dT=dG=dG=dG=dT=d5C=LT=LA=MG |
| 511 | CO-18360 | TCTTTCTCTGTGGGTCTAGG | 973 | MT=M5C=MT=MT=MT=M5C=dT=d5C=dT=dG=dT=dG=dG=dG=dT=d5C=MT=MA=MG=MG |
| 512 | CO-18361 | ATCTTTCTCTGTGGGTCTAG | 974 | MA=MT=M5C=MT=MT=MT=M5C=dT=d5C=dT=dG=dT=dG=dG=dG=dT=d5C=MT=MA=MG |
| 513 | CO-18362 | TTTCTCTGTGGGTCTAGGGT | 975 | MT=MT=MT=M5C=dT=d5C=dT=dG=dT=dG=dG=dG=dT=d5C=MT=MA=MG=MG=MG=MT |
| 514 | CO-18363 | TTCTCTGTGGGTCTAGGGTC | 976 | MT=MT=M5C=dT=d5C=dT=dG=dT=dG=dG=dG=dT=d5C=MT=MA=MG=MG=MG=MT=M5C |
| 515 | CO-18365 | GACCTCTGTCTCTCTCTG | 977 | MG=MA=M5C=M5C=dT=d5C=dT=dG=dT=d5C=dT=d5C=dT=d5C=MT=M5C=MT=MG |
| 516 | CO-18366 | TTAGGACCTCTGTCTCTCTCTGGTGT | 978 | MT=MT=MA=MG=MG=MA=M5C=M5C=dT=d5C=dT=dG=dT=d5C=dT=d5C=dT=d5C=MT=M5C=MT=MG=MG= |
| | | | | MT=MG=MT |
| 517 | CO-18367 | AGGACCTCTGTCTCTCTCTGGT | 979 | MA=MG=MG=MA-M5C=M5C=dT=d5C=dT=dG=dT=d5C=dT=d5C=dT=d5C=MT=M5C-MT=MG=MG=MT |
| 518 | CO-18368 | AGGACCTCTGTCTCTCTCTGGT | 980 | MA=MG=MG=MA=M5C=M5C=dT=d5C=dT=dG=dT=d5C=dT=d5C=dT=d5C=MT=M5C-MT=MG=MG=MT |
| 519 | CO-18369 | AGGACCTCTGTCTCTCTCTGGT | 981 | MA=MG=MG=MA-M5C=M5C=dT=d5C=dT=dG=dT=d5C=dT=d5C=dT=d5C=MT=M5C=MT=MG=MG=MT |
| 520 | CO-18370 | AGGACCTCTGTCTCTCTCTGGT | 982 | MA=MG=MG=MA=M5C-M5C=dT=d5C=dT=dG=dT=d5C=dT=d5C=dT=d5C=MT-M5C=MT=MG=MG=MT |
| 521 | CO-18371 | AGGACCTCTGTCTCTCTCTGGT | 983 | MA=MG=MG=MA=M5C=M5C=dT=d5C=dT=dG=dT=d5C=dT=d5C=dT=d5C=MT-M5C=MT=MG=MG=MT |
| 522 | CO-18372 | AGGACCTCTGTCTCTCTCTGGT | 984 | MA=MG=MG=MA=M5C-M5C=dT=d5C=dT=dG=dT=d5C=dT=d5C=dT=d5C=MT=M5C=MT=MG=MG=MT |
| 523 | CO-18373 | AGGACCTCTGTCTCTCTCTGGT | 985 | MA=MG=MG=MA=M5C=M5C-dT=d5C=dT=dG=dT=d5C=dT=d5C=dT=d5C-MT=M5C=MT=MG=MG=MT |
| 524 | CO-18374 | AGGACCTCTGTCTCTCTCTGGT | 986 | MA=MG=MG=MA=M5C=M5C=dT=d5C=dT=dG=dT=d5C=dT=d5C=dT=d5C-MT=M5C=MT=MG=MG=MT |
| 525 | CO-18375 | AGGACCTCTGTCTCTCTCTGGT | 987 | MA=MG=MG=MA=M5C=M5C-dT=d5C=dT=dG=dT=d5C=dT=d5C=dT=d5C=MT=M5C=MT=MG=MG=MT |
| 526 | CO-18376 | CAAGTGCCAGGCCCACATGG | 988 | M5C=MA=MA=MG=dT=dG=d5C=d5C=dA=dG=dG=d5C=d5C=d5C=MA=M5C=MA=MT=MG=MG |
| 527 | CO-18377 | GGCAAGTGCCAGGCCCACAT | 989 | MG=MG=M5C=MA=MA=MG=dT=dG=d5C=d5C=dA=dG=dG=d5C=d5C=d5C=MA=M5C=MA=MT |
| 528 | CO-18378 | GGGCAAGTGCCAGGCCCACA | 990 | MG=MG=MG=M5C=MA=MA=MG=dT=dG=d5C=d5C=dA=dG=dG=d5C=d5C=d5C=MA=M5C=MA |
| 529 | CO-18379 | TCTCTCTGGTGTGTTTTTGAGT | 991 | MT=M5C=MT=M5C=MT=M5C=dT=dG=dG=dT=dG=dT=dG=dT=dT=dT=MT=MT=MG=MA=MG=MT |
| 530 | CO-18380 | TCTCTCTGGTGTGTTTTTGAGT | 992 | MT=M5C=MT=M5C=MT=M5C=MT=MG=MG=MT=MG=MT=MG=MT=MT=MT=MT=MT=MG=MA=MG=MT |
| 531 | CO-18381 | GGCAAGTGCCAGGCCCACAT | 993 | MG=MG=M5C=MA=MA=dG=dT=dG=d5C=d5C=dA=dG=dG=d5C=d5C=M5C=MA=M5C=MA=MT |
| 532 | CO-18382 | TGAAGTATCTTTCTCTGTGG | 994 | MT=MG=MA=LA=MG=dT=dA=dT=d5C=dT=dT=dT=d5C=dT=d5C=MT=LG=MT=MG=MG |
| 533 | CO-18383 | TGAAGTATCTTTCTCTGTGG | 995 | MT=MG=MA=LA=MG=dT=dA=dT=d5C=dT=dT=dT=d5C=dT=d5C=MT=MG=MT=MG=MG |
| 534 | CO-18384 | TGAAGTATCTTTCTCTGTGG | 996 | MT=MG=MA=MA=MG=dT=dA=dT=d5C=dT=dT=dT=d5C=dT=d5C=MT=LG=MT=MG=MG |
| 535 | CO-18385 | TGAAGTATCTTTCTCTGTGG | 997 | MT=MG=MA=MA=LG=dT=dA=dT=d5C=dT=dT=dT=d5C=dT=d5C=LT=MG=MT=MG=MG |
| 536 | CO-18386 | TGAAGTATCTTTCTCTGTGG | 998 | MT=MG=MA=MA=LG=dT=dA=dT=d5C=dT=dT=dT=d5C=dT=d5C=MT=MG=MT=MG=MG |
| 537 | CO-18387 | TGAAGTATCTTTCTCTGTGG | 999 | MT=MG=MA=MA=MG=dT=dA=dT=d5C=dT=dT=dT=d5C=dT=d5C=LT=MG=MT=MG=MG |
| 538 | CO-18388 | TGAAGTATCTTTCTCTGTGG | 1000 | MT=MG=MA=LA=LG=dT=dA=dT=d5C=dT=dT=dT=d5C=dT=d5C=MT=MG=MT=MG=MG |
| 539 | CO-18389 | TGAAGTATCTTTCTCTGTGG | 1001 | MT=MG=MA=MA=MG=dT=dA=dT=d5C=dT=dT=dT=d5C=dT=d5C=LT=LG=MT=MG=MG |
| 540 | CO-18390 | GGACCTCTGTCTCTCTCTGGTGTGTTTTTGAG | 1002 | MG=MG=MA=M5C=M5C=dT=d5C=dT=dG=dT=d5C=dT=d5C=dT=d5C=MT=M5C=MT=MG=MG=MT=MG=MT= |
| | | | | MG=MT=MT=MT=MT=MT=MG=MA=MG |
| 541 | CO-18391 | TCTCTGGTGTGTTTTTGAG | 1003 | MT=M5C=MT=M5C=MT=MG=MG=MT=MG=MT=MG=MT=MT=MT=MT=MT=MG=MA=MG |

TABLE 3
Exemplary SYNGAP1 ASO sequences that target RR86_v1 and/or RR86_v2

| SEQ | | SEQ | | SEQ | |
|---|---|---|---|---|---|
| ID NO | Sequence | ID NO | Sequence | ID NO | Sequence |
| 1004 | TACCGGGGGCGGGTGCTACA | 1657 | TGGTAAGTACCCAACTAATCT | 2310 | ATTAGCTGAGTGACGCTTCCAGCC |
| 1005 | AGGACCTACCGGGGGGGGT | 1658 | GGTAAGTACCCAACTAATCTC | 2311 | TTAGCTGAGTGACGCTTCCAGCCC |
| 1006 | CCCCTCTCAGGACCTACCGG | 1659 | GTAAGTACCCAACTAATCTCG | 2312 | TAGCTGAGTGACGCTTCCAGCCCG |
| 1007 | GGGCCTGTGCCCTCCTACTA | 1660 | TAAGTACCCAACTAATCTCGC | 2313 | AGCTGAGTGACGCTTCCAGCCCGT |
| 1008 | AGGACACCCTGGGCCTGTGC | 1661 | AAGTACCCAACTAATCTCGCC | 2314 | GCTGAGTGACGCTTCCAGCCCGTT |
| 1009 | TTGCGAGGACACCCTGGGCC | 1662 | AGTACCCAACTAATCTCGCCC | 2315 | CTGAGTGACGCTTCCAGCCCGTTT |
| 1010 | GCCCGTTGCGAGGACACCCT | 1663 | GTACCCAACTAATCTCGCCCC | 2316 | TGAGTGACGCTTCCAGCCCGTTTC |
| 1011 | GGGACGCCCGTTGCGAGGAC | 1664 | TACCCAACTAATCTCGCCCCT | 2317 | GAGTGACGCTTCCAGCCCGTTTCC |
| 1012 | TCCCCGGGACGCCCGTTGCG | 1665 | ACCCAACTAATCTCGCCCCTC | 2318 | AGTGACGCTTCCAGCCCGTTTCCC |
| 1013 | TTCAGCAGCTCCCCGGGACG | 1666 | CCCAACTAATCTCGCCCCTCT | 2319 | GTGACGCTTCCAGCCCGTTTCCCC |
| 1014 | AGGACTTCAGCAGCTCCCCG | 1667 | CCAACTAATCTCGCCCCTCTC | 2320 | TGACGCTTCCAGCCCGTTTCCCCC |
| 1015 | CGGGGAGGACTTCAGCAGCT | 1668 | CAACTAATCTCGCCCCTCTCA | 2321 | GACGCTTCCAGCCCGTTTCCCCCG |
| 1016 | TCTGCAGCGGGGAGGACTTC | 1669 | AACTAATCTCGCCCCTCTCAG | 2322 | ACGCTTCCAGCCCGTTTCCCCCGC |
| 1017 | CGGTCCTCTGCAGCGGGGAG | 1670 | ACTAATCTCGCCCCTCTCAGG | 2323 | CGCTTCCAGCCCGTTTCCCCCGCC |
| 1018 | CCTCTCGGTCCTCTGCAGCG | 1671 | CTAATCTCGCCCCTCTCAGGA | 2324 | GCTTCCAGCCCGTTTCCCCCGCCT |
| 1019 | GCCCTGCCTCTCGGTCCTCT | 1672 | TAATCTCGCCCCTCTCAGGAC | 2325 | CTTCCAGCCCGTTTCCCCCGCCTG |
| 1020 | CCCGGGCCCTGCCTCTCGGT | 1673 | AATCTCGCCCCTCTCAGGACC | 2326 | TTCCAGCCCGTTTCCCCCGCCTGG |
| 1021 | TGTTCTCTCCCGGGCCCTGC | 1674 | ATCTCGCCCCTCTCAGGACCT | 2327 | TCCAGCCCGTTTCCCCCGCCTGGA |
| 1022 | CGGGCTGTTCTCTCCCGGGC | 1675 | TCTCGCCCCTCTCAGGACCTA | 2328 | CCAGCCCGTTTCCCCCGCCTGGAA |
| 1023 | TCCGACCGGGCTGTTCTCTC | 1676 | CTCGCCCCTCTCAGGACCTAC | 2329 | CAGCCCGTTTCCCCCGCCTGGAAA |
| 1024 | CACCCCCTCCGACCGGGCTG | 1677 | TCTTTTAAAAATTACCTAGAAA | 2330 | AGCCCGTTTCCCCCGCCTGGAAAC |
| 1025 | GACGGCCCACCCCCTCCGAC | 1678 | TTAAAAATTACCTAGAAATAAG | 2331 | GCCCGTTTCCCCCGCCTGGAAACT |
| 1026 | CGGCTGACGGCCCACCCCCT | 1679 | TAAAAATTACCTAGAAATAAGG | 2332 | CCCGTTTCCCCCGCCTGGAAACTG |
| 1027 | TTTCCGGGGCGGCTGACGGC | 1680 | AAAAATTACCTAGAAATAAGGT | 2333 | CCGTTTCCCCCGCCTGGAAACTGA |
| 1028 | GCAGCTTTCCGGGGCGGCTG | 1681 | AAAATTACCTAGAAATAAGGTT | 2334 | CGTTTCCCCCGCCTGGAAACTGAG |
| 1029 | GAAACGCAGCTTTCCGGGGC | 1682 | AAATTACCTAGAAATAAGGTTT | 2335 | GTTTCCCCCGCCTGGAAACTGAGG |
| 1030 | ATCACCGGGAAACGCAGCTT | 1683 | AATTACCTAGAAATAAGGTTTC | 2336 | TTTCCCCCGCCTGGAAACTGAGGA |
| 1031 | ACTTAATCACCGGGAAACGC | 1684 | ATTACCTAGAAATAAGGTTTCT | 2337 | TTCCCCCGCCTGGAAACTGAGGAC |
| 1032 | GTGCTACACTTAATCACCGG | 1685 | TTACCTAGAAATAAGGTTTCTT | 2338 | TCCCCCGCCTGGAAACTGAGGACG |
| 1033 | GGCGGGTGCTACACTTAATC | 1686 | TACCTAGAAATAAGGTTTCTTT | 2339 | CCCCCGCCTGGAAACTGAGGACGG |
| 1034 | CTAATCTCGCCCCTCTCAGG | 1687 | ACCTAGAAATAAGGTTTCTTTA | 2340 | CCCCGCCTGGAAACTGAGGACGGC |
| 1035 | AGTACCCAACTAATCTCGCC | 1688 | CCTAGAAATAAGGTTTCTTTAA | 2341 | CCCGCCTGGAAACTGAGGACGGCA |
| 1036 | TGGTAAGTACCCAACTAATC | 1689 | CTAGAAATAAGGTTTCTTTAAT | 2342 | CCGCCTGGAAACTGAGGACGGCAA |
| 1037 | GCCCACATGGTAAGTACCCA | 1690 | TAGAAATAAGGTTTCTTTAATT | 2343 | CGCCTGGAAACTGAGGACGGCAAC |
| 1038 | GCCAGGCCCACATGGTAAGT | 1691 | AGAAATAAGGTTTCTTTAATTG | 2344 | GCCTGGAAACTGAGGACGGCAACC |
| 1039 | GATGTGGGCAAGTGCCAGGC | 1692 | GAAATAAGGTTTCTTTAATTGC | 2345 | CCTGGAAACTGAGGACGGCAACCC |
| 1040 | AAAGTGATGTGGGCAAGTGC | 1693 | AAATAAGGTTTCTTTAATTGCT | 2346 | CTGGAAACTGAGGACGGCAACCCC |
| 1041 | GACACAAAGTGATGTGGGCA | 1694 | AATAAGGTTTCTTTAATTGCTG | 2347 | TGGAAACTGAGGACGGCAACCCCC |
| 1042 | CCGGTGGGAAGACACAAAGT | 1695 | ATAAGGTTTCTTTAATTGCTGG | 2348 | GGAAACTGAGGACGGCAACCCCCA |
| 1043 | CGCAGGGCCGGTGGGAAGAC | 1696 | TAAGGTTTCTTTAATTGCTGGT | 2349 | GAAACTGAGGACGGCAACCCCCAC |
| 1044 | ACCCTCGCAGGGCCGGTGGG | 1697 | AAGGTTTCTTTAATTGCTGGTT | 2350 | AAACTGAGGACGGCAACCCCCACC |
| 1045 | CCCCCACCCTCGCAGGGCCG | 1698 | AGGTTTCTTTAATTGCTGGTTA | 2351 | AACTGAGGACGGCAACCCCCACCC |
| 1046 | GGCAACCCCCACCCTCGCAG | 1699 | GGTTTCTTTAATTGCTGGTTAA | 2352 | ACTGAGGACGGCAACCCCCACCCT |
| 1047 | TGAGGACGGCAACCCCCACC | 1700 | GTTTCTTTAATTGCTGGTTAAA | 2353 | CTGAGGACGGCAACCCCCACCCTC |
| 1048 | GGAAACTGAGGACGGCAACC | 1701 | TTTCTTTAATTGCTGGTTAAAA | 2354 | TGAGGACGGCAACCCCCACCCTCG |
| 1049 | CGCCTGGAAACTGAGGACGG | 1702 | TTCTTTAATTGCTGGTTAAAAT | 2355 | GAGGACGGCAACCCCCACCCTCGC |
| 1050 | TTCCCCCGCCTGGAAACTGA | 1703 | TCTTTAATTGCTGGTTAAAATG | 2356 | AGGACGGCAACCCCCACCCTCGCA |
| 1051 | GCCCGTTTCCCCCGCCTGGA | 1704 | CTTTAATTGCTGGTTAAAATGC | 2357 | GGACGGCAACCCCCACCCTCGCAG |
| 1052 | TTCCAGCCCGTTTCCCCCGC | 1705 | TTTAATTGCTGGTTAAAATGCT | 2358 | GACGGCAACCCCCACCCTCGCAGG |
| 1053 | AGTGACGCTTCCAGCCCGTT | 1706 | TTAATTGCTGGTTAAAATGCTC | 2359 | ACGGCAACCCCCACCCTCGCAGGG |
| 1054 | TTAGCTGAGTGACGCTTCCA | 1707 | TAATTGCTGGTTAAAATGCTCC | 2360 | CGGCAACCCCCACCCTCGCAGGGC |
| 1055 | CTACTATTAGCTGAGTGACG | 1708 | AATTGCTGGTTAAAATGCTCCC | 2361 | GGCAACCCCCACCCTCGCAGGGCC |
| 1056 | GTCCACTACTATTAGCTGAG | 1709 | ATTGCTGGTTAAAATGCTCCCG | 2362 | GCAACCCCCACCCTCGCAGGGCCG |
| 1057 | AGTCCCAGGTCCACTACTAT | 1710 | TTGCTGGTTAAAATGCTCCCGT | 2363 | CAACCCCCACCCTCGCAGGGCCGG |
| 1058 | GCAAATCTGAGTTCCAGTCC | 1711 | TGCTGGTTAAAATGCTCCCGTG | 2364 | AACCCCCACCCTCGCAGGGCCGGT |
| 1059 | TCCTTAGCAAATCTGAGTTC | 1712 | GCTGGTTAAAATGCTCCCGTGC | 2365 | ACCCCCACCCTCGCAGGGCCGGTG |
| 1060 | CCTGGTTCCTTAGCAAATCT | 1713 | CTGGTTAAAATGCTCCCGTGCA | 2366 | CCCCCACCCTCGCAGGGCCGGTGG |
| 1061 | AACCTGCCTGGTTCCTTAGC | 1714 | TGGTTAAAATGCTCCCGTGCAG | 2367 | CCCCACCCTCGCAGGGCCGGTGGG |
| 1062 | GGAAAGAACCTGCCTGGTTC | 1715 | GGTTAAAATGCTCCCGTGCAGA | 2368 | CCCACCCTCGCAGGGCCGGTGGGA |
| 1063 | TCCCGTGCAGAAAACAGGAA | 1716 | GTTAAAATGCTCCCGTGCAGAA | 2369 | CCACCCTCGCAGGGCCGGTGGGAA |
| 1064 | AATGCTCCCGTGCAGAAAAC | 1717 | TTAAAATGCTCCCGTGCAGAAA | 2370 | CACCCTCGCAGGGCCGGTGGGAAG |
| 1065 | GGTTAAAATGCTCCCGTGCA | 1718 | TAAAATGCTCCCGTGCAGAAAA | 2371 | ACCCTCGCAGGGCCGGTGGGAAGA |
| 1066 | ATTGCTGGTTAAAATGCTCC | 1719 | AAAATGCTCCCGTGCAGAAAAC | 2372 | CCCTCGCAGGGCCGGTGGGAAGAC |
| 1067 | CTTTAATTGCTGGTTAAAAT | 1720 | AAATGCTCCCGTGCAGAAAACA | 2373 | CCTCGCAGGGCCGGTGGGAAGACA |
| 1068 | AGGTTTCTTTAATTGCTGGT | 1721 | AATGCTCCCGTGCAGAAAACAG | 2374 | CTCGCAGGGCCGGTGGGAAGACAC |
| 1069 | GAAATAAGGTTTCTTTAATT | 1722 | ATGCTCCCGTGCAGAAAACAGG | 2375 | TCGCAGGGCCGGTGGGAAGACACA |
| 1070 | TTACCTAGAAATAAGGTTTC | 1723 | TGCTCCCGTGCAGAAAACAGGA | 2376 | CGCAGGGCCGGTGGGAAGACACAA |
| 1071 | TGGTTAAAATGCTCCCGT | 1724 | GCTCCCGTGCAGAAAACAGGAA | 2377 | GCAGGGCCGGTGGGAAGACACAAA |
| 1072 | GGTTAAAATGCTCCCGTG | 1725 | CTCCCGTGCAGAAAACAGGAAA | 2378 | CAGGGCCGGTGGGAAGACACAAAG |
| 1073 | GTTAAAATGCTCCCGTGC | 1726 | TCCCGTGCAGAAAACAGGAAAG | 2379 | AGGGCCGGTGGGAAGACACAAAGT |
| 1074 | AATGCTCCCGTGCAGAAA | 1727 | CCCGTGCAGAAAACAGGAAAGA | 2380 | GGGCCGGTGGGAAGACACAAAGTG |
| 1075 | ATGCTCCCGTGCAGAAAA | 1728 | CCGTGCAGAAAACAGGAAAGAA | 2381 | GGCCGGTGGGAAGACACAAAGTGA |
| 1076 | CTCCCGTGCAGAAAACAG | 1729 | CGTGCAGAAAACAGGAAAGAAC | 2382 | GCCGGTGGGAAGACACAAAGTGAT |
| 1077 | CCCGTGCAGAAAACAGGA | 1730 | GTGCAGAAAACAGGAAAGAACC | 2383 | CCGGTGGGAAGACACAAAGTGATG |
| 1078 | AAAGAACCTGCCTGGTTC | 1731 | GCAGAAAACAGGAAAGAACCTG | 2384 | CGGTGGGAAGACACAAAGTGATGT |
| 1079 | AACCTGCCTGGTTCCTTA | 1732 | CAGAAAACAGGAAAGAACCTGC | 2385 | GGTGGGAAGACACAAAGTGATGTG |
| 1080 | ACCTGCCTGGTTCCTTAG | 1733 | AGAAAACAGGAAAGAACCTGCC | 2386 | GTGGGAAGACACAAAGTGATGTGG |
| 1081 | TTAGCAAATCTGAGTTCC | 1734 | GAAAACAGGAAAGAACCTGCCT | 2387 | TGGGAAGACACAAAGTGATGTGGG |
| 1082 | CCAGTCCCAGGTCCACTA | 1735 | AAAACAGGAAAGAACCTGCCTG | 2388 | GGGAAGACACAAAGTGATGTGGGC |
| 1083 | AGGTCCACTACTATTAGC | 1736 | AAACAGGAAAGAACCTGCCTGG | 2389 | GGAAGACACAAAGTGATGTGGGCA |
| 1084 | GGTCCACTACTATTAGCT | 1737 | AACAGGAAAGAACCTGCCTGGT | 2390 | GAAGACACAAAGTGATGTGGGCAA |
| 1085 | TCCACTACTATTAGCTGA | 1738 | ACAGGAAAGAACCTGCCTGGTT | 2391 | AAGACACAAAGTGATGTGGGCAAG |
| 1086 | CTATTAGCTGAGTGACGC | 1739 | CAGGAAAGAACCTGCCTGGTTC | 2392 | AGACACAAAGTGATGTGGGCAAGT |
| 1087 | ATTAGCTGAGTGACGCTT | 1740 | AGGAAAGAACCTGCCTGGTTCC | 2393 | GACACAAAGTGATGTGGGCAAGTG |
| 1088 | TTAGCTGAGTGACGCTTC | 1741 | GGAAAGAACCTGCCTGGTTCCT | 2394 | ACACAAAGTGATGTGGGCAAGTGC |
| 1089 | TAGCTGAGTGACGCTTCC | 1742 | GAAAGAACCTGCCTGGTTCCTT | 2395 | CACAAAGTGATGTGGGCAAGTGCC |
| 1090 | TGAGTGACGCTTCCAGCC | 1743 | AAAGAACCTGCCTGGTTCCTTA | 2396 | ACAAAGTGATGTGGGCAAGTGCCA |
| 1091 | GAGTGACGCTTCCAGCCC | 1744 | AAGAACCTGCCTGGTTCCTTAG | 2397 | CAAAGTGATGTGGGCAAGTGCCAG |
| 1092 | AGTGACGCTTCCAGCCCG | 1745 | AGAACCTGCCTGGTTCCTTAGC | 2398 | AAAGTGATGTGGGCAAGTGCCAGG |
| 1093 | GTGACGCTTCCAGCCCGT | 1746 | GAACCTGCCTGGTTCCTTAGCA | 2399 | AAGTGATGTGGGCAAGTGCCAGGC |
| 1094 | GACGCTTCCAGCCCGTTT | 1747 | AACCTGCCTGGTTCCTTAGCAA | 2400 | AGTGATGTGGGCAAGTGCCAGGCC |
| 1095 | CGCTTCCAGCCCGTTTCC | 1748 | ACCTGCCTGGTTCCTTAGCAAA | 2401 | GTGATGTGGGCAAGTGCCAGGCCC |
| 1096 | CAGCCCGTTTCCCCCGCC | 1749 | CCTGCCTGGTTCCTTAGCAAAT | 2402 | TGATGTGGGCAAGTGCCAGGCCCA |
| 1097 | CGTTTCCCCCGCCTGGAA | 1750 | CTGCCTGGTTCCTTAGCAAATC | 2403 | GATGTGGGCAAGTGCCAGGCCCAC |
| 1098 | TTTCCCCCGCCTGGAAAC | 1751 | TGCCTGGTTCCTTAGCAAATCT | 2404 | ATGTGGGCAAGTGCCAGGCCCACA |
| 1099 | CCCCCGCCTGGAAACTGA | 1752 | GCCTGGTTCCTTAGCAAATCTG | 2405 | TGTGGGCAAGTGCCAGGCCCACAT |
| 1100 | CGCCTGGAAACTGAGGAC | 1753 | CCTGGTTCCTTAGCAAATCTGA | 2406 | GTGGGCAAGTGCCAGGCCCACATG |
| 1101 | GCCTGGAAACTGAGGACG | 1754 | CTGGTTCCTTAGCAAATCTGAG | 2407 | TGGGCAAGTGCCAGGCCCACATGG |
| 1102 | CCTGGAAACTGAGGACGG | 1755 | TGGTTCCTTAGCAAATCTGAGT | 2408 | GGGCAAGTGCCAGGCCCACATGGT |
| 1103 | CTGAGGACGGCAACCCCC | 1756 | GGTTCCTTAGCAAATCTGAGTT | 2409 | GGCAAGTGCCAGGCCCACATGGTA |
| 1104 | TGAGGACGGCAACCCCCA | 1757 | GTTCCTTAGCAAATCTGAGTTC | 2410 | GCAAGTGCCAGGCCCACATGGTAA |
| 1105 | AGGACGGCAACCCCCACC | 1758 | TTCCTTAGCAAATCTGAGTTCC | 2411 | CAAGTGCCAGGCCCACATGGTAAG |
| 1106 | CACCCTCGCAGGGCCGGT | 1759 | TCCTTAGCAAATCTGAGTTCCA | 2412 | AAGTGCCAGGCCCACATGGTAAGT |
| 1107 | ACCCTCGCAGGGCCGGTG | 1760 | CCTTAGCAAATCTGAGTTCCAG | 2413 | AGTGCCAGGCCCACATGGTAAGTA |
| 1108 | CTCGCAGGGCCGGTGGGA | 1761 | CTTAGCAAATCTGAGTTCCAGT | 2414 | GTGCCAGGCCCACATGGTAAGTAC |
| 1109 | TCGCAGGGCCGGTGGGAA | 1762 | TTAGCAAATCTGAGTTCCAGTC | 2415 | TGCCAGGCCCACATGGTAAGTACC |
| 1110 | GGGCCGGTGGGAAGACAC | 1763 | TAGCAAATCTGAGTTCCAGTCC | 2416 | GCCAGGCCCACATGGTAAGTACCC |
| 1111 | GGCCGGTGGGAAGACACA | 1764 | AGCAAATCTGAGTTCCAGTCCC | 2417 | CCAGGCCCACATGGTAAGTACCCA |
| 1112 | ACACAAAGTGATGTGGGC | 1765 | GCAAATCTGAGTTCCAGTCCCA | 2418 | CAGGCCCACATGGTAAGTACCCAA |
| 1113 | TGATGTGGGCAAGTGCCA | 1766 | CAAATCTGAGTTCCAGTCCCAG | 2419 | AGGCCCACATGGTAAGTACCCAAC |
| 1114 | GATGTGGGCAAGTGCCAG | 1767 | AAATCTGAGTTCCAGTCCCAGG | 2420 | GGCCCACATGGTAAGTACCCAACT |
| 1115 | AGTACCCAACTAATCTCG | 1768 | AATCTGAGTTCCAGTCCCAGGT | 2421 | GCCCACATGGTAAGTACCCAACTA |
| 1116 | GTACCCAACTAATCTCGC | 1769 | TCTGAGTTCCAGTCCCAGGTCC | 2422 | CCCACATGGTAAGTACCCAACTAA |
| 1117 | TACCCAACTAATCTCGCC | 1770 | GAGTTCCAGTCCCAGGTCCACT | 2423 | CCACATGGTAAGTACCCAACTAAT |
| 1118 | CCCAACTAATCTCGCCCC | 1771 | AGTTCCAGTCCCAGGTCCACTA | 2424 | CACATGGTAAGTACCCAACTAATC |
| 1119 | CCAACTAATCTCGCCCCT | 1772 | GTTCCAGTCCCAGGTCCACTAC | 2425 | ACATGGTAAGTACCCAACTAATCT |
| 1120 | CAACTAATCTCGCCCCTC | 1773 | TTCCAGTCCCAGGTCCACTACT | 2426 | CATGGTAAGTACCCAACTAATCTC |
| 1121 | AACTAATCTCGCCCCTCT | 1774 | TCCAGTCCCAGGTCCACTACTA | 2427 | ATGGTAAGTACCCAACTAATCTCG |
| 1122 | ACTAATCTCGCCCCTCTC | 1775 | CCAGTCCCAGGTCCACTACTAT | 2428 | TGGTAAGTACCCAACTAATCTCGC |
| 1123 | TAATCTCGCCCCTCTCAG | 1776 | CAGTCCCAGGTCCACTACTATT | 2429 | GGTAAGTACCCAACTAATCTCGCC |
| 1124 | AATCTCGCCCCTCTCAGG | 1777 | AGTCCCAGGTCCACTACTATTA | 2430 | GTAAGTACCCAACTAATCTCGCCC |
| 1125 | CGCCCCTCTCAGGACCTA | 1778 | GTCCCAGGTCCACTACTATTAG | 2431 | TAAGTACCCAACTAATCTCGCCCC |
| 1126 | ATTACCTAGAAATAAGGTT | 1779 | TCCCAGGTCCACTACTATTAGC | 2432 | AAGTACCCAACTAATCTCGCCCCT |
| 1127 | TACCTAGAAATAAGGTTTC | 1780 | CCCAGGTCCACTACTATTAGCT | 2433 | AGTACCCAACTAATCTCGCCCCTC |
| 1128 | ACCTAGAAATAAGGTTTCT | 1781 | CCAGGTCCACTACTATTAGCTG | 2434 | GTACCCAACTAATCTCGCCCCTCT |
| 1129 | CCTAGAAATAAGGTTTCTT | 1782 | CAGGTCCACTACTATTAGCTGA | 2435 | TACCCAACTAATCTCGCCCCTCTC |
| 1130 | TAGAAATAAGGTTTCTTTA | 1783 | AGGTCCACTACTATTAGCTGAG | 2436 | ACCCAACTAATCTCGCCCCTCTCA |
| 1131 | ATAAGGTTTCTTTAATTGC | 1784 | GGTCCACTACTATTAGCTGAGT | 2437 | CCCAACTAATCTCGCCCCTCTCAG |
| 1132 | AAGGTTTCTTTAATTGCTG | 1785 | GTCCACTACTATTAGCTGAGTG | 2438 | CCAACTAATCTCGCCCCTCTCAGG |
| 1133 | GTTTCTTTAATTGCTGGTT | 1786 | TCCACTACTATTAGCTGAGTGA | 2439 | CAACTAATCTCGCCCCTCTCAGGA |
| 1134 | TTCTTTAATTGCTGGTTAA | 1787 | CACTACTATTAGCTGAGTGACG | 2440 | AACTAATCTCGCCCCTCTCAGGAC |
| 1135 | TCTTTAATTGCTGGTTAAA | 1788 | ACTACTATTAGCTGAGTGACGC | 2441 | ACTAATCTCGCCCCTCTCAGGACC |
| 1136 | CTTTAATTGCTGGTTAAAA | 1789 | CTACTATTAGCTGAGTGACGCT | 2442 | CTAATCTCGCCCCTCTCAGGACCT |
| 1137 | TAATTGCTGGTTAAAATGC | 1790 | TACTATTAGCTGAGTGACGCTT | 2443 | TAATCTCGCCCCTCTCAGGACCTA |
| 1138 | AATTGCTGGTTAAAATGCT | 1791 | ACTATTAGCTGAGTGACGCTTC | 2444 | AATCTCGCCCCTCTCAGGACCTAC |
| 1139 | ATTGCTGGTTAAAATGCTC | 1792 | CTATTAGCTGAGTGACGCTTCC | 2445 | TCTTTTAAAAATTACCTAGAAATAA |
| 1140 | GCTGGTTAAAATGCTCCCG | 1793 | TATTAGCTGAGTGACGCTTCCA | 2446 | CTTTTAAAAATTACCTAGAAATAAG |
| 1141 | CTGGTTAAAATGCTCCCGT | 1794 | ATTAGCTGAGTGACGCTTCCAG | 2447 | TTTTAAAAATTACCTAGAAATAAGG |
| 1142 | TGGTTAAAATGCTCCCGTG | 1795 | TTAGCTGAGTGACGCTTCCAGC | 2448 | TTTAAAAATTACCTAGAAATAAGGT |
| 1143 | GGTTAAAATGCTCCCGTGC | 1796 | TAGCTGAGTGACGCTTCCAGCC | 2449 | TTAAAAATTACCTAGAAATAAGGTT |
| 1144 | GTTAAAATGCTCCCGTGCA | 1797 | AGCTGAGTGACGCTTCCAGCCC | 2450 | TAAAAATTACCTAGAAATAAGGTTT |
| 1145 | AAATGCTCCCGTGCAGAAA | 1798 | GCTGAGTGACGCTTCCAGCCCG | 2451 | AAAAATTACCTAGAAATAAGGTTTC |
| 1146 | AATGCTCCCGTGCAGAAAA | 1799 | CTGAGTGACGCTTCCAGCCCGT | 2452 | AAAATTACCTAGAAATAAGGTTTCT |
| 1147 | ATGCTCCCGTGCAGAAAAC | 1800 | TGAGTGACGCTTCCAGCCCGTT | 2453 | AAATTACCTAGAAATAAGGTTTCTT |
| 1148 | GCTCCCGTGCAGAAAACAG | 1801 | GAGTGACGCTTCCAGCCCGTTT | 2454 | AATTACCTAGAAATAAGGTTTCTTT |
| 1149 | CTCCCGTGCAGAAAACAGG | 1802 | AGTGACGCTTCCAGCCCGTTTC | 2455 | ATTACCTAGAAATAAGGTTTCTTTA |
| 1150 | TCCCGTGCAGAAAACAGGA | 1803 | GTGACGCTTCCAGCCCGTTTCC | 2456 | TTACCTAGAAATAAGGTTTCTTTAA |
| 1151 | CCCGTGCAGAAAACAGGAA | 1804 | TGACGCTTCCAGCCCGTTTCCC | 2457 | TACCTAGAAATAAGGTTTCTTTAAT |
| 1152 | CGTGCAGAAAACAGGAAAG | 1805 | GACGCTTCCAGCCCGTTTCCCC | 2458 | ACCTAGAAATAAGGTTTCTTTAATT |
| 1153 | ACAGGAAAGAACCTGCCTG | 1806 | ACGCTTCCAGCCCGTTTCCCCC | 2459 | CCTAGAAATAAGGTTTCTTTAATTG |
| 1154 | GAAAGAACCTGCCTGGTTC | 1807 | CGCTTCCAGCCCGTTTCCCCCG | 2460 | CTAGAAATAAGGTTTCTTTAATTGC |
| 1155 | AAAGAACCTGCCTGGTTCC | 1808 | GCTTCCAGCCCGTTTCCCCCGC | 2461 | TAGAAATAAGGTTTCTTTAATTGCT |
| 1156 | AGAACCTGCCTGGTTCCTT | 1809 | CTTCCAGCCCGTTTCCCCCGCC | 2462 | AGAAATAAGGTTTCTTTAATTGCTG |
| 1157 | GAACCTGCCTGGTTCCTTA | 1810 | TTCCAGCCCGTTTCCCCCGCCT | 2463 | GAAATAAGGTTTCTTTAATTGCTGG |
| 1158 | AACCTGCCTGGTTCCTTAG | 1811 | TCCAGCCCGTTTCCCCCGCCTG | 2464 | AAATAAGGTTTCTTTAATTGCTGGT |
| 1159 | ACCTGCCTGGTTCCTTAGC | 1812 | CCAGCCCGTTTCCCCCGCCTGG | 2465 | AATAAGGTTTCTTTAATTGCTGGTT |
| 1160 | CTGCCTGGTTCCTTAGCAA | 1813 | CAGCCCGTTTCCCCCGCCTGGA | 2466 | ATAAGGTTTCTTTAATTGCTGGTTA |
| 1161 | TGCCTGGTTCCTTAGCAAA | 1814 | AGCCCGTTTCCCCCGCCTGGAA | 2467 | TAAGGTTTCTTTAATTGCTGGTTAA |
| 1162 | GCCTGGTTCCTTAGCAAAT | 1815 | GCCCGTTTCCCCCGCCTGGAAA | 2468 | AAGGTTTCTTTAATTGCTGGTTAAA |
| 1163 | CCTGGTTCCTTAGCAAATC | 1816 | CCCGTTTCCCCCGCCTGGAAAC | 2469 | AGGTTTCTTTAATTGCTGGTTAAAA |
| 1164 | TGGTTCCTTAGCAAATCTG | 1817 | CCGTTTCCCCCGCCTGGAAACT | 2470 | GGTTTCTTTAATTGCTGGTTAAAAT |
| 1165 | CCTTAGCAAATCTGAGTTC | 1818 | CGTTTCCCCCGCCTGGAAACTG | 2471 | GTTTCTTTAATTGCTGGTTAAAATG |
| 1166 | CTTAGCAAATCTGAGTTCC | 1819 | GTTTCCCCCGCCTGGAAACTGA | 2472 | TTTCTTTAATTGCTGGTTAAAATGC |
| 1167 | TTAGCAAATCTGAGTTCCA | 1820 | TTTCCCCCGCCTGGAAACTGAG | 2473 | TTCTTTAATTGCTGGTTAAAATGCT |
| 1168 | TAGCAAATCTGAGTTCCAG | 1821 | TTCCCCCGCCTGGAAACTGAGG | 2474 | TCTTTAATTGCTGGTTAAAATGCTC |
| 1169 | TTCCAGTCCCAGGTCCACT | 1822 | TCCCCCGCCTGGAAACTGAGGA | 2475 | CTTTAATTGCTGGTTAAAATGCTCC |
| 1170 | TCCAGTCCCAGGTCCACTA | 1823 | CCCCCGCCTGGAAACTGAGGAC | 2476 | TTTAATTGCTGGTTAAAATGCTCCC |
| 1171 | CCAGTCCCAGGTCCACTAC | 1824 | CCCCGCCTGGAAACTGAGGACG | 2477 | TTAATTGCTGGTTAAAATGCTCCCG |
| 1172 | GTCCCAGGTCCACTACTAT | 1825 | CCCGCCTGGAAACTGAGGACGG | 2478 | TAATTGCTGGTTAAAATGCTCCCGT |
| 1173 | CCAGGTCCACTACTATTAG | 1826 | CCGCCTGGAAACTGAGGACGGC | 2479 | AATTGCTGGTTAAAATGCTCCCGTG |
| 1174 | CAGGTCCACTACTATTAGC | 1827 | CGCCTGGAAACTGAGGACGGCA | 2480 | ATTGCTGGTTAAAATGCTCCCGTGC |
| 1175 | AGGTCCACTACTATTAGCT | 1828 | GCCTGGAAACTGAGGACGGCAA | 2481 | TTGCTGGTTAAAATGCTCCCGTGCA |
| 1176 | GGTCCACTACTATTAGCTG | 1829 | CCTGGAAACTGAGGACGGCAAC | 2482 | TGCTGGTTAAAATGCTCCCGTGCAG |
| 1177 | GTCCACTACTATTAGCTGA | 1830 | CTGGAAACTGAGGACGGCAACC | 2483 | GCTGGTTAAAATGCTCCCGTGCAGA |
| 1178 | TCCACTACTATTAGCTGAG | 1831 | TGGAAACTGAGGACGGCAACCC | 2484 | CTGGTTAAAATGCTCCCGTGCAGAA |
| 1179 | TACTATTAGCTGAGTGACG | 1832 | GGAAACTGAGGACGGCAACCCC | 2485 | TGGTTAAAATGCTCCCGTGCAGAAA |
| 1180 | ACTATTAGCTGAGTGACGC | 1833 | GAAACTGAGGACGGCAACCCCC | 2486 | GGTTAAAATGCTCCCGTGCAGAAAA |
| 1181 | CTATTAGCTGAGTGACGCT | 1834 | AAACTGAGGACGGCAACCCCCA | 2487 | GTTAAAATGCTCCCGTGCAGAAAAC |
| 1182 | TATTAGCTGAGTGACGCTT | 1835 | AACTGAGGACGGCAACCCCCAC | 2488 | TTAAAATGCTCCCGTGCAGAAAACA |
| 1183 | ATTAGCTGAGTGACGCTTC | 1836 | ACTGAGGACGGCAACCCCCACC | 2489 | TAAAATGCTCCCGTGCAGAAAACAG |
| 1184 | TTAGCTGAGTGACGCTTCC | 1837 | CTGAGGACGGCAACCCCCACCC | 2490 | AAAATGCTCCCGTGCAGAAAACAGG |
| 1185 | TAGCTGAGTGACGCTTCCA | 1838 | TGAGGACGGCAACCCCCACCCT | 2491 | AAATGCTCCCGTGCAGAAAACAGGA |
| 1186 | AGCTGAGTGACGCTTCCAG | 1839 | GAGGACGGCAACCCCCACCCTC | 2492 | AATGCTCCCGTGCAGAAAACAGGAA |
| 1187 | GCTGAGTGACGCTTCCAGC | 1840 | AGGACGGCAACCCCCACCCTCG | 2493 | ATGCTCCCGTGCAGAAAACAGGAAA |
| 1188 | CTGAGTGACGCTTCCAGCC | 1841 | GGACGGCAACCCCCACCCTCGC | 2494 | TGCTCCCGTGCAGAAAACAGGAAAG |
| 1189 | TGAGTGACGCTTCCAGCCC | 1842 | GACGGCAACCCCCACCCTCGCA | 2495 | GCTCCCGTGCAGAAAACAGGAAAGA |
| 1190 | GAGTGACGCTTCCAGCCCG | 1843 | ACGGCAACCCCCACCCTCGCAG | 2496 | CTCCCGTGCAGAAAACAGGAAAGAA |
| 1191 | AGTGACGCTTCCAGCCCGT | 1844 | CGGCAACCCCCACCCTCGCAGG | 2497 | TCCCGTGCAGAAAACAGGAAAGAAC |
| 1192 | GTGACGCTTCCAGCCCGTT | 1845 | GGCAACCCCCACCCTCGCAGGG | 2498 | CCCGTGCAGAAAACAGGAAAGAACC |
| 1193 | TGACGCTTCCAGCCCGTTT | 1846 | GCAACCCCCACCCTCGCAGGGC | 2499 | CCGTGCAGAAAACAGGAAAGAACCT |
| 1194 | GACGCTTCCAGCCCGTTTC | 1847 | CAACCCCCACCCTCGCAGGGCC | 2500 | CGTGCAGAAAACAGGAAAGAACCTG |
| 1195 | ACGCTTCCAGCCCGTTTCC | 1848 | AACCCCCACCCTCGCAGGGCCG | 2501 | GTGCAGAAAACAGGAAAGAACCTGC |
| 1196 | CGCTTCCAGCCCGTTTCCC | 1849 | ACCCCCACCCTCGCAGGGCCGG | 2502 | TGCAGAAAACAGGAAAGAACCTGCC |
| 1197 | CTTCCAGCCCGTTTCCCCC | 1850 | CCCCCACCCTCGCAGGGCCGGT | 2503 | GCAGAAAACAGGAAAGAACCTGCCT |
| 1198 | TTCCAGCCCGTTTCCCCCG | 1851 | CCCCACCCTCGCAGGGCCGGTG | 2504 | CAGAAAACAGGAAAGAACCTGCCTG |
| 1199 | TCCAGCCCGTTTCCCCCGC | 1852 | CCCACCCTCGCAGGGCCGGTGG | 2505 | AGAAAACAGGAAAGAACCTGCCTGG |
| 1200 | CCAGCCCGTTTCCCCCGCC | 1853 | CCACCCTCGCAGGGCCGGTGGG | 2506 | GAAAACAGGAAAGAACCTGCCTGGT |
| 1201 | CAGCCCGTTTCCCCCGCCT | 1854 | CACCCTCGCAGGGCCGGTGGGA | 2507 | AAAACAGGAAAGAACCTGCCTGGTT |
| 1202 | CCGTTTCCCCCGCCTGGAA | 1855 | ACCCTCGCAGGGCCGGTGGGAA | 2508 | AAACAGGAAAGAACCTGCCTGGTTC |
| 1203 | CGTTTCCCCCGCCTGGAAA | 1856 | CCCTCGCAGGGCCGGTGGGAAG | 2509 | AACAGGAAAGAACCTGCCTGGTTCC |
| 1204 | GTTTCCCCCGCCTGGAAAC | 1857 | CCTCGCAGGGCCGGTGGGAAGA | 2510 | ACAGGAAAGAACCTGCCTGGTTCCT |
| 1205 | TTTCCCCCGCCTGGAAACT | 1858 | CTCGCAGGGCCGGTGGGAAGAC | 2511 | CAGGAAAGAACCTGCCTGGTTCCTT |
| 1206 | TCCCCCGCCTGGAAACTGA | 1859 | TCGCAGGGCCGGTGGGAAGACA | 2512 | AGGAAAGAACCTGCCTGGTTCCTTA |
| 1207 | CCCCCGCCTGGAAACTGAG | 1860 | CGCAGGGCCGGTGGGAAGACAC | 2513 | GGAAAGAACCTGCCTGGTTCCTTAG |
| 1208 | CCGCCTGGAAACTGAGGAC | 1861 | GCAGGGCCGGTGGGAAGACACA | 2514 | GAAAGAACCTGCCTGGTTCCTTAGC |
| 1209 | CGCCTGGAAACTGAGGACG | 1862 | CAGGGCCGGTGGGAAGACACAA | 2515 | AAAGAACCTGCCTGGTTCCTTAGCA |
| 1210 | GCCTGGAAACTGAGGACGG | 1863 | AGGGCCGGTGGGAAGACACAAA | 2516 | AAGAACCTGCCTGGTTCCTTAGCAA |
| 1211 | CCTGGAAACTGAGGACGGC | 1864 | GGGCCGGTGGGAAGACACAAAG | 2517 | AGAACCTGCCTGGTTCCTTAGCAAA |
| 1212 | CTGGAAACTGAGGACGGCA | 1865 | GGCCGGTGGGAAGACACAAAGT | 2518 | GAACCTGCCTGGTTCCTTAGCAAAT |
| 1213 | TGGAAACTGAGGACGGCAA | 1866 | GCCGGTGGGAAGACACAAAGTG | 2519 | AACCTGCCTGGTTCCTTAGCAAATC |
| 1214 | GGAAACTGAGGACGGCAAC | 1867 | CCGGTGGGAAGACACAAAGTGA | 2520 | ACCTGCCTGGTTCCTTAGCAAATCT |
| 1215 | ACTGAGGACGGCAACCCCC | 1868 | CGGTGGGAAGACACAAAGTGAT | 2521 | CCTGCCTGGTTCCTTAGCAAATCTG |
| 1216 | CTGAGGACGGCAACCCCCA | 1869 | GGTGGGAAGACACAAAGTGATG | 2522 | CTGCCTGGTTCCTTAGCAAATCTGA |
| 1217 | TGAGGACGGCAACCCCCAC | 1870 | GTGGGAAGACACAAAGTGATGT | 2523 | TGCCTGGTTCCTTAGCAAATCTGAG |
| 1218 | GAGGACGGCAACCCCCACC | 1871 | TGGGAAGACACAAAGTGATGTG | 2524 | GCCTGGTTCCTTAGCAAATCTGAGT |
| 1219 | AGGACGGCAACCCCCACCC | 1872 | GGGAAGACACAAAGTGATGTGG | 2525 | CCTGGTTCCTTAGCAAATCTGAGTT |
| 1220 | GGCAACCCCCACCCTCGCA | 1873 | GGAAGACACAAAGTGATGTGGG | 2526 | CTGGTTCCTTAGCAAATCTGAGTTC |
| 1221 | AACCCCCACCCTCGCAGGG | 1874 | GAAGACACAAAGTGATGTGGGC | 2527 | TGGTTCCTTAGCAAATCTGAGTTCC |
| 1222 | CCACCCTCGCAGGGCCGGT | 1875 | AAGACACAAAGTGATGTGGGCA | 2528 | GGTTCCTTAGCAAATCTGAGTTCCA |
| 1223 | CACCCTCGCAGGGCCGGTG | 1876 | AGACACAAAGTGATGTGGGCAA | 2529 | GTTCCTTAGCAAATCTGAGTTCCAG |
| 1224 | ACCCTCGCAGGGCCGGTGG | 1877 | GACACAAAGTGATGTGGGCAAG | 2530 | TTCCTTAGCAAATCTGAGTTCCAGT |
| 1225 | CCTCGCAGGGCCGGTGGGA | 1878 | ACACAAAGTGATGTGGGCAAGT | 2531 | TCCTTAGCAAATCTGAGTTCCAGTC |
| 1226 | CTCGCAGGGCCGGTGGGAA | 1879 | CACAAAGTGATGTGGGCAAGTG | 2532 | CCTTAGCAAATCTGAGTTCCAGTCC |
| 1227 | TCGCAGGGCCGGTGGGAAG | 1880 | ACAAAGTGATGTGGGCAAGTGC | 2533 | CTTAGCAAATCTGAGTTCCAGTCCC |
| 1228 | CGCAGGGCCGGTGGGAAGA | 1881 | CAAAGTGATGTGGGCAAGTGCC | 2534 | TTAGCAAATCTGAGTTCCAGTCCCA |
| 1229 | GCAGGGCCGGTGGGAAGAC | 1882 | AAAGTGATGTGGGCAAGTGCCA | 2535 | TAGCAAATCTGAGTTCCAGTCCCAG |
| 1230 | CAGGGCCGGTGGGAAGACA | 1883 | AAGTGATGTGGGCAAGTGCCAG | 2536 | AGCAAATCTGAGTTCCAGTCCCAGG |
| 1231 | AGGGCCGGTGGGAAGACAC | 1884 | AGTGATGTGGGCAAGTGCCAGG | 2537 | GCAAATCTGAGTTCCAGTCCCAGGT |
| 1232 | GGGCCGGTGGGAAGACACA | 1885 | GTGATGTGGGCAAGTGCCAGGC | 2538 | CAAATCTGAGTTCCAGTCCCAGGTC |
| 1233 | GGCCGGTGGGAAGACACAA | 1886 | TGATGTGGGCAAGTGCCAGGCC | 2539 | AAATCTGAGTTCCAGTCCCAGGTCC |
| 1234 | GCCGGTGGGAAGACACAAA | 1887 | GATGTGGGCAAGTGCCAGGCCC | 2540 | AATCTGAGTTCCAGTCCCAGGTCCA |
| 1235 | CCGGTGGGAAGACACAAAG | 1888 | ATGTGGGCAAGTGCCAGGCCCA | 2541 | ATCTGAGTTCCAGTCCCAGGTCCAC |
| 1236 | CGGTGGGAAGACACAAAGT | 1889 | TGTGGGCAAGTGCCAGGCCCAC | 2542 | TCTGAGTTCCAGTCCCAGGTCCACT |
| 1237 | GACACAAAGTGATGTGGGC | 1890 | GTGGGCAAGTGCCAGGCCCACA | 2543 | CTGAGTTCCAGTCCCAGGTCCACTA |
| 1238 | ACACAAAGTGATGTGGGCA | 1891 | TGGGCAAGTGCCAGGCCCACAT | 2544 | TGAGTTCCAGTCCCAGGTCCACTAC |
| 1239 | CACAAAGTGATGTGGGCAA | 1892 | GGGCAAGTGCCAGGCCCACATG | 2545 | GAGTTCCAGTCCCAGGTCCACTACT |
| 1240 | AAGTGATGTGGGCAAGTGC | 1893 | GGCAAGTGCCAGGCCCACATGG | 2546 | AGTTCCAGTCCCAGGTCCACTACTA |
| 1241 | AGTGATGTGGGCAAGTGCC | 1894 | GCAAGTGCCAGGCCCACATGGT | 2547 | GTTCCAGTCCCAGGTCCACTACTAT |
| 1242 | GTGATGTGGGCAAGTGCCA | 1895 | CAAGTGCCAGGCCCACATGGTA | 2548 | TTCCAGTCCCAGGTCCACTACTATT |
| 1243 | TGATGTGGGCAAGTGCCAG | 1896 | AAGTGCCAGGCCCACATGGTAA | 2549 | TCCAGTCCCAGGTCCACTACTATTA |
| 1244 | GATGTGGGCAAGTGCCAGG | 1897 | AGTGCCAGGCCCACATGGTAAG | 2550 | CCAGTCCCAGGTCCACTACTATTAG |
| 1245 | TGTGGGCAAGTGCCAGGCC | 1898 | GTGCCAGGCCCACATGGTAAGT | 2551 | CAGTCCCAGGTCCACTACTATTAGC |
| 1246 | GGGCAAGTGCCAGGCCCAC | 1899 | TGCCAGGCCCACATGGTAAGTA | 2552 | AGTCCCAGGTCCACTACTATTAGCT |
| 1247 | GCAAGTGCCAGGCCCACAT | 1900 | GCCAGGCCCACATGGTAAGTAC | 2553 | GTCCCAGGTCCACTACTATTAGCTG |
| 1248 | CAAGTGCCAGGCCCACATG | 1901 | CCAGGCCCACATGGTAAGTACC | 2554 | TCCCAGGTCCACTACTATTAGCTGA |
| 1249 | CAGGCCCACATGGTAAGTA | 1902 | CAGGCCCACATGGTAAGTACCC | 2555 | CCCAGGTCCACTACTATTAGCTGAG |
| 1250 | GGTAAGTACCCAACTAATC | 1903 | AGGCCCACATGGTAAGTACCCA | 2556 | CCAGGTCCACTACTATTAGCTGAGT |
| 1251 | GTAAGTACCCAACTAATCT | 1904 | GGCCCACATGGTAAGTACCCAA | 2557 | CAGGTCCACTACTATTAGCTGAGTG |
| 1252 | TAAGTACCCAACTAATCTC | 1905 | GCCCACATGGTAAGTACCCAAC | 2558 | AGGTCCACTACTATTAGCTGAGTGA |
| 1253 | AAGTACCCAACTAATCTCG | 1906 | CCCACATGGTAAGTACCCAACT | 2559 | GGTCCACTACTATTAGCTGAGTGAC |
| 1254 | AGTACCCAACTAATCTCGC | 1907 | CCACATGGTAAGTACCCAACTA | 2560 | GTCCACTACTATTAGCTGAGTGACG |
| 1255 | GTACCCAACTAATCTCGCC | 1908 | CATGGTAAGTACCCAACTAATC | 2561 | TCCACTACTATTAGCTGAGTGACGC |
| 1256 | TACCCAACTAATCTCGCCC | 1909 | ATGGTAAGTACCCAACTAATCT | 2562 | CCACTACTATTAGCTGAGTGACGCT |
| 1257 | ACCCAACTAATCTCGCCCC | 1910 | TGGTAAGTACCCAACTAATCTC | 2563 | CACTACTATTAGCTGAGTGACGCTT |
| 1258 | CCCAACTAATCTCGCCCCT | 1911 | GGTAAGTACCCAACTAATCTCG | 2564 | ACTACTATTAGCTGAGTGACGCTTC |
| 1259 | CCAACTAATCTCGCCCCTC | 1912 | GTAAGTACCCAACTAATCTCGC | 2565 | CTACTATTAGCTGAGTGACGCTTCC |
| 1260 | CAACTAATCTCGCCCCTCT | 1913 | TAAGTACCCAACTAATCTCGCC | 2566 | TACTATTAGCTGAGTGACGCTTCCA |
| 1261 | AACTAATCTCGCCCCTCTC | 1914 | AAGTACCCAACTAATCTCGCCC | 2567 | ACTATTAGCTGAGTGACGCTTCCAG |
| 1262 | ACTAATCTCGCCCCTCTCA | 1915 | AGTACCCAACTAATCTCGCCCC | 2568 | CTATTAGCTGAGTGACGCTTCCAGC |
| 1263 | CTAATCTCGCCCCTCTCAG | 1916 | GTACCCAACTAATCTCGCCCCT | 2569 | TATTAGCTGAGTGACGCTTCCAGCC |
| 1264 | TAATCTCGCCCCTCTCAGG | 1917 | TACCCAACTAATCTCGCCCCTC | 2570 | ATTAGCTGAGTGACGCTTCCAGCCC |
| 1265 | AATCTCGCCCCTCTCAGGA | 1918 | ACCCAACTAATCTCGCCCCTCT | 2571 | TTAGCTGAGTGACGCTTCCAGCCCG |
| 1266 | ATCTCGCCCCTCTCAGGAC | 1919 | CCCAACTAATCTCGCCCCTCTC | 2572 | TAGCTGAGTGACGCTTCCAGCCCGT |
| 1267 | TCTCGCCCCTCTCAGGACC | 1920 | CCAACTAATCTCGCCCCTCTCA | 2573 | AGCTGAGTGACGCTTCCAGCCCGTT |
| 1268 | TCGCCCCTCTCAGGACCTA | 1921 | CAACTAATCTCGCCCCTCTCAG | 2574 | GCTGAGTGACGCTTCCAGCCCGTTT |
| 1269 | CGCCCCTCTCAGGACCTAC | 1922 | AACTAATCTCGCCCCTCTCAGG | 2575 | CTGAGTGACGCTTCCAGCCCGTTTC |
| 1270 | AAATTACCTAGAAATAAGGT | 1923 | ACTAATCTCGCCCCTCTCAGGA | 2576 | TGAGTGACGCTTCCAGCCCGTTTCC |
| 1271 | AATTACCTAGAAATAAGGTT | 1924 | CTAATCTCGCCCCTCTCAGGAC | 2577 | GAGTGACGCTTCCAGCCCGTTTCCC |
| 1272 | ATTACCTAGAAATAAGGTTT | 1925 | TAATCTCGCCCCTCTCAGGACC | 2578 | AGTGACGCTTCCAGCCCGTTTCCCC |
| 1273 | TACCTAGAAATAAGGTTTCT | 1926 | AATCTCGCCCCTCTCAGGACCT | 2579 | GTGACGCTTCCAGCCCGTTTCCCCC |
| 1274 | ACCTAGAAATAAGGTTTCTT | 1927 | ATCTCGCCCCTCTCAGGACCTA | 2580 | TGACGCTTCCAGCCCGTTTCCCCCG |
| 1275 | CCTAGAAATAAGGTTTCTTT | 1928 | TCTCGCCCCTCTCAGGACCTAC | 2581 | GACGCTTCCAGCCCGTTTCCCCCGC |
| 1276 | CTAGAAATAAGGTTTCTTTA | 1929 | TCTTTTAAAAATTACCTAGAAAT | 2582 | ACGCTTCCAGCCCGTTTCCCCCGCC |
| 1277 | TAGAAATAAGGTTTCTTTAA | 1930 | TTTAAAAATTACCTAGAAATAAG | 2583 | CGCTTCCAGCCCGTTTCCCCCGCCT |
| 1278 | AAATAAGGTTTCTTTAATTG | 1931 | TTAAAAATTACCTAGAAATAAGG | 2584 | GCTTCCAGCCCGTTTCCCCCGCCTG |
| 1279 | AATAAGGTTTCTTTAATTGC | 1932 | TAAAAATTACCTAGAAATAAGGT | 2585 | CTTCCAGCCCGTTTCCCCCGCCTGG |
| 1280 | ATAAGGTTTCTTTAATTGCT | 1933 | AAAAATTACCTAGAAATAAGGTT | 2586 | TTCCAGCCCGTTTCCCCCGCCTGGA |
| 1281 | TAAGGTTTCTTTAATTGCTG | 1934 | AAAATTACCTAGAAATAAGGTTT | 2587 | TCCAGCCCGTTTCCCCCGCCTGGAA |
| 1282 | AAGGTTTCTTTAATTGCTGG | 1935 | AAATTACCTAGAAATAAGGTTTC | 2588 | CCAGCCCGTTTCCCCCGCCTGGAAA |
| 1283 | GGTTTCTTTAATTGCTGGTT | 1936 | AATTACCTAGAAATAAGGTTTCT | 2589 | CAGCCCGTTTCCCCCGCCTGGAAAC |
| 1284 | GTTTCTTTAATTGCTGGTTA | 1937 | ATTACCTAGAAATAAGGTTTCTT | 2590 | AGCCCGTTTCCCCCGCCTGGAAACT |
| 1285 | TTTCTTTAATTGCTGGTTAA | 1938 | TTACCTAGAAATAAGGTTTCTTT | 2591 | GCCCGTTTCCCCCGCCTGGAAACTG |
| 1286 | TTCTTTAATTGCTGGTTAAA | 1939 | TACCTAGAAATAAGGTTTCTTTA | 2592 | CCCGTTTCCCCCGCCTGGAAACTGA |
| 1287 | TCTTTAATTGCTGGTTAAAA | 1940 | ACCTAGAAATAAGGTTTCTTTAA | 2593 | CCGTTTCCCCCGCCTGGAAACTGAG |
| 1288 | TTAATTGCTGGTTAAAATGC | 1941 | CCTAGAAATAAGGTTTCTTTAAT | 2594 | CGTTTCCCCCGCCTGGAAACTGAGG |
| 1289 | TAATTGCTGGTTAAAATGCT | 1942 | CTAGAAATAAGGTTTCTTTAATT | 2595 | GTTTCCCCCGCCTGGAAACTGAGGA |
| 1290 | AATTGCTGGTTAAAATGCTC | 1943 | TAGAAATAAGGTTTCTTTAATTG | 2596 | TTTCCCCCGCCTGGAAACTGAGGAC |
| 1291 | TTGCTGGTTAAAATGCTCCC | 1944 | AGAAATAAGGTTTCTTTAATTGC | 2597 | TTCCCCCGCCTGGAAACTGAGGACG |
| 1292 | TGCTGGTTAAAATGCTCCCG | 1945 | GAAATAAGGTTTCTTTAATTGCT | 2598 | TCCCCCGCCTGGAAACTGAGGACGG |
| 1293 | GCTGGTTAAAATGCTCCCGT | 1946 | AAATAAGGTTTCTTTAATTGCTG | 2599 | CCCCCGCCTGGAAACTGAGGACGGC |
| 1294 | CTGGTTAAAATGCTCCCGTG | 1947 | AATAAGGTTTCTTTAATTGCTGG | 2600 | CCCCGCCTGGAAACTGAGGACGGCA |
| 1295 | TGGTTAAAATGCTCCCGTGC | 1948 | ATAAGGTTTCTTTAATTGCTGGT | 2601 | CCCGCCTGGAAACTGAGGACGGCAA |
| 1296 | GTTAAAATGCTCCCGTGCAG | 1949 | TAAGGTTTCTTTAATTGCTGGTT | 2602 | CCGCCTGGAAACTGAGGACGGCAAC |
| 1297 | AAAATGCTCCCGTGCAGAAA | 1950 | AAGGTTTCTTTAATTGCTGGTTA | 2603 | CGCCTGGAAACTGAGGACGGCAACC |
| 1298 | AAATGCTCCCGTGCAGAAAA | 1951 | AGGTTTCTTTAATTGCTGGTTAA | 2604 | GCCTGGAAACTGAGGACGGCAACCC |
| 1299 | ATGCTCCCGTGCAGAAAACA | 1952 | GGTTTCTTTAATTGCTGGTTAAA | 2605 | CCTGGAAACTGAGGACGGCAACCCC |
| 1300 | TGCTCCCGTGCAGAAAACAG | 1953 | GTTTCTTTAATTGCTGGTTAAAA | 2606 | CTGGAAACTGAGGACGGCAACCCCC |
| 1301 | GCTCCCGTGCAGAAAACAGG | 1954 | TTTCTTTAATTGCTGGTTAAAAT | 2607 | TGGAAACTGAGGACGGCAACCCCCA |
| 1302 | CTCCCGTGCAGAAAACAGGA | 1955 | TTCTTTAATTGCTGGTTAAAATG | 2608 | GGAAACTGAGGACGGCAACCCCCAC |
| 1303 | CCCGTGCAGAAAACAGGAAA | 1956 | TCTTTAATTGCTGGTTAAAATGC | 2609 | GAAACTGAGGACGGCAACCCCCACC |
| 1304 | CCGTGCAGAAAACAGGAAAG | 1957 | CTTTAATTGCTGGTTAAAATGCT | 2610 | AAACTGAGGACGGCAACCCCCACCC |
| 1305 | CGTGCAGAAAACAGGAAAGA | 1958 | TTTAATTGCTGGTTAAAATGCTC | 2611 | AACTGAGGACGGCAACCCCCACCCT |
| 1306 | GTGCAGAAAACAGGAAAGAA | 1959 | TTAATTGCTGGTTAAAATGCTCC | 2612 | ACTGAGGACGGCAACCCCCACCCTC |
| 1307 | AACAGGAAAGAACCTGCCTG | 1960 | TAATTGCTGGTTAAAATGCTCCC | 2613 | CTGAGGACGGCAACCCCCACCCTCG |
| 1308 | ACAGGAAAGAACCTGCCTGG | 1961 | AATTGCTGGTTAAAATGCTCCCG | 2614 | TGAGGACGGCAACCCCCACCCTCGC |
| 1309 | CAGGAAAGAACCTGCCTGGT | 1962 | ATTGCTGGTTAAAATGCTCCCGT | 2615 | GAGGACGGCAACCCCCACCCTCGCA |
| 1310 | AGGAAAGAACCTGCCTGGTT | 1963 | TTGCTGGTTAAAATGCTCCCGTG | 2616 | AGGACGGCAACCCCCACCCTCGCAG |
| 1311 | GAAAGAACCTGCCTGGTTCC | 1964 | TGCTGGTTAAAATGCTCCCGTGC | 2617 | GGACGGCAACCCCCACCCTCGCAGG |
| 1312 | AAAGAACCTGCCTGGTTCCT | 1965 | GCTGGTTAAAATGCTCCCGTGCA | 2618 | GACGGCAACCCCCACCCTCGCAGGG |
| 1313 | AAGAACCTGCCTGGTTCCTT | 1966 | CTGGTTAAAATGCTCCCGTGCAG | 2619 | ACGGCAACCCCCACCCTCGCAGGGC |
| 1314 | AGAACCTGCCTGGTTCCTTA | 1967 | TGGTTAAAATGCTCCCGTGCAGA | 2620 | CGGCAACCCCCACCCTCGCAGGGCC |
| 1315 | GAACCTGCCTGGTTCCTTAG | 1968 | GGTTAAAATGCTCCCGTGCAGAA | 2621 | GGCAACCCCCACCCTCGCAGGGCCG |
| 1316 | ACCTGCCTGGTTCCTTAGCA | 1969 | GTTAAAATGCTCCCGTGCAGAAA | 2622 | GCAACCCCCACCCTCGCAGGGCCGG |
| 1317 | CCTGCCTGGTTCCTTAGCAA | 1970 | TTAAAATGCTCCCGTGCAGAAAA | 2623 | CAACCCCCACCCTCGCAGGGCCGGT |
| 1318 | CTGCCTGGTTCCTTAGCAAA | 1971 | TAAAATGCTCCCGTGCAGAAAAC | 2624 | AACCCCCACCCTCGCAGGGCCGGTG |
| 1319 | TGCCTGGTTCCTTAGCAAAT | 1972 | AAAATGCTCCCGTGCAGAAAACA | 2625 | ACCCCCACCCTCGCAGGGCCGGTGG |
| 1320 | GCCTGGTTCCTTAGCAAATC | 1973 | AAATGCTCCCGTGCAGAAAACAG | 2626 | CCCCCACCCTCGCAGGGCCGGTGGG |
| 1321 | CTGGTTCCTTAGCAAATCTG | 1974 | AATGCTCCCGTGCAGAAAACAGG | 2627 | CCCCACCCTCGCAGGGCCGGTGGGA |
| 1322 | TGGTTCCTTAGCAAATCTGA | 1975 | ATGCTCCCGTGCAGAAAACAGGA | 2628 | CCCACCCTCGCAGGGCCGGTGGGAA |
| 1323 | GTTCCTTAGCAAATCTGAGT | 1976 | TGCTCCCGTGCAGAAAACAGGAA | 2629 | CCACCCTCGCAGGGCCGGTGGGAAG |
| 1324 | CCTTAGCAAATCTGAGTTCC | 1977 | GCTCCCGTGCAGAAAACAGGAAA | 2630 | CACCCTCGCAGGGCCGGTGGGAAGA |
| 1325 | CTTAGCAAATCTGAGTTCCA | 1978 | CTCCCGTGCAGAAAACAGGAAAG | 2631 | ACCCTCGCAGGGCCGGTGGGAAGAC |
| 1326 | TTAGCAAATCTGAGTTCCAG | 1979 | TCCCGTGCAGAAAACAGGAAAGA | 2632 | CCCTCGCAGGGCCGGTGGGAAGACA |
| 1327 | TAGCAAATCTGAGTTCCAGT | 1980 | CCCGTGCAGAAAACAGGAAAGAA | 2633 | CCTCGCAGGGCCGGTGGGAAGACAC |
| 1328 | AATCTGAGTTCCAGTCCCAG | 1981 | CCGTGCAGAAAACAGGAAAGAAC | 2634 | CTCGCAGGGCCGGTGGGAAGACACA |
| 1329 | GTTCCAGTCCCAGGTCCACT | 1982 | CGTGCAGAAAACAGGAAAGAACC | 2635 | TCGCAGGGCCGGTGGGAAGACACAA |
| 1330 | TTCCAGTCCCAGGTCCACTA | 1983 | GTGCAGAAAACAGGAAAGAACCT | 2636 | CGCAGGGCCGGTGGGAAGACACAAA |
| 1331 | TCCAGTCCCAGGTCCACTAC | 1984 | TGCAGAAAACAGGAAAGAACCTG | 2637 | GCAGGGCCGGTGGGAAGACACAAAG |
| 1332 | CCAGTCCCAGGTCCACTACT | 1985 | GCAGAAAACAGGAAAGAACCTGC | 2638 | CAGGGCCGGTGGGAAGACACAAAGT |
| 1333 | GTCCCAGGTCCACTACTATT | 1986 | CAGAAAACAGGAAAGAACCTGCC | 2639 | AGGGCCGGTGGGAAGACACAAAGTG |
| 1334 | CCCAGGTCCACTACTATTAG | 1987 | AGAAAACAGGAAAGAACCTGCCT | 2640 | GGGCCGGTGGGAAGACACAAAGTGA |
| 1335 | CCAGGTCCACTACTATTAGC | 1988 | GAAAACAGGAAAGAACCTGCCTG | 2641 | GGCCGGTGGGAAGACACAAAGTGAT |
| 1336 | CAGGTCCACTACTATTAGCT | 1989 | AAAACAGGAAAGAACCTGCCTGG | 2642 | GCCGGTGGGAAGACACAAAGTGATG |
| 1337 | AGGTCCACTACTATTAGCTG | 1990 | AAACAGGAAAGAACCTGCCTGGT | 2643 | CCGGTGGGAAGACACAAAGTGATGT |
| 1338 | GGTCCACTACTATTAGCTGA | 1991 | AACAGGAAAGAACCTGCCTGGTT | 2644 | CGGTGGGAAGACACAAAGTGATGTG |
| 1339 | TCCACTACTATTAGCTGAGT | 1992 | ACAGGAAAGAACCTGCCTGGTTC | 2645 | GGTGGGAAGACACAAAGTGATGTGG |
| 1340 | TACTATTAGCTGAGTGACGC | 1993 | CAGGAAAGAACCTGCCTGGTTCC | 2646 | GTGGGAAGACACAAAGTGATGTGGG |
| 1341 | ACTATTAGCTGAGTGACGCT | 1994 | AGGAAAGAACCTGCCTGGTTCCT | 2647 | TGGGAAGACACAAAGTGATGTGGGC |
| 1342 | CTATTAGCTGAGTGACGCTT | 1995 | GGAAAGAACCTGCCTGGTTCCTT | 2648 | GGGAAGACACAAAGTGATGTGGGCA |
| 1343 | TATTAGCTGAGTGACGCTTC | 1996 | GAAAGAACCTGCCTGGTTCCTTA | 2649 | GGAAGACACAAAGTGATGTGGGCAA |
| 1344 | ATTAGCTGAGTGACGCTTCC | 1997 | AAAGAACCTGCCTGGTTCCTTAG | 2650 | GAAGACACAAAGTGATGTGGGCAAG |
| 1345 | TAGCTGAGTGACGCTTCCAG | 1998 | AAGAACCTGCCTGGTTCCTTAGC | 2651 | AAGACACAAAGTGATGTGGGCAAGT |
| 1346 | AGCTGAGTGACGCTTCCAGC | 1999 | AGAACCTGCCTGGTTCCTTAGCA | 2652 | AGACACAAAGTGATGTGGGCAAGTG |
| 1347 | GCTGAGTGACGCTTCCAGCC | 2000 | GAACCTGCCTGGTTCCTTAGCAA | 2653 | GACACAAAGTGATGTGGGCAAGTGC |
| 1348 | CTGAGTGACGCTTCCAGCCC | 2001 | AACCTGCCTGGTTCCTTAGCAAA | 2654 | ACACAAAGTGATGTGGGCAAGTGCC |
| 1349 | TGAGTGACGCTTCCAGCCCG | 2002 | ACCTGCCTGGTTCCTTAGCAAAT | 2655 | CACAAAGTGATGTGGGCAAGTGCCA |
| 1350 | GAGTGACGCTTCCAGCCCGT | 2003 | CCTGCCTGGTTCCTTAGCAAATC | 2656 | ACAAAGTGATGTGGGCAAGTGCCAG |
| 1351 | GTGACGCTTCCAGCCCGTTT | 2004 | CTGCCTGGTTCCTTAGCAAATCT | 2657 | CAAAGTGATGTGGGCAAGTGCCAGG |
| 1352 | TGACGCTTCCAGCCCGTTTC | 2005 | TGCCTGGTTCCTTAGCAAATCTG | 2658 | AAAGTGATGTGGGCAAGTGCCAGGC |
| 1353 | GACGCTTCCAGCCCGTTTCC | 2006 | GCCTGGTTCCTTAGCAAATCTGA | 2659 | AAGTGATGTGGGCAAGTGCCAGGCC |
| 1354 | ACGCTTCCAGCCCGTTTCCC | 2007 | CCTGGTTCCTTAGCAAATCTGAG | 2660 | AGTGATGTGGGCAAGTGCCAGGCCC |
| 1355 | CGCTTCCAGCCCGTTTCCCC | 2008 | CTGGTTCCTTAGCAAATCTGAGT | 2661 | GTGATGTGGGCAAGTGCCAGGCCCA |
| 1356 | GCTTCCAGCCCGTTTCCCCC | 2009 | TGGTTCCTTAGCAAATCTGAGTT | 2662 | TGATGTGGGCAAGTGCCAGGCCCAC |
| 1357 | CTTCCAGCCCGTTTCCCCCG | 2010 | GGTTCCTTAGCAAATCTGAGTTC | 2663 | GATGTGGGCAAGTGCCAGGCCCACA |
| 1358 | TCCAGCCCGTTTCCCCCGCC | 2011 | GTTCCTTAGCAAATCTGAGTTCC | 2664 | ATGTGGGCAAGTGCCAGGCCCACAT |
| 1359 | CCAGCCCGTTTCCCCCGCCT | 2012 | TTCCTTAGCAAATCTGAGTTCCA | 2665 | TGTGGGCAAGTGCCAGGCCCACATG |
| 1360 | CAGCCCGTTTCCCCCGCCTG | 2013 | TCCTTAGCAAATCTGAGTTCCAG | 2666 | GTGGGCAAGTGCCAGGCCCACATGG |
| 1361 | AGCCCGTTTCCCCCGCCTGG | 2014 | CCTTAGCAAATCTGAGTTCCAGT | 2667 | TGGGCAAGTGCCAGGCCCACATGGT |
| 1362 | CCCGTTTCCCCCGCCTGGAA | 2015 | CTTAGCAAATCTGAGTTCCAGTC | 2668 | GGGCAAGTGCCAGGCCCACATGGTA |
| 1363 | CCGTTTCCCCCGCCTGGAAA | 2016 | TTAGCAAATCTGAGTTCCAGTCC | 2669 | GGCAAGTGCCAGGCCCACATGGTAA |
| 1364 | CGTTTCCCCCGCCTGGAAAC | 2017 | TAGCAAATCTGAGTTCCAGTCCC | 2670 | GCAAGTGCCAGGCCCACATGGTAAG |
| 1365 | GTTTCCCCCGCCTGGAAACT | 2018 | AGCAAATCTGAGTTCCAGTCCCA | 2671 | CAAGTGCCAGGCCCACATGGTAAGT |
| 1366 | TTTCCCCCGCCTGGAAACTG | 2019 | GCAAATCTGAGTTCCAGTCCCAG | 2672 | AAGTGCCAGGCCCACATGGTAAGTA |
| 1367 | TCCCCCGCCTGGAAACTGAG | 2020 | CAAATCTGAGTTCCAGTCCCAGG | 2673 | AGTGCCAGGCCCACATGGTAAGTAC |
| 1368 | CCCCCGCCTGGAAACTGAGG | 2021 | AAATCTGAGTTCCAGTCCCAGGT | 2674 | GTGCCAGGCCCACATGGTAAGTACC |
| 1369 | CCCGCCTGGAAACTGAGGAC | 2022 | AATCTGAGTTCCAGTCCCAGGTC | 2675 | TGCCAGGCCCACATGGTAAGTACCC |
| 1370 | CCGCCTGGAAACTGAGGACG | 2023 | ATCTGAGTTCCAGTCCCAGGTCC | 2676 | GCCAGGCCCACATGGTAAGTACCCA |
| 1371 | GCCTGGAAACTGAGGACGGC | 2024 | TCTGAGTTCCAGTCCCAGGTCCA | 2677 | CCAGGCCCACATGGTAAGTACCCAA |
| 1372 | CCTGGAAACTGAGGACGGCA | 2025 | TGAGTTCCAGTCCCAGGTCCACT | 2678 | CAGGCCCACATGGTAAGTACCCAAC |
| 1373 | CTGGAAACTGAGGACGGCAA | 2026 | GAGTTCCAGTCCCAGGTCCACTA | 2679 | AGGCCCACATGGTAAGTACCCAACT |
| 1374 | TGGAAACTGAGGACGGCAAC | 2027 | AGTTCCAGTCCCAGGTCCACTAC | 2680 | GGCCCACATGGTAAGTACCCAACTA |
| 1375 | AACTGAGGACGGCAACCCCC | 2028 | GTTCCAGTCCCAGGTCCACTACT | 2681 | GCCCACATGGTAAGTACCCAACTAA |
| 1376 | ACTGAGGACGGCAACCCCCA | 2029 | TTCCAGTCCCAGGTCCACTACTA | 2682 | CCCACATGGTAAGTACCCAACTAAT |
| 1377 | CTGAGGACGGCAACCCCCAC | 2030 | TCCAGTCCCAGGTCCACTACTAT | 2683 | CCACATGGTAAGTACCCAACTAATC |
| 1378 | GAGGACGGCAACCCCCACCC | 2031 | CCAGTCCCAGGTCCACTACTATT | 2684 | CACATGGTAAGTACCCAACTAATCT |
| 1379 | AGGACGGCAACCCCCACCCT | 2032 | CAGTCCCAGGTCCACTACTATTA | 2685 | ACATGGTAAGTACCCAACTAATCTC |
| 1380 | GGACGGCAACCCCCACCCTC | 2033 | AGTCCCAGGTCCACTACTATTAG | 2686 | CATGGTAAGTACCCAACTAATCTCG |
| 1381 | CGGCAACCCCCACCCTCGCA | 2034 | GTCCCAGGTCCACTACTATTAGC | 2687 | ATGGTAAGTACCCAACTAATCTCGC |
| 1382 | CAACCCCCACCCTCGCAGGG | 2035 | TCCCAGGTCCACTACTATTAGCT | 2688 | TGGTAAGTACCCAACTAATCTCGCC |
| 1383 | AACCCCCACCCTCGCAGGGC | 2036 | CCCAGGTCCACTACTATTAGCTG | 2689 | GGTAAGTACCCAACTAATCTCGCCC |
| 1384 | ACCCCCACCCTCGCAGGGCC | 2037 | CCAGGTCCACTACTATTAGCTGA | 2690 | GTAAGTACCCAACTAATCTCGCCCC |
| 1385 | CCCACCCTCGCAGGGCCGGT | 2038 | CAGGTCCACTACTATTAGCTGAG | 2691 | TAAGTACCCAACTAATCTCGCCCCT |
| 1386 | CCACCCTCGCAGGGCCGGTG | 2039 | AGGTCCACTACTATTAGCTGAGT | 2692 | AAGTACCCAACTAATCTCGCCCCTC |
| 1387 | CACCCTCGCAGGGCCGGTGG | 2040 | GGTCCACTACTATTAGCTGAGTG | 2693 | AGTACCCAACTAATCTCGCCCCTCT |
| 1388 | CCCTCGCAGGGCCGGTGGGA | 2041 | GTCCACTACTATTAGCTGAGTGA | 2694 | GTACCCAACTAATCTCGCCCCTCTC |
| 1389 | CCTCGCAGGGCCGGTGGGAA | 2042 | TCCACTACTATTAGCTGAGTGAC | 2695 | TACCCAACTAATCTCGCCCCTCTCA |
| 1390 | CTCGCAGGGCCGGTGGGAAG | 2043 | CCACTACTATTAGCTGAGTGACG | 2696 | ACCCAACTAATCTCGCCCCTCTCAG |
| 1391 | TCGCAGGGCCGGTGGGAAGA | 2044 | CACTACTATTAGCTGAGTGACGC | 2697 | CCCAACTAATCTCGCCCCTCTCAGG |
| 1392 | GCAGGGCCGGTGGGAAGACA | 2045 | ACTACTATTAGCTGAGTGACGCT | 2698 | CCAACTAATCTCGCCCCTCTCAGGA |
| 1393 | CAGGGCCGGTGGGAAGACAC | 2046 | CTACTATTAGCTGAGTGACGCTT | 2699 | CAACTAATCTCGCCCCTCTCAGGAC |
| 1394 | AGGGCCGGTGGGAAGACACA | 2047 | TACTATTAGCTGAGTGACGCTTC | 2700 | AACTAATCTCGCCCCTCTCAGGACC |
| 1395 | GGGCCGGTGGGAAGACACAA | 2048 | ACTATTAGCTGAGTGACGCTTCC | 2701 | ACTAATCTCGCCCCTCTCAGGACCT |
| 1396 | GGCCGGTGGGAAGACACAAA | 2049 | CTATTAGCTGAGTGACGCTTCCA | 2702 | CTAATCTCGCCCCTCTCAGGACCTA |
| 1397 | GCCGGTGGGAAGACACAAAG | 2050 | TATTAGCTGAGTGACGCTTCCAG | 2703 | TAATCTCGCCCCTCTCAGGACCTAC |
| 1398 | CGGTGGGAAGACACAAAGTG | 2051 | ATTAGCTGAGTGACGCTTCCAGC | 2704 | TCTTTTAAAAATTACCTAGAAATAAG |
| 1399 | TGGGAAGACACAAAGTGATG | 2052 | TTAGCTGAGTGACGCTTCCAGCC | 2705 | CTTTTAAAAATTACCTAGAAATAAGG |
| 1400 | AAGACACAAAGTGATGTGGG | 2053 | TAGCTGAGTGACGCTTCCAGCCC | 2706 | TTTTAAAAATTACCTAGAAATAAGGT |
| 1401 | AGACACAAAGTGATGTGGGC | 2054 | AGCTGAGTGACGCTTCCAGCCCG | 2707 | TTTAAAAATTACCTAGAAATAAGGTT |
| 1402 | ACACAAAGTGATGTGGGCAA | 2055 | GCTGAGTGACGCTTCCAGCCCGT | 2708 | TTAAAAATTACCTAGAAATAAGGTTT |
| 1403 | CACAAAGTGATGTGGGCAAG | 2056 | CTGAGTGACGCTTCCAGCCCGTT | 2709 | TAAAAATTACCTAGAAATAAGGTTTC |
| 1404 | CAAAGTGATGTGGGCAAGTG | 2057 | TGAGTGACGCTTCCAGCCCGTTT | 2710 | AAAAATTACCTAGAAATAAGGTTTCT |
| 1405 | AAGTGATGTGGGCAAGTGCC | 2058 | GAGTGACGCTTCCAGCCCGTTTC | 2711 | AAAATTACCTAGAAATAAGGTTTCTT |
| 1406 | AGTGATGTGGGCAAGTGCCA | 2059 | AGTGACGCTTCCAGCCCGTTTCC | 2712 | AAATTACCTAGAAATAAGGTTTCTTT |
| 1407 | GTGATGTGGGCAAGTGCCAG | 2060 | GTGACGCTTCCAGCCCGTTTCCC | 2713 | AATTACCTAGAAATAAGGTTTCTTTA |
| 1408 | TGATGTGGGCAAGTGCCAGG | 2061 | TGACGCTTCCAGCCCGTTTCCCC | 2714 | ATTACCTAGAAATAAGGTTTCTTTAA |
| 1409 | ATGTGGGCAAGTGCCAGGCC | 2062 | GACGCTTCCAGCCCGTTTCCCCC | 2715 | TTACCTAGAAATAAGGTTTCTTTAAT |
| 1410 | TGTGGGCAAGTGCCAGGCCC | 2063 | ACGCTTCCAGCCCGTTTCCCCCG | 2716 | TACCTAGAAATAAGGTTTCTTTAATT |
| 1411 | GTGGGCAAGTGCCAGGCCCA | 2064 | CGCTTCCAGCCCGTTTCCCCCGC | 2717 | ACCTAGAAATAAGGTTTCTTTAATTG |
| 1412 | TGGGCAAGTGCCAGGCCCAC | 2065 | GCTTCCAGCCCGTTTCCCCCGCC | 2718 | CCTAGAAATAAGGTTTCTTTAATTGC |
| 1413 | GGGCAAGTGCCAGGCCCACA | 2066 | CTTCCAGCCCGTTTCCCCCGCCT | 2719 | CTAGAAATAAGGTTTCTTTAATTGCT |
| 1414 | GGCAAGTGCCAGGCCCACAT | 2067 | TTCCAGCCCGTTTCCCCCGCCTG | 2720 | TAGAAATAAGGTTTCTTTAATTGCTG |
| 1415 | GCAAGTGCCAGGCCCACATG | 2068 | TCCAGCCCGTTTCCCCCGCCTGG | 2721 | AGAAATAAGGTTTCTTTAATTGCTGG |
| 1416 | CAAGTGCCAGGCCCACATGG | 2069 | CCAGCCCGTTTCCCCCGCCTGGA | 2722 | GAAATAAGGTTTCTTTAATTGCTGGT |
| 1417 | AGTGCCAGGCCCACATGGTA | 2070 | CAGCCCGTTTCCCCCGCCTGGAA | 2723 | AAATAAGGTTTCTTTAATTGCTGGTT |
| 1418 | GTGCCAGGCCCACATGGTAA | 2071 | AGCCCGTTTCCCCCGCCTGGAAA | 2724 | AATAAGGTTTCTTTAATTGCTGGTTA |
| 1419 | CCAGGCCCACATGGTAAGTA | 2072 | GCCCGTTTCCCCCGCCTGGAAAC | 2725 | ATAAGGTTTCTTTAATTGCTGGTTAA |
| 1420 | CAGGCCCACATGGTAAGTAC | 2073 | CCCGTTTCCCCCGCCTGGAAACT | 2726 | TAAGGTTTCTTTAATTGCTGGTTAAA |
| 1421 | AGGCCCACATGGTAAGTACC | 2074 | CCGTTTCCCCCGCCTGGAAACTG | 2727 | AAGGTTTCTTTAATTGCTGGTTAAAA |
| 1422 | CCCACATGGTAAGTACCCAA | 2075 | CGTTTCCCCCGCCTGGAAACTGA | 2728 | AGGTTTCTTTAATTGCTGGTTAAAAT |
| 1423 | GGTAAGTACCCAACTAATCT | 2076 | GTTTCCCCCGCCTGGAAACTGAG | 2729 | GGTTTCTTTAATTGCTGGTTAAAATG |
| 1424 | GTAAGTACCCAACTAATCTC | 2077 | TTTCCCCCGCCTGGAAACTGAGG | 2730 | GTTTCTTTAATTGCTGGTTAAAATGC |
| 1425 | TAAGTACCCAACTAATCTCG | 2078 | TTCCCCCGCCTGGAAACTGAGGA | 2731 | TTTCTTTAATTGCTGGTTAAAATGCT |
| 1426 | AAGTACCCAACTAATCTCGC | 2079 | TCCCCCGCCTGGAAACTGAGGAC | 2732 | TTCTTTAATTGCTGGTTAAAATGCTC |
| 1427 | GTACCCAACTAATCTCGCCC | 2080 | CCCCCGCCTGGAAACTGAGGACG | 2733 | TCTTTAATTGCTGGTTAAAATGCTCC |
| 1428 | TACCCAACTAATCTCGCCCC | 2081 | CCCCGCCTGGAAACTGAGGACGG | 2734 | CTTTAATTGCTGGTTAAAATGCTCCC |
| 1429 | ACCCAACTAATCTCGCCCCT | 2082 | CCCGCCTGGAAACTGAGGACGGC | 2735 | TTTAATTGCTGGTTAAAATGCTCCCG |
| 1430 | CCCAACTAATCTCGCCCCTC | 2083 | CCGCCTGGAAACTGAGGACGGCA | 2736 | TTAATTGCTGGTTAAAATGCTCCCGT |
| 1431 | CCAACTAATCTCGCCCCTCT | 2084 | CGCCTGGAAACTGAGGACGGCAA | 2737 | TAATTGCTGGTTAAAATGCTCCCGTG |
| 1432 | CAACTAATCTCGCCCCTCTC | 2085 | GCCTGGAAACTGAGGACGGCAAC | 2738 | AATTGCTGGTTAAAATGCTCCCGTGC |
| 1433 | AACTAATCTCGCCCCTCTCA | 2086 | CCTGGAAACTGAGGACGGCAACC | 2739 | ATTGCTGGTTAAAATGCTCCCGTGCA |
| 1434 | ACTAATCTCGCCCCTCTCAG | 2087 | CTGGAAACTGAGGACGGCAACCC | 2740 | TTGCTGGTTAAAATGCTCCCGTGCAG |
| 1435 | TAATCTCGCCCCTCTCAGGA | 2088 | TGGAAACTGAGGACGGCAACCCC | 2741 | TGCTGGTTAAAATGCTCCCGTGCAGA |
| 1436 | AATCTCGCCCCTCTCAGGAC | 2089 | GGAAACTGAGGACGGCAACCCCC | 2742 | GCTGGTTAAAATGCTCCCGTGCAGAA |
| 1437 | ATCTCGCCCCTCTCAGGACC | 2090 | GAAACTGAGGACGGCAACCCCCA | 2743 | CTGGTTAAAATGCTCCCGTGCAGAAA |
| 1438 | TCTCGCCCCTCTCAGGACCT | 2091 | AAACTGAGGACGGCAACCCCCAC | 2744 | TGGTTAAAATGCTCCCGTGCAGAAAA |
| 1439 | CTCGCCCCTCTCAGGACCTA | 2092 | AACTGAGGACGGCAACCCCCACC | 2745 | GGTTAAAATGCTCCCGTGCAGAAAAC |
| 1440 | TCGCCCCTCTCAGGACCTAC | 2093 | ACTGAGGACGGCAACCCCCACCC | 2746 | GTTAAAATGCTCCCGTGCAGAAAACA |
| 1441 | TCTTTTAAAAATTACCTAGAA | 2094 | CTGAGGACGGCAACCCCCACCCT | 2747 | TTAAAATGCTCCCGTGCAGAAAACAG |
| 1442 | AAAATTACCTAGAAATAAGGT | 2095 | TGAGGACGGCAACCCCCACCCTC | 2748 | TAAAATGCTCCCGTGCAGAAAACAGG |
| 1443 | AAATTACCTAGAAATAAGGTT | 2096 | GAGGACGGCAACCCCCACCCTCG | 2749 | AAAATGCTCCCGTGCAGAAAACAGGA |
| 1444 | AATTACCTAGAAATAAGGTTT | 2097 | AGGACGGCAACCCCCACCCTCGC | 2750 | AAATGCTCCCGTGCAGAAAACAGGAA |
| 1445 | ATTACCTAGAAATAAGGTTTC | 2098 | GGACGGCAACCCCCACCCTCGCA | 2751 | AATGCTCCCGTGCAGAAAACAGGAAA |
| 1446 | TTACCTAGAAATAAGGTTTCT | 2099 | GACGGCAACCCCCACCCTCGCAG | 2752 | ATGCTCCCGTGCAGAAAACAGGAAAG |
| 1447 | TACCTAGAAATAAGGTTTCTT | 2100 | ACGGCAACCCCCACCCTCGCAGG | 2753 | TGCTCCCGTGCAGAAAACAGGAAAGA |
| 1448 | ACCTAGAAATAAGGTTTCTTT | 2101 | CGGCAACCCCCACCCTCGCAGGG | 2754 | GCTCCCGTGCAGAAAACAGGAAAGAA |
| 1449 | CCTAGAAATAAGGTTTCTTTA | 2102 | GGCAACCCCCACCCTCGCAGGGC | 2755 | CTCCCGTGCAGAAAACAGGAAAGAAC |
| 1450 | CTAGAAATAAGGTTTCTTTAA | 2103 | GCAACCCCCACCCTCGCAGGGCC | 2756 | TCCCGTGCAGAAAACAGGAAAGAACC |
| 1451 | TAGAAATAAGGTTTCTTTAAT | 2104 | CAACCCCCACCCTCGCAGGGCCG | 2757 | CCCGTGCAGAAAACAGGAAAGAACCT |
| 1452 | AGAAATAAGGTTTCTTTAATT | 2105 | AACCCCCACCCTCGCAGGGCCGG | 2758 | CCGTGCAGAAAACAGGAAAGAACCTG |
| 1453 | GAAATAAGGTTTCTTTAATTG | 2106 | ACCCCCACCCTCGCAGGGCCGGT | 2759 | CGTGCAGAAAACAGGAAAGAACCTGC |
| 1454 | AAATAAGGTTTCTTTAATTGC | 2107 | CCCCCACCCTCGCAGGGCCGGTG | 2760 | GTGCAGAAAACAGGAAAGAACCTGCC |
| 1455 | AATAAGGTTTCTTTAATTGCT | 2108 | CCCCACCCTCGCAGGGCCGGTGG | 2761 | TGCAGAAAACAGGAAAGAACCTGCCT |
| 1456 | ATAAGGTTTCTTTAATTGCTG | 2109 | CCCACCCTCGCAGGGCCGGTGGG | 2762 | GCAGAAAACAGGAAAGAACCTGCCTG |
| 1457 | TAAGGTTTCTTTAATTGCTGG | 2110 | CCACCCTCGCAGGGCCGGTGGGA | 2763 | CAGAAAACAGGAAAGAACCTGCCTGG |
| 1458 | AAGGTTTCTTTAATTGCTGGT | 2111 | CACCCTCGCAGGGCCGGTGGGAA | 2764 | AGAAAACAGGAAAGAACCTGCCTGGT |
| 1459 | AGGTTTCTTTAATTGCTGGTT | 2112 | ACCCTCGCAGGGCCGGTGGGAAG | 2765 | GAAAACAGGAAAGAACCTGCCTGGTT |
| 1460 | GGTTTCTTTAATTGCTGGTTA | 2113 | CCCTCGCAGGGCCGGTGGGAAGA | 2766 | AAAACAGGAAAGAACCTGCCTGGTTC |
| 1461 | GTTTCTTTAATTGCTGGTTAA | 2114 | CCTCGCAGGGCCGGTGGGAAGAC | 2767 | AAACAGGAAAGAACCTGCCTGGTTCC |
| 1462 | TTTCTTTAATTGCTGGTTAAA | 2115 | CTCGCAGGGCCGGTGGGAAGACA | 2768 | AACAGGAAAGAACCTGCCTGGTTCCT |
| 1463 | TTCTTTAATTGCTGGTTAAAA | 2116 | TCGCAGGGCCGGTGGGAAGACAC | 2769 | ACAGGAAAGAACCTGCCTGGTTCCTT |
| 1464 | TCTTTAATTGCTGGTTAAAAT | 2117 | CGCAGGGCCGGTGGGAAGACACA | 2770 | CAGGAAAGAACCTGCCTGGTTCCTTA |
| 1465 | CTTTAATTGCTGGTTAAAATG | 2118 | GCAGGGCCGGTGGGAAGACACAA | 2771 | AGGAAAGAACCTGCCTGGTTCCTTAG |
| 1466 | TTTAATTGCTGGTTAAAATGC | 2119 | CAGGGCCGGTGGGAAGACACAAA | 2772 | GGAAAGAACCTGCCTGGTTCCTTAGC |
| 1467 | TTAATTGCTGGTTAAAATGCT | 2120 | AGGGCCGGTGGGAAGACACAAAG | 2773 | GAAAGAACCTGCCTGGTTCCTTAGCA |
| 1468 | TAATTGCTGGTTAAAATGCTC | 2121 | GGGCCGGTGGGAAGACACAAAGT | 2774 | AAAGAACCTGCCTGGTTCCTTAGCAA |
| 1469 | AATTGCTGGTTAAAATGCTCC | 2122 | GGCCGGTGGGAAGACACAAAGTG | 2775 | AAGAACCTGCCTGGTTCCTTAGCAAA |
| 1470 | ATTGCTGGTTAAAATGCTCCC | 2123 | GCCGGTGGGAAGACACAAAGTGA | 2776 | AGAACCTGCCTGGTTCCTTAGCAAAT |
| 1471 | TTGCTGGTTAAAATGCTCCCG | 2124 | CCGGTGGGAAGACACAAAGTGAT | 2777 | GAACCTGCCTGGTTCCTTAGCAAATC |
| 1472 | TGCTGGTTAAAATGCTCCCGT | 2125 | CGGTGGGAAGACACAAAGTGATG | 2778 | AACCTGCCTGGTTCCTTAGCAAATCT |
| 1473 | GCTGGTTAAAATGCTCCCGTG | 2126 | GGTGGGAAGACACAAAGTGATGT | 2779 | ACCTGCCTGGTTCCTTAGCAAATCTG |
| 1474 | CTGGTTAAAATGCTCCCGTGC | 2127 | GTGGGAAGACACAAAGTGATGTG | 2780 | CCTGCCTGGTTCCTTAGCAAATCTGA |
| 1475 | TGGTTAAAATGCTCCCGTGCA | 2128 | TGGGAAGACACAAAGTGATGTGG | 2781 | CTGCCTGGTTCCTTAGCAAATCTGAG |
| 1476 | GGTTAAAATGCTCCCGTGCAG | 2129 | GGGAAGACACAAAGTGATGTGGG | 2782 | TGCCTGGTTCCTTAGCAAATCTGAGT |
| 1477 | GTTAAAATGCTCCCGTGCAGA | 2130 | GGAAGACACAAAGTGATGTGGGC | 2783 | GCCTGGTTCCTTAGCAAATCTGAGTT |
| 1478 | TAAAATGCTCCCGTGCAGAAA | 2131 | GAAGACACAAAGTGATGTGGGCA | 2784 | CCTGGTTCCTTAGCAAATCTGAGTTC |
| 1479 | AAAATGCTCCCGTGCAGAAAA | 2132 | AAGACACAAAGTGATGTGGGCAA | 2785 | CTGGTTCCTTAGCAAATCTGAGTTCC |
| 1480 | AAATGCTCCCGTGCAGAAAAC | 2133 | AGACACAAAGTGATGTGGGCAAG | 2786 | TGGTTCCTTAGCAAATCTGAGTTCCA |
| 1481 | AATGCTCCCGTGCAGAAAACA | 2134 | GACACAAAGTGATGTGGGCAAGT | 2787 | GGTTCCTTAGCAAATCTGAGTTCCAG |
| 1482 | ATGCTCCCGTGCAGAAAACAG | 2135 | ACACAAAGTGATGTGGGCAAGTG | 2788 | GTTCCTTAGCAAATCTGAGTTCCAGT |
| 1483 | TGCTCCCGTGCAGAAAACAGG | 2136 | CACAAAGTGATGTGGGCAAGTGC | 2789 | TTCCTTAGCAAATCTGAGTTCCAGTC |
| 1484 | GCTCCCGTGCAGAAAACAGGA | 2137 | ACAAAGTGATGTGGGCAAGTGCC | 2790 | TCCTTAGCAAATCTGAGTTCCAGTCC |
| 1485 | CTCCCGTGCAGAAAACAGGAA | 2138 | CAAAGTGATGTGGGCAAGTGCCA | 2791 | CCTTAGCAAATCTGAGTTCCAGTCCC |
| 1486 | TCCCGTGCAGAAAACAGGAAA | 2139 | AAAGTGATGTGGGCAAGTGCCAG | 2792 | CTTAGCAAATCTGAGTTCCAGTCCCA |
| 1487 | CCCGTGCAGAAAACAGGAAAG | 2140 | AAGTGATGTGGGCAAGTGCCAGG | 2793 | TTAGCAAATCTGAGTTCCAGTCCCAG |
| 1488 | CCGTGCAGAAAACAGGAAAGA | 2141 | AGTGATGTGGGCAAGTGCCAGGC | 2794 | TAGCAAATCTGAGTTCCAGTCCCAGG |
| 1489 | CGTGCAGAAAACAGGAAAGAA | 2142 | GTGATGTGGGCAAGTGCCAGGCC | 2795 | AGCAAATCTGAGTTCCAGTCCCAGGT |
| 1490 | GTGCAGAAAACAGGAAAGAAC | 2143 | TGATGTGGGCAAGTGCCAGGCCC | 2796 | GCAAATCTGAGTTCCAGTCCCAGGTC |
| 1491 | AGAAAACAGGAAAGAACCTGC | 2144 | GATGTGGGCAAGTGCCAGGCCCA | 2797 | CAAATCTGAGTTCCAGTCCCAGGTCC |
| 1492 | AAACAGGAAAGAACCTGCCTG | 2145 | ATGTGGGCAAGTGCCAGGCCCAC | 2798 | AAATCTGAGTTCCAGTCCCAGGTCCA |
| 1493 | AACAGGAAAGAACCTGCCTGG | 2146 | TGTGGGCAAGTGCCAGGCCCACA | 2799 | AATCTGAGTTCCAGTCCCAGGTCCAC |
| 1494 | ACAGGAAAGAACCTGCCTGGT | 2147 | GTGGGCAAGTGCCAGGCCCACAT | 2800 | ATCTGAGTTCCAGTCCCAGGTCCACT |
| 1495 | CAGGAAAGAACCTGCCTGGTT | 2148 | TGGGCAAGTGCCAGGCCCACATG | 2801 | TCTGAGTTCCAGTCCCAGGTCCACTA |
| 1496 | AGGAAAGAACCTGCCTGGTTC | 2149 | GGGCAAGTGCCAGGCCCACATGG | 2802 | CTGAGTTCCAGTCCCAGGTCCACTAC |
| 1497 | GGAAAGAACCTGCCTGGTTCC | 2150 | GGCAAGTGCCAGGCCCACATGGT | 2803 | TGAGTTCCAGTCCCAGGTCCACTACT |
| 1498 | GAAAGAACCTGCCTGGTTCCT | 2151 | GCAAGTGCCAGGCCCACATGGTA | 2804 | GAGTTCCAGTCCCAGGTCCACTACTA |
| 1499 | AAAGAACCTGCCTGGTTCCTT | 2152 | CAAGTGCCAGGCCCACATGGTAA | 2805 | AGTTCCAGTCCCAGGTCCACTACTAT |
| 1500 | AAGAACCTGCCTGGTTCCTTA | 2153 | AAGTGCCAGGCCCACATGGTAAG | 2806 | GTTCCAGTCCCAGGTCCACTACTATT |
| 1501 | AGAACCTGCCTGGTTCCTTAG | 2154 | AGTGCCAGGCCCACATGGTAAGT | 2807 | TTCCAGTCCCAGGTCCACTACTATTA |
| 1502 | GAACCTGCCTGGTTCCTTAGC | 2155 | GTGCCAGGCCCACATGGTAAGTA | 2808 | TCCAGTCCCAGGTCCACTACTATTAG |
| 1503 | AACCTGCCTGGTTCCTTAGCA | 2156 | TGCCAGGCCCACATGGTAAGTAC | 2809 | CCAGTCCCAGGTCCACTACTATTAGC |
| 1504 | ACCTGCCTGGTTCCTTAGCAA | 2157 | GCCAGGCCCACATGGTAAGTACC | 2810 | CAGTCCCAGGTCCACTACTATTAGCT |
| 1505 | CCTGCCTGGTTCCTTAGCAAA | 2158 | CCAGGCCCACATGGTAAGTACCC | 2811 | AGTCCCAGGTCCACTACTATTAGCTG |
| 1506 | CTGCCTGGTTCCTTAGCAAAT | 2159 | CAGGCCCACATGGTAAGTACCCA | 2812 | GTCCCAGGTCCACTACTATTAGCTGA |
| 1507 | TGCCTGGTTCCTTAGCAAATC | 2160 | AGGCCCACATGGTAAGTACCCAA | 2813 | TCCCAGGTCCACTACTATTAGCTGAG |
| 1508 | GCCTGGTTCCTTAGCAAATCT | 2161 | GGCCCACATGGTAAGTACCCAAC | 2814 | CCCAGGTCCACTACTATTAGCTGAGT |
| 1509 | CCTGGTTCCTTAGCAAATCTG | 2162 | GCCCACATGGTAAGTACCCAACT | 2815 | CCAGGTCCACTACTATTAGCTGAGTG |
| 1510 | CTGGTTCCTTAGCAAATCTGA | 2163 | CCCACATGGTAAGTACCCAACTA | 2816 | CAGGTCCACTACTATTAGCTGAGTGA |
| 1511 | TGGTTCCTTAGCAAATCTGAG | 2164 | CCACATGGTAAGTACCCAACTAA | 2817 | AGGTCCACTACTATTAGCTGAGTGAC |
| 1512 | GGTTCCTTAGCAAATCTGAGT | 2165 | ACATGGTAAGTACCCAACTAATC | 2818 | GGTCCACTACTATTAGCTGAGTGACG |
| 1513 | GTTCCTTAGCAAATCTGAGTT | 2166 | CATGGTAAGTACCCAACTAATCT | 2819 | GTCCACTACTATTAGCTGAGTGACGC |
| 1514 | TTCCTTAGCAAATCTGAGTTC | 2167 | ATGGTAAGTACCCAACTAATCTC | 2820 | TCCACTACTATTAGCTGAGTGACGCT |
| 1515 | TCCTTAGCAAATCTGAGTTCC | 2168 | TGGTAAGTACCCAACTAATCTCG | 2821 | CCACTACTATTAGCTGAGTGACGCTT |
| 1516 | CCTTAGCAAATCTGAGTTCCA | 2169 | GGTAAGTACCCAACTAATCTCGC | 2822 | CACTACTATTAGCTGAGTGACGCTTC |
| 1517 | CTTAGCAAATCTGAGTTCCAG | 2170 | GTAAGTACCCAACTAATCTCGCC | 2823 | ACTACTATTAGCTGAGTGACGCTTCC |
| 1518 | TTAGCAAATCTGAGTTCCAGT | 2171 | TAAGTACCCAACTAATCTCGCCC | 2824 | CTACTATTAGCTGAGTGACGCTTCCA |
| 1519 | TAGCAAATCTGAGTTCCAGTC | 2172 | AAGTACCCAACTAATCTCGCCCC | 2825 | TACTATTAGCTGAGTGACGCTTCCAG |
| 1520 | AGCAAATCTGAGTTCCAGTCC | 2173 | AGTACCCAACTAATCTCGCCCCT | 2826 | ACTATTAGCTGAGTGACGCTTCCAGC |
| 1521 | GCAAATCTGAGTTCCAGTCCC | 2174 | GTACCCAACTAATCTCGCCCCTC | 2827 | CTATTAGCTGAGTGACGCTTCCAGCC |
| 1522 | AAATCTGAGTTCCAGTCCCAG | 2175 | TACCCAACTAATCTCGCCCCTCT | 2828 | TATTAGCTGAGTGACGCTTCCAGCCC |
| 1523 | AATCTGAGTTCCAGTCCCAGG | 2176 | ACCCAACTAATCTCGCCCCTCTC | 2829 | ATTAGCTGAGTGACGCTTCCAGCCCG |
| 1524 | AGTTCCAGTCCCAGGTCCACT | 2177 | CCCAACTAATCTCGCCCCTCTCA | 2830 | TTAGCTGAGTGACGCTTCCAGCCCGT |
| 1525 | GTTCCAGTCCCAGGTCCACTA | 2178 | CCAACTAATCTCGCCCCTCTCAG | 2831 | TAGCTGAGTGACGCTTCCAGCCCGTT |
| 1526 | TTCCAGTCCCAGGTCCACTAC | 2179 | CAACTAATCTCGCCCCTCTCAGG | 2832 | AGCTGAGTGACGCTTCCAGCCCGTTT |
| 1527 | TCCAGTCCCAGGTCCACTACT | 2180 | AACTAATCTCGCCCCTCTCAGGA | 2833 | GCTGAGTGACGCTTCCAGCCCGTTTC |
| 1528 | CCAGTCCCAGGTCCACTACTA | 2181 | ACTAATCTCGCCCCTCTCAGGAC | 2834 | CTGAGTGACGCTTCCAGCCCGTTTCC |
| 1529 | CAGTCCCAGGTCCACTACTAT | 2182 | CTAATCTCGCCCCTCTCAGGACC | 2835 | TGAGTGACGCTTCCAGCCCGTTTCCC |
| 1530 | AGTCCCAGGTCCACTACTATT | 2183 | TAATCTCGCCCCTCTCAGGACCT | 2836 | GAGTGACGCTTCCAGCCCGTTTCCCC |
| 1531 | GTCCCAGGTCCACTACTATTA | 2184 | AATCTCGCCCCTCTCAGGACCTA | 2837 | AGTGACGCTTCCAGCCCGTTTCCCCC |
| 1532 | TCCCAGGTCCACTACTATTAG | 2185 | ATCTCGCCCCTCTCAGGACCTAC | 2838 | GTGACGCTTCCAGCCCGTTTCCCCCG |
| 1533 | CCCAGGTCCACTACTATTAGC | 2186 | TCTTTTAAAAATTACCTAGAAATA | 2839 | TGACGCTTCCAGCCCGTTTCCCCCGC |
| 1534 | CCAGGTCCACTACTATTAGCT | 2187 | TTTTAAAAATTACCTAGAAATAAG | 2840 | GACGCTTCCAGCCCGTTTCCCCCGCC |
| 1535 | CAGGTCCACTACTATTAGCTG | 2188 | TTTAAAAATTACCTAGAAATAAGG | 2841 | ACGCTTCCAGCCCGTTTCCCCCGCCT |
| 1536 | AGGTCCACTACTATTAGCTGA | 2189 | TTAAAAATTACCTAGAAATAAGGT | 2842 | CGCTTCCAGCCCGTTTCCCCCGCCTG |
| 1537 | GGTCCACTACTATTAGCTGAG | 2190 | TAAAAATTACCTAGAAATAAGGTT | 2843 | GCTTCCAGCCCGTTTCCCCCGCCTGG |
| 1538 | GTCCACTACTATTAGCTGAGT | 2191 | AAAAATTACCTAGAAATAAGGTTT | 2844 | CTTCCAGCCCGTTTCCCCCGCCTGGA |
| 1539 | TCCACTACTATTAGCTGAGTG | 2192 | AAAATTACCTAGAAATAAGGTTTC | 2845 | TTCCAGCCCGTTTCCCCCGCCTGGAA |
| 1540 | ACTACTATTAGCTGAGTGACG | 2193 | AAATTACCTAGAAATAAGGTTTCT | 2846 | TCCAGCCCGTTTCCCCCGCCTGGAAA |
| 1541 | CTACTATTAGCTGAGTGACGC | 2194 | AATTACCTAGAAATAAGGTTTCTT | 2847 | CCAGCCCGTTTCCCCCGCCTGGAAAC |
| 1542 | TACTATTAGCTGAGTGACGCT | 2195 | ATTACCTAGAAATAAGGTTTCTTT | 2848 | CAGCCCGTTTCCCCCGCCTGGAAACT |
| 1543 | ACTATTAGCTGAGTGACGCTT | 2196 | TTACCTAGAAATAAGGTTTCTTTA | 2849 | AGCCCGTTTCCCCCGCCTGGAAACTG |
| 1544 | CTATTAGCTGAGTGACGCTTC | 2197 | TACCTAGAAATAAGGTTTCTTTAA | 2850 | GCCCGTTTCCCCCGCCTGGAAACTGA |
| 1545 | TATTAGCTGAGTGACGCTTCC | 2198 | ACCTAGAAATAAGGTTTCTTTAAT | 2851 | CCCGTTTCCCCCGCCTGGAAACTGAG |
| 1546 | ATTAGCTGAGTGACGCTTCCA | 2199 | CCTAGAAATAAGGTTTCTTTAATT | 2852 | CCGTTTCCCCCGCCTGGAAACTGAGG |
| 1547 | TTAGCTGAGTGACGCTTCCAG | 2200 | CTAGAAATAAGGTTTCTTTAATTG | 2853 | CGTTTCCCCCGCCTGGAAACTGAGGA |
| 1548 | TAGCTGAGTGACGCTTCCAGC | 2201 | TAGAAATAAGGTTTCTTTAATTGC | 2854 | GTTTCCCCCGCCTGGAAACTGAGGAC |
| 1549 | AGCTGAGTGACGCTTCCAGCC | 2202 | AGAAATAAGGTTTCTTTAATTGCT | 2855 | TTTCCCCCGCCTGGAAACTGAGGACG |
| 1550 | GCTGAGTGACGCTTCCAGCCC | 2203 | GAAATAAGGTTTCTTTAATTGCTG | 2856 | TTCCCCCGCCTGGAAACTGAGGACGG |
| 1551 | CTGAGTGACGCTTCCAGCCCG | 2204 | AAATAAGGTTTCTTTAATTGCTGG | 2857 | TCCCCCGCCTGGAAACTGAGGACGGC |
| 1552 | TGAGTGACGCTTCCAGCCCGT | 2205 | AATAAGGTTTCTTTAATTGCTGGT | 2858 | CCCCCGCCTGGAAACTGAGGACGGCA |
| 1553 | GAGTGACGCTTCCAGCCCGTT | 2206 | ATAAGGTTTCTTTAATTGCTGGTT | 2859 | CCCCGCCTGGAAACTGAGGACGGCAA |
| 1554 | AGTGACGCTTCCAGCCCGTTT | 2207 | TAAGGTTTCTTTAATTGCTGGTTA | 2860 | CCCGCCTGGAAACTGAGGACGGCAAC |
| 1555 | GTGACGCTTCCAGCCCGTTTC | 2208 | AAGGTTTCTTTAATTGCTGGTTAA | 2861 | CCGCCTGGAAACTGAGGACGGCAACC |
| 1556 | TGACGCTTCCAGCCCGTTTCC | 2209 | AGGTTTCTTTAATTGCTGGTTAAA | 2862 | CGCCTGGAAACTGAGGACGGCAACCC |
| 1557 | GACGCTTCCAGCCCGTTTCCC | 2210 | GGTTTCTTTAATTGCTGGTTAAAA | 2863 | GCCTGGAAACTGAGGACGGCAACCCC |
| 1558 | ACGCTTCCAGCCCGTTTCCCC | 2211 | GTTTCTTTAATTGCTGGTTAAAAT | 2864 | CCTGGAAACTGAGGACGGCAACCCCC |
| 1559 | CGCTTCCAGCCCGTTTCCCCC | 2212 | TTTCTTTAATTGCTGGTTAAAATG | 2865 | CTGGAAACTGAGGACGGCAACCCCCA |
| 1560 | GCTTCCAGCCCGTTTCCCCCG | 2213 | TTCTTTAATTGCTGGTTAAAATGC | 2866 | TGGAAACTGAGGACGGCAACCCCCAC |
| 1561 | CTTCCAGCCCGTTTCCCCCGC | 2214 | TCTTTAATTGCTGGTTAAAATGCT | 2867 | GGAAACTGAGGACGGCAACCCCCACC |
| 1562 | TTCCAGCCCGTTTCCCCCGCC | 2215 | CTTTAATTGCTGGTTAAAATGCTC | 2868 | GAAACTGAGGACGGCAACCCCCACCC |
| 1563 | TCCAGCCCGTTTCCCCCGCCT | 2216 | TTTAATTGCTGGTTAAAATGCTCC | 2869 | AAACTGAGGACGGCAACCCCCACCCT |
| 1564 | CCAGCCCGTTTCCCCCGCCTG | 2217 | TTAATTGCTGGTTAAAATGCTCCC | 2870 | AACTGAGGACGGCAACCCCCACCCTC |
| 1565 | CAGCCCGTTTCCCCCGCCTGG | 2218 | TAATTGCTGGTTAAAATGCTCCCG | 2871 | ACTGAGGACGGCAACCCCCACCCTCG |
| 1566 | AGCCCGTTTCCCCCGCCTGGA | 2219 | AATTGCTGGTTAAAATGCTCCCGT | 2872 | CTGAGGACGGCAACCCCCACCCTCGC |
| 1567 | GCCCGTTTCCCCCGCCTGGAA | 2220 | ATTGCTGGTTAAAATGCTCCCGTG | 2873 | TGAGGACGGCAACCCCCACCCTCGCA |
| 1568 | CCCGTTTCCCCCGCCTGGAAA | 2221 | TTGCTGGTTAAAATGCTCCCGTGC | 2874 | GAGGACGGCAACCCCCACCCTCGCAG |
| 1569 | CCGTTTCCCCCGCCTGGAAAC | 2222 | TGCTGGTTAAAATGCTCCCGTGCA | 2875 | AGGACGGCAACCCCCACCCTCGCAGG |
| 1570 | CGTTTCCCCCGCCTGGAAACT | 2223 | GCTGGTTAAAATGCTCCCGTGCAG | 2876 | GGACGGCAACCCCCACCCTCGCAGGG |
| 1571 | GTTTCCCCCGCCTGGAAACTG | 2224 | CTGGTTAAAATGCTCCCGTGCAGA | 2877 | GACGGCAACCCCCACCCTCGCAGGGC |
| 1572 | TTTCCCCCGCCTGGAAACTGA | 2225 | TGGTTAAAATGCTCCCGTGCAGAA | 2878 | ACGGCAACCCCCACCCTCGCAGGGCC |
| 1573 | TTCCCCCGCCTGGAAACTGAG | 2226 | GGTTAAAATGCTCCCGTGCAGAAA | 2879 | CGGCAACCCCCACCCTCGCAGGGCCG |
| 1574 | TCCCCCGCCTGGAAACTGAGG | 2227 | GTTAAAATGCTCCCGTGCAGAAAA | 2880 | GGCAACCCCCACCCTCGCAGGGCCGG |
| 1575 | CCCCCGCCTGGAAACTGAGGA | 2228 | TTAAAATGCTCCCGTGCAGAAAAC | 2881 | GCAACCCCCACCCTCGCAGGGCCGGT |
| 1576 | CCCCGCCTGGAAACTGAGGAC | 2229 | TAAAATGCTCCCGTGCAGAAAACA | 2882 | CAACCCCCACCCTCGCAGGGCCGGTG |
| 1577 | CCCGCCTGGAAACTGAGGACG | 2230 | AAAATGCTCCCGTGCAGAAAACAG | 2883 | AACCCCCACCCTCGCAGGGCCGGTGG |
| 1578 | CCGCCTGGAAACTGAGGACGG | 2231 | AAATGCTCCCGTGCAGAAAACAGG | 2884 | ACCCCCACCCTCGCAGGGCCGGTGGG |
| 1579 | CGCCTGGAAACTGAGGACGGC | 2232 | AATGCTCCCGTGCAGAAAACAGGA | 2885 | CCCCCACCCTCGCAGGGCCGGTGGGA |
| 1580 | GCCTGGAAACTGAGGACGGCA | 2233 | ATGCTCCCGTGCAGAAAACAGGAA | 2886 | CCCCACCCTCGCAGGGCCGGTGGGAA |
| 1581 | CCTGGAAACTGAGGACGGCAA | 2234 | TGCTCCCGTGCAGAAAACAGGAAA | 2887 | CCCACCCTCGCAGGGCCGGTGGGAAG |
| 1582 | CTGGAAACTGAGGACGGCAAC | 2235 | GCTCCCGTGCAGAAAACAGGAAAG | 2888 | CCACCCTCGCAGGGCCGGTGGGAAGA |
| 1583 | TGGAAACTGAGGACGGCAACC | 2236 | CTCCCGTGCAGAAAACAGGAAAGA | 2889 | CACCCTCGCAGGGCCGGTGGGAAGAC |
| 1584 | GGAAACTGAGGACGGCAACCC | 2237 | TCCCGTGCAGAAAACAGGAAAGAA | 2890 | ACCCTCGCAGGGCCGGTGGGAAGACA |
| 1585 | AAACTGAGGACGGCAACCCCC | 2238 | CCCGTGCAGAAAACAGGAAAGAAC | 2891 | CCCTCGCAGGGCCGGTGGGAAGACAC |
| 1586 | AACTGAGGACGGCAACCCCCA | 2239 | CCGTGCAGAAAACAGGAAAGAACC | 2892 | CCTCGCAGGGCCGGTGGGAAGACACA |
| 1587 | ACTGAGGACGGCAACCCCCAC | 2240 | CGTGCAGAAAACAGGAAAGAACCT | 2893 | CTCGCAGGGCCGGTGGGAAGACACAA |
| 1588 | CTGAGGACGGCAACCCCCACC | 2241 | GTGCAGAAAACAGGAAAGAACCTG | 2894 | TCGCAGGGCCGGTGGGAAGACACAAA |
| 1589 | TGAGGACGGCAACCCCCACCC | 2242 | TGCAGAAAACAGGAAAGAACCTGC | 2895 | CGCAGGGCCGGTGGGAAGACACAAAG |
| 1590 | GAGGACGGCAACCCCCACCCT | 2243 | GCAGAAAACAGGAAAGAACCTGCC | 2896 | GCAGGGCCGGTGGGAAGACACAAAGT |
| 1591 | AGGACGGCAACCCCCACCCTC | 2244 | CAGAAAACAGGAAAGAACCTGCCT | 2897 | CAGGGCCGGTGGGAAGACACAAAGTG |
| 1592 | GGACGGCAACCCCCACCCTCG | 2245 | AGAAAACAGGAAAGAACCTGCCTG | 2898 | AGGGCCGGTGGGAAGACACAAAGTGA |
| 1593 | ACGGCAACCCCCACCCTCGCA | 2246 | GAAAACAGGAAAGAACCTGCCTGG | 2899 | GGGCCGGTGGGAAGACACAAAGTGAT |
| 1594 | CGGCAACCCCCACCCTCGCAG | 2247 | AAAACAGGAAAGAACCTGCCTGGT | 2900 | GGCCGGTGGGAAGACACAAAGTGATG |
| 1595 | GGCAACCCCCACCCTCGCAGG | 2248 | AAACAGGAAAGAACCTGCCTGGTT | 2901 | GCCGGTGGGAAGACACAAAGTGATGT |
| 1596 | GCAACCCCCACCCTCGCAGGG | 2249 | AACAGGAAAGAACCTGCCTGGTTC | 2902 | CCGGTGGGAAGACACAAAGTGATGTG |
| 1597 | CAACCCCCACCCTCGCAGGGC | 2250 | ACAGGAAAGAACCTGCCTGGTTCC | 2903 | CGGTGGGAAGACACAAAGTGATGTGG |
| 1598 | AACCCCCACCCTCGCAGGGCC | 2251 | CAGGAAAGAACCTGCCTGGTTCCT | 2904 | GGTGGGAAGACACAAAGTGATGTGGG |
| 1599 | ACCCCCACCCTCGCAGGGCCG | 2252 | AGGAAAGAACCTGCCTGGTTCCTT | 2905 | GTGGGAAGACACAAAGTGATGTGGGC |
| 1600 | CCCCCACCCTCGCAGGGCCGG | 2253 | GGAAAGAACCTGCCTGGTTCCTTA | 2906 | TGGGAAGACACAAAGTGATGTGGGCA |
| 1601 | CCCCACCCTCGCAGGGCCGGT | 2254 | GAAAGAACCTGCCTGGTTCCTTAG | 2907 | GGGAAGACACAAAGTGATGTGGGCAA |
| 1602 | CCCACCCTCGCAGGGCCGGTG | 2255 | AAAGAACCTGCCTGGTTCCTTAGC | 2908 | GGAAGACACAAAGTGATGTGGGCAAG |
| 1603 | CCACCCTCGCAGGGCCGGTGG | 2256 | AAGAACCTGCCTGGTTCCTTAGCA | 2909 | GAAGACACAAAGTGATGTGGGCAAGT |
| 1604 | CACCCTCGCAGGGCCGGTGGG | 2257 | AGAACCTGCCTGGTTCCTTAGCAA | 2910 | AAGACACAAAGTGATGTGGGCAAGTG |
| 1605 | ACCCTCGCAGGGCCGGTGGGA | 2258 | GAACCTGCCTGGTTCCTTAGCAAA | 2911 | AGACACAAAGTGATGTGGGCAAGTGC |
| 1606 | CCCTCGCAGGGCCGGTGGGAA | 2259 | AACCTGCCTGGTTCCTTAGCAAAT | 2912 | GACACAAAGTGATGTGGGCAAGTGCC |
| 1607 | CCTCGCAGGGCCGGTGGGAAG | 2260 | ACCTGCCTGGTTCCTTAGCAAATC | 2913 | ACACAAAGTGATGTGGGCAAGTGCCA |
| 1608 | CTCGCAGGGCCGGTGGGAAGA | 2261 | CCTGCCTGGTTCCTTAGCAAATCT | 2914 | CACAAAGTGATGTGGGCAAGTGCCAG |
| 1609 | TCGCAGGGCCGGTGGGAAGAC | 2262 | CTGCCTGGTTCCTTAGCAAATCTG | 2915 | ACAAAGTGATGTGGGCAAGTGCCAGG |
| 1610 | CGCAGGGCCGGTGGGAAGACA | 2263 | TGCCTGGTTCCTTAGCAAATCTGA | 2916 | CAAAGTGATGTGGGCAAGTGCCAGGC |
| 1611 | GCAGGGCCGGTGGGAAGACAC | 2264 | GCCTGGTTCCTTAGCAAATCTGAG | 2917 | AAAGTGATGTGGGCAAGTGCCAGGCC |
| 1612 | CAGGGCCGGTGGGAAGACACA | 2265 | CCTGGTTCCTTAGCAAATCTGAGT | 2918 | AAGTGATGTGGGCAAGTGCCAGGCCC |
| 1613 | AGGGCCGGTGGGAAGACACAA | 2266 | CTGGTTCCTTAGCAAATCTGAGTT | 2919 | AGTGATGTGGGCAAGTGCCAGGCCCA |
| 1614 | GGGCCGGTGGGAAGACACAAA | 2267 | TGGTTCCTTAGCAAATCTGAGTTC | 2920 | GTGATGTGGGCAAGTGCCAGGCCCAC |
| 1615 | GGCCGGTGGGAAGACACAAAG | 2268 | GGTTCCTTAGCAAATCTGAGTTCC | 2921 | TGATGTGGGCAAGTGCCAGGCCCACA |
| 1616 | GCCGGTGGGAAGACACAAAGT | 2269 | GTTCCTTAGCAAATCTGAGTTCCA | 2922 | GATGTGGGCAAGTGCCAGGCCCACAT |
| 1617 | CCGGTGGGAAGACACAAAGTG | 2270 | TTCCTTAGCAAATCTGAGTTCCAG | 2923 | ATGTGGGCAAGTGCCAGGCCCACATG |
| 1618 | CGGTGGGAAGACACAAAGTGA | 2271 | TCCTTAGCAAATCTGAGTTCCAGT | 2924 | TGTGGGCAAGTGCCAGGCCCACATGG |
| 1619 | GGTGGGAAGACACAAAGTGAT | 2272 | CCTTAGCAAATCTGAGTTCCAGTC | 2925 | GTGGGCAAGTGCCAGGCCCACATGGT |
| 1620 | GTGGGAAGACACAAAGTGATG | 2273 | CTTAGCAAATCTGAGTTCCAGTCC | 2926 | TGGGCAAGTGCCAGGCCCACATGGTA |
| 1621 | TGGGAAGACACAAAGTGATGT | 2274 | TTAGCAAATCTGAGTTCCAGTCCC | 2927 | GGGCAAGTGCCAGGCCCACATGGTAA |
| 1622 | GAAGACACAAAGTGATGTGGG | 2275 | TAGCAAATCTGAGTTCCAGTCCCA | 2928 | GGCAAGTGCCAGGCCCACATGGTAAG |
| 1623 | AAGACACAAAGTGATGTGGGC | 2276 | AGCAAATCTGAGTTCCAGTCCCAG | 2929 | GCAAGTGCCAGGCCCACATGGTAAGT |
| 1624 | AGACACAAAGTGATGTGGGCA | 2277 | GCAAATCTGAGTTCCAGTCCCAGG | 2930 | CAAGTGCCAGGCCCACATGGTAAGTA |
| 1625 | GACACAAAGTGATGTGGGCAA | 2278 | CAAATCTGAGTTCCAGTCCCAGGT | 2931 | AAGTGCCAGGCCCACATGGTAAGTAC |
| 1626 | ACACAAAGTGATGTGGGCAAG | 2279 | AAATCTGAGTTCCAGTCCCAGGTC | 2932 | AGTGCCAGGCCCACATGGTAAGTACC |
| 1627 | CACAAAGTGATGTGGGCAAGT | 2280 | AATCTGAGTTCCAGTCCCAGGTCC | 2933 | GTGCCAGGCCCACATGGTAAGTACCC |
| 1628 | ACAAAGTGATGTGGGCAAGTG | 2281 | ATCTGAGTTCCAGTCCCAGGTCCA | 2934 | TGCCAGGCCCACATGGTAAGTACCCA |
| 1629 | CAAAGTGATGTGGGCAAGTGC | 2282 | TCTGAGTTCCAGTCCCAGGTCCAC | 2935 | GCCAGGCCCACATGGTAAGTACCCAA |
| 1630 | AAAGTGATGTGGGCAAGTGCC | 2283 | CTGAGTTCCAGTCCCAGGTCCACT | 2936 | CCAGGCCCACATGGTAAGTACCCAAC |
| 1631 | AAGTGATGTGGGCAAGTGCCA | 2284 | TGAGTTCCAGTCCCAGGTCCACTA | 2937 | CAGGCCCACATGGTAAGTACCCAACT |
| 1632 | AGTGATGTGGGCAAGTGCCAG | 2285 | GAGTTCCAGTCCCAGGTCCACTAC | 2938 | AGGCCCACATGGTAAGTACCCAACTA |
| 1633 | GTGATGTGGGCAAGTGCCAGG | 2286 | AGTTCCAGTCCCAGGTCCACTACT | 2939 | GGCCCACATGGTAAGTACCCAACTAA |
| 1634 | TGATGTGGGCAAGTGCCAGGC | 2287 | GTTCCAGTCCCAGGTCCACTACTA | 2940 | GCCCACATGGTAAGTACCCAACTAAT |
| 1635 | GATGTGGGCAAGTGCCAGGCC | 2288 | TTCCAGTCCCAGGTCCACTACTAT | 2941 | CCCACATGGTAAGTACCCAACTAATC |
| 1636 | ATGTGGGCAAGTGCCAGGCCC | 2289 | TCCAGTCCCAGGTCCACTACTATT | 2942 | CCACATGGTAAGTACCCAACTAATCT |
| 1637 | TGTGGGCAAGTGCCAGGCCCA | 2290 | CCAGTCCCAGGTCCACTACTATTA | 2943 | CACATGGTAAGTACCCAACTAATCTC |
| 1638 | GTGGGCAAGTGCCAGGCCCAC | 2291 | CAGTCCCAGGTCCACTACTATTAG | 2944 | ACATGGTAAGTACCCAACTAATCTCG |
| 1639 | TGGGCAAGTGCCAGGCCCACA | 2292 | AGTCCCAGGTCCACTACTATTAGC | 2945 | CATGGTAAGTACCCAACTAATCTCGC |
| 1640 | GGGCAAGTGCCAGGCCCACAT | 2293 | GTCCCAGGTCCACTACTATTAGCT | 2946 | ATGGTAAGTACCCAACTAATCTCGCC |
| 1641 | GGCAAGTGCCAGGCCCACATG | 2294 | TCCCAGGTCCACTACTATTAGCTG | 2947 | TGGTAAGTACCCAACTAATCTCGCCC |
| 1642 | GCAAGTGCCAGGCCCACATGG | 2295 | CCCAGGTCCACTACTATTAGCTGA | 2948 | GGTAAGTACCCAACTAATCTCGCCCC |
| 1643 | CAAGTGCCAGGCCCACATGGT | 2296 | CCAGGTCCACTACTATTAGCTGAG | 2949 | GTAAGTACCCAACTAATCTCGCCCCT |
| 1644 | AAGTGCCAGGCCCACATGGTA | 2297 | CAGGTCCACTACTATTAGCTGAGT | 2950 | TAAGTACCCAACTAATCTCGCCCCTC |
| 1645 | AGTGCCAGGCCCACATGGTAA | 2298 | AGGTCCACTACTATTAGCTGAGTG | 2951 | AAGTACCCAACTAATCTCGCCCCTCT |
| 1646 | GTGCCAGGCCCACATGGTAAG | 2299 | GGTCCACTACTATTAGCTGAGTGA | 2952 | AGTACCCAACTAATCTCGCCCCTCTC |
| 1647 | TGCCAGGCCCACATGGTAAGT | 2300 | GTCCACTACTATTAGCTGAGTGAC | 2953 | GTACCCAACTAATCTCGCCCCTCTCA |
| 1648 | GCCAGGCCCACATGGTAAGTA | 2301 | TCCACTACTATTAGCTGAGTGACG | 2954 | TACCCAACTAATCTCGCCCCTCTCAG |
| 1649 | CCAGGCCCACATGGTAAGTAC | 2302 | CCACTACTATTAGCTGAGTGACGC | 2955 | ACCCAACTAATCTCGCCCCTCTCAGG |
| 1650 | CAGGCCCACATGGTAAGTACC | 2303 | CACTACTATTAGCTGAGTGACGCT | 2956 | CCCAACTAATCTCGCCCCTCTCAGGA |
| 1651 | AGGCCCACATGGTAAGTACCC | 2304 | ACTACTATTAGCTGAGTGACGCTT | 2957 | CCAACTAATCTCGCCCCTCTCAGGAC |
| 1652 | GGCCCACATGGTAAGTACCCA | 2305 | CTACTATTAGCTGAGTGACGCTTC | 2958 | CAACTAATCTCGCCCCTCTCAGGACC |
| 1653 | GCCCACATGGTAAGTACCCAA | 2306 | TACTATTAGCTGAGTGACGCTTCC | 2959 | AACTAATCTCGCCCCTCTCAGGACCT |
| 1654 | CCCACATGGTAAGTACCCAAC | 2307 | ACTATTAGCTGAGTGACGCTTCCA | 2960 | ACTAATCTCGCCCCTCTCAGGACCTA |
| 1655 | CCACATGGTAAGTACCCAACT | 2308 | CTATTAGCTGAGTGACGCTTCCAG | 2961 | CTAATCTCGCCCCTCTCAGGACCTAC |
| 1656 | ATGGTAAGTACCCAACTAATC | 2309 | TATTAGCTGAGTGACGCTTCCAGC | | |

TABLE 4
Exemplary SYNGAP1 ASO sequences that target RR93_v1 and/or RR93_v2

| SEQ ID | | SEQ | | SEQ | |
|---|---|---|---|---|---|
| NO | Sequence | ID NO | Sequence | ID NO | Sequence |
| 2962 | GCTCTTATTCTCCTCCTCCT | 3593 | TGGGTCTAGGGTCTCTCTGTC | 4224 | TTTTTTCCTCAAATCAGGAGTCTC |
| 2963 | TGCTGCCGTTGGCTCTTATT | 3594 | GGGTCTAGGGTCTCTCTGTCT | 4225 | TTTTTCCTCAAATCAGGAGTCTCT |
| 2964 | TGCCGCTGCTGCCGTTGGCT | 3595 | GGTCTAGGGTCTCTCTGTCTC | 4226 | TTTTCCTCAAATCAGGAGTCTCTT |
| 2965 | TCGGCTGCCGCTGCTGCCGT | 3596 | GTCTAGGGTCTCTCTGTCTCA | 4227 | TTTCCTCAAATCAGGAGTCTCTTT |
| 2966 | TCTCTCTCGGCTGCCGCTGC | 3597 | TCTAGGGTCTCTCTGTCTCAG | 4228 | TTCCTCAAATCAGGAGTCTCTTTT |
| 2967 | CCGCCCCCCCTCTCTCTCGG | 3598 | CTAGGGTCTCTCTGTCTCAGG | 4229 | TCCTCAAATCAGGAGTCTCTTTTC |
| 2968 | CGCTCCCCCGCCCCCCCTCT | 3599 | TAGGGTCTCTCTGTCTCAGGG | 4230 | CCTCAAATCAGGAGTCTCTTTTCC |
| 2969 | GCTCTCGCTCGCTCCCCCGC | 3600 | AGGGTCTCTCTGTCTCAGGGT | 4231 | CTCAAATCAGGAGTCTCTTTTCCT |
| 2970 | TCTTCGCTCTCGCTCGCTCC | 3601 | GGGTCTCTCTGTCTCAGGGTC | 4232 | TCAAATCAGGAGTCTCTTTTCCTC |
| 2971 | CTGCTCTCTTCGCTCTCGCT | 3602 | GGTCTCTCTGTCTCAGGGTCT | 4233 | CAAATCAGGAGTCTCTTTTCCTCT |
| 2972 | TGCAGAGTCTCTCCCTCCTC | 3603 | GTCTCTCTGTCTCAGGGTCTG | 4234 | AAATCAGGAGTCTCTTTTCCTCTA |
| 2973 | GGGGGCTGCAGAGTCTCTCC | 3604 | TCTCTCTGTCTCAGGGTCTGG | 4235 | AATCAGGAGTCTCTTTTCCTCTAG |
| 2974 | GGGGTGGGGGCTGCAGAGTC | 3605 | CTCTCTGTCTCAGGGTCTGGG | 4236 | ATCAGGAGTCTCTTTTCCTCTAGA |
| 2975 | GAGTAGGGGTGGGGGCTGCA | 3606 | TCTCTGTCTCAGGGTCTGGGG | 4237 | TCAGGAGTCTCTTTTCCTCTAGAT |
| 2976 | TCCCGGAGTAGGGGTGGGGG | 3607 | CTCTGTCTCAGGGTCTGGGGT | 4238 | CAGGAGTCTCTTTTCCTCTAGATT |
| 2977 | GGGCCTCCCGGAGTAGGGGT | 3608 | TCTGTCTCAGGGTCTGGGGTC | 4239 | AGGAGTCTCTTTTCCTCTAGATTT |
| 2978 | AATCTGGGCCTCCCGGAGTA | 3609 | CTGTCTCAGGGTCTGGGGTCT | 4240 | GGAGTCTCTTTTCCTCTAGATTTT |
| 2979 | CTCTCTCACAATCTGGGCCT | 3610 | TGTCTCAGGGTCTGGGGTCTG | 4241 | GAGTCTCTTTTCCTCTAGATTTTG |
| 2980 | GTGTTTTTGAGTCAGGGGTC | 3611 | GTCTCAGGGTCTGGGGTCTGC | 4242 | AGTCTCTTTTCCTCTAGATTTTGG |
| 2981 | GGGGTCTGCATCTTTAGGAC | 3612 | TCTCAGGGTCTGGGGTCTGCA | 4243 | GTCTCTTTTCCTCTAGATTTTGGC |
| 2982 | CAGGGTCTGGGGTCTGCATC | 3613 | CTCAGGGTCTGGGGTCTGCAT | 4244 | TCTCTTTTCCTCTAGATTTTGGCC |
| 2983 | TGTCTCAGGGTCTGGGGTCT | 3614 | TCAGGGTCTGGGGTCTGCATC | 4245 | CTCTTTTCCTCTAGATTTTGGCCC |
| 2984 | GGTCTCTCTGTCTCAGGGTC | 3615 | CAGGGTCTGGGGTCTGCATCT | 4246 | TCTTTTCCTCTAGATTTTGGCCCT |
| 2985 | TATCTTTCTCTGTGGGTCTA | 3616 | AGGGTCTGGGGTCTGCATCTT | 4247 | CTTTTCCTCTAGATTTTGGCCCTG |
| 2986 | TGGGGGGTGGTGAAGTATCT | 3617 | GGTCTGGGGTCTGCATCTTTA | 4248 | TTTTCCTCTAGATTTTGGCCCTGT |
| 2987 | CCACAGTGGGGGGTGGTGAA | 3618 | GTCTGGGGTCTGCATCTTTAG | 4249 | TTTCCTCTAGATTTTGGCCCTGTG |
| 2988 | ATTACCCCACAGTGGGGGGT | 3619 | TCTGGGGTCTGCATCTTTAGG | 4250 | TTCCTCTAGATTTTGGCCCTGTGT |
| 2989 | TAGATTTTGGCCCTGTGTCT | 3620 | CTGGGGTCTGCATCTTTAGGA | 4251 | TCCTCTAGATTTTGGCCCTGTGTC |
| 2990 | GGAGTCTCTTTTCCTCTAGA | 3621 | TGGGGTCTGCATCTTTAGGAC | 4252 | CCTCTAGATTTTGGCCCTGTGTCT |
| 2991 | CTCAAATCAGGAGTCTCTTT | 3622 | GGGGTCTGCATCTTTAGGACC | 4253 | CTCTAGATTTTGGCCCTGTGTCTC |
| 2992 | TTTTCCTCAAATCAGGAGTC | 3623 | GGGTCTGCATCTTTAGGACCT | 4254 | TCTAGATTTTGGCCCTGTGTCTCA |
| 2993 | GGGTCGTTTTTTCCTCAAAT | 3624 | GGTCTGCATCTTTAGGACCTC | 4255 | CTAGATTTTGGCCCTGTGTCTCAA |
| 2994 | GGATCTGGGGTCGTTTTTTC | 3625 | CTGCATCTTTAGGACCTCTGT | 4256 | TAGATTTTGGCCCTGTGTCTCAAA |
| 2995 | CTGGGGGATCTGGGGTCGTT | 3626 | TGCATCTTTAGGACCTCTGTC | 4257 | AGATTTTGGCCCTGTGTCTCAAAT |
| 2996 | TCTCTCTGGGGGATCTGGGG | 3627 | GCATCTTTAGGACCTCTGTCT | 4258 | GATTTTGGCCCTGTGTCTCAAATT |
| 2997 | GCTTCTCTCTCTGGGGGATC | 3628 | CATCTTTAGGACCTCTGTCTC | 4259 | ATTTTGGCCCTGTGTCTCAAATTA |
| 2998 | ATTTTGATATTGAGCTTCTC | 3629 | ATCTTTAGGACCTCTGTCTCT | 4260 | TTTTGGCCCTGTGTCTCAAATTAC |
| 2999 | TTCAGATTTTGATATTGAGC | 3630 | TCTTTAGGACCTCTGTCTCTC | 4261 | TTTGGCCCTGTGTCTCAAATTACC |
| 3000 | GACTGGATTTTCAGATTTTG | 3631 | CTTTAGGACCTCTGTCTCTCT | 4262 | TTGGCCCTGTGTCTCAAATTACCC |
| 3001 | CCTTTTTAGACTGGATTTTC | 3632 | TTAGGACCTCTGTCTCTCTCT | 4263 | TGGCCCTGTGTCTCAAATTACCCC |
| 3002 | AGGGTCTGGAGTCTAAGATA | 3633 | TAGGACCTCTGTCTCTCTCTG | 4264 | GGCCCTGTGTCTCAAATTACCCCA |
| 3003 | ATCTCAGGGTCTGGAGTCTA | 3634 | AGGACCTCTGTCTCTCTCTGG | 4265 | GCCCTGTGTCTCAAATTACCCCAC |
| 3004 | TCATCATCTCAGGGTCTGGA | 3635 | GGACCTCTGTCTCTCTCTGGT | 4266 | CCCTGTGTCTCAAATTACCCCACA |
| 3005 | TGAAATCATCATCTCAGGGT | 3636 | CCTCTGTCTCTCTCTGGTGTG | 4267 | CCTGTGTCTCAAATTACCCCACAG |
| 3006 | TGGTCCCTGAAATCATCATC | 3637 | TCTGTCTCTCTCTGGTGTGTT | 4268 | CTGTGTCTCAAATTACCCCACAGT |
| 3007 | GGTCCTGGTCCCTGAAATCA | 3638 | CTGTCTCTCTCTGGTGTGTTT | 4269 | TGTGTCTCAAATTACCCCACAGTG |
| 3008 | AGGTCTCAGGTCCTGGTCCC | 3639 | GGGTCTGGAGTCTAAGATATCT | 4270 | GTGTCTCAAATTACCCCACAGTGG |
| 3009 | AGTCTAGGTCTCAGGTCCTG | 3640 | GGTCTGGAGTCTAAGATATCTG | 4271 | TGTCTCAAATTACCCCACAGTGGG |
| 3010 | TTCTGAGTCTAGGTCTCAGG | 3641 | GTCTGGAGTCTAAGATATCTGT | 4272 | GTCTCAAATTACCCCACAGTGGGG |
| 3011 | CATCTTTTTCTGAGTCTAGG | 3642 | TCTGGAGTCTAAGATATCTGTA | 4273 | TCTCAAATTACCCCACAGTGGGGG |
| 3012 | GGGCCTCATCTTTTTCTGAG | 3643 | CTGGAGTCTAAGATATCTGTAG | 4274 | CTCAAATTACCCCACAGTGGGGGG |
| 3013 | TCTGAATCTGGGCCTCATCT | 3644 | TGGAGTCTAAGATATCTGTAGG | 4275 | TCAAATTACCCCACAGTGGGGGGT |
| 3014 | GCTCTTTCTGAATCTGGGCC | 3645 | GGAGTCTAAGATATCTGTAGGT | 4276 | CAAATTACCCCACAGTGGGGGGTG |
| 3015 | CATCTGGCTCTTTCTGAATC | 3646 | GAGTCTAAGATATCTGTAGGTA | 4277 | AAATTACCCCACAGTGGGGGGTGG |
| 3016 | TCTAATTCTACATCTGGCTC | 3647 | AGTCTAAGATATCTGTAGGTAT | 4278 | AATTACCCCACAGTGGGGGGTGGT |
| 3017 | CTCTAAGCTCTAATTCTACA | 3648 | GTCTAAGATATCTGTAGGTATG | 4279 | ATTACCCCACAGTGGGGGGTGGTG |
| 3018 | GAGTTCTCTAAGCTCTAATT | 3649 | TCTAAGATATCTGTAGGTATGG | 4280 | TTACCCCACAGTGGGGGGTGGTGA |
| 3019 | TAGGTCTGAGTTCTCTAAGC | 3650 | CTAAGATATCTGTAGGTATGGA | 4281 | TACCCCACAGTGGGGGGTGGTGAA |
| 3020 | GGGGTCTCTTTCTAGGTCTG | 3651 | TAAGATATCTGTAGGTATGGAG | 4282 | ACCCCACAGTGGGGGGTGGTGAAG |
| 3021 | AGTCTGGGGTCTCTTTCTAG | 3652 | AAGATATCTGTAGGTATGGAGA | 4283 | CCCCACAGTGGGGGGTGGTGAAGT |
| 3022 | ATCTGAGTCTGGGGTCTCTT | 3653 | AGATATCTGTAGGTATGGAGAA | 4284 | CCCACAGTGGGGGGTGGTGAAGTA |
| 3023 | TCTGAGATCTGAGTCTGGGG | 3654 | GATATCTGTAGGTATGGAGAAC | 4285 | CCACAGTGGGGGGTGGTGAAGTAT |
| 3024 | CTAAGTTATCAGTCTCTGAG | 3655 | ATATCTGTAGGTATGGAGAACC | 4286 | CACAGTGGGGGGTGGTGAAGTATC |
| 3025 | CTGTCTCTAAGTTATCAGTC | 3656 | TATCTGTAGGTATGGAGAACCC | 4287 | ACAGTGGGGGGTGGTGAAGTATCT |
| 3026 | GTGTCTCTGTCTCTAAGTTA | 3657 | ATCTGTAGGTATGGAGAACCCT | 4288 | CAGTGGGGGGTGGTGAAGTATCTT |
| 3027 | CAGGAGTGTCTCTGTCTCTA | 3658 | TCTGTAGGTATGGAGAACCCTT | 4289 | AGTGGGGGGTGGTGAAGTATCTTT |
| 3028 | CCCCTTAAGTCAGGAGTGTC | 3659 | CTGTAGGTATGGAGAACCCTTT | 4290 | GTGGGGGGTGGTGAAGTATCTTTC |
| 3029 | TCTATCTCCCCTTAAGTCAG | 3660 | TGTAGGTATGGAGAACCCTTTT | 4291 | TGGGGGGTGGTGAAGTATCTTTCT |
| 3030 | GAAGTCTCTATCTCCCCTTA | 3661 | GTAGGTATGGAGAACCCTTTTT | 4292 | GGGGGGTGGTGAAGTATCTTTCTC |
| 3031 | CAGCTGAAGTCTCTATCTCC | 3662 | TAGGTATGGAGAACCCTTTTTA | 4293 | GGGGGTGGTGAAGTATCTTTCTCT |
| 3032 | CGGACCAGCTGAAGTCTCTA | 3663 | AGGTATGGAGAACCCTTTTTAG | 4294 | GGGGTGGTGAAGTATCTTTCTCTG |
| 3033 | CCATCTCCGGACCAGCTGAA | 3664 | GGTATGGAGAACCCTTTTTAGA | 4295 | GGGTGGTGAAGTATCTTTCTCTGT |
| 3034 | GAGTTGTCCATCTCCGGACC | 3665 | GTATGGAGAACCCTTTTTAGAC | 4296 | GGTGGTGAAGTATCTTTCTCTGTG |
| 3035 | TTTGGGTCTCTGAGTTGTCC | 3666 | TATGGAGAACCCTTTTTAGACT | 4297 | GTGGTGAAGTATCTTTCTCTGTGG |
| 3036 | ATGAATTTTGGGTCTCTGAG | 3667 | ATGGAGAACCCTTTTTAGACTG | 4298 | TGGTGAAGTATCTTTCTCTGTGGG |
| 3037 | TTCTTCATGAATTTTGGGTC | 3668 | TGGAGAACCCTTTTTAGACTGG | 4299 | GGTGAAGTATCTTTCTCTGTGGGT |
| 3038 | TCGAAGTCTTCTTCATGAAT | 3669 | GGAGAACCCTTTTTAGACTGGA | 4300 | GTGAAGTATCTTTCTCTGTGGGTC |
| 3039 | GTCTTTCGAAGTCTTCTTCA | 3670 | GAGAACCCTTTTTAGACTGGAT | 4301 | TGAAGTATCTTTCTCTGTGGGTCT |
| 3040 | TCTGAGGTCTTTCGAAGTCT | 3671 | AGAACCCTTTTTAGACTGGATT | 4302 | GAAGTATCTTTCTCTGTGGGTCTA |
| 3041 | TCGCACTCTGAGGTCTTTCG | 3672 | GAACCCTTTTTAGACTGGATTT | 4303 | AAGTATCTTTCTCTGTGGGTCTAG |
| 3042 | CTGTGGTCGCACTCTGAGGT | 3673 | AACCCTTTTTAGACTGGATTTT | 4304 | AGTATCTTTCTCTGTGGGTCTAGG |
| 3043 | CAGGGTCTGTGGTCGCACTC | 3674 | ACCCTTTTTAGACTGGATTTTC | 4305 | GTATCTTTCTCTGTGGGTCTAGGG |
| 3044 | TGTTTCAGGGTCTGTGGTCG | 3675 | CCCTTTTTAGACTGGATTTTCA | 4306 | TATCTTTCTCTGTGGGTCTAGGGT |
| 3045 | TTTCTATGTTTCAGGGTCTG | 3676 | CCTTTTTAGACTGGATTTTCAG | 4307 | ATCTTTCTCTGTGGGTCTAGGGTC |
| 3046 | GGGGGTTTCTATGTTTCAGG | 3677 | CTTTTTAGACTGGATTTTCAGA | 4308 | TCTTTCTCTGTGGGTCTAGGGTCT |
| 3047 | CTGGTTTGGGGGGTTTCTAT | 3678 | TTTTTAGACTGGATTTTCAGAT | 4309 | CTTTCTCTGTGGGTCTAGGGTCTC |
| 3048 | TATGCTTCTGGTTTGGGGGG | 3679 | TTTTAGACTGGATTTTCAGATT | 4310 | TTTCTCTGTGGGTCTAGGGTCTCT |
| 3049 | GTTTCCTTTTATGCTTCTGG | 3680 | TTTAGACTGGATTTTCAGATTT | 4311 | TTCTCTGTGGGTCTAGGGTCTCTC |
| 3050 | GATTGAGTTTCCTTTTATGC | 3681 | TTAGACTGGATTTTCAGATTTT | 4312 | TCTCTGTGGGTCTAGGGTCTCTCT |
| 3051 | TCTTAGGATTGAGTTTCCTT | 3682 | TAGACTGGATTTTCAGATTTTG | 4313 | CTCTGTGGGTCTAGGGTCTCTCTG |
| 3052 | GATCTTTCTTAGGATTGAGT | 3683 | AGACTGGATTTTCAGATTTTGA | 4314 | TCTGTGGGTCTAGGGTCTCTCTGT |
| 3053 | GTGGAGATCTTTCTTAGGAT | 3684 | GACTGGATTTTCAGATTTTGAT | 4315 | CTGTGGGTCTAGGGTCTCTCTGTC |
| 3054 | TTTGTGTGGAGATCTTTCTT | 3685 | ACTGGATTTTCAGATTTTGATA | 4316 | TGTGGGTCTAGGGTCTCTCTGTCT |
| 3055 | TCAGTATTTGTGTGGAGATC | 3686 | CTGGATTTTCAGATTTTGATAT | 4317 | GTGGGTCTAGGGTCTCTCTGTCTC |
| 3056 | GTCTTTCAGTATTTGTGTGG | 3687 | TGGATTTTCAGATTTTGATATT | 4318 | TGGGTCTAGGGTCTCTCTGTCTCA |
| 3057 | CGGAGGGGTCTTTCAGTATT | 3688 | GGATTTTCAGATTTTGATATTG | 4319 | GGGTCTAGGGTCTCTCTGTCTCAG |
| 3058 | GATTTCGGAGGGGTCTTTCA | 3689 | GATTTTCAGATTTTGATATTGA | 4320 | GGTCTAGGGTCTCTCTGTCTCAGG |
| 3059 | GAGACAGATTTCGGAGGGGT | 3690 | ATTTTCAGATTTTGATATTGAG | 4321 | GTCTAGGGTCTCTCTGTCTCAGGG |
| 3060 | TCTCTGAGACAGATTTCGGA | 3691 | TTTTCAGATTTTGATATTGAGC | 4322 | TCTAGGGTCTCTCTGTCTCAGGGT |
| 3061 | TTGGAGTTTGTCTCTGAGAC | 3692 | TTTCAGATTTTGATATTGAGCT | 4323 | CTAGGGTCTCTCTGTCTCAGGGTC |
| 3062 | TTGAGTTTGGAGTTTGTCTC | 3693 | TTCAGATTTTGATATTGAGCTT | 4324 | TAGGGTCTCTCTGTCTCAGGGTCT |
| 3063 | CTGTCTTTGAGTTTGGAGTT | 3694 | TCAGATTTTGATATTGAGCTTC | 4325 | AGGGTCTCTCTGTCTCAGGGTCTG |
| 3064 | GAGATCTCTGTCTTTGAGTT | 3695 | CAGATTTTGATATTGAGCTTCT | 4326 | GGGTCTCTCTGTCTCAGGGTCTGG |
| 3065 | GTTCCCTGAGATCTCTGTCT | 3696 | AGATTTTGATATTGAGCTTCTC | 4327 | GGTCTCTCTGTCTCAGGGTCTGGG |
| 3066 | GGGAGGTTCCCTGAGATCTC | 3697 | GATTTTGATATTGAGCTTCTCT | 4328 | GTCTCTCTGTCTCAGGGTCTGGGG |
| 3067 | GGGAGGGGAGGTTCCCTGAG | 3698 | ATTTTGATATTGAGCTTCTCTC | 4329 | TCTCTCTGTCTCAGGGTCTGGGGT |
| 3068 | AAGTGGGGAGGGGAGGTTCC | 3699 | TTTTGATATTGAGCTTCTCTCT | 4330 | CTCTCTGTCTCAGGGTCTGGGGTC |
| 3069 | GGTTCTAGGGCAGGGAAGTG | 3700 | TTTGATATTGAGCTTCTCTCTC | 4331 | TCTCTGTCTCAGGGTCTGGGGTCT |
| 3070 | TCTCGGAGGTTCTAGGGCAG | 3701 | TTGATATTGAGCTTCTCTCTCT | 4332 | CTCTGTCTCAGGGTCTGGGGTCTG |
| 3071 | GTTATACCTCTCGGAGGTTC | 3702 | TGATATTGAGCTTCTCTCTCTG | 4333 | TCTGTCTCAGGGTCTGGGGTCTGC |
| 3072 | GTCAGGGTTATACCTCTCGG | 3703 | GATATTGAGCTTCTCTCTCTGG | 4334 | CTGTCTCAGGGTCTGGGGTCTGCA |
| 3073 | GGCTGACGTCAGGGTTATAC | 3704 | ATATTGAGCTTCTCTCTCTGGG | 4335 | TGTCTCAGGGTCTGGGGTCTGCAT |
| 3074 | TCCCAGGCTGACGTCAGGGT | 3705 | TATTGAGCTTCTCTCTCTGGGG | 4336 | GTCTCAGGGTCTGGGGTCTGCATC |
| 3075 | TCGGAGTTTCCCAGGCTGAC | 3706 | ATTGAGCTTCTCTCTCTGGGGG | 4337 | TCTCAGGGTCTGGGGTCTGCATCT |
| 3076 | GATGCCTCGGAGTTTCCCAG | 3707 | TTGAGCTTCTCTCTCTGGGGGA | 4338 | CTCAGGGTCTGGGGTCTGCATCTT |
| 3077 | GGTGGGGATGCCTCGGAGTT | 3708 | TGAGCTTCTCTCTCTGGGGGAT | 4339 | TCAGGGTCTGGGGTCTGCATCTTT |
| 3078 | CTGGTGGTGGGGATGCCTCG | 3709 | GAGCTTCTCTCTCTGGGGGATC | 4340 | CAGGGTCTGGGGTCTGCATCTTTA |
| 3079 | ATTGGTCTGGTGGTGGGGAT | 3710 | AGCTTCTCTCTCTGGGGGATCT | 4341 | AGGGTCTGGGGTCTGCATCTTTAG |
| 3080 | GAGGTCATTGGTCTGGTGGT | 3711 | GCTTCTCTCTCTGGGGGATCTG | 4342 | GGGTCTGGGGTCTGCATCTTTAGG |
| 3081 | AGGTCTGAGGTCATTGGTCT | 3712 | CTTCTCTCTCTGGGGGATCTGG | 4343 | GGTCTGGGGTCTGCATCTTTAGGA |
| 3082 | CCTTCAAGGTCTGAGGTCAT | 3713 | TTCTCTCTCTGGGGGATCTGGG | 4344 | GTCTGGGGTCTGCATCTTTAGGAC |
| 3083 | TCCCCTCCCTTCAAGGTCTG | 3714 | TCTCTCTCTGGGGGATCTGGGG | 4345 | TCTGGGGTCTGCATCTTTAGGACC |
| 3084 | GGTCTGGAGTCTAAGATA | 3715 | CTCTCTCTGGGGGATCTGGGGT | 4346 | CTGGGGTCTGCATCTTTAGGACCT |
| 3085 | AGTCTAAGATATCTGTAG | 3716 | TCTCTCTGGGGGATCTGGGGTC | 4347 | TGGGGTCTGCATCTTTAGGACCTC |
| 3086 | GTCTAAGATATCTGTAGG | 3717 | CTCTCTGGGGGATCTGGGGTCG | 4348 | GGGGTCTGCATCTTTAGGACCTCT |
| 3087 | AAGATATCTGTAGGTATG | 3718 | TCTCTGGGGGATCTGGGGTCGT | 4349 | GGGTCTGCATCTTTAGGACCTCTG |
| 3088 | ATATCTGTAGGTATGGAG | 3719 | CTCTGGGGGATCTGGGGTCGTT | 4350 | GGTCTGCATCTTTAGGACCTCTGT |
| 3089 | TGTAGGTATGGAGAACCC | 3720 | TCTGGGGGATCTGGGGTCGTTT | 4351 | GTCTGCATCTTTAGGACCTCTGTC |
| 3090 | GTATGGAGAACCCTTTTT | 3721 | CTGGGGGATCTGGGGTCGTTTT | 4352 | TCTGCATCTTTAGGACCTCTGTCT |
| 3091 | GGGGGATCTGGGGTCGTT | 3722 | TGGGGGATCTGGGGTCGTTTTT | 4353 | CTGCATCTTTAGGACCTCTGTCTC |
| 3092 | GGGGATCTGGGGTCGTTT | 3723 | GGGGGATCTGGGGTCGTTTTTT | 4354 | TGCATCTTTAGGACCTCTGTCTCT |
| 3093 | GGGATCTGGGGTCGTTTT | 3724 | GGGGATCTGGGGTCGTTTTTTC | 4355 | GCATCTTTAGGACCTCTGTCTCTC |
| 3094 | GGATCTGGGGTCGTTTTT | 3725 | GGGATCTGGGGTCGTTTTTTCC | 4356 | CATCTTTAGGACCTCTGTCTCTCT |
| 3095 | GATCTGGGGTCGTTTTTT | 3726 | GGATCTGGGGTCGTTTTTTCCT | 4357 | ATCTTTAGGACCTCTGTCTCTCTC |
| 3096 | ATCTGGGGTCGTTTTTTC | 3727 | GATCTGGGGTCGTTTTTTCCTC | 4358 | TCTTTAGGACCTCTGTCTCTCTCT |
| 3097 | GGTCGTTTTTTCCTCAAA | 3728 | ATCTGGGGTCGTTTTTTCCTCA | 4359 | CTTTAGGACCTCTGTCTCTCTCTG |
| 3098 | TCGTTTTTTCCTCAAATC | 3729 | TCTGGGGTCGTTTTTTCCTCAA | 4360 | TTTAGGACCTCTGTCTCTCTCTGG |
| 3099 | TTTCCTCAAATCAGGAGT | 3730 | CTGGGGTCGTTTTTTCCTCAAA | 4361 | TTAGGACCTCTGTCTCTCTCTGGT |
| 3100 | CCTCTAGATTTTGGCCCT | 3731 | TGGGGTCGTTTTTTCCTCAAAT | 4362 | TAGGACCTCTGTCTCTCTCTGGTG |
| 3101 | TAGATTTTGGCCCTGTGT | 3732 | GGGGTCGTTTTTTCCTCAAATC | 4363 | AGGACCTCTGTCTCTCTCTGGTGT |
| 3102 | AGATTTTGGCCCTGTGTC | 3733 | GGGTCGTTTTTTCCTCAAATCA | 4364 | GGACCTCTGTCTCTCTCTGGTGTG |
| 3103 | CCTGTGTCTCAAATTACC | 3734 | GGTCGTTTTTTCCTCAAATCAG | 4365 | GACCTCTGTCTCTCTCTGGTGTGT |
| 3104 | GTGTCTCAAATTACCCCA | 3735 | GTCGTTTTTTCCTCAAATCAGG | 4366 | ACCTCTGTCTCTCTCTGGTGTGTT |
| 3105 | GTCTCAAATTACCCCACA | 3736 | TCGTTTTTTCCTCAAATCAGGA | 4367 | CCTCTGTCTCTCTCTGGTGTGTTT |
| 3106 | AAATTACCCCACAGTGGG | 3737 | CGTTTTTTCCTCAAATCAGGAG | 4368 | GGGTCTGGAGTCTAAGATATCTGTA |
| 3107 | TACCCCACAGTGGGGGGT | 3738 | GTTTTTTCCTCAAATCAGGAGT | 4369 | GGTCTGGAGTCTAAGATATCTGTAG |
| 3108 | GGGGGGTGGTGAAGTATC | 3739 | TTTTTTCCTCAAATCAGGAGTC | 4370 | GTCTGGAGTCTAAGATATCTGTAGG |
| 3109 | GGGGGTGGTGAAGTATCT | 3740 | TTTTTCCTCAAATCAGGAGTCT | 4371 | TCTGGAGTCTAAGATATCTGTAGGT |
| 3110 | GGGGTGGTGAAGTATCTT | 3741 | TTTTCCTCAAATCAGGAGTCTC | 4372 | CTGGAGTCTAAGATATCTGTAGGTA |
| 3111 | GGGTGGTGAAGTATCTTT | 3742 | TTTCCTCAAATCAGGAGTCTCT | 4373 | TGGAGTCTAAGATATCTGTAGGTAT |
| 3112 | GGTGGTGAAGTATCTTTC | 3743 | TTCCTCAAATCAGGAGTCTCTT | 4374 | GGAGTCTAAGATATCTGTAGGTATG |
| 3113 | GGTGAAGTATCTTTCTCT | 3744 | TCCTCAAATCAGGAGTCTCTTT | 4375 | GAGTCTAAGATATCTGTAGGTATGG |
| 3114 | TCTGTGGGTCTAGGGTCT | 3745 | CCTCAAATCAGGAGTCTCTTTT | 4376 | AGTCTAAGATATCTGTAGGTATGGA |
| 3115 | CTGTGGGTCTAGGGTCTC | 3746 | CTCAAATCAGGAGTCTCTTTTC | 4377 | GTCTAAGATATCTGTAGGTATGGAG |
| 3116 | TGTGGGTCTAGGGTCTCT | 3747 | TCAAATCAGGAGTCTCTTTTCC | 4378 | TCTAAGATATCTGTAGGTATGGAGA |
| 3117 | GTCTAGGGTCTCTCTGTC | 3748 | CAAATCAGGAGTCTCTTTTCCT | 4379 | CTAAGATATCTGTAGGTATGGAGAA |
| 3118 | TCTAGGGTCTCTCTGTCT | 3749 | ATCAGGAGTCTCTTTTCCTCTA | 4380 | TAAGATATCTGTAGGTATGGAGAAC |
| 3119 | GGGTCTGCATCTTTAGGA | 3750 | TCAGGAGTCTCTTTTCCTCTAG | 4381 | AAGATATCTGTAGGTATGGAGAACC |
| 3120 | GGTCTGCATCTTTAGGAC | 3751 | CAGGAGTCTCTTTTCCTCTAGA | 4382 | AGATATCTGTAGGTATGGAGAACCC |
| 3121 | GGGTCTGGAGTCTAAGATA | 3752 | AGGAGTCTCTTTTCCTCTAGAT | 4383 | GATATCTGTAGGTATGGAGAACCCT |
| 3122 | GGTCTGGAGTCTAAGATAT | 3753 | GGAGTCTCTTTTCCTCTAGATT | 4384 | ATATCTGTAGGTATGGAGAACCCTT |
| 3123 | GTCTGGAGTCTAAGATATC | 3754 | GAGTCTCTTTTCCTCTAGATTT | 4385 | TATCTGTAGGTATGGAGAACCCTTT |
| 3124 | GAGTCTAAGATATCTGTAG | 3755 | AGTCTCTTTTCCTCTAGATTTT | 4386 | ATCTGTAGGTATGGAGAACCCTTTT |
| 3125 | AGTCTAAGATATCTGTAGG | 3756 | TCTCTTTTCCTCTAGATTTTGG | 4387 | TCTGTAGGTATGGAGAACCCTTTTT |
| 3126 | GTCTAAGATATCTGTAGGT | 3757 | CTCTTTTCCTCTAGATTTTGGC | 4388 | CTGTAGGTATGGAGAACCCTTTTTA |
| 3127 | TCTAAGATATCTGTAGGTA | 3758 | TCTTTTCCTCTAGATTTTGGCC | 4389 | TGTAGGTATGGAGAACCCTTTTTAG |
| 3128 | TAAGATATCTGTAGGTATG | 3759 | CTTTTCCTCTAGATTTTGGCCC | 4390 | GTAGGTATGGAGAACCCTTTTTAGA |
| 3129 | AAGATATCTGTAGGTATGG | 3760 | TTTTCCTCTAGATTTTGGCCCT | 4391 | TAGGTATGGAGAACCCTTTTTAGAC |
| 3130 | GATATCTGTAGGTATGGAG | 3761 | TTTCCTCTAGATTTTGGCCCTG | 4392 | AGGTATGGAGAACCCTTTTTAGACT |
| 3131 | ATATCTGTAGGTATGGAGA | 3762 | TTCCTCTAGATTTTGGCCCTGT | 4393 | GGTATGGAGAACCCTTTTTAGACTG |
| 3132 | CTGTAGGTATGGAGAACCC | 3763 | TCCTCTAGATTTTGGCCCTGTG | 4394 | GTATGGAGAACCCTTTTTAGACTGG |
| 3133 | TGTAGGTATGGAGAACCCT | 3764 | CCTCTAGATTTTGGCCCTGTGT | 4395 | TATGGAGAACCCTTTTTAGACTGGA |
| 3134 | GGTATGGAGAACCCTTTTT | 3765 | CTCTAGATTTTGGCCCTGTGTC | 4396 | ATGGAGAACCCTTTTTAGACTGGAT |
| 3135 | GTATGGAGAACCCTTTTTA | 3766 | TCTAGATTTTGGCCCTGTGTCT | 4397 | TGGAGAACCCTTTTTAGACTGGATT |
| 3136 | TGGAGAACCCTTTTTAGAC | 3767 | CTAGATTTTGGCCCTGTGTCTC | 4398 | GGAGAACCCTTTTTAGACTGGATTT |
| 3137 | GAGAACCCTTTTTAGACTG | 3768 | TAGATTTTGGCCCTGTGTCTCA | 4399 | GAGAACCCTTTTTAGACTGGATTTT |
| 3138 | AGAACCCTTTTTAGACTGG | 3769 | AGATTTTGGCCCTGTGTCTCAA | 4400 | AGAACCCTTTTTAGACTGGATTTTC |
| 3139 | GAACCCTTTTTAGACTGGA | 3770 | GATTTTGGCCCTGTGTCTCAAA | 4401 | GAACCCTTTTTAGACTGGATTTTCA |
| 3140 | AACCCTTTTTAGACTGGAT | 3771 | ATTTTGGCCCTGTGTCTCAAAT | 4402 | AACCCTTTTTAGACTGGATTTTCAG |
| 3141 | CCCTTTTTAGACTGGATTT | 3772 | TTTTGGCCCTGTGTCTCAAATT | 4403 | ACCCTTTTTAGACTGGATTTTCAGA |
| 3142 | TTTTTAGACTGGATTTTCA | 3773 | TTTGGCCCTGTGTCTCAAATTA | 4404 | CCCTTTTTAGACTGGATTTTCAGAT |
| 3143 | TTAGACTGGATTTTCAGAT | 3774 | TTGGCCCTGTGTCTCAAATTAC | 4405 | CCTTTTTAGACTGGATTTTCAGATT |
| 3144 | TAGACTGGATTTTCAGATT | 3775 | TGGCCCTGTGTCTCAAATTACC | 4406 | CTTTTTAGACTGGATTTTCAGATTT |
| 3145 | GACTGGATTTTCAGATTTT | 3776 | GGCCCTGTGTCTCAAATTACCC | 4407 | TTTTTAGACTGGATTTTCAGATTTT |
| 3146 | ACTGGATTTTCAGATTTTG | 3777 | GCCCTGTGTCTCAAATTACCCC | 4408 | TTTTAGACTGGATTTTCAGATTTTG |
| 3147 | TCAGATTTTGATATTGAGC | 3778 | CCCTGTGTCTCAAATTACCCCA | 4409 | TTTAGACTGGATTTTCAGATTTTGA |
| 3148 | GATTTTGATATTGAGCTTC | 3779 | CCTGTGTCTCAAATTACCCCAC | 4410 | TTAGACTGGATTTTCAGATTTTGAT |
| 3149 | TTTTGATATTGAGCTTCTC | 3780 | CTGTGTCTCAAATTACCCCACA | 4411 | TAGACTGGATTTTCAGATTTTGATA |
| 3150 | TTGATATTGAGCTTCTCTC | 3781 | TGTGTCTCAAATTACCCCACAG | 4412 | AGACTGGATTTTCAGATTTTGATAT |
| 3151 | TGATATTGAGCTTCTCTCT | 3782 | GTGTCTCAAATTACCCCACAGT | 4413 | GACTGGATTTTCAGATTTTGATATT |
| 3152 | GATATTGAGCTTCTCTCTC | 3783 | TGTCTCAAATTACCCCACAGTG | 4414 | ACTGGATTTTCAGATTTTGATATTG |
| 3153 | AGCTTCTCTCTCTGGGGGA | 3784 | GTCTCAAATTACCCCACAGTGG | 4415 | CTGGATTTTCAGATTTTGATATTGA |
| 3154 | CTTCTCTCTCTGGGGGATC | 3785 | TCTCAAATTACCCCACAGTGGG | 4416 | TGGATTTTCAGATTTTGATATTGAG |
| 3155 | CTCTCTCTGGGGGATCTGG | 3786 | CTCAAATTACCCCACAGTGGGG | 4417 | GGATTTTCAGATTTTGATATTGAGC |
| 3156 | TCTCTCTGGGGGATCTGGG | 3787 | TCAAATTACCCCACAGTGGGGG | 4418 | GATTTTCAGATTTTGATATTGAGCT |
| 3157 | CTGGGGGATCTGGGGTCGT | 3788 | CAAATTACCCCACAGTGGGGGG | 4419 | ATTTTCAGATTTTGATATTGAGCTT |
| 3158 | TGGGGGATCTGGGGTCGTT | 3789 | AAATTACCCCACAGTGGGGGGT | 4420 | TTTTCAGATTTTGATATTGAGCTTC |
| 3159 | GGGGGATCTGGGGTCGTTT | 3790 | AATTACCCCACAGTGGGGGGTG | 4421 | TTTCAGATTTTGATATTGAGCTTCT |
| 3160 | GGGGATCTGGGGTCGTTTT | 3791 | ATTACCCCACAGTGGGGGGTGG | 4422 | TTCAGATTTTGATATTGAGCTTCTC |
| 3161 | GGGATCTGGGGTCGTTTTT | 3792 | TTACCCCACAGTGGGGGGTGGT | 4423 | TCAGATTTTGATATTGAGCTTCTCT |
| 3162 | GGATCTGGGGTCGTTTTTT | 3793 | TACCCCACAGTGGGGGGTGGTG | 4424 | CAGATTTTGATATTGAGCTTCTCTC |
| 3163 | GATCTGGGGTCGTTTTTTC | 3794 | ACCCCACAGTGGGGGGTGGTGA | 4425 | AGATTTTGATATTGAGCTTCTCTCT |
| 3164 | ATCTGGGGTCGTTTTTTCC | 3795 | CCCCACAGTGGGGGGTGGTGAA | 4426 | GATTTTGATATTGAGCTTCTCTCTC |
| 3165 | TCTGGGGTCGTTTTTTCCT | 3796 | CCCACAGTGGGGGGTGGTGAAG | 4427 | ATTTTGATATTGAGCTTCTCTCTCT |
| 3166 | GGGTCGTTTTTTCCTCAAA | 3797 | CCACAGTGGGGGGTGGTGAAGT | 4428 | TTTTGATATTGAGCTTCTCTCTCTG |
| 3167 | GGTCGTTTTTTCCTCAAAT | 3798 | CACAGTGGGGGGTGGTGAAGTA | 4429 | TTTGATATTGAGCTTCTCTCTCTGG |
| 3168 | GTCGTTTTTTCCTCAAATC | 3799 | ACAGTGGGGGGTGGTGAAGTAT | 4430 | TTGATATTGAGCTTCTCTCTCTGGG |
| 3169 | TCGTTTTTTCCTCAAATCA | 3800 | CAGTGGGGGGTGGTGAAGTATC | 4431 | TGATATTGAGCTTCTCTCTCTGGGG |
| 3170 | CGTTTTTTCCTCAAATCAG | 3801 | AGTGGGGGGTGGTGAAGTATCT | 4432 | GATATTGAGCTTCTCTCTCTGGGGG |
| 3171 | TTTTCCTCAAATCAGGAGT | 3802 | GTGGGGGGTGGTGAAGTATCTT | 4433 | ATATTGAGCTTCTCTCTCTGGGGGA |
| 3172 | TTTCCTCAAATCAGGAGTC | 3803 | TGGGGGGTGGTGAAGTATCTTT | 4434 | TATTGAGCTTCTCTCTCTGGGGGAT |
| 3173 | CTCAAATCAGGAGTCTCTT | 3804 | GGGGGGTGGTGAAGTATCTTTC | 4435 | ATTGAGCTTCTCTCTCTGGGGGATC |
| 3174 | GAGTCTCTTTTCCTCTAGA | 3805 | GGGGGTGGTGAAGTATCTTTCT | 4436 | TTGAGCTTCTCTCTCTGGGGGATCT |
| 3175 | TTTTCCTCTAGATTTTGGC | 3806 | GGGGTGGTGAAGTATCTTTCTC | 4437 | TGAGCTTCTCTCTCTGGGGGATCTG |
| 3176 | TCCTCTAGATTTTGGCCCT | 3807 | GGGTGGTGAAGTATCTTTCTCT | 4438 | GAGCTTCTCTCTCTGGGGGATCTGG |
| 3177 | CCTCTAGATTTTGGCCCTG | 3808 | GGTGGTGAAGTATCTTTCTCTG | 4439 | AGCTTCTCTCTCTGGGGGATCTGGG |
| 3178 | CTCTAGATTTTGGCCCTGT | 3809 | GTGGTGAAGTATCTTTCTCTGT | 4440 | GCTTCTCTCTCTGGGGGATCTGGGG |
| 3179 | TCTAGATTTTGGCCCTGTG | 3810 | TGGTGAAGTATCTTTCTCTGTG | 4441 | CTTCTCTCTCTGGGGGATCTGGGGT |
| 3180 | CTAGATTTTGGCCCTGTGT | 3811 | GGTGAAGTATCTTTCTCTGTGG | 4442 | TTCTCTCTCTGGGGGATCTGGGGTC |
| 3181 | TAGATTTTGGCCCTGTGTC | 3812 | GTGAAGTATCTTTCTCTGTGGG | 4443 | TCTCTCTCTGGGGGATCTGGGGTCG |
| 3182 | AGATTTTGGCCCTGTGTCT | 3813 | TGAAGTATCTTTCTCTGTGGGT | 4444 | CTCTCTCTGGGGGATCTGGGGTCGT |
| 3183 | GATTTTGGCCCTGTGTCTC | 3814 | GAAGTATCTTTCTCTGTGGGTC | 4445 | TCTCTCTGGGGGATCTGGGGTCGTT |
| 3184 | ATTTTGGCCCTGTGTCTCA | 3815 | AAGTATCTTTCTCTGTGGGTCT | 4446 | CTCTCTGGGGGATCTGGGGTCGTTT |
| 3185 | TTTGGCCCTGTGTCTCAAA | 3816 | AGTATCTTTCTCTGTGGGTCTA | 4447 | TCTCTGGGGGATCTGGGGTCGTTTT |
| 3186 | TTGGCCCTGTGTCTCAAAT | 3817 | GTATCTTTCTCTGTGGGTCTAG | 4448 | CTCTGGGGGATCTGGGGTCGTTTTT |
| 3187 | GCCCTGTGTCTCAAATTAC | 3818 | TATCTTTCTCTGTGGGTCTAGG | 4449 | TCTGGGGGATCTGGGGTCGTTTTTT |
| 3188 | CCCTGTGTCTCAAATTACC | 3819 | ATCTTTCTCTGTGGGTCTAGGG | 4450 | CTGGGGGATCTGGGGTCGTTTTTTC |
| 3189 | CCTGTGTCTCAAATTACCC | 3820 | TCTTTCTCTGTGGGTCTAGGGT | 4451 | TGGGGGATCTGGGGTCGTTTTTTCC |
| 3190 | TGTGTCTCAAATTACCCCA | 3821 | CTTTCTCTGTGGGTCTAGGGTC | 4452 | GGGGGATCTGGGGTCGTTTTTTCCT |
| 3191 | GTGTCTCAAATTACCCCAC | 3822 | TTTCTCTGTGGGTCTAGGGTCT | 4453 | GGGGATCTGGGGTCGTTTTTTCCTC |
| 3192 | TGTCTCAAATTACCCCACA | 3823 | TTCTCTGTGGGTCTAGGGTCTC | 4454 | GGGATCTGGGGTCGTTTTTTCCTCA |
| 3193 | GTCTCAAATTACCCCACAG | 3824 | TCTCTGTGGGTCTAGGGTCTCT | 4455 | GGATCTGGGGTCGTTTTTTCCTCAA |
| 3194 | TCTCAAATTACCCCACAGT | 3825 | CTCTGTGGGTCTAGGGTCTCTC | 4456 | GATCTGGGGTCGTTTTTTCCTCAAA |
| 3195 | TCAAATTACCCCACAGTGG | 3826 | TCTGTGGGTCTAGGGTCTCTCT | 4457 | ATCTGGGGTCGTTTTTTCCTCAAAT |
| 3196 | CAAATTACCCCACAGTGGG | 3827 | CTGTGGGTCTAGGGTCTCTCTG | 4458 | TCTGGGGTCGTTTTTTCCTCAAATC |
| 3197 | AAATTACCCCACAGTGGGG | 3828 | TGTGGGTCTAGGGTCTCTCTGT | 4459 | CTGGGGTCGTTTTTTCCTCAAATCA |
| 3198 | ATTACCCCACAGTGGGGGG | 3829 | GTGGGTCTAGGGTCTCTCTGTC | 4460 | TGGGGTCGTTTTTTCCTCAAATCAG |
| 3199 | TTACCCCACAGTGGGGGGT | 3830 | TGGGTCTAGGGTCTCTCTGTCT | 4461 | GGGGTCGTTTTTTCCTCAAATCAGG |
| 3200 | TACCCCACAGTGGGGGGTG | 3831 | GGGTCTAGGGTCTCTCTGTCTC | 4462 | GGGTCGTTTTTTCCTCAAATCAGGA |
| 3201 | ACCCCACAGTGGGGGGTGG | 3832 | GGTCTAGGGTCTCTCTGTCTCA | 4463 | GGTCGTTTTTTCCTCAAATCAGGAG |
| 3202 | CCCCACAGTGGGGGGTGGT | 3833 | GTCTAGGGTCTCTCTGTCTCAG | 4464 | GTCGTTTTTTCCTCAAATCAGGAGT |
| 3203 | CCCACAGTGGGGGGTGGTG | 3834 | TCTAGGGTCTCTCTGTCTCAGG | 4465 | TCGTTTTTTCCTCAAATCAGGAGTC |
| 3204 | CACAGTGGGGGGTGGTGAA | 3835 | CTAGGGTCTCTCTGTCTCAGGG | 4466 | CGTTTTTTCCTCAAATCAGGAGTCT |
| 3205 | GTGGGGGGTGGTGAAGTAT | 3836 | TAGGGTCTCTCTGTCTCAGGGT | 4467 | GTTTTTTCCTCAAATCAGGAGTCTC |
| 3206 | TGGGGGGTGGTGAAGTATC | 3837 | AGGGTCTCTCTGTCTCAGGGTC | 4468 | TTTTTTCCTCAAATCAGGAGTCTCT |
| 3207 | GGGGGGTGGTGAAGTATCT | 3838 | GGGTCTCTCTGTCTCAGGGTCT | 4469 | TTTTTCCTCAAATCAGGAGTCTCTT |
| 3208 | GGGGGTGGTGAAGTATCTT | 3839 | GGTCTCTCTGTCTCAGGGTCTG | 4470 | TTTTCCTCAAATCAGGAGTCTCTTT |
| 3209 | GGGGTGGTGAAGTATCTTT | 3840 | GTCTCTCTGTCTCAGGGTCTGG | 4471 | TTTCCTCAAATCAGGAGTCTCTTTT |
| 3210 | GGGTGGTGAAGTATCTTTC | 3841 | TCTCTCTGTCTCAGGGTCTGGG | 4472 | TTCCTCAAATCAGGAGTCTCTTTTC |
| 3211 | GGTGGTGAAGTATCTTTCT | 3842 | CTCTCTGTCTCAGGGTCTGGGG | 4473 | TCCTCAAATCAGGAGTCTCTTTTCC |
| 3212 | TGGTGAAGTATCTTTCTCT | 3843 | TCTCTGTCTCAGGGTCTGGGGT | 4474 | CCTCAAATCAGGAGTCTCTTTTCCT |
| 3213 | GGTGAAGTATCTTTCTCTG | 3844 | CTCTGTCTCAGGGTCTGGGGTC | 4475 | CTCAAATCAGGAGTCTCTTTTCCTC |
| 3214 | CTTTCTCTGTGGGTCTAGG | 3845 | TCTGTCTCAGGGTCTGGGGTCT | 4476 | TCAAATCAGGAGTCTCTTTTCCTCT |
| 3215 | CTCTGTGGGTCTAGGGTCT | 3846 | CTGTCTCAGGGTCTGGGGTCTG | 4477 | CAAATCAGGAGTCTCTTTTCCTCTA |
| 3216 | TCTGTGGGTCTAGGGTCTC | 3847 | TGTCTCAGGGTCTGGGGTCTGC | 4478 | AAATCAGGAGTCTCTTTTCCTCTAG |
| 3217 | CTGTGGGTCTAGGGTCTCT | 3848 | GTCTCAGGGTCTGGGGTCTGCA | 4479 | AATCAGGAGTCTCTTTTCCTCTAGA |
| 3218 | TGTGGGTCTAGGGTCTCTC | 3849 | TCTCAGGGTCTGGGGTCTGCAT | 4480 | ATCAGGAGTCTCTTTTCCTCTAGAT |
| 3219 | GGGTCTAGGGTCTCTCTGT | 3850 | CTCAGGGTCTGGGGTCTGCATC | 4481 | TCAGGAGTCTCTTTTCCTCTAGATT |
| 3220 | GGTCTAGGGTCTCTCTGTC | 3851 | TCAGGGTCTGGGGTCTGCATCT | 4482 | CAGGAGTCTCTTTTCCTCTAGATTT |
| 3221 | GTCTAGGGTCTCTCTGTCT | 3852 | CAGGGTCTGGGGTCTGCATCTT | 4483 | AGGAGTCTCTTTTCCTCTAGATTTT |
| 3222 | TCTAGGGTCTCTCTGTCTC | 3853 | AGGGTCTGGGGTCTGCATCTTT | 4484 | GGAGTCTCTTTTCCTCTAGATTTTG |
| 3223 | CTAGGGTCTCTCTGTCTCA | 3854 | GGGTCTGGGGTCTGCATCTTTA | 4485 | GAGTCTCTTTTCCTCTAGATTTTGG |
| 3224 | GTCTCTCTGTCTCAGGGTC | 3855 | GGTCTGGGGTCTGCATCTTTAG | 4486 | AGTCTCTTTTCCTCTAGATTTTGGC |
| 3225 | CTGTCTCAGGGTCTGGGGT | 3856 | GTCTGGGGTCTGCATCTTTAGG | 4487 | GTCTCTTTTCCTCTAGATTTTGGCC |
| 3226 | TGTCTCAGGGTCTGGGGTC | 3857 | TCTGGGGTCTGCATCTTTAGGA | 4488 | TCTCTTTTCCTCTAGATTTTGGCCC |
| 3227 | TCAGGGTCTGGGGTCTGCA | 3858 | CTGGGGTCTGCATCTTTAGGAC | 4489 | CTCTTTTCCTCTAGATTTTGGCCCT |
| 3228 | TCTGGGGTCTGCATCTTTA | 3859 | TGGGGTCTGCATCTTTAGGACC | 4490 | TCTTTTCCTCTAGATTTTGGCCCTG |
| 3229 | CTGGGGTCTGCATCTTTAG | 3860 | GGGGTCTGCATCTTTAGGACCT | 4491 | CTTTTCCTCTAGATTTTGGCCCTGT |
| 3230 | GGGGTCTGCATCTTTAGGA | 3861 | GGGTCTGCATCTTTAGGACCTC | 4492 | TTTTCCTCTAGATTTTGGCCCTGTG |
| 3231 | GGGTCTGCATCTTTAGGAC | 3862 | GGTCTGCATCTTTAGGACCTCT | 4493 | TTTCCTCTAGATTTTGGCCCTGTGT |
| 3232 | GGTCTGCATCTTTAGGACC | 3863 | TCTGCATCTTTAGGACCTCTGT | 4494 | TTCCTCTAGATTTTGGCCCTGTGTC |
| 3233 | ATCTTTAGGACCTCTGTCT | 3864 | CTGCATCTTTAGGACCTCTGTC | 4495 | TCCTCTAGATTTTGGCCCTGTGTCT |
| 3234 | GGGTCTGGAGTCTAAGATAT | 3865 | TGCATCTTTAGGACCTCTGTCT | 4496 | CCTCTAGATTTTGGCCCTGTGTCTC |
| 3235 | GGTCTGGAGTCTAAGATATC | 3866 | GCATCTTTAGGACCTCTGTCTC | 4497 | CTCTAGATTTTGGCCCTGTGTCTCA |
| 3236 | GTCTGGAGTCTAAGATATCT | 3867 | CATCTTTAGGACCTCTGTCTCT | 4498 | TCTAGATTTTGGCCCTGTGTCTCAA |
| 3237 | TCTGGAGTCTAAGATATCTG | 3868 | ATCTTTAGGACCTCTGTCTCTC | 4499 | CTAGATTTTGGCCCTGTGTCTCAAA |
| 3238 | CTGGAGTCTAAGATATCTGT | 3869 | TCTTTAGGACCTCTGTCTCTCT | 4500 | TAGATTTTGGCCCTGTGTCTCAAAT |
| 3239 | TGGAGTCTAAGATATCTGTA | 3870 | CTTTAGGACCTCTGTCTCTCTC | 4501 | AGATTTTGGCCCTGTGTCTCAAATT |
| 3240 | GGAGTCTAAGATATCTGTAG | 3871 | TTTAGGACCTCTGTCTCTCTCT | 4502 | GATTTTGGCCCTGTGTCTCAAATTA |
| 3241 | GAGTCTAAGATATCTGTAGG | 3872 | TTAGGACCTCTGTCTCTCTCTG | 4503 | ATTTTGGCCCTGTGTCTCAAATTAC |
| 3242 | AGTCTAAGATATCTGTAGGT | 3873 | TAGGACCTCTGTCTCTCTCTGG | 4504 | TTTTGGCCCTGTGTCTCAAATTACC |
| 3243 | GTCTAAGATATCTGTAGGTA | 3874 | AGGACCTCTGTCTCTCTCTGGT | 4505 | TTTGGCCCTGTGTCTCAAATTACCC |
| 3244 | TCTAAGATATCTGTAGGTAT | 3875 | GGACCTCTGTCTCTCTCTGGTG | 4506 | TTGGCCCTGTGTCTCAAATTACCCC |
| 3245 | CTAAGATATCTGTAGGTATG | 3876 | ACCTCTGTCTCTCTCTGGTGTG | 4507 | TGGCCCTGTGTCTCAAATTACCCCA |
| 3246 | TAAGATATCTGTAGGTATGG | 3877 | CCTCTGTCTCTCTCTGGTGTGT | 4508 | GGCCCTGTGTCTCAAATTACCCCAC |
| 3247 | AAGATATCTGTAGGTATGGA | 3878 | CTCTGTCTCTCTCTGGTGTGTT | 4509 | GCCCTGTGTCTCAAATTACCCCACA |
| 3248 | AGATATCTGTAGGTATGGAG | 3879 | TCTGTCTCTCTCTGGTGTGTTT | 4510 | CCCTGTGTCTCAAATTACCCCACAG |
| 3249 | GATATCTGTAGGTATGGAGA | 3880 | GGGTCTGGAGTCTAAGATATCTG | 4511 | CCTGTGTCTCAAATTACCCCACAGT |
| 3250 | ATATCTGTAGGTATGGAGAA | 3881 | GGTCTGGAGTCTAAGATATCTGT | 4512 | CTGTGTCTCAAATTACCCCACAGTG |
| 3251 | TCTGTAGGTATGGAGAACCC | 3882 | GTCTGGAGTCTAAGATATCTGTA | 4513 | TGTGTCTCAAATTACCCCACAGTGG |
| 3252 | CTGTAGGTATGGAGAACCCT | 3883 | TCTGGAGTCTAAGATATCTGTAG | 4514 | GTGTCTCAAATTACCCCACAGTGGG |
| 3253 | TGTAGGTATGGAGAACCCTT | 3884 | CTGGAGTCTAAGATATCTGTAGG | 4515 | TGTCTCAAATTACCCCACAGTGGGG |
| 3254 | GTAGGTATGGAGAACCCTTT | 3885 | TGGAGTCTAAGATATCTGTAGGT | 4516 | GTCTCAAATTACCCCACAGTGGGGG |
| 3255 | AGGTATGGAGAACCCTTTTT | 3886 | GGAGTCTAAGATATCTGTAGGTA | 4517 | TCTCAAATTACCCCACAGTGGGGGG |
| 3256 | GGTATGGAGAACCCTTTTTA | 3887 | GAGTCTAAGATATCTGTAGGTAT | 4518 | CTCAAATTACCCCACAGTGGGGGGT |
| 3257 | GTATGGAGAACCCTTTTTAG | 3888 | AGTCTAAGATATCTGTAGGTATG | 4519 | TCAAATTACCCCACAGTGGGGGGTG |
| 3258 | TATGGAGAACCCTTTTTAGA | 3889 | GTCTAAGATATCTGTAGGTATGG | 4520 | CAAATTACCCCACAGTGGGGGGTGG |
| 3259 | ATGGAGAACCCTTTTTAGAC | 3890 | TCTAAGATATCTGTAGGTATGGA | 4521 | AAATTACCCCACAGTGGGGGGTGGT |
| 3260 | TGGAGAACCCTTTTTAGACT | 3891 | CTAAGATATCTGTAGGTATGGAG | 4522 | AATTACCCCACAGTGGGGGGTGGTG |
| 3261 | GGAGAACCCTTTTTAGACTG | 3892 | TAAGATATCTGTAGGTATGGAGA | 4523 | ATTACCCCACAGTGGGGGGTGGTGA |
| 3262 | GAGAACCCTTTTTAGACTGG | 3893 | AAGATATCTGTAGGTATGGAGAA | 4524 | TTACCCCACAGTGGGGGGTGGTGAA |
| 3263 | AGAACCCTTTTTAGACTGGA | 3894 | AGATATCTGTAGGTATGGAGAAC | 4525 | TACCCCACAGTGGGGGGTGGTGAAG |
| 3264 | GAACCCTTTTTAGACTGGAT | 3895 | GATATCTGTAGGTATGGAGAACC | 4526 | ACCCCACAGTGGGGGGTGGTGAAGT |
| 3265 | AACCCTTTTTAGACTGGATT | 3896 | ATATCTGTAGGTATGGAGAACCC | 4527 | CCCCACAGTGGGGGGTGGTGAAGTA |
| 3266 | ACCCTTTTTAGACTGGATTT | 3897 | TATCTGTAGGTATGGAGAACCCT | 4528 | CCCACAGTGGGGGGTGGTGAAGTAT |
| 3267 | CCCTTTTTAGACTGGATTTT | 3898 | ATCTGTAGGTATGGAGAACCCTT | 4529 | CCACAGTGGGGGGTGGTGAAGTATC |
| 3268 | CTTTTTAGACTGGATTTTCA | 3899 | TCTGTAGGTATGGAGAACCCTTT | 4530 | CACAGTGGGGGGTGGTGAAGTATCT |
| 3269 | TTTTTAGACTGGATTTTCAG | 3900 | CTGTAGGTATGGAGAACCCTTTT | 4531 | ACAGTGGGGGGTGGTGAAGTATCTT |
| 3270 | TTTAGACTGGATTTTCAGAT | 3901 | TGTAGGTATGGAGAACCCTTTTT | 4532 | CAGTGGGGGGTGGTGAAGTATCTTT |
| 3271 | TTAGACTGGATTTTCAGATT | 3902 | GTAGGTATGGAGAACCCTTTTTA | 4533 | AGTGGGGGGTGGTGAAGTATCTTTC |
| 3272 | TAGACTGGATTTTCAGATTT | 3903 | TAGGTATGGAGAACCCTTTTTAG | 4534 | GTGGGGGGTGGTGAAGTATCTTTCT |
| 3273 | AGACTGGATTTTCAGATTTT | 3904 | AGGTATGGAGAACCCTTTTTAGA | 4535 | TGGGGGGTGGTGAAGTATCTTTCTC |
| 3274 | ACTGGATTTTCAGATTTTGA | 3905 | GGTATGGAGAACCCTTTTTAGAC | 4536 | GGGGGGTGGTGAAGTATCTTTCTCT |
| 3275 | CTGGATTTTCAGATTTTGAT | 3906 | GTATGGAGAACCCTTTTTAGACT | 4537 | GGGGGTGGTGAAGTATCTTTCTCTG |
| 3276 | TTTTCAGATTTTGATATTGA | 3907 | TATGGAGAACCCTTTTTAGACTG | 4538 | GGGGTGGTGAAGTATCTTTCTCTGT |
| 3277 | TTTCAGATTTTGATATTGAG | 3908 | ATGGAGAACCCTTTTTAGACTGG | 4539 | GGGTGGTGAAGTATCTTTCTCTGTG |
| 3278 | TCAGATTTTGATATTGAGCT | 3909 | TGGAGAACCCTTTTTAGACTGGA | 4540 | GGTGGTGAAGTATCTTTCTCTGTGG |
| 3279 | AGATTTTGATATTGAGCTTC | 3910 | GGAGAACCCTTTTTAGACTGGAT | 4541 | GTGGTGAAGTATCTTTCTCTGTGGG |
| 3280 | GATTTTGATATTGAGCTTCT | 3911 | GAGAACCCTTTTTAGACTGGATT | 4542 | TGGTGAAGTATCTTTCTCTGTGGGT |
| 3281 | TTTTGATATTGAGCTTCTCT | 3912 | AGAACCCTTTTTAGACTGGATTT | 4543 | GGTGAAGTATCTTTCTCTGTGGGTC |
| 3282 | TTTGATATTGAGCTTCTCTC | 3913 | GAACCCTTTTTAGACTGGATTTT | 4544 | GTGAAGTATCTTTCTCTGTGGGTCT |
| 3283 | TTGATATTGAGCTTCTCTCT | 3914 | AACCCTTTTTAGACTGGATTTTC | 4545 | TGAAGTATCTTTCTCTGTGGGTCTA |
| 3284 | TGATATTGAGCTTCTCTCTC | 3915 | ACCCTTTTTAGACTGGATTTTCA | 4546 | GAAGTATCTTTCTCTGTGGGTCTAG |
| 3285 | GATATTGAGCTTCTCTCTCT | 3916 | CCCTTTTTAGACTGGATTTTCAG | 4547 | AAGTATCTTTCTCTGTGGGTCTAGG |
| 3286 | TTGAGCTTCTCTCTCTGGGG | 3917 | CCTTTTTAGACTGGATTTTCAGA | 4548 | AGTATCTTTCTCTGTGGGTCTAGGG |
| 3287 | TGAGCTTCTCTCTCTGGGGG | 3918 | CTTTTTAGACTGGATTTTCAGAT | 4549 | GTATCTTTCTCTGTGGGTCTAGGGT |
| 3288 | GAGCTTCTCTCTCTGGGGGA | 3919 | TTTTTAGACTGGATTTTCAGATT | 4550 | TATCTTTCTCTGTGGGTCTAGGGTC |
| 3289 | AGCTTCTCTCTCTGGGGGAT | 3920 | TTTTAGACTGGATTTTCAGATTT | 4551 | ATCTTTCTCTGTGGGTCTAGGGTCT |
| 3290 | CTTCTCTCTCTGGGGGATCT | 3921 | TTTAGACTGGATTTTCAGATTTT | 4552 | TCTTTCTCTGTGGGTCTAGGGTCTC |
| 3291 | TTCTCTCTCTGGGGGATCTG | 3922 | TTAGACTGGATTTTCAGATTTTG | 4553 | CTTTCTCTGTGGGTCTAGGGTCTCT |
| 3292 | TCTCTCTCTGGGGGATCTGG | 3923 | TAGACTGGATTTTCAGATTTTGA | 4554 | TTTCTCTGTGGGTCTAGGGTCTCTC |
| 3293 | CTCTCTCTGGGGGATCTGGG | 3924 | AGACTGGATTTTCAGATTTTGAT | 4555 | TTCTCTGTGGGTCTAGGGTCTCTCT |
| 3294 | CTCTCTGGGGGATCTGGGGT | 3925 | GACTGGATTTTCAGATTTTGATA | 4556 | TCTCTGTGGGTCTAGGGTCTCTCTG |
| 3295 | TCTCTGGGGGATCTGGGGTC | 3926 | ACTGGATTTTCAGATTTTGATAT | 4557 | CTCTGTGGGTCTAGGGTCTCTCTGT |
| 3296 | CTCTGGGGGATCTGGGGTCG | 3927 | CTGGATTTTCAGATTTTGATATT | 4558 | TCTGTGGGTCTAGGGTCTCTCTGTC |
| 3297 | TCTGGGGGATCTGGGGTCGT | 3928 | TGGATTTTCAGATTTTGATATTG | 4559 | CTGTGGGTCTAGGGTCTCTCTGTCT |
| 3298 | TGGGGGATCTGGGGTCGTTT | 3929 | GGATTTTCAGATTTTGATATTGA | 4560 | TGTGGGTCTAGGGTCTCTCTGTCTC |
| 3299 | GGGGGATCTGGGGTCGTTTT | 3930 | GATTTTCAGATTTTGATATTGAG | 4561 | GTGGGTCTAGGGTCTCTCTGTCTCA |
| 3300 | GGGGATCTGGGGTCGTTTTT | 3931 | ATTTTCAGATTTTGATATTGAGC | 4562 | TGGGTCTAGGGTCTCTCTGTCTCAG |
| 3301 | GGGATCTGGGGTCGTTTTTT | 3932 | TTTTCAGATTTTGATATTGAGCT | 4563 | GGGTCTAGGGTCTCTCTGTCTCAGG |
| 3302 | GATCTGGGGTCGTTTTTTCC | 3933 | TTTCAGATTTTGATATTGAGCTT | 4564 | GGTCTAGGGTCTCTCTGTCTCAGGG |
| 3303 | ATCTGGGGTCGTTTTTTCCT | 3934 | TTCAGATTTTGATATTGAGCTTC | 4565 | GTCTAGGGTCTCTCTGTCTCAGGGT |
| 3304 | TCTGGGGTCGTTTTTTCCTC | 3935 | TCAGATTTTGATATTGAGCTTCT | 4566 | TCTAGGGTCTCTCTGTCTCAGGGTC |
| 3305 | GGGGTCGTTTTTTCCTCAAA | 3936 | CAGATTTTGATATTGAGCTTCTC | 4567 | CTAGGGTCTCTCTGTCTCAGGGTCT |
| 3306 | GGTCGTTTTTTCCTCAAATC | 3937 | AGATTTTGATATTGAGCTTCTCT | 4568 | TAGGGTCTCTCTGTCTCAGGGTCTG |
| 3307 | GTCGTTTTTTCCTCAAATCA | 3938 | GATTTTGATATTGAGCTTCTCTC | 4569 | AGGGTCTCTCTGTCTCAGGGTCTGG |
| 3308 | TCGTTTTTTCCTCAAATCAG | 3939 | ATTTTGATATTGAGCTTCTCTCT | 4570 | GGGTCTCTCTGTCTCAGGGTCTGGG |
| 3309 | CGTTTTTTCCTCAAATCAGG | 3940 | TTTTGATATTGAGCTTCTCTCTC | 4571 | GGTCTCTCTGTCTCAGGGTCTGGGG |
| 3310 | TTTTTCCTCAAATCAGGAGT | 3941 | TTTGATATTGAGCTTCTCTCTCT | 4572 | GTCTCTCTGTCTCAGGGTCTGGGGT |
| 3311 | TTTCCTCAAATCAGGAGTCT | 3942 | TTGATATTGAGCTTCTCTCTCTG | 4573 | TCTCTCTGTCTCAGGGTCTGGGGTC |
| 3312 | TCCTCAAATCAGGAGTCTCT | 3943 | TGATATTGAGCTTCTCTCTCTGG | 4574 | CTCTCTGTCTCAGGGTCTGGGGTCT |
| 3313 | CCTCAAATCAGGAGTCTCTT | 3944 | GATATTGAGCTTCTCTCTCTGGG | 4575 | TCTCTGTCTCAGGGTCTGGGGTCTG |
| 3314 | TCAAATCAGGAGTCTCTTTT | 3945 | ATATTGAGCTTCTCTCTCTGGGG | 4576 | CTCTGTCTCAGGGTCTGGGGTCTGC |
| 3315 | CAAATCAGGAGTCTCTTTTC | 3946 | TATTGAGCTTCTCTCTCTGGGGG | 4577 | TCTGTCTCAGGGTCTGGGGTCTGCA |
| 3316 | CAGGAGTCTCTTTTCCTCTA | 3947 | ATTGAGCTTCTCTCTCTGGGGGA | 4578 | CTGTCTCAGGGTCTGGGGTCTGCAT |
| 3317 | GAGTCTCTTTTCCTCTAGAT | 3948 | TTGAGCTTCTCTCTCTGGGGGAT | 4579 | TGTCTCAGGGTCTGGGGTCTGCATC |
| 3318 | AGTCTCTTTTCCTCTAGATT | 3949 | TGAGCTTCTCTCTCTGGGGGATC | 4580 | GTCTCAGGGTCTGGGGTCTGCATCT |
| 3319 | CTTTTCCTCTAGATTTTGGC | 3950 | GAGCTTCTCTCTCTGGGGGATCT | 4581 | TCTCAGGGTCTGGGGTCTGCATCTT |
| 3320 | TTTTCCTCTAGATTTTGGCC | 3951 | AGCTTCTCTCTCTGGGGGATCTG | 4582 | CTCAGGGTCTGGGGTCTGCATCTTT |
| 3321 | TTCCTCTAGATTTTGGCCCT | 3952 | GCTTCTCTCTCTGGGGGATCTGG | 4583 | TCAGGGTCTGGGGTCTGCATCTTTA |
| 3322 | TCCTCTAGATTTTGGCCCTG | 3953 | CTTCTCTCTCTGGGGGATCTGGG | 4584 | CAGGGTCTGGGGTCTGCATCTTTAG |
| 3323 | CCTCTAGATTTTGGCCCTGT | 3954 | TTCTCTCTCTGGGGGATCTGGGG | 4585 | AGGGTCTGGGGTCTGCATCTTTAGG |
| 3324 | CTCTAGATTTTGGCCCTGTG | 3955 | TCTCTCTCTGGGGGATCTGGGGT | 4586 | GGGTCTGGGGTCTGCATCTTTAGGA |
| 3325 | TCTAGATTTTGGCCCTGTGT | 3956 | CTCTCTCTGGGGGATCTGGGGTC | 4587 | GGTCTGGGGTCTGCATCTTTAGGAC |
| 3326 | CTAGATTTTGGCCCTGTGTC | 3957 | TCTCTCTGGGGGATCTGGGGTCG | 4588 | GTCTGGGGTCTGCATCTTTAGGACC |
| 3327 | AGATTTTGGCCCTGTGTCTC | 3958 | CTCTCTGGGGGATCTGGGGTCGT | 4589 | TCTGGGGTCTGCATCTTTAGGACCT |
| 3328 | GATTTTGGCCCTGTGTCTCA | 3959 | TCTCTGGGGGATCTGGGGTCGTT | 4590 | CTGGGGTCTGCATCTTTAGGACCTC |
| 3329 | ATTTTGGCCCTGTGTCTCAA | 3960 | CTCTGGGGGATCTGGGGTCGTTT | 4591 | TGGGGTCTGCATCTTTAGGACCTCT |
| 3330 | TTTTGGCCCTGTGTCTCAAA | 3961 | TCTGGGGGATCTGGGGTCGTTTT | 4592 | GGGGTCTGCATCTTTAGGACCTCTG |
| 3331 | TTTGGCCCTGTGTCTCAAAT | 3962 | CTGGGGGATCTGGGGTCGTTTTT | 4593 | GGGTCTGCATCTTTAGGACCTCTGT |
| 3332 | TTGGCCCTGTGTCTCAAATT | 3963 | TGGGGGATCTGGGGTCGTTTTTT | 4594 | GGTCTGCATCTTTAGGACCTCTGTC |
| 3333 | GGCCCTGTGTCTCAAATTAC | 3964 | GGGGGATCTGGGGTCGTTTTTTC | 4595 | GTCTGCATCTTTAGGACCTCTGTCT |
| 3334 | GCCCTGTGTCTCAAATTACC | 3965 | GGGGATCTGGGGTCGTTTTTTCC | 4596 | TCTGCATCTTTAGGACCTCTGTCTC |
| 3335 | CCCTGTGTCTCAAATTACCC | 3966 | GGGATCTGGGGTCGTTTTTTCCT | 4597 | CTGCATCTTTAGGACCTCTGTCTCT |
| 3336 | CCTGTGTCTCAAATTACCCC | 3967 | GGATCTGGGGTCGTTTTTTCCTC | 4598 | TGCATCTTTAGGACCTCTGTCTCTC |
| 3337 | CTGTGTCTCAAATTACCCCA | 3968 | GATCTGGGGTCGTTTTTTCCTCA | 4599 | GCATCTTTAGGACCTCTGTCTCTCT |
| 3338 | TGTGTCTCAAATTACCCCAC | 3969 | ATCTGGGGTCGTTTTTTCCTCAA | 4600 | CATCTTTAGGACCTCTGTCTCTCTC |
| 3339 | GTGTCTCAAATTACCCCACA | 3970 | TCTGGGGTCGTTTTTTCCTCAAA | 4601 | ATCTTTAGGACCTCTGTCTCTCTCT |
| 3340 | TGTCTCAAATTACCCCACAG | 3971 | CTGGGGTCGTTTTTTCCTCAAAT | 4602 | TCTTTAGGACCTCTGTCTCTCTCTG |
| 3341 | GTCTCAAATTACCCCACAGT | 3972 | TGGGGTCGTTTTTTCCTCAAATC | 4603 | CTTTAGGACCTCTGTCTCTCTCTGG |
| 3342 | TCTCAAATTACCCCACAGTG | 3973 | GGGGTCGTTTTTTCCTCAAATCA | 4604 | TTTAGGACCTCTGTCTCTCTCTGGT |
| 3343 | CTCAAATTACCCCACAGTGG | 3974 | GGGTCGTTTTTTCCTCAAATCAG | 4605 | TTAGGACCTCTGTCTCTCTCTGGTG |
| 3344 | TCAAATTACCCCACAGTGGG | 3975 | GGTCGTTTTTTCCTCAAATCAGG | 4606 | TAGGACCTCTGTCTCTCTCTGGTGT |
| 3345 | CAAATTACCCCACAGTGGGG | 3976 | GTCGTTTTTTCCTCAAATCAGGA | 4607 | AGGACCTCTGTCTCTCTCTGGTGTG |
| 3346 | AAATTACCCCACAGTGGGGG | 3977 | TCGTTTTTTCCTCAAATCAGGAG | 4608 | GGACCTCTGTCTCTCTCTGGTGTGT |
| 3347 | AATTACCCCACAGTGGGGGG | 3978 | CGTTTTTTCCTCAAATCAGGAGT | 4609 | GACCTCTGTCTCTCTCTGGTGTGTT |
| 3348 | TTACCCCACAGTGGGGGGTG | 3979 | GTTTTTTCCTCAAATCAGGAGTC | 4610 | ACCTCTGTCTCTCTCTGGTGTGTTT |
| 3349 | TACCCCACAGTGGGGGGTGG | 3980 | TTTTTTCCTCAAATCAGGAGTCT | 4611 | GGGTCTGGAGTCTAAGATATCTGTAG |
| 3350 | ACCCCACAGTGGGGGGTGGT | 3981 | TTTTTCCTCAAATCAGGAGTCTC | 4612 | GGTCTGGAGTCTAAGATATCTGTAGG |
| 3351 | CCCCACAGTGGGGGGTGGTG | 3982 | TTTTCCTCAAATCAGGAGTCTCT | 4613 | GTCTGGAGTCTAAGATATCTGTAGGT |
| 3352 | CCCACAGTGGGGGGTGGTGA | 3983 | TTTCCTCAAATCAGGAGTCTCTT | 4614 | TCTGGAGTCTAAGATATCTGTAGGTA |
| 3353 | CACAGTGGGGGGTGGTGAAG | 3984 | TTCCTCAAATCAGGAGTCTCTTT | 4615 | CTGGAGTCTAAGATATCTGTAGGTAT |
| 3354 | ACAGTGGGGGGTGGTGAAGT | 3985 | TCCTCAAATCAGGAGTCTCTTTT | 4616 | TGGAGTCTAAGATATCTGTAGGTATG |
| 3355 | AGTGGGGGGTGGTGAAGTAT | 3986 | CCTCAAATCAGGAGTCTCTTTTC | 4617 | GGAGTCTAAGATATCTGTAGGTATGG |
| 3356 | GTGGGGGGTGGTGAAGTATC | 3987 | CTCAAATCAGGAGTCTCTTTTCC | 4618 | GAGTCTAAGATATCTGTAGGTATGGA |
| 3357 | GGGGGGTGGTGAAGTATCTT | 3988 | TCAAATCAGGAGTCTCTTTTCCT | 4619 | AGTCTAAGATATCTGTAGGTATGGAG |
| 3358 | GGGGGTGGTGAAGTATCTTT | 3989 | CAAATCAGGAGTCTCTTTTCCTC | 4620 | GTCTAAGATATCTGTAGGTATGGAGA |
| 3359 | GGGGTGGTGAAGTATCTTTC | 3990 | AATCAGGAGTCTCTTTTCCTCTA | 4621 | TCTAAGATATCTGTAGGTATGGAGAA |
| 3360 | GGGTGGTGAAGTATCTTTCT | 3991 | ATCAGGAGTCTCTTTTCCTCTAG | 4622 | CTAAGATATCTGTAGGTATGGAGAAC |
| 3361 | GGTGGTGAAGTATCTTTCTC | 3992 | TCAGGAGTCTCTTTTCCTCTAGA | 4623 | TAAGATATCTGTAGGTATGGAGAACC |
| 3362 | GTGGTGAAGTATCTTTCTCT | 3993 | CAGGAGTCTCTTTTCCTCTAGAT | 4624 | AAGATATCTGTAGGTATGGAGAACCC |
| 3363 | TGGTGAAGTATCTTTCTCTG | 3994 | AGGAGTCTCTTTTCCTCTAGATT | 4625 | AGATATCTGTAGGTATGGAGAACCCT |
| 3364 | GGTGAAGTATCTTTCTCTGT | 3995 | GGAGTCTCTTTTCCTCTAGATTT | 4626 | GATATCTGTAGGTATGGAGAACCCTT |
| 3365 | GTGAAGTATCTTTCTCTGTG | 3996 | GAGTCTCTTTTCCTCTAGATTTT | 4627 | ATATCTGTAGGTATGGAGAACCCTTT |
| 3366 | TGAAGTATCTTTCTCTGTGG | 3997 | AGTCTCTTTTCCTCTAGATTTTG | 4628 | TATCTGTAGGTATGGAGAACCCTTTT |
| 3367 | GAAGTATCTTTCTCTGTGGG | 3998 | GTCTCTTTTCCTCTAGATTTTGG | 4629 | ATCTGTAGGTATGGAGAACCCTTTTT |
| 3368 | AAGTATCTTTCTCTGTGGGT | 3999 | TCTCTTTTCCTCTAGATTTTGGC | 4630 | TCTGTAGGTATGGAGAACCCTTTTTA |
| 3369 | ATCTTTCTCTGTGGGTCTAG | 4000 | CTCTTTTCCTCTAGATTTTGGCC | 4631 | CTGTAGGTATGGAGAACCCTTTTTAG |
| 3370 | TCTTTCTCTGTGGGTCTAGG | 4001 | TCTTTTCCTCTAGATTTTGGCCC | 4632 | TGTAGGTATGGAGAACCCTTTTTAGA |
| 3371 | CTTTCTCTGTGGGTCTAGGG | 4002 | CTTTTCCTCTAGATTTTGGCCCT | 4633 | GTAGGTATGGAGAACCCTTTTTAGAC |
| 3372 | TTTCTCTGTGGGTCTAGGGT | 4003 | TTTTCCTCTAGATTTTGGCCCTG | 4634 | TAGGTATGGAGAACCCTTTTTAGACT |
| 3373 | TTCTCTGTGGGTCTAGGGTC | 4004 | TTTCCTCTAGATTTTGGCCCTGT | 4635 | AGGTATGGAGAACCCTTTTTAGACTG |
| 3374 | TCTCTGTGGGTCTAGGGTCT | 4005 | TTCCTCTAGATTTTGGCCCTGTG | 4636 | GGTATGGAGAACCCTTTTTAGACTGG |
| 3375 | CTCTGTGGGTCTAGGGTCTC | 4006 | TCCTCTAGATTTTGGCCCTGTGT | 4637 | GTATGGAGAACCCTTTTTAGACTGGA |
| 3376 | TCTGTGGGTCTAGGGTCTCT | 4007 | CCTCTAGATTTTGGCCCTGTGTC | 4638 | TATGGAGAACCCTTTTTAGACTGGAT |
| 3377 | CTGTGGGTCTAGGGTCTCTC | 4008 | CTCTAGATTTTGGCCCTGTGTCT | 4639 | ATGGAGAACCCTTTTTAGACTGGATT |
| 3378 | TGTGGGTCTAGGGTCTCTCT | 4009 | TCTAGATTTTGGCCCTGTGTCTC | 4640 | TGGAGAACCCTTTTTAGACTGGATTT |
| 3379 | GTGGGTCTAGGGTCTCTCTG | 4010 | CTAGATTTTGGCCCTGTGTCTCA | 4641 | GGAGAACCCTTTTTAGACTGGATTTT |
| 3380 | TGGGTCTAGGGTCTCTCTGT | 4011 | TAGATTTTGGCCCTGTGTCTCAA | 4642 | GAGAACCCTTTTTAGACTGGATTTTC |
| 3381 | GGGTCTAGGGTCTCTCTGTC | 4012 | AGATTTTGGCCCTGTGTCTCAAA | 4643 | AGAACCCTTTTTAGACTGGATTTTCA |
| 3382 | GGTCTAGGGTCTCTCTGTCT | 4013 | GATTTTGGCCCTGTGTCTCAAAT | 4644 | GAACCCTTTTTAGACTGGATTTTCAG |
| 3383 | GTCTAGGGTCTCTCTGTCTC | 4014 | ATTTTGGCCCTGTGTCTCAAATT | 4645 | AACCCTTTTTAGACTGGATTTTCAGA |
| 3384 | TCTAGGGTCTCTCTGTCTCA | 4015 | TTTTGGCCCTGTGTCTCAAATTA | 4646 | ACCCTTTTTAGACTGGATTTTCAGAT |
| 3385 | CTAGGGTCTCTCTGTCTCAG | 4016 | TTTGGCCCTGTGTCTCAAATTAC | 4647 | CCCTTTTTAGACTGGATTTTCAGATT |
| 3386 | AGGGTCTCTCTGTCTCAGGG | 4017 | TTGGCCCTGTGTCTCAAATTACC | 4648 | CCTTTTTAGACTGGATTTTCAGATTT |
| 3387 | GTCTCTCTGTCTCAGGGTCT | 4018 | TGGCCCTGTGTCTCAAATTACCC | 4649 | CTTTTTAGACTGGATTTTCAGATTTT |
| 3388 | TCTCTGTCTCAGGGTCTGGG | 4019 | GGCCCTGTGTCTCAAATTACCCC | 4650 | TTTTTAGACTGGATTTTCAGATTTTG |
| 3389 | CTCTGTCTCAGGGTCTGGGG | 4020 | GCCCTGTGTCTCAAATTACCCCA | 4651 | TTTTAGACTGGATTTTCAGATTTTGA |
| 3390 | TCTGTCTCAGGGTCTGGGGT | 4021 | CCCTGTGTCTCAAATTACCCCAC | 4652 | TTTAGACTGGATTTTCAGATTTTGAT |
| 3391 | CTGTCTCAGGGTCTGGGGTC | 4022 | CCTGTGTCTCAAATTACCCCACA | 4653 | TTAGACTGGATTTTCAGATTTTGATA |
| 3392 | GTCTCAGGGTCTGGGGTCTG | 4023 | CTGTGTCTCAAATTACCCCACAG | 4654 | TAGACTGGATTTTCAGATTTTGATAT |
| 3393 | CTCAGGGTCTGGGGTCTGCA | 4024 | TGTGTCTCAAATTACCCCACAGT | 4655 | AGACTGGATTTTCAGATTTTGATATT |
| 3394 | TCAGGGTCTGGGGTCTGCAT | 4025 | GTGTCTCAAATTACCCCACAGTG | 4656 | GACTGGATTTTCAGATTTTGATATTG |
| 3395 | GTCTGGGGTCTGCATCTTTA | 4026 | TGTCTCAAATTACCCCACAGTGG | 4657 | ACTGGATTTTCAGATTTTGATATTGA |
| 3396 | TCTGGGGTCTGCATCTTTAG | 4027 | GTCTCAAATTACCCCACAGTGGG | 4658 | CTGGATTTTCAGATTTTGATATTGAG |
| 3397 | CTGGGGTCTGCATCTTTAGG | 4028 | TCTCAAATTACCCCACAGTGGGG | 4659 | TGGATTTTCAGATTTTGATATTGAGC |
| 3398 | TGGGGTCTGCATCTTTAGGA | 4029 | CTCAAATTACCCCACAGTGGGGG | 4660 | GGATTTTCAGATTTTGATATTGAGCT |
| 3399 | GGGTCTGCATCTTTAGGACC | 4030 | TCAAATTACCCCACAGTGGGGGG | 4661 | GATTTTCAGATTTTGATATTGAGCTT |
| 3400 | GGTCTGCATCTTTAGGACCT | 4031 | CAAATTACCCCACAGTGGGGGGT | 4662 | ATTTTCAGATTTTGATATTGAGCTTC |
| 3401 | TGCATCTTTAGGACCTCTGT | 4032 | AAATTACCCCACAGTGGGGGGTG | 4663 | TTTTCAGATTTTGATATTGAGCTTCT |
| 3402 | CATCTTTAGGACCTCTGTCT | 4033 | AATTACCCCACAGTGGGGGGTGG | 4664 | TTTCAGATTTTGATATTGAGCTTCTC |
| 3403 | ATCTTTAGGACCTCTGTCTC | 4034 | ATTACCCCACAGTGGGGGGTGGT | 4665 | TTCAGATTTTGATATTGAGCTTCTCT |
| 3404 | CTTTAGGACCTCTGTCTCTC | 4035 | TTACCCCACAGTGGGGGGTGGTG | 4666 | TCAGATTTTGATATTGAGCTTCTCTC |
| 3405 | AGGACCTCTGTCTCTCTCTG | 4036 | TACCCCACAGTGGGGGGTGGTGA | 4667 | CAGATTTTGATATTGAGCTTCTCTCT |
| 3406 | GGACCTCTGTCTCTCTCTGG | 4037 | ACCCCACAGTGGGGGGTGGTGAA | 4668 | AGATTTTGATATTGAGCTTCTCTCTC |
| 3407 | CTGTCTCTCTCTGGTGTGTT | 4038 | CCCCACAGTGGGGGGTGGTGAAG | 4669 | GATTTTGATATTGAGCTTCTCTCTCT |
| 3408 | GGGTCTGGAGTCTAAGATATC | 4039 | CCCACAGTGGGGGGTGGTGAAGT | 4670 | ATTTTGATATTGAGCTTCTCTCTCTG |
| 3409 | GGTCTGGAGTCTAAGATATCT | 4040 | CCACAGTGGGGGGTGGTGAAGTA | 4671 | TTTTGATATTGAGCTTCTCTCTCTGG |
| 3410 | GTCTGGAGTCTAAGATATCTG | 4041 | CACAGTGGGGGGTGGTGAAGTAT | 4672 | TTTGATATTGAGCTTCTCTCTCTGGG |
| 3411 | TCTGGAGTCTAAGATATCTGT | 4042 | ACAGTGGGGGGTGGTGAAGTATC | 4673 | TTGATATTGAGCTTCTCTCTCTGGGG |
| 3412 | CTGGAGTCTAAGATATCTGTA | 4043 | CAGTGGGGGGTGGTGAAGTATCT | 4674 | TGATATTGAGCTTCTCTCTCTGGGGG |
| 3413 | TGGAGTCTAAGATATCTGTAG | 4044 | AGTGGGGGGTGGTGAAGTATCTT | 4675 | GATATTGAGCTTCTCTCTCTGGGGGA |
| 3414 | GGAGTCTAAGATATCTGTAGG | 4045 | GTGGGGGGTGGTGAAGTATCTTT | 4676 | ATATTGAGCTTCTCTCTCTGGGGGAT |
| 3415 | GAGTCTAAGATATCTGTAGGT | 4046 | TGGGGGGTGGTGAAGTATCTTTC | 4677 | TATTGAGCTTCTCTCTCTGGGGGATC |
| 3416 | AGTCTAAGATATCTGTAGGTA | 4047 | GGGGGGTGGTGAAGTATCTTTCT | 4678 | ATTGAGCTTCTCTCTCTGGGGGATCT |
| 3417 | GTCTAAGATATCTGTAGGTAT | 4048 | GGGGGTGGTGAAGTATCTTTCTC | 4679 | TTGAGCTTCTCTCTCTGGGGGATCTG |
| 3418 | TCTAAGATATCTGTAGGTATG | 4049 | GGGGTGGTGAAGTATCTTTCTCT | 4680 | TGAGCTTCTCTCTCTGGGGGATCTGG |
| 3419 | CTAAGATATCTGTAGGTATGG | 4050 | GGGTGGTGAAGTATCTTTCTCTG | 4681 | GAGCTTCTCTCTCTGGGGGATCTGGG |
| 3420 | TAAGATATCTGTAGGTATGGA | 4051 | GGTGGTGAAGTATCTTTCTCTGT | 4682 | AGCTTCTCTCTCTGGGGGATCTGGGG |
| 3421 | AAGATATCTGTAGGTATGGAG | 4052 | GTGGTGAAGTATCTTTCTCTGTG | 4683 | GCTTCTCTCTCTGGGGGATCTGGGGT |
| 3422 | AGATATCTGTAGGTATGGAGA | 4053 | TGGTGAAGTATCTTTCTCTGTGG | 4684 | CTTCTCTCTCTGGGGGATCTGGGGTC |
| 3423 | GATATCTGTAGGTATGGAGAA | 4054 | GGTGAAGTATCTTTCTCTGTGGG | 4685 | TTCTCTCTCTGGGGGATCTGGGGTCG |
| 3424 | ATATCTGTAGGTATGGAGAAC | 4055 | GTGAAGTATCTTTCTCTGTGGGT | 4686 | TCTCTCTCTGGGGGATCTGGGGTCGT |
| 3425 | ATCTGTAGGTATGGAGAACCC | 4056 | TGAAGTATCTTTCTCTGTGGGTC | 4687 | CTCTCTCTGGGGGATCTGGGGTCGTT |
| 3426 | TCTGTAGGTATGGAGAACCCT | 4057 | GAAGTATCTTTCTCTGTGGGTCT | 4688 | TCTCTCTGGGGGATCTGGGGTCGTTT |
| 3427 | CTGTAGGTATGGAGAACCCTT | 4058 | AAGTATCTTTCTCTGTGGGTCTA | 4689 | CTCTCTGGGGGATCTGGGGTCGTTTT |
| 3428 | TGTAGGTATGGAGAACCCTTT | 4059 | AGTATCTTTCTCTGTGGGTCTAG | 4690 | TCTCTGGGGGATCTGGGGTCGTTTTT |
| 3429 | GTAGGTATGGAGAACCCTTTT | 4060 | GTATCTTTCTCTGTGGGTCTAGG | 4691 | CTCTGGGGGATCTGGGGTCGTTTTTT |
| 3430 | TAGGTATGGAGAACCCTTTTT | 4061 | TATCTTTCTCTGTGGGTCTAGGG | 4692 | TCTGGGGGATCTGGGGTCGTTTTTTC |
| 3431 | AGGTATGGAGAACCCTTTTTA | 4062 | ATCTTTCTCTGTGGGTCTAGGGT | 4693 | CTGGGGGATCTGGGGTCGTTTTTTCC |
| 3432 | GGTATGGAGAACCCTTTTTAG | 4063 | TCTTTCTCTGTGGGTCTAGGGTC | 4694 | TGGGGGATCTGGGGTCGTTTTTTCCT |
| 3433 | GTATGGAGAACCCTTTTTAGA | 4064 | CTTTCTCTGTGGGTCTAGGGTCT | 4695 | GGGGGATCTGGGGTCGTTTTTTCCTC |
| 3434 | TATGGAGAACCCTTTTTAGAC | 4065 | TTTCTCTGTGGGTCTAGGGTCTC | 4696 | GGGGATCTGGGGTCGTTTTTTCCTCA |
| 3435 | ATGGAGAACCCTTTTTAGACT | 4066 | TTCTCTGTGGGTCTAGGGTCTCT | 4697 | GGGATCTGGGGTCGTTTTTTCCTCAA |
| 3436 | TGGAGAACCCTTTTTAGACTG | 4067 | TCTCTGTGGGTCTAGGGTCTCTC | 4698 | GGATCTGGGGTCGTTTTTTCCTCAAA |
| 3437 | GGAGAACCCTTTTTAGACTGG | 4068 | CTCTGTGGGTCTAGGGTCTCTCT | 4699 | GATCTGGGGTCGTTTTTTCCTCAAAT |
| 3438 | GAGAACCCTTTTTAGACTGGA | 4069 | TCTGTGGGTCTAGGGTCTCTCTG | 4700 | ATCTGGGGTCGTTTTTTCCTCAAATC |
| 3439 | AGAACCCTTTTTAGACTGGAT | 4070 | CTGTGGGTCTAGGGTCTCTCTGT | 4701 | TCTGGGGTCGTTTTTTCCTCAAATCA |
| 3440 | GAACCCTTTTTAGACTGGATT | 4071 | TGTGGGTCTAGGGTCTCTCTGTC | 4702 | CTGGGGTCGTTTTTTCCTCAAATCAG |
| 3441 | AACCCTTTTTAGACTGGATTT | 4072 | GTGGGTCTAGGGTCTCTCTGTCT | 4703 | TGGGGTCGTTTTTTCCTCAAATCAGG |
| 3442 | ACCCTTTTTAGACTGGATTTT | 4073 | TGGGTCTAGGGTCTCTCTGTCTC | 4704 | GGGGTCGTTTTTTCCTCAAATCAGGA |
| 3443 | CCCTTTTTAGACTGGATTTTC | 4074 | GGGTCTAGGGTCTCTCTGTCTCA | 4705 | GGGTCGTTTTTTCCTCAAATCAGGAG |
| 3444 | CCTTTTTAGACTGGATTTTCA | 4075 | GGTCTAGGGTCTCTCTGTCTCAG | 4706 | GGTCGTTTTTTCCTCAAATCAGGAGT |
| 3445 | CTTTTTAGACTGGATTTTCAG | 4076 | GTCTAGGGTCTCTCTGTCTCAGG | 4707 | GTCGTTTTTTCCTCAAATCAGGAGTC |
| 3446 | TTTTTAGACTGGATTTTCAGA | 4077 | TCTAGGGTCTCTCTGTCTCAGGG | 4708 | TCGTTTTTTCCTCAAATCAGGAGTCT |
| 3447 | TTTTAGACTGGATTTTCAGAT | 4078 | CTAGGGTCTCTCTGTCTCAGGGT | 4709 | CGTTTTTTCCTCAAATCAGGAGTCTC |
| 3448 | TTTAGACTGGATTTTCAGATT | 4079 | TAGGGTCTCTCTGTCTCAGGGTC | 4710 | GTTTTTTCCTCAAATCAGGAGTCTCT |
| 3449 | TTAGACTGGATTTTCAGATTT | 4080 | AGGGTCTCTCTGTCTCAGGGTCT | 4711 | TTTTTTCCTCAAATCAGGAGTCTCTT |
| 3450 | TAGACTGGATTTTCAGATTTT | 4081 | GGGTCTCTCTGTCTCAGGGTCTG | 4712 | TTTTTCCTCAAATCAGGAGTCTCTTT |
| 3451 | AGACTGGATTTTCAGATTTTG | 4082 | GGTCTCTCTGTCTCAGGGTCTGG | 4713 | TTTTCCTCAAATCAGGAGTCTCTTTT |
| 3452 | GACTGGATTTTCAGATTTTGA | 4083 | GTCTCTCTGTCTCAGGGTCTGGG | 4714 | TTTCCTCAAATCAGGAGTCTCTTTTC |
| 3453 | ACTGGATTTTCAGATTTTGAT | 4084 | TCTCTCTGTCTCAGGGTCTGGGG | 4715 | TTCCTCAAATCAGGAGTCTCTTTTCC |
| 3454 | CTGGATTTTCAGATTTTGATA | 4085 | CTCTCTGTCTCAGGGTCTGGGGT | 4716 | TCCTCAAATCAGGAGTCTCTTTTCCT |
| 3455 | GGATTTTCAGATTTTGATATT | 4086 | TCTCTGTCTCAGGGTCTGGGGTC | 4717 | CCTCAAATCAGGAGTCTCTTTTCCTC |
| 3456 | GATTTTCAGATTTTGATATTG | 4087 | CTCTGTCTCAGGGTCTGGGGTCT | 4718 | CTCAAATCAGGAGTCTCTTTTCCTCT |
| 3457 | ATTTTCAGATTTTGATATTGA | 4088 | TCTGTCTCAGGGTCTGGGGTCTG | 4719 | TCAAATCAGGAGTCTCTTTTCCTCTA |
| 3458 | TTTTCAGATTTTGATATTGAG | 4089 | CTGTCTCAGGGTCTGGGGTCTGC | 4720 | CAAATCAGGAGTCTCTTTTCCTCTAG |
| 3459 | TTTCAGATTTTGATATTGAGC | 4090 | TGTCTCAGGGTCTGGGGTCTGCA | 4721 | AAATCAGGAGTCTCTTTTCCTCTAGA |
| 3460 | TTCAGATTTTGATATTGAGCT | 4091 | GTCTCAGGGTCTGGGGTCTGCAT | 4722 | AATCAGGAGTCTCTTTTCCTCTAGAT |
| 3461 | TCAGATTTTGATATTGAGCTT | 4092 | TCTCAGGGTCTGGGGTCTGCATC | 4723 | ATCAGGAGTCTCTTTTCCTCTAGATT |
| 3462 | CAGATTTTGATATTGAGCTTC | 4093 | CTCAGGGTCTGGGGTCTGCATCT | 4724 | TCAGGAGTCTCTTTTCCTCTAGATTT |
| 3463 | AGATTTTGATATTGAGCTTCT | 4094 | TCAGGGTCTGGGGTCTGCATCTT | 4725 | CAGGAGTCTCTTTTCCTCTAGATTTT |
| 3464 | GATTTTGATATTGAGCTTCTC | 4095 | CAGGGTCTGGGGTCTGCATCTTT | 4726 | AGGAGTCTCTTTTCCTCTAGATTTTG |
| 3465 | ATTTTGATATTGAGCTTCTCT | 4096 | AGGGTCTGGGGTCTGCATCTTTA | 4727 | GGAGTCTCTTTTCCTCTAGATTTTGG |
| 3466 | TTTTGATATTGAGCTTCTCTC | 4097 | GGGTCTGGGGTCTGCATCTTTAG | 4728 | GAGTCTCTTTTCCTCTAGATTTTGGC |
| 3467 | TTTGATATTGAGCTTCTCTCT | 4098 | GGTCTGGGGTCTGCATCTTTAGG | 4729 | AGTCTCTTTTCCTCTAGATTTTGGCC |
| 3468 | TTGATATTGAGCTTCTCTCTC | 4099 | GTCTGGGGTCTGCATCTTTAGGA | 4730 | GTCTCTTTTCCTCTAGATTTTGGCCC |
| 3469 | TGATATTGAGCTTCTCTCTCT | 4100 | TCTGGGGTCTGCATCTTTAGGAC | 4731 | TCTCTTTTCCTCTAGATTTTGGCCCT |
| 3470 | GATATTGAGCTTCTCTCTCTG | 4101 | CTGGGGTCTGCATCTTTAGGACC | 4732 | CTCTTTTCCTCTAGATTTTGGCCCTG |
| 3471 | ATATTGAGCTTCTCTCTCTGG | 4102 | TGGGGTCTGCATCTTTAGGACCT | 4733 | TCTTTTCCTCTAGATTTTGGCCCTGT |
| 3472 | TATTGAGCTTCTCTCTCTGGG | 4103 | GGGGTCTGCATCTTTAGGACCTC | 4734 | CTTTTCCTCTAGATTTTGGCCCTGTG |
| 3473 | ATTGAGCTTCTCTCTCTGGGG | 4104 | GGGTCTGCATCTTTAGGACCTCT | 4735 | TTTTCCTCTAGATTTTGGCCCTGTGT |
| 3474 | TTGAGCTTCTCTCTCTGGGGG | 4105 | GGTCTGCATCTTTAGGACCTCTG | 4736 | TTTCCTCTAGATTTTGGCCCTGTGTC |
| 3475 | TGAGCTTCTCTCTCTGGGGGA | 4106 | GTCTGCATCTTTAGGACCTCTGT | 4737 | TTCCTCTAGATTTTGGCCCTGTGTCT |
| 3476 | GAGCTTCTCTCTCTGGGGGAT | 4107 | TCTGCATCTTTAGGACCTCTGTC | 4738 | TCCTCTAGATTTTGGCCCTGTGTCTC |
| 3477 | AGCTTCTCTCTCTGGGGGATC | 4108 | CTGCATCTTTAGGACCTCTGTCT | 4739 | CCTCTAGATTTTGGCCCTGTGTCTCA |
| 3478 | GCTTCTCTCTCTGGGGGATCT | 4109 | TGCATCTTTAGGACCTCTGTCTC | 4740 | CTCTAGATTTTGGCCCTGTGTCTCAA |
| 3479 | CTTCTCTCTCTGGGGGATCTG | 4110 | GCATCTTTAGGACCTCTGTCTCT | 4741 | TCTAGATTTTGGCCCTGTGTCTCAAA |
| 3480 | TTCTCTCTCTGGGGGATCTGG | 4111 | CATCTTTAGGACCTCTGTCTCTC | 4742 | CTAGATTTTGGCCCTGTGTCTCAAAT |
| 3481 | TCTCTCTCTGGGGGATCTGGG | 4112 | ATCTTTAGGACCTCTGTCTCTCT | 4743 | TAGATTTTGGCCCTGTGTCTCAAATT |
| 3482 | CTCTCTCTGGGGGATCTGGGG | 4113 | TCTTTAGGACCTCTGTCTCTCTC | 4744 | AGATTTTGGCCCTGTGTCTCAAATTA |
| 3483 | TCTCTCTGGGGGATCTGGGGT | 4114 | CTTTAGGACCTCTGTCTCTCTCT | 4745 | GATTTTGGCCCTGTGTCTCAAATTAC |
| 3484 | CTCTCTGGGGGATCTGGGGTC | 4115 | TTTAGGACCTCTGTCTCTCTCTG | 4746 | ATTTTGGCCCTGTGTCTCAAATTACC |
| 3485 | TCTCTGGGGGATCTGGGGTCG | 4116 | TTAGGACCTCTGTCTCTCTCTGG | 4747 | TTTTGGCCCTGTGTCTCAAATTACCC |
| 3486 | CTCTGGGGGATCTGGGGTCGT | 4117 | TAGGACCTCTGTCTCTCTCTGGT | 4748 | TTTGGCCCTGTGTCTCAAATTACCCC |
| 3487 | TCTGGGGGATCTGGGGTCGTT | 4118 | AGGACCTCTGTCTCTCTCTGGTG | 4749 | TTGGCCCTGTGTCTCAAATTACCCCA |
| 3488 | CTGGGGGATCTGGGGTCGTTT | 4119 | GGACCTCTGTCTCTCTCTGGTGT | 4750 | TGGCCCTGTGTCTCAAATTACCCCAC |
| 3489 | TGGGGGATCTGGGGTCGTTTT | 4120 | GACCTCTGTCTCTCTCTGGTGTG | 4751 | GGCCCTGTGTCTCAAATTACCCCACA |
| 3490 | GGGGGATCTGGGGTCGTTTTT | 4121 | ACCTCTGTCTCTCTCTGGTGTGT | 4752 | GCCCTGTGTCTCAAATTACCCCACAG |
| 3491 | GGGGATCTGGGGTCGTTTTTT | 4122 | CCTCTGTCTCTCTCTGGTGTGTT | 4753 | CCCTGTGTCTCAAATTACCCCACAGT |
| 3492 | GGGATCTGGGGTCGTTTTTTC | 4123 | CTCTGTCTCTCTCTGGTGTGTTT | 4754 | CCTGTGTCTCAAATTACCCCACAGTG |
| 3493 | GGATCTGGGGTCGTTTTTTCC | 4124 | GGGTCTGGAGTCTAAGATATCTGT | 4755 | CTGTGTCTCAAATTACCCCACAGTGG |
| 3494 | GATCTGGGGTCGTTTTTTCCT | 4125 | GGTCTGGAGTCTAAGATATCTGTA | 4756 | TGTGTCTCAAATTACCCCACAGTGGG |
| 3495 | ATCTGGGGTCGTTTTTTCCTC | 4126 | GTCTGGAGTCTAAGATATCTGTAG | 4757 | GTGTCTCAAATTACCCCACAGTGGGG |
| 3496 | TCTGGGGTCGTTTTTTCCTCA | 4127 | TCTGGAGTCTAAGATATCTGTAGG | 4758 | TGTCTCAAATTACCCCACAGTGGGGG |
| 3497 | TGGGGTCGTTTTTTCCTCAAA | 4128 | CTGGAGTCTAAGATATCTGTAGGT | 4759 | GTCTCAAATTACCCCACAGTGGGGGG |
| 3498 | GGGGTCGTTTTTTCCTCAAAT | 4129 | TGGAGTCTAAGATATCTGTAGGTA | 4760 | TCTCAAATTACCCCACAGTGGGGGGT |
| 3499 | GGGTCGTTTTTTCCTCAAATC | 4130 | GGAGTCTAAGATATCTGTAGGTAT | 4761 | CTCAAATTACCCCACAGTGGGGGGTG |
| 3500 | GGTCGTTTTTTCCTCAAATCA | 4131 | GAGTCTAAGATATCTGTAGGTATG | 4762 | TCAAATTACCCCACAGTGGGGGGTGG |
| 3501 | GTCGTTTTTTCCTCAAATCAG | 4132 | AGTCTAAGATATCTGTAGGTATGG | 4763 | CAAATTACCCCACAGTGGGGGGTGGT |
| 3502 | TCGTTTTTTCCTCAAATCAGG | 4133 | GTCTAAGATATCTGTAGGTATGGA | 4764 | AAATTACCCCACAGTGGGGGGTGGTG |
| 3503 | CGTTTTTTCCTCAAATCAGGA | 4134 | TCTAAGATATCTGTAGGTATGGAG | 4765 | AATTACCCCACAGTGGGGGGTGGTGA |
| 3504 | GTTTTTTCCTCAAATCAGGAG | 4135 | CTAAGATATCTGTAGGTATGGAGA | 4766 | ATTACCCCACAGTGGGGGGTGGTGAA |
| 3505 | TTTTTTCCTCAAATCAGGAGT | 4136 | TAAGATATCTGTAGGTATGGAGAA | 4767 | TTACCCCACAGTGGGGGGTGGTGAAG |
| 3506 | TTTTTCCTCAAATCAGGAGTC | 4137 | AAGATATCTGTAGGTATGGAGAAC | 4768 | TACCCCACAGTGGGGGGTGGTGAAGT |
| 3507 | TTTTCCTCAAATCAGGAGTCT | 4138 | AGATATCTGTAGGTATGGAGAACC | 4769 | ACCCCACAGTGGGGGGTGGTGAAGTA |
| 3508 | TTTCCTCAAATCAGGAGTCTC | 4139 | GATATCTGTAGGTATGGAGAACCC | 4770 | CCCCACAGTGGGGGGTGGTGAAGTAT |
| 3509 | TTCCTCAAATCAGGAGTCTCT | 4140 | ATATCTGTAGGTATGGAGAACCCT | 4771 | CCCACAGTGGGGGGTGGTGAAGTATC |
| 3510 | TCCTCAAATCAGGAGTCTCTT | 4141 | TATCTGTAGGTATGGAGAACCCTT | 4772 | CCACAGTGGGGGGTGGTGAAGTATCT |
| 3511 | CCTCAAATCAGGAGTCTCTTT | 4142 | ATCTGTAGGTATGGAGAACCCTTT | 4773 | CACAGTGGGGGGTGGTGAAGTATCTT |
| 3512 | CTCAAATCAGGAGTCTCTTTT | 4143 | TCTGTAGGTATGGAGAACCCTTTT | 4774 | ACAGTGGGGGGTGGTGAAGTATCTTT |
| 3513 | TCAAATCAGGAGTCTCTTTTC | 4144 | CTGTAGGTATGGAGAACCCTTTTT | 4775 | CAGTGGGGGGTGGTGAAGTATCTTTC |
| 3514 | CAAATCAGGAGTCTCTTTTCC | 4145 | TGTAGGTATGGAGAACCCTTTTTA | 4776 | AGTGGGGGGTGGTGAAGTATCTTTCT |
| 3515 | TCAGGAGTCTCTTTTCCTCTA | 4146 | GTAGGTATGGAGAACCCTTTTTAG | 4777 | GTGGGGGGTGGTGAAGTATCTTTCTC |
| 3516 | CAGGAGTCTCTTTTCCTCTAG | 4147 | TAGGTATGGAGAACCCTTTTTAGA | 4778 | TGGGGGGTGGTGAAGTATCTTTCTCT |
| 3517 | AGGAGTCTCTTTTCCTCTAGA | 4148 | AGGTATGGAGAACCCTTTTTAGAC | 4779 | GGGGGGTGGTGAAGTATCTTTCTCTG |
| 3518 | GGAGTCTCTTTTCCTCTAGAT | 4149 | GGTATGGAGAACCCTTTTTAGACT | 4780 | GGGGGTGGTGAAGTATCTTTCTCTGT |
| 3519 | GAGTCTCTTTTCCTCTAGATT | 4150 | GTATGGAGAACCCTTTTTAGACTG | 4781 | GGGGTGGTGAAGTATCTTTCTCTGTG |
| 3520 | AGTCTCTTTTCCTCTAGATTT | 4151 | TATGGAGAACCCTTTTTAGACTGG | 4782 | GGGTGGTGAAGTATCTTTCTCTGTGG |
| 3521 | CTCTTTTCCTCTAGATTTTGG | 4152 | ATGGAGAACCCTTTTTAGACTGGA | 4783 | GGTGGTGAAGTATCTTTCTCTGTGGG |
| 3522 | TCTTTTCCTCTAGATTTTGGC | 4153 | TGGAGAACCCTTTTTAGACTGGAT | 4784 | GTGGTGAAGTATCTTTCTCTGTGGGT |
| 3523 | CTTTTCCTCTAGATTTTGGCC | 4154 | GGAGAACCCTTTTTAGACTGGATT | 4785 | TGGTGAAGTATCTTTCTCTGTGGGTC |
| 3524 | TTTTCCTCTAGATTTTGGCCC | 4155 | GAGAACCCTTTTTAGACTGGATTT | 4786 | GGTGAAGTATCTTTCTCTGTGGGTCT |
| 3525 | TTTCCTCTAGATTTTGGCCCT | 4156 | AGAACCCTTTTTAGACTGGATTTT | 4787 | GTGAAGTATCTTTCTCTGTGGGTCTA |
| 3526 | TTCCTCTAGATTTTGGCCCTG | 4157 | GAACCCTTTTTAGACTGGATTTTC | 4788 | TGAAGTATCTTTCTCTGTGGGTCTAG |
| 3527 | TCCTCTAGATTTTGGCCCTGT | 4158 | AACCCTTTTTAGACTGGATTTTCA | 4789 | GAAGTATCTTTCTCTGTGGGTCTAGG |
| 3528 | CCTCTAGATTTTGGCCCTGTG | 4159 | ACCCTTTTTAGACTGGATTTTCAG | 4790 | AAGTATCTTTCTCTGTGGGTCTAGGG |
| 3529 | CTCTAGATTTTGGCCCTGTGT | 4160 | CCCTTTTTAGACTGGATTTTCAGA | 4791 | AGTATCTTTCTCTGTGGGTCTAGGGT |
| 3530 | TCTAGATTTTGGCCCTGTGTC | 4161 | CCTTTTTAGACTGGATTTTCAGAT | 4792 | GTATCTTTCTCTGTGGGTCTAGGGTC |
| 3531 | CTAGATTTTGGCCCTGTGTCT | 4162 | CTTTTTAGACTGGATTTTCAGATT | 4793 | TATCTTTCTCTGTGGGTCTAGGGTCT |
| 3532 | TAGATTTTGGCCCTGTGTCTC | 4163 | TTTTTAGACTGGATTTTCAGATTT | 4794 | ATCTTTCTCTGTGGGTCTAGGGTCTC |
| 3533 | AGATTTTGGCCCTGTGTCTCA | 4164 | TTTTAGACTGGATTTTCAGATTTT | 4795 | TCTTTCTCTGTGGGTCTAGGGTCTCT |
| 3534 | GATTTTGGCCCTGTGTCTCAA | 4165 | TTTAGACTGGATTTTCAGATTTTG | 4796 | CTTTCTCTGTGGGTCTAGGGTCTCTC |
| 3535 | ATTTTGGCCCTGTGTCTCAAA | 4166 | TTAGACTGGATTTTCAGATTTTGA | 4797 | TTTCTCTGTGGGTCTAGGGTCTCTCT |
| 3536 | TTTTGGCCCTGTGTCTCAAAT | 4167 | TAGACTGGATTTTCAGATTTTGAT | 4798 | TTCTCTGTGGGTCTAGGGTCTCTCTG |
| 3537 | TTTGGCCCTGTGTCTCAAATT | 4168 | AGACTGGATTTTCAGATTTTGATA | 4799 | TCTCTGTGGGTCTAGGGTCTCTCTGT |
| 3538 | TTGGCCCTGTGTCTCAAATTA | 4169 | GACTGGATTTTCAGATTTTGATAT | 4800 | CTCTGTGGGTCTAGGGTCTCTCTGTC |
| 3539 | TGGCCCTGTGTCTCAAATTAC | 4170 | ACTGGATTTTCAGATTTTGATATT | 4801 | TCTGTGGGTCTAGGGTCTCTCTGTCT |
| 3540 | GGCCCTGTGTCTCAAATTACC | 4171 | CTGGATTTTCAGATTTTGATATTG | 4802 | CTGTGGGTCTAGGGTCTCTCTGTCTC |
| 3541 | GCCCTGTGTCTCAAATTACCC | 4172 | TGGATTTTCAGATTTTGATATTGA | 4803 | TGTGGGTCTAGGGTCTCTCTGTCTCA |
| 3542 | CCCTGTGTCTCAAATTACCCC | 4173 | GGATTTTCAGATTTTGATATTGAG | 4804 | GTGGGTCTAGGGTCTCTCTGTCTCAG |
| 3543 | CCTGTGTCTCAAATTACCCCA | 4174 | GATTTTCAGATTTTGATATTGAGC | 4805 | TGGGTCTAGGGTCTCTCTGTCTCAGG |
| 3544 | CTGTGTCTCAAATTACCCCAC | 4175 | ATTTTCAGATTTTGATATTGAGCT | 4806 | GGGTCTAGGGTCTCTCTGTCTCAGGG |
| 3545 | TGTGTCTCAAATTACCCCACA | 4176 | TTTTCAGATTTTGATATTGAGCTT | 4807 | GGTCTAGGGTCTCTCTGTCTCAGGGT |
| 3546 | GTGTCTCAAATTACCCCACAG | 4177 | TTTCAGATTTTGATATTGAGCTTC | 4808 | GTCTAGGGTCTCTCTGTCTCAGGGTC |
| 3547 | TGTCTCAAATTACCCCACAGT | 4178 | TTCAGATTTTGATATTGAGCTTCT | 4809 | TCTAGGGTCTCTCTGTCTCAGGGTCT |
| 3548 | GTCTCAAATTACCCCACAGTG | 4179 | TCAGATTTTGATATTGAGCTTCTC | 4810 | CTAGGGTCTCTCTGTCTCAGGGTCTG |
| 3549 | TCTCAAATTACCCCACAGTGG | 4180 | CAGATTTTGATATTGAGCTTCTCT | 4811 | TAGGGTCTCTCTGTCTCAGGGTCTGG |
| 3550 | CTCAAATTACCCCACAGTGGG | 4181 | AGATTTTGATATTGAGCTTCTCTC | 4812 | AGGGTCTCTCTGTCTCAGGGTCTGGG |
| 3551 | TCAAATTACCCCACAGTGGGG | 4182 | GATTTTGATATTGAGCTTCTCTCT | 4813 | GGGTCTCTCTGTCTCAGGGTCTGGGG |
| 3552 | CAAATTACCCCACAGTGGGGG | 4183 | ATTTTGATATTGAGCTTCTCTCTC | 4814 | GGTCTCTCTGTCTCAGGGTCTGGGGT |
| 3553 | AAATTACCCCACAGTGGGGGG | 4184 | TTTTGATATTGAGCTTCTCTCTCT | 4815 | GTCTCTCTGTCTCAGGGTCTGGGGTC |
| 3554 | AATTACCCCACAGTGGGGGGT | 4185 | TTTGATATTGAGCTTCTCTCTCTG | 4816 | TCTCTCTGTCTCAGGGTCTGGGGTCT |
| 3555 | ATTACCCCACAGTGGGGGGTG | 4186 | TTGATATTGAGCTTCTCTCTCTGG | 4817 | CTCTCTGTCTCAGGGTCTGGGGTCTG |
| 3556 | TTACCCCACAGTGGGGGGTGG | 4187 | TGATATTGAGCTTCTCTCTCTGGG | 4818 | TCTCTGTCTCAGGGTCTGGGGTCTGC |
| 3557 | TACCCCACAGTGGGGGGTGGT | 4188 | GATATTGAGCTTCTCTCTCTGGGG | 4819 | CTCTGTCTCAGGGTCTGGGGTCTGCA |
| 3558 | ACCCCACAGTGGGGGGTGGTG | 4189 | ATATTGAGCTTCTCTCTCTGGGGG | 4820 | TCTGTCTCAGGGTCTGGGGTCTGCAT |
| 3559 | CCCCACAGTGGGGGGTGGTGA | 4190 | TATTGAGCTTCTCTCTCTGGGGGA | 4821 | CTGTCTCAGGGTCTGGGGTCTGCATC |
| 3560 | CCCACAGTGGGGGGTGGTGAA | 4191 | ATTGAGCTTCTCTCTCTGGGGGAT | 4822 | TGTCTCAGGGTCTGGGGTCTGCATCT |
| 3561 | CCACAGTGGGGGGTGGTGAAG | 4192 | TTGAGCTTCTCTCTCTGGGGGATC | 4823 | GTCTCAGGGTCTGGGGTCTGCATCTT |
| 3562 | CACAGTGGGGGGTGGTGAAGT | 4193 | TGAGCTTCTCTCTCTGGGGGATCT | 4824 | TCTCAGGGTCTGGGGTCTGCATCTTT |
| 3563 | ACAGTGGGGGGTGGTGAAGTA | 4194 | GAGCTTCTCTCTCTGGGGGATCTG | 4825 | CTCAGGGTCTGGGGTCTGCATCTTTA |
| 3564 | CAGTGGGGGGTGGTGAAGTAT | 4195 | AGCTTCTCTCTCTGGGGGATCTGG | 4826 | TCAGGGTCTGGGGTCTGCATCTTTAG |
| 3565 | AGTGGGGGGTGGTGAAGTATC | 4196 | GCTTCTCTCTCTGGGGGATCTGGG | 4827 | CAGGGTCTGGGGTCTGCATCTTTAGG |
| 3566 | GTGGGGGGTGGTGAAGTATCT | 4197 | CTTCTCTCTCTGGGGGATCTGGGG | 4828 | AGGGTCTGGGGTCTGCATCTTTAGGA |
| 3567 | TGGGGGGTGGTGAAGTATCTT | 4198 | TTCTCTCTCTGGGGGATCTGGGGT | 4829 | GGGTCTGGGGTCTGCATCTTTAGGAC |
| 3568 | GGGGGGTGGTGAAGTATCTTT | 4199 | TCTCTCTCTGGGGGATCTGGGGTC | 4830 | GGTCTGGGGTCTGCATCTTTAGGACC |
| 3569 | GGGGGTGGTGAAGTATCTTTC | 4200 | CTCTCTCTGGGGGATCTGGGGTCG | 4831 | GTCTGGGGTCTGCATCTTTAGGACCT |
| 3570 | GGGGTGGTGAAGTATCTTTCT | 4201 | TCTCTCTGGGGGATCTGGGGTCGT | 4832 | TCTGGGGTCTGCATCTTTAGGACCTC |
| 3571 | GGGTGGTGAAGTATCTTTCTC | 4202 | CTCTCTGGGGGATCTGGGGTCGTT | 4833 | CTGGGGTCTGCATCTTTAGGACCTCT |
| 3572 | GGTGGTGAAGTATCTTTCTCT | 4203 | TCTCTGGGGGATCTGGGGTCGTTT | 4834 | TGGGGTCTGCATCTTTAGGACCTCTG |
| 3573 | GTGGTGAAGTATCTTTCTCTG | 4204 | CTCTGGGGGATCTGGGGTCGTTTT | 4835 | GGGGTCTGCATCTTTAGGACCTCTGT |
| 3574 | TGGTGAAGTATCTTTCTCTGT | 4205 | TCTGGGGGATCTGGGGTCGTTTTT | 4836 | GGGTCTGCATCTTTAGGACCTCTGTC |
| 3575 | GGTGAAGTATCTTTCTCTGTG | 4206 | CTGGGGGATCTGGGGTCGTTTTTT | 4837 | GGTCTGCATCTTTAGGACCTCTGTCT |
| 3576 | GTGAAGTATCTTTCTCTGTGG | 4207 | TGGGGGATCTGGGGTCGTTTTTTC | 4838 | GTCTGCATCTTTAGGACCTCTGTCTC |
| 3577 | TGAAGTATCTTTCTCTGTGGG | 4208 | GGGGGATCTGGGGTCGTTTTTTCC | 4839 | TCTGCATCTTTAGGACCTCTGTCTCT |
| 3578 | GAAGTATCTTTCTCTGTGGGT | 4209 | GGGGATCTGGGGTCGTTTTTTCCT | 4840 | CTGCATCTTTAGGACCTCTGTCTCTC |
| 3579 | AAGTATCTTTCTCTGTGGGTC | 4210 | GGGATCTGGGGTCGTTTTTTCCTC | 4841 | TGCATCTTTAGGACCTCTGTCTCTCT |
| 3580 | GTATCTTTCTCTGTGGGTCTA | 4211 | GGATCTGGGGTCGTTTTTTCCTCA | 4842 | GCATCTTTAGGACCTCTGTCTCTCTC |
| 3581 | TATCTTTCTCTGTGGGTCTAG | 4212 | GATCTGGGGTCGTTTTTTCCTCAA | 4843 | CATCTTTAGGACCTCTGTCTCTCTCT |
| 3582 | ATCTTTCTCTGTGGGTCTAGG | 4213 | ATCTGGGGTCGTTTTTTCCTCAAA | 4844 | ATCTTTAGGACCTCTGTCTCTCTCTG |
| 3583 | TCTTTCTCTGTGGGTCTAGGG | 4214 | TCTGGGGTCGTTTTTTCCTCAAAT | 4845 | TCTTTAGGACCTCTGTCTCTCTCTGG |
| 3584 | CTTTCTCTGTGGGTCTAGGGT | 4215 | CTGGGGTCGTTTTTTCCTCAAATC | 4846 | CTTTAGGACCTCTGTCTCTCTCTGGT |
| 3585 | TTTCTCTGTGGGTCTAGGGTC | 4216 | TGGGGTCGTTTTTTCCTCAAATCA | 4847 | TTTAGGACCTCTGTCTCTCTCTGGTG |
| 3586 | TTCTCTGTGGGTCTAGGGTCT | 4217 | GGGGTCGTTTTTTCCTCAAATCAG | 4848 | TTAGGACCTCTGTCTCTCTCTGGTGT |
| 3587 | TCTCTGTGGGTCTAGGGTCTC | 4218 | GGGTCGTTTTTTCCTCAAATCAGG | 4849 | TAGGACCTCTGTCTCTCTCTGGTGTG |
| 3588 | CTCTGTGGGTCTAGGGTCTCT | 4219 | GGTCGTTTTTTCCTCAAATCAGGA | 4850 | AGGACCTCTGTCTCTCTCTGGTGTGT |
| 3589 | TCTGTGGGTCTAGGGTCTCTC | 4220 | GTCGTTTTTTCCTCAAATCAGGAG | 4851 | GGACCTCTGTCTCTCTCTGGTGTGTT |
| 3590 | CTGTGGGTCTAGGGTCTCTCT | 4221 | TCGTTTTTTCCTCAAATCAGGAGT | 4852 | GACCTCTGTCTCTCTCTGGTGTGTTT |
| 3591 | TGTGGGTCTAGGGTCTCTCTG | 4222 | CGTTTTTTCCTCAAATCAGGAGTC | | |
| 3592 | GTGGGTCTAGGGTCTCTCTGT | 4223 | GTTTTTTCCTCAAATCAGGAGTCT | | |

In some embodiments, an ASO provided herein comprises 16, 17, 18, 19, 20, 21, 22, 23, 24, 26, or 26 of the nucleotide sequence of an ASO provided in Table 2-4. For example, an ASO can comprise the first (from 5′ to 3′) 16, 17, 18, 19, 20, 21, 22, 23, 24, 25, or 26 nucleotides of any one of SEQ ID NOs: 10-4852, e.g., nucleotides at positions 1 to any one of positions 16, 17, 18, 19, 21, 22, 23, 24, 25, or 26 of any one of SEQ ID NOs: 10-4852. Alternatively, an ASO can comprise the last 16, 17, 18, 19, 20, 21, 22, 23, 24, or 25, nucleotides of any one of SEQ ID NOs: 10-4852, e.g., nucleotides at positions 2, 3, 4, 5, 6, 7, 8, 9, or 10 to positions 16, 17, 18, 19, 20, 21, 22, 23, 24, 25, or 26 of any one of SEQ ID NOs: 10-4852. For example, an ASO provided herein comprises the nucleotide sequence of nucleotides at positions 1 to 16, 1 to 17, 1 to 18, 1 to 19, 1 to 20, 1 to 21, 1 to 22, 1 to 23, 1 to 24, 1 to 25, 1 to 26, 2 to 17, 2 to 18, 2 to 19, 2 to 20, 2 to 21, 2 to 22, 2 to 23, 2 to 24, 2 to 25, 2 to 26, 3 to 18, 3 to 19, 3 to 20, 3 to 21, 3 to 22, 3 to 23, 3 to 24, 3 to 25, 3 to 26, 4 to 19, 4 to 20, 4 to 21, 4 to 22, 4 to 23, 4 to 24, 4 to 25, 4 to 26, 5 to 20, 5 to 21, 5 to 22, 5 to 23, 5 to 24, 5 to 25, 5 to 26, 6 to 21, 6 to 22, 6 to 23, 6 to 24, 6 to 25, 6 to 26, 7 to 22, 7 to 23, 7 to 24, 7 to 25, 7 to 26, 8 to 23, 8 to 24, 8 to 25, 8 to 26, 9 to 24, 9 to 25, 9 to 26, 10 to 25, or 10 to 26 of any one of SEQ ID NOs: 10-4852. In some embodiments, an ASO provided herein comprises the nucleotide sequence of nucleotides at positions 1 to 16, 2 to 17, 3 to 18, 4 to 19, 5 to 20, 6 to 21, 7 to 23, 8 to 24, 9 to 25, or 10-26 of any one of SEQ ID NOs: 10-4852. In such embodiments, the ASO is at least 16, 17, 18, 19, or 20 nucleotides in length.

In some embodiments, the ASO comprises the nucleotide sequence of at least 8 contiguous nucleotides of chr6:33419695-33419939. In some embodiments, the ASO comprises the nucleotide sequence of at least 8 contiguous nucleotides of chr6:33453987-33454269. In some embodiments, the ASO comprises the nucleotide sequence of at least 8 contiguous nucleotides of chr6:33419674-33419940. In such embodiments, the at least 8 contiguous nucleotides of chromosome 6 (chr6) are the plus-strand nucleotides of chromosome 6 as compared to a reference genome.

In some aspects, an ASO provided herein comprises the nucleotide sequence of any one of SEQ ID NOs: 10-4852, plus up to four additional nucleotides at the 5′ end of said nucleotide sequence that are complementary to the target SYNGAP1 regRNA. For example, the ASO can comprise one, two, three, or four additional nucleotides at the 5′ end of any one of SEQ ID NOs: 10-4852 that are complementary to the human SYNGAP1 regRNA (e.g., any one of the regRNAs described in Table 1, including RR86_v1 (SEQ ID NO: 1), RR87 (SEQ ID NO: 2), RR88 (SEQ ID NO: 3), RR93_v1 (SEQ ID NO: 4), RR86_v2 (SEQ ID NO: 5), RR93_v1 (SEQ ID NO: 6) or the mouse SYNGAP1 regRNA (e.g., the regRNA described in Table 1 as RR121 (SEQ ID NO: 7)). In some embodiments, if the ASO includes up to four (e.g., 1, 2, 3, or 4) additional nucleotides at the 5′ end of the nucleotide sequence any one of SEQ ID NOs: 10-4852 that are complementary to the target SYNGAP1 regRNA, the ASO also can exclude up to four (e.g., 1, 2, 3 or 4) nucleotides from the 3′ end of the nucleotide sequence of any one of SEQ ID NOs: 10-4852. For example, the ASO can exclude one, two, three, or four 3′ end nucleotides from the nucleotide sequence of any one of SEQ ID NOs: 10-4852 if it includes one, two, three or four 5′ nucleotides that are complementary to the target SYNGAP1 regRNA.

In some aspects, an ASO provided herein comprises the nucleotide sequence of any one of SEQ ID NOs: 10-4852, plus up to four additional nucleotides at the 3′ end of said nucleotide sequence that are complementary to the target SYNGAP1 regRNA. For example, the ASO can comprise one, two, three, or four additional nucleotides at the 3′ end of any one of SEQ ID NOs: 10-4852 that are complementary to the target SYNGAP1 regRNA (e.g., any one of the regRNAs described in Table 1, including RR86_v1 (SEQ ID NO: 1), RR87 (SEQ ID NO: 2), RR88 (SEQ ID NO: 3), RR93_v1 (SEQ ID NO: 4), RR86_v2 (SEQ ID NO:5) or RR93_v1 (SEQ ID NO: 6). In some embodiments, if the ASO includes up to four (e.g., 1, 2, 3, or 4) additional nucleotides at the 3′ end of the nucleotide sequence of any one of SEQ ID NOs: 10-4852 that are complementary to the target SYNGAP1 regRNA, the ASO also can exclude up to four (e.g., 1, 2, 3, or 4) nucleotides from the 5′ end of the nucleotide sequence any one of SEQ ID NOs: 10-4852. For example, the ASO can exclude one, two, three, or four 5′ end nucleotides from the nucleotide sequence of any one of SEQ ID NOs: 10-4852 if it includes one, two, three or four 3′ nucleotides that are complementary to the target SYNGAP1 regRNA.

In some embodiments, the regulatory RNA has a nucleotide sequence of SEQ ID NO: 1, and the ASO comprises a nucleotide sequence selected from the group consisting of SEQ ID NOs: 10-59, 220-250, 261-267, 272-278, 526-528, 542-591, 702-728, 729-735-741, 988-990, and 1004-2961.

In some embodiments, the regulatory RNA has a nucleotide sequence of SEQ ID NO: 1, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961. In some embodiments, the regulatory RNA has a nucleotide sequence comprising nucleotides 185-467 of SEQ ID NO: 1, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961. In some embodiments, the regulatory RNA does not comprise or consist of a nucleotide sequence comprising nucleotides 1-184 of SEQ ID NO: 1, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961.

In some embodiments, the regulatory RNA has a nucleotide sequence of SEQ ID NO: 5, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961.

In some embodiments, the regulatory RNA has a nucleotide sequence of SEQ ID NO: 2, and the ASO comprises a nucleotide sequence selected from the group consisting of SEQ ID NOs: 60-73.

In some embodiments, the regulatory RNA has a nucleotide sequence of SEQ ID NO: 3, and the ASO comprises a nucleotide sequence selected from the group consisting of SEQ ID NOs: 74-109, 251-260, 268-271, and 279.

In some embodiments, the regulatory RNA has a nucleotide sequence of SEQ ID NO: 4 or 6, and the ASO comprises a nucleotide sequence selected from the group consisting of SEQ ID NOs: 110-219, 280-525, 529-541, 592-701, 742-987, and 991-1003.

In some embodiments, the regulatory RNA has a nucleotide sequence of SEQ ID NO: 7, and the ASO comprises a nucleotide sequence selected from the group consisting of SEQ ID NOs: 430-444 and 892-905.

### Hybridization and ΔG

The term “hybridizing” or “hybridizes” as used herein is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ, is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the free energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem, Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Aced Sci USA. 95: 1460-1465 using appropriately derived thermodynamic parameters described by Sugimoto et al., 1995, Biochemistry 34:11211-11216 and McTigue et al., 2004, Biochemistry 43:5388-5405. To have the possibility of modulating its intended nucleic acid target by hybridization, oligonucleotides of the present disclosure hybridize to a target nucleic acid with estimated ΔG° values below −10 kcal/mol for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal/mol, such as below −15 kcal/mol, such as below −20 kcal/mol and such as below −25 kcal/mol for oligonucleotides that are 8-30 nucleotides in length. In some embodiments the oligonucleotides hybridize to a target nucleic acid with an estimated ΔG° value of −10 to −60 kcal/mol, such as −12 to −40 kcal/mol, −15 to −30 kcal/mol, −16 to −27 kcal/mol, or −18 to −25 kcal/mol.

### Duplex Region

The phrase “duplex region” refers to the region in two complementary or substantially complementary polynucleotides that form base pairs with one another, either by Watson-Crick base pairing or any other manner that allows for a stabilized duplex between polynucleotide strands that are complementary or substantially complementary. For example, a polynucleotide strand having 21 nucleotide units can base pair with another polynucleotide of 21 nucleotide units, yet only 19 bases on each strand are complementary or substantially complementary, such that the “duplex region” has 19 base pairs. The remaining bases may, for example, exist as 5′ and 3′ overhangs. Further, within the duplex region, 100% complementarity is not required; substantial complementarity is allowable within a duplex region. Substantial complementarity refers to 70% or greater complementarity. For example, a mismatch in a duplex region consisting of 19 base pairs results in 94.7% complementarity, rendering the duplex region substantially complementary. Duplex regions can be formed by two separate oligonucleotide strands, as well as by single oligonucleotide strands that can form hairpin structures comprising a duplex region.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of a SYNGAP1 regRNA, such as an eRNA or paRNA. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides. Generally, the duplex structure is between 5 and 50 base pairs in length, e.g., between 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-1315-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 base pairs in length. Ranges and lengths intermediate to the above recited ranges and lengths are also contemplated to be part of the disclosure.

Similarly, the region of complementarity to the target sequence can be between 5 and 50 nucleotides in length, e.g., between 5-50, 5-49, 5-48, 5-47, 5-46, 5-45, 5-44, 5-43, 5-42, 5-41, 5-40, 5-39, 5-38, 5-37, 5-36, 5-35, 5-34, 5-33, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 6-50, 6-49, 6-48, 6-47, 6-46, 6-45, 6-44, 6-43, 6-42, 6-41, 6-40, 6-39, 6-38, 6-37, 6-36, 6-35, 6-34, 6-33, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 6-9, 6-8, 6-7, 8-50, 8-49, 8-48, 8-47, 8-46, 8-45, 8-44, 8-43, 8-42, 8-41, 8-40, 8-39, 8-38, 8-37, 8-36, 8-35, 8-34, 8-33, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 8-11, 8-10, 8-9, 10-50, 10-49, 10-48, 10-47, 10-46, 10-45, 10-44, 10-43, 10-42, 10-41, 10-40, 10-39, 10-38, 10-37, 10-36, 10-35, 10-34, 10-33, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 10-13, 10-12, 10-11, 10-10, 10-9, 12-50, 12-49, 12-48, 12-47, 12-46, 12-45, 12-44, 12-43, 12-42, 12-41, 12-40, 12-39, 12-38, 12-37, 12-36, 12-35, 12-34, 12-33, 12-32, 12-31, 12-30, 12-29, 12-28, 12-27, 12-26, 12-25, 12-24, 12-23, 12-22, 12-21, 12-20, 12-19, 12-18, 12-17, 12-16, 12-15, 12-14, 12-13, 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-30, 18-30, 18-29, 18-28, 18-27, 18-26, 18-25, 18-24, 18-23, 18-22, 18-21, 18-20, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-30, 19-30, 19-29, 19-28, 19-27, 19-26, 19-25, 19-24, 19-23, 19-22, 19-21, 19-20, 20-50, 20-49, 20-48, 20-47, 20-46, 20-45, 20-44, 20-43, 20-42, 20-41, 20-40, 20-39, 20-38, 20-37, 20-36, 20-35, 20-34, 20-33, 20-32, 20-31, 20-30, 20-30, 20-29, 20-28, 20-27, 20-26, 20-25, 20-24, 20-23, 20-22, 20-21, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-30, 21-29, 21-28, 21-27, 21-26, 21-25, 21-24, 21-23, 21-22, 22-50, 22-49, 22-48, 22-47, 22-46, 22-45, 22-44, 22-43, 22-42, 22-41, 22-40, 22-39, 22-38, 22-37, 22-36, 22-35, 22-34, 22-33, 22-32, 22-31, 22-30, 22-29, 22-28, 22-27, 22-26, 22-25, 22-24, 22-23, 23-50, 23-49, 23-48, 23-47, 23-46, 23-45, 23-44, 23-43, 23-42, 23-41, 23-40, 23-39, 23-38, 23-37, 23-36, 23-35, 23-34, 23-33, 23-32, 23-31, 23-30, 23-29, 23-28, 23-27, 23-26, 23-25, or 23-24 nucleotides in length. Ranges and lengths intermediate to the above recited ranges and lengths are also contemplated to be part of the disclosure.

### Chemical Modifications of ASOs

In certain embodiments, the ASO does not consist of only DNA. In certain embodiments, the ASO comprises at least one chemical modification relative to a natural nucleotide (e.g., ribonucleotide, e.g., 2′-deoxy-2′-ribonucleotide). Various chemical modifications can be included in the ASOs of the present disclosure. The modifications can include one or more modifications in a sugar group (e.g., ribose) group, one or more modifications in a phosphate group, one or more modifications in a nucleobase, one or more terminal modifications, or a combination thereof. In some embodiments, an exemplary ASO comprising or consisting of a nucleotide sequence targeting a regRNA as shown in any one of Tables 2-4 is chemically modified. Such modifications can be, but are not limited to, 2′-O-(2-methoxyethyl) (2′-MOE), locked nucleic acid (LNA), 5-methyl on the cytidine, constrained ethyl (cET), phosphorothioate (PS) linkage, and/or a phosphodiester (PO) linkage, or any combination thereof. Chemical modifications of RNA are known in the art and described in, for example, PCT Application Publication No. WO2013/177248, incorporated herein by reference. In certain embodiments, each cytidine in an ASO provided herein is modified by 5-methyl.

Various chemical modifications for use with ASOs of the present disclosure include, but are not limited to: 3′-terminal deoxy-thymine (dT) nucleotides, 2′-O-methyl modified nucleotides, 2′-fluoro modified nucleotides, 2′-deoxy-modified nucleotides, locked nucleotides, unlocked nucleotides, conformationally restricted nucleotides, constrained ethyl nucleotides, abasic nucleotides, 2′-amino-modified nucleotides, 2′-O-allyl-modified nucleotides, 2′-C-alkyl-modified nucleotides, 2′-hydroxyl-modified nucleotides, 2′-methoxyethyl modified nucleotides, 2′-O-alkyl-modified nucleotides, morpholino nucleotides, phosphoramidates, non-natural base comprising nucleotides, tetrahydropyran modified nucleotides, 1,5-anhydrohexitol modified nucleotides, cyclohexenyl modified nucleotides, nucleotides comprising a phosphorothioate group, nucleotides comprising a methylphosphonate group, nucleotides comprising a 5′-phosphate, and nucleotides comprising a 5′-phosphate mimic.

In certain embodiments, the ASO comprises an RNA polynucleotide chemically modified to be resistant to one or more nucleases (e.g., nuclear RNases (e.g., the exosome complex or RNaseH)). In some embodiments, all nucleotide bases are modified in the ASO. In certain embodiments, the chemical modifications comprises β-D-ribonucleotides, 2-modified nucleotides (e.g., 2′-O-(2-Methoxyethyl) (2′-MOE), 2′-O—CH₃, or 2′-fluoro-arabino (FANA)), bicyclic sugar modified nucleotides (e.g., having a constrained ethyl or locked nucleic acid (LNA)), and/or one or more modified internucleotide bonds (e.g., phosphorothioate internucleotide linkage). In certain embodiments, the chemical modification comprises 2′-MOE and a phosphorothioate internucleotide bond. In certain embodiments, at least 8, 9, 10, 11, 12, 13, 14, 15, 16, 17, 18, 19, 20, or more consecutive nucleotides of the ASO are modified by 2′-MOE. In certain embodiments, each nucleotide of the ASO is modified by 2′-MOE. In certain embodiments, at least 7, 8, 9, 10, 11, 12, 13, 14, 15, 16, 17, 18, 19, 20, or more consecutive internucleotide bonds of the ASO are phosphorothioate internucleotide bonds. In certain embodiments, each internucleotide bond of the ASO is a phosphorothioate internucleotide bond.

Internucleotide linkage modifications that can be used with the ASOs of the present disclosure include, but are not limited to, phosphorothioate “PS” (P(S)), phosphoramidate (P(NR₁R₂) such as dimethylaminophosphoramidate(P(N(CH₃)₂)), phosphonocarboxylate (P(CH₂)ₙCOOR) such as phosphonoacetate “PACE” (P(CH₂COO⁻)), thiophosphonocarboxylate ((S)P(CH₂)nCOOR) such as thiophosphonoacetate “thioPACE” ((S)P(CH₂COO⁻)), alkylphosphonate (P(C₁₋₃alkyl) such as methylphosphonate —P(CH₃), boranophosphonate (P(BH₃)), and phosphorodithioate (P(S)₂).

In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PO bonds. In some embodiments, all internucleotide bonds of an ASO provided herein are PO internucleotide bonds. In some embodiments, an ASO provided herein does not comprise PO internucleotide bonds. In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS internucleotide bonds. In some embodiments, all internucleotide bonds of an ASO provided herein are PS bonds. In some embodiments, an ASO provided herein does not comprise PS internucleotide bonds.

In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS bonds. In some embodiments, all internucleotide bonds of an ASO provided herein are PS internucleotide bonds. In some embodiments, an ASO provided herein does not comprise PS internucleotide bonds. In some embodiments, an ASO provided herein comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more PS internucleotide bonds. In some embodiments, all internucleotide bonds of an ASO provided herein are PO bonds. In some embodiments, an ASO provided herein does not comprise PO internucleotide bonds.

In certain embodiments, the ASO comprises one or more chemical modifications at the 5′ end, the 3′ end, or both. Without wishing to be bound by theory, chemical modifications at one or both termini of a polynucleotide (e.g., polyribonucleotide) may stabilize the polynucleotide. In certain embodiments, the ASO comprises one or more chemical modifications in at least 1, 2, 3, 4, or 5 nucleotides at the 5′ end of the ASO. In certain embodiments, the ASO comprises one or more chemical modifications in at least 1, 2, 3, 4, or 5 nucleotides at the 3′ end of the ASO. In certain embodiments, the ASO comprises one or more chemical modifications in at least 1, 2, 3, 4, or 5 nucleotides at the 5′ end of the ASO and one or more chemical modifications in at least 1, 2, 3, 4, or 5 nucleotides at the 3′ end of the ASO.

The chemical structures can also be described in writing. In such cases, ‘M’ indicates MOE; ‘d’ indicates DNA, ‘L’ indicates LNA, “m” indicates 2′ OMethyl, ‘═’ indicates a phosphorothioate (PS) linkage, ‘—’ indicates a phosphodiester (PO) linkage; ‘*’ or ‘5C’ indicates 5-MethylCytosine, ‘ag’ indicates GalNAc, ‘tg’ or ‘teg’ indicates Teg-GalNAc, and ‘\hat{ }’ indicates FANA, “BioTeg” indicates Biotin; “Palm” indicates Palmitic acid; and “C18” indicates a Spacer 18 moiety.

To avoid ambiguity, this LNA has the formula:

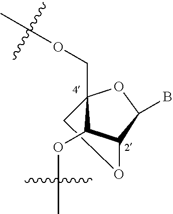

wherein B is the particular designated base.

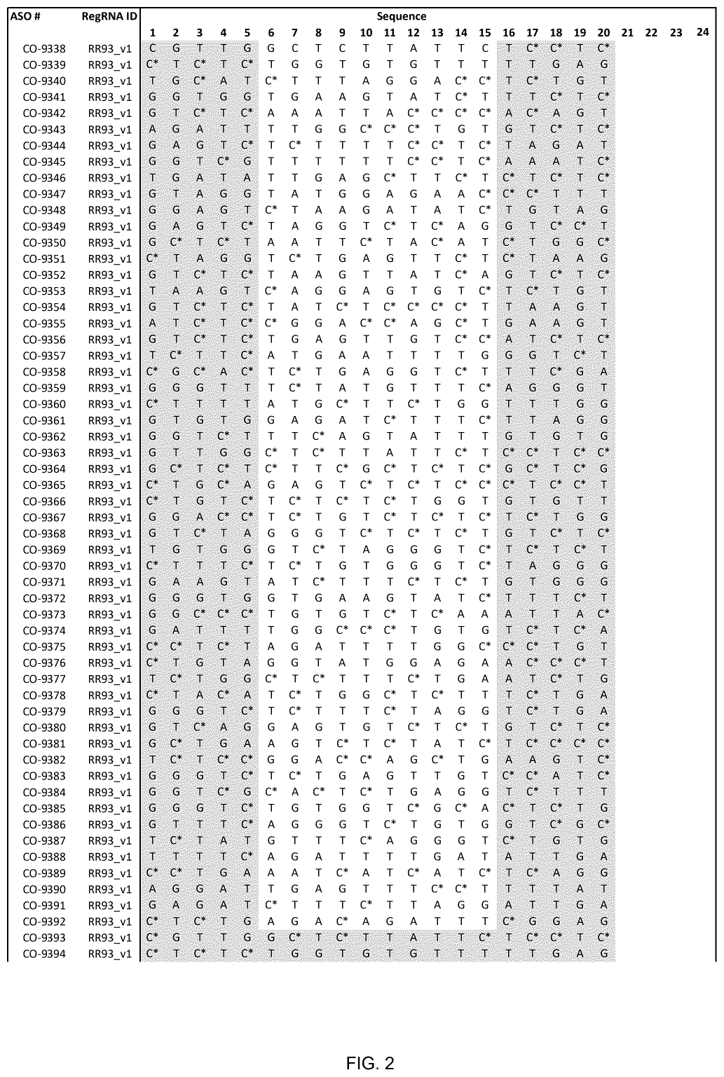

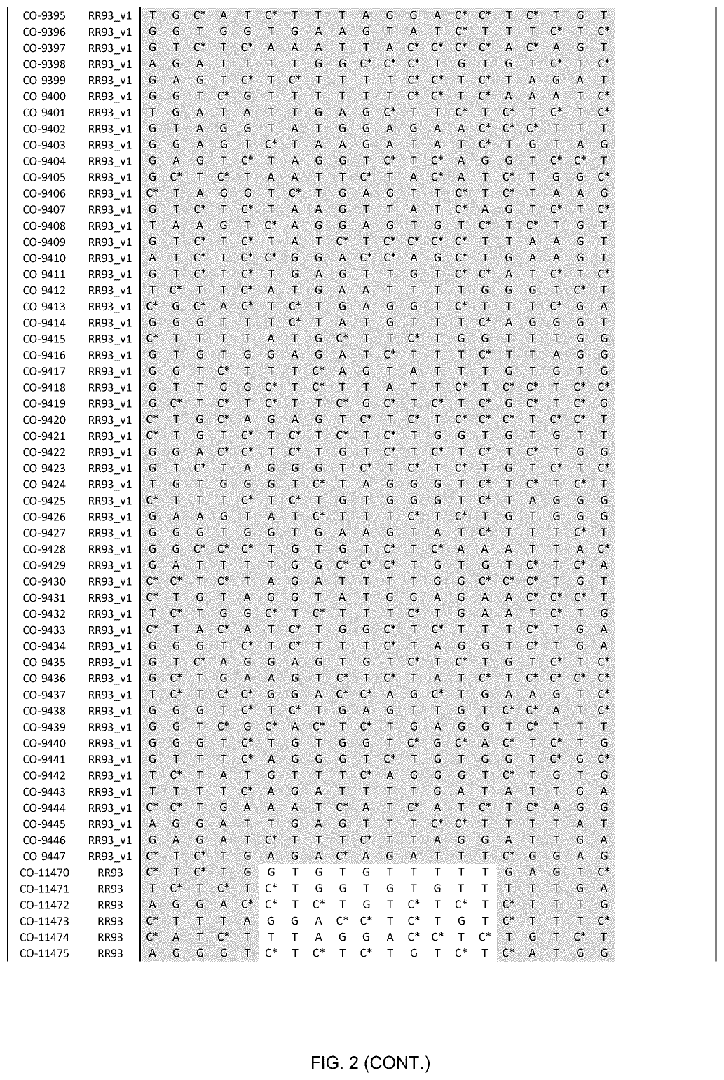

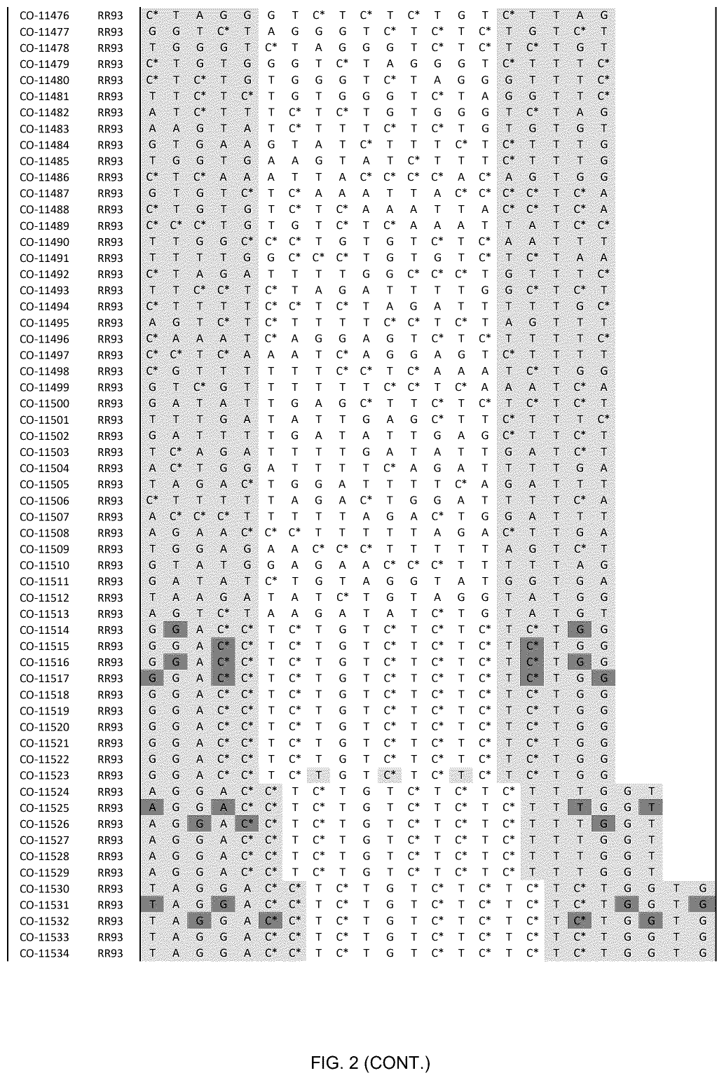

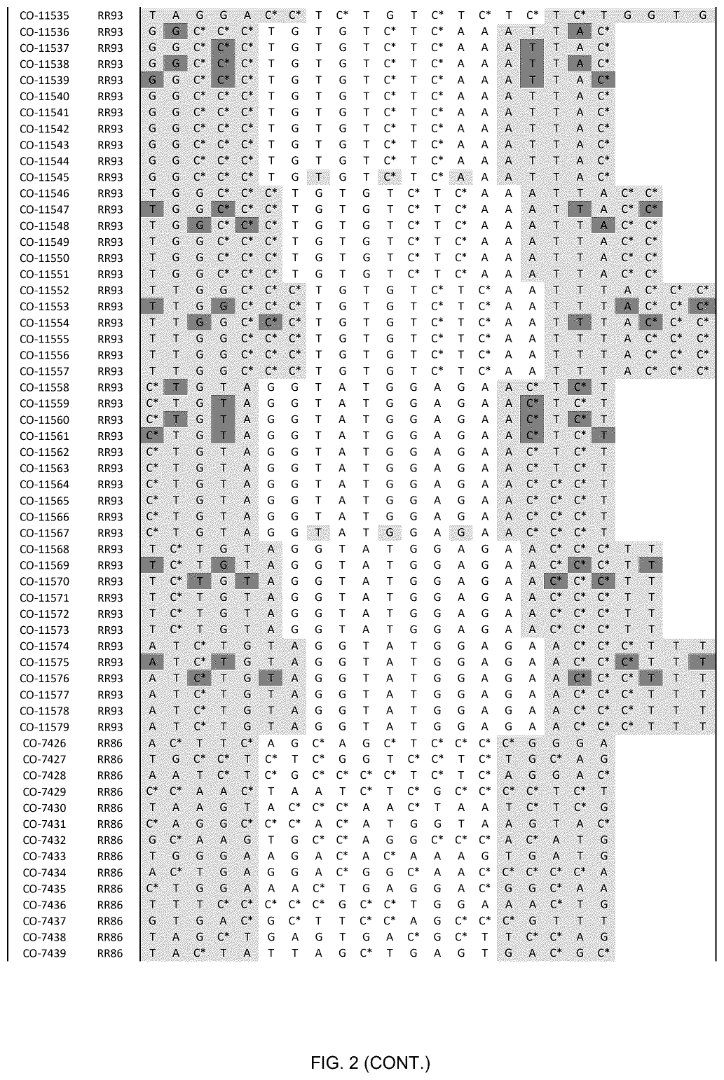

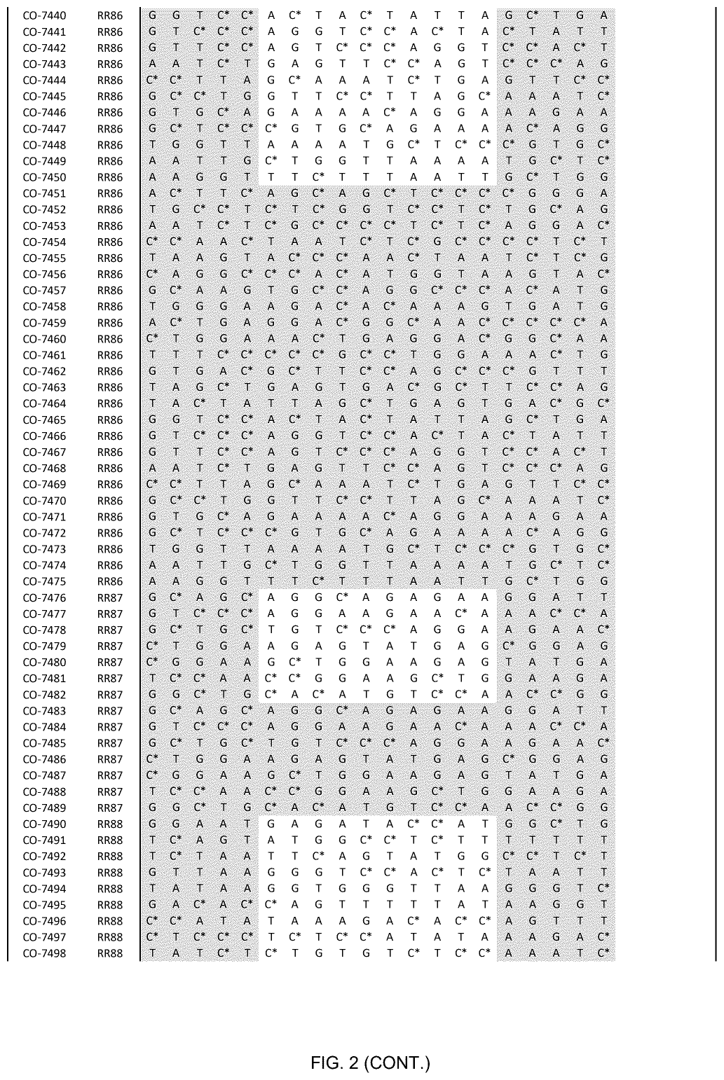

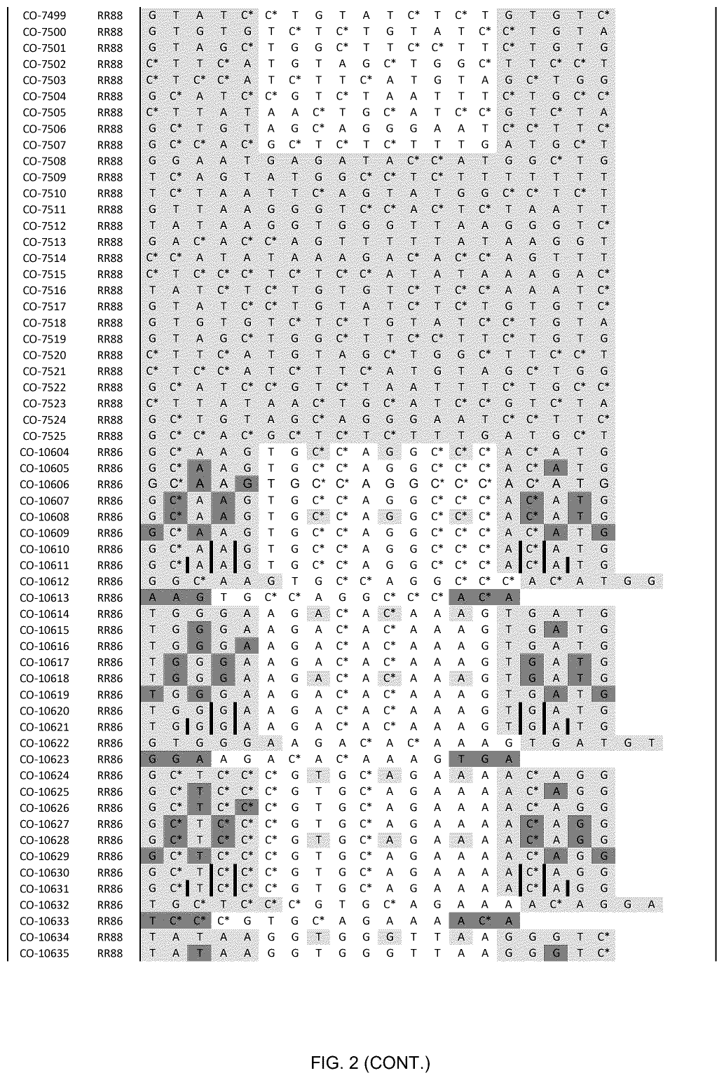

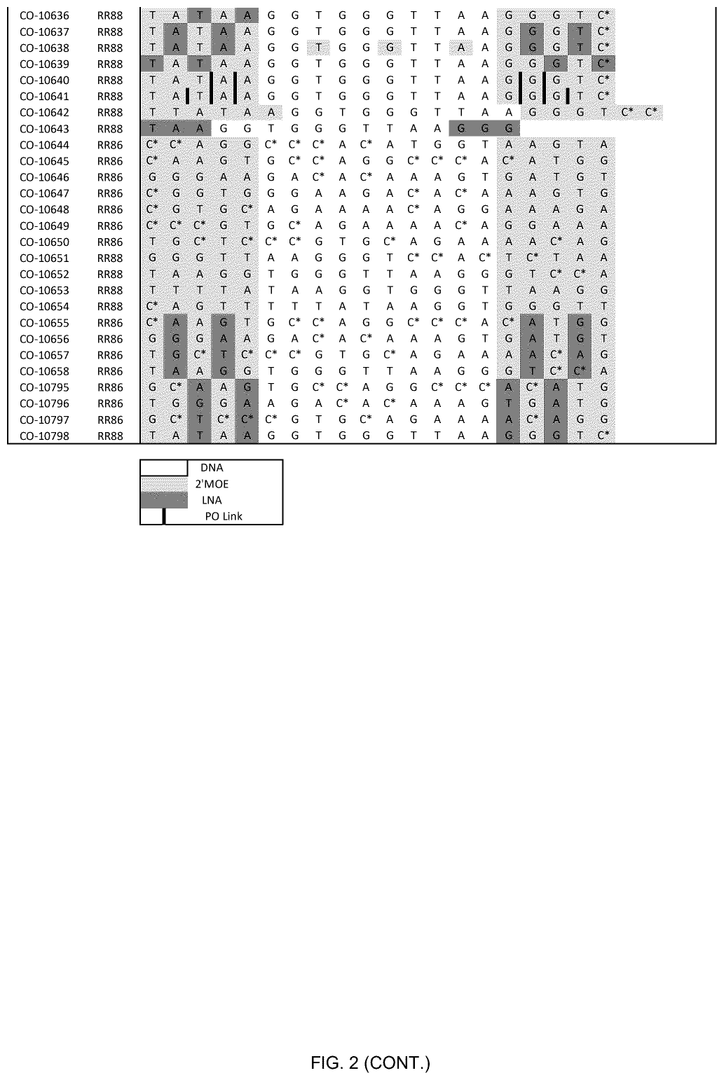

Exemplary visual representation of ASOs with chemical modifications are provided in FIG. 2. Additional exemplary ASOs with chemical modifications are provided in Table 2. In some embodiments, an ASO provided herein comprises a nucleotide sequence and/or a chemical modification any one of the ASOs provided in Tables 2-4.

In some embodiments, an ASO comprises a sequence selected from the group consisting of SEQ ID NOs: 10-4852. In some embodiments, an ASO comprises a sequence and chemical modification selected from the group consisting of SEQ ID NOs: 542-1003.

In some embodiments, an ASO provided herein comprises a nucleotide sequence of any one of the ASOs provided in Tables 2-4. In some embodiments, the ASO comprises a sequence and/or chemical modification selected from the group consisting of SEQ ID NOs: 10-4852. In some embodiments, the ASO comprising a sequence selected from the group consisting of SEQ ID NOs: 10-541 or 1004-4852 further comprises any chemical modification as disclosed herein.

### High Affinity Modified Nucleotides

A high affinity modified nucleotide is a modified nucleotide which, when incorporated into the oligonucleotide enhances the affinity of the oligonucleotide for its complementary target, for example as measured by the melting temperature (Tm). A high affinity modified nucleotide of the present disclosure preferably result in an increase in melting temperature between +0.5 to +12° C., such as between +1.5 to +10° C. or +3 to +8° C. per modified nucleotide. Numerous high affinity modified nucleotides are known in the art and include for example, many 2′ substituted nucleotides as well as locked nucleic acids (LNA) (see e.g. Freier & Altmann (1997) Nucl. Acid Res., 25, 4429-4443 and Uhlmann (2000) Curr. Opinion in Drug Development, 3(2), 293-213), each of which are hereby incorporated by reference.

### Sugar Modifications

The ASOs described herein may comprise one or more nucleotides which have a modified sugar moiety, i.e. a modification of the sugar moiety when compared to the ribose sugar moiety found in DNA and RNA. Numerous nucleotides with modification of the ribose sugar moiety have been made, primarily with the aim of improving certain properties of oligonucleotides, such as affinity and/or nuclease resistance. Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleotides include, for example, bicyclohexose nucleic acids (WO2011/017521) or tricyclic nucleic acids (WO2013/154798), both of which are hereby incorporated by reference. Modified nucleotides also include nucleotides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in RNA nucleotides. Substituents may, for example be introduced at the 2′, 3′, 4′ or 5′ positions.

In some embodiments, oligonucleotides comprise modified sugar moieties, such as any one of a 2′-O-methyl (2′OMe) moeity, a 2′-O-methoxyethyl moeity, a bicyclic sugar moeity, PNA (e.g., an oligonucleotide comprising one or more N-(2-aminoethyl)-glycine units linked by amide bonds or carbonyl methylene linkage as repeating units in place of a sugar-phosphate backbone), locked nucleotide (LNA) (e.g., an oligonucleotide comprising one or more locked ribose, and can be a mixture of 2′-deoxy nucleotides or 2′OMe nucleotides), cET (e.g., an oligonucleotide comprising one or more cET sugars), cMOE (e.g., an oligonucleotide comprising one or more cMOE sugar), morpholino oligomer (e.g., an oligonucleotide comprising a backbone comprising one or more phosphorodiamidate morpholiono oligomers), 2′-deoxy-2′-fluoro nucleotide (e.g., an oligonucleotide comprising one or more 2′-fluoro-β-D-arabinonucleotide), tcDNA (e.g., an oligonucleotide comprising one or more tcDNA modified sugar), constrained ethyl 2′-4′-bridged nucleic acid (cEt), S-cEt, ethylene bridged nucleic acid (ENA) (e.g., an oligonucleotide comprising one or more ENA modified sugar), hexitol nucleic acids (HNA) (e.g., an oligonucleotide comprising one or more HNA modified sugar), or tricyclic analog (tcDNA) (e.g., an oligonucleotide comprising one or more tcDNA modified sugar).

In some embodiments, oligonucleotides comprise nucleobase modifications selected from the group consisting of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), and isocytosine (“isoC”), glycerol nucleic acid (GNA), thiomorpholino (C4H9NS) or thiophosphoramidate morpholinos (TMOs). Synthesis of glycerol nucleic acid (GNA) (also known as glycol nucleic acids) is described in Zhang et al, (2010) Current Protocols in Nucleic Acid Chemistry 4.40.1-4.40.18, hereby incorporated by reference. Synthesis of thiophosphoramidate Morpholino Oligonucleotides is described in Langer et al, J. Am. Chem. Soc. 2020, 142(38): 16240-53.

### 2′ Sugar Modified Nucleotides

A 2′ sugar modified nucleotide is a nucleotide which has a substituent other than H or —OH at the 2′ position (2′ substituted nucleotide) or comprises a 2′ linked biradical capable of forming a bridge between the 2′ carbon and a second carbon in the ribose ring, such as LNA (2′-4′ biradical bridged) nucleotides.

Without wishing to be bound by theory, the 2′ modified sugar may provide enhanced binding affinity and/or increased nuclease resistance to the oligonucleotide. Examples of 2′ substituted modified nucleotides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleotide. For further examples, see e.g. Freier & Altmann (1997) Nucl. Acid Res., 25, 4429-4443 and Uhlmann (2000) Curr. Opinion in Drug Development, 3(2), 293-213, and Deleavey and Damha, Chemistry and Biology 2012, 19, 937, each of which are hereby incorporated by reference.

### Locked Nucleic Acid Nucleotides (LNA Nucleotide)

A “LNA nucleotide” is a 2′-sugar modified nucleotide which comprises a biradical linking the C2′ and C4′ of the ribose sugar ring of said nucleotide (also referred to as a “2′-4′ bridge”), which restricts or locks the conformation of the ribose ring. In other words, a locked nucleotide is a nucleotide comprising a bicyclic sugar moiety comprising a 4′-CH₂—O-2′ bridge. This structure effectively “locks” the ribose in the 3′-endo structural conformation. The addition of locked nucleotides to oligonucleotides has been shown to increase oligonucleotide stability in serum, and to reduce off-target effects (Grunweller, A. et al., (2003) Nucleic Acids Research 31(12):3185-3193). These nucleotides are also sometimes termed bridged nucleic acid or bicyclic nucleic acid (BNA). The locking of the conformation of the ribose is associated with an enhanced affinity of hybridization (duplex stabilization) when the LNA is incorporated into an oligonucleotide with complementarity to an RNA or DNA molecule. This can be routinely determined by measuring the melting temperature of the oligonucleotide/complement duplex. Exemplary LNA nucleotides include beta-D-oxy-LNA, 6′-methyl-beta-D-oxy LNA such as (S)-6′-methyl-beta-D-oxy-LNA (ScET) and ENA.

Examples of bicyclic nucleotides for use in the polynucleotides of the disclosure include without limitation nucleotides comprising a bridge between the 4′ and the 2′ ribosyl ring atoms. In certain embodiments, the polynucleotide agents of the disclosure include one or more bicyclic nucleotides comprising a 4′ to 2′ bridge. Examples of such 4′ to 2′ bridged bicyclic nucleotides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof, see, e.g., U.S. Pat. No. 7,399,845); 4′-C(CH₃)(CH₃)—O-2′ (and analogs thereof, see e.g., U.S. Pat. No. 8,278,283); 4′-CH₂—N(OCH3)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof, see, e.g., U.S. Pat. No. 8,278,426). The entire contents of each of the foregoing are hereby incorporated herein by reference.

Additional representative U.S. Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleotides can be prepared having one or more stereochemical sugar configurations including for example α-L-ribofuranose and 3-D-ribofuranose (see International Publication No. WO 99/14226, contents of which are incorporated by reference herein).

An oligonucleotide of the disclosure can also be modified to include one or more constrained ethyl nucleotides. As used herein, a “constrained ethyl nucleotide” or “cEt” is a locked nucleotide comprising a bicyclic sugar moiety comprising a 4′-CH(CH₃)—O-2′ bridge. In one embodiment, a constrained ethyl nucleotide is in the S conformation referred to herein as “S-cEt.”

An oligonucleotide of the disclosure may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to an RNA (e.g., a regRNA or a mRNA). The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide of the disclosure comprises one or more monomers that are UNA (unlocked nucleotide) nucleotides. UNA is unlocked acyclic nucleotide, wherein any of the bonds of the sugar has been removed, forming an unlocked “sugar” residue. In one example, UNA also encompasses monomer with bonds between C1′—C4′ have been removed (i.e., the covalent carbon-oxygen-carbon bond between the C1′ and C4′ carbons). In another example, the C2′—C3′ bond (i.e., the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleotide (TNA), or arabinose to produce an arabino nucleotide. The ribose molecule can also be replaced with non-sugars such as cyclohexene to produce cyclohexene nucleotide or glycol to produce glycol nucleotides.

Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the disclosure include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Additional non-limiting, exemplary LNA nucleotides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, Mitsuoka et al., Nucleic Acids Research 2009, 37(4), 1225-1238, and Wan and Seth, J. Medical Chemistry 2016, 59, 9645-9667, each of which are hereby incorporated by reference.

In some embodiments, the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

In some embodiments, the nucleotide sugar modification is 2′-O C1-4alkyl such as 2′-O-methyl (2′-OMe), 2′-deoxy (2′-H), 2′-OC1-3alkyl-O-C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), or 4′-thioribosyl nucleotide.

### Mixmers and Gapmers

The ASO can have a mixmer and/or gapmer structure, for example, in a pattern disclosed by the ASOs in FIG. 2.

In certain embodiments, the ASO is a mixmer. As used herein, the term “mixmer” refers to an oligonucleotide comprising an alternating composition of DNA monomers and nucleotide analogue monomers across at least a portion of the oligonucleotide sequence. In certain embodiments, the ASO is a mixmer based on the gapmer structure, comprising a mixture of DNA nucleotides and 2′-MOE nucleotides in the gap, flanked by RNA sequences (e.g., 2′-modified RNA sequences) in the wings. Mixmers may be designed to comprise a mixture of affinity enhancing nucleotide analogues, such as in non-limiting example 2′-O-alkyl-RNA monomers, 2′-amino-DNA monomers, 2′-fluoro-DNA monomers, LNA monomers, arabino nucleic acid (ANA) monomers, 2′-fluoro-ANA monomers, HNA monomers, INA monomers, 2′-MOE-RNA (2′-O-methoxyethyl-RNA), 2′Fluoro-DNA, and LNA. In some embodiments, the mixmer is incapable of recruiting RNase H. In some embodiments, the mixmer comprises one type of affinity enhancing nucleotide analogue together with DNA and/or RNA.

Multiple different modifications can be interspaced in a mixmer. For example, the ASO can comprise LNA modification in a plurality of nucleotides and a different modification in some or all of the rest of the nucleotides. In some embodiments, any two adjacent LNA-modified nucleotides are separated by at least 1, 2, 3, 4, or 5 nucleotides. Throughout the ASO, the distance between adjacent LNA-modified nucleotides can either be constant (e.g., any two adjacent LNA-modified nucleotides are separated by 1, 2, 3, 4, or 5 nucleotides) or variable. In some embodiments, the length of the ASO is 3×n, 3×n−1, or 3×n−2 nucleotides (n is an integer of 6 or greater), wherein (a) (i) the nucleotides at positions 3×m−2 (m is an integer from 1 to n) are nucleotides (e.g., ribonucleotides or deoxyribonucleotides) comprising a first modification (e.g., LNA), (ii) the nucleotides at positions 3×m−1 (m is an integer from 1 to n) are nucleotides (e.g., ribonucleotides or deoxyribonucleotides) comprising a first modification (e.g., LNA), or (iii) the nucleotides at positions 3×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). In some embodiments, the length of the ASO is 2×n or 2×n−1 nucleotides (n is an integer of 9 or greater), wherein (a) (i) the nucleotides at positions 2×m−1 (m is an integer from 1 to n) are nucleotides (e.g., ribonucleotides or deoxyribonucleotides) comprising a first modification (e.g., LNA), or (ii) the nucleotides at positions 2×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). Similar modification patterns, for example, where the first modification is repeated very 4, 5, or more nucleotides, are also contemplated. In some embodiments, the length of the ASO is 4×n, 4×n−1, or 4×n−2 nucleotides (n is an integer of 6 or greater), wherein (a) (i) the nucleotides at positions 4×m−2 (m is an integer from 1 to n) are nucleotides (e.g., ribonucleotides or deoxyribonucleotides) comprising a first modification (e.g., LNA), (ii) the nucleotides at positions 4×m−1 (m is an integer from 1 to n) are nucleotides (e.g., ribonucleotides or deoxyribonucleotides) comprising a first modification (e.g., LNA), or (iii) the nucleotides at positions 3×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl). In some embodiments, the length of the ASO is 5×n, 5×n−1, or 5×n−2 nucleotides (n is an integer of 6 or greater), wherein (a) (i) the nucleotides at positions 5×m−2 (m is an integer from 1 to n) are nucleotides (e.g., ribonucleotides or deoxyribonucleotides) comprising a first modification (e.g., LNA), (ii) the nucleotides at positions 5×m−1 (m is an integer from 1 to n) are nucleotides (e.g., ribonucleotides or deoxyribonucleotides) comprising a first modification (e.g., LNA), or (iii) the nucleotides at positions 5×m (m is an integer from 1 to n) are nucleotides (e.g., ribonucleotides or deoxyribonucleotides) comprising a first modification (e.g., LNA); and (b) the nucleotides at the remaining positions comprise a second, different modification (e.g., 2′-O-methoxyethyl).

In some embodiments, the ASO further comprises a GalNAc or Teg-GalNAc moiety at the 5′ or 3′ end of the ASO.

In certain embodiments, the ASO comprises a DNA sequence (e.g., having at least 1, 2, 3, 4, 5, 6, 7, 8, 9, 10, 11, 12, 13, 14, or 15 contiguous nucleotides of unmodified DNA) flanked on both sides by RNA sequences. Such structure is known as “gapmer,” in which the DNA region is referred to as the “gap” and the RNA regions is referred to as the “wings” (see, e.g., PCT Application Publication No. WO2013/177248). Gapmers were known to facilitate degradation of the target RNA by recruiting nucleases (e.g., nuclear RNAses (e.g., RNase H)). Surprisingly, in some embodiments of the present disclosure, it has been discovered that a gapmer that binds to a regRNA having the same sequence as a parent ASO but having different chemical modifications, can also increase target gene expression. In certain embodiments, the ASO comprises a DNA sequence flanked by RNA sequences and does not induce RNAse- or RNAse H-mediated degradation.

In some embodiments, the ASO gapmer comprises an internal DNA region flanked by two external RNA “wings.” For example, the internal DNA gap can comprise at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 nucleotide(s), while each of the external RNA wing(s) can independently comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Exemplary gapmer structures include, but are not limited to a 1-10-9, 2-10-8, 3-10-7, 4-10-6, 6-10-4, 7-10-3, 8-10-2, 9-10-1, 1-18-1, 2-16-2, 3-14-3, 4-12-4, 5-10-5, 6-8-6, 7-6-7, 8-5-7, 7-5-8, 8-4-8, or 9-2-9 structure where the first and third number indicate the number of external RNA nucleotides and the second number indicates the number of internal DNA nucleotides.

The ASO can also be a mixmer comprising one DNA region linked to one RNA region. In some embodiments, the mixmer comprises at least 10 DNA nucleotides linked to at least 10 RNA nucleotides, wherein the DNA nucleotides are at the 5′ end of the mixmer or the 3′ end of the mixmer. In some embodiments, the mixmer comprises at least 1, 2, 3, 4, 5, 6, 7, 8, 9, 10, 11, 12, 13, 14, 15, 16, 17, 18, or 19 DNA nucleotide(s) linked to at least 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 RNA nucleotide(s), wherein the DNA nucleotides are at the 5′ end of the mixmer or the 3′ end of the mixmer. In some embodiment, the RNA regions of the gapmer or mixmer can comprise any additional chemical modification as disclosed herein.

In certain embodiments, the ASO (e.g., the gapmer or mixmer) is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides in length. In certain embodiments, the gap is about 1, 2, 3, 4, 5, 6, 7, 8, 9, 10, 11, 12, 13, 14, 15, 16, 17, 18, 19, or more nucleotides in length. In certain embodiments, one or both wings are about 2, 3, 4, 5, 6, 7, 8, 9, 10, 11, 12, 13, 14, 15, 16, 17, 18, or 19 or more nucleotides in length. In certain embodiments, one or both wings comprises RNA modifications, for example, 3-D-ribonucleotides, 2′-modified nucleotides (e.g., 2′-O-(2-Methoxyethyl) (2′-MOE), 2′-O—CH₃, or 2′-fluoro-arabino (FANA)), and bicyclic sugar modified nucleotides (e.g., having a constrained ethyl or locked nucleic acid (LNA)). In certain embodiments, each ribonucleotide in the mixmer or gapmer is modified by 2′-MOE. In certain embodiments, the mixmer or gapmer comprises one or more modified internucleotide bonds, e.g., phosphorothioate (PS) internucleotide linkage. In certain embodiments, each two adjacent nucleotides in the mixmer or gapmer are linked by a phosphorothioate internucleotide bond.

In certain embodiments, the ASO does not comprise 1 or more, 2 or more, 3 or more, 4 or more, 5 or more, 6 or more, 7 or more, 8 or more, 9 or more, 10 or more, 10 or more, 11 or more, 12 or more, 13 or more, 14 or more, 15 or more, 20 or more, 25 or more, 30 or more, 35 or more, 40 or more, or 45 or more contiguous nucleotides of unmodified DNA. In some embodiments, such a DNA sequence is disrupted by modified (e.g., 2′-MOE modified) ribonucleotides every 2, 3, 4, 5, or more nucleotides. In some embodiments, the ASO comprises only ribonucleotides and no deoxyribonucleotides.

The structural features of mixmer and gapmer can be combined. In certain embodiments, the ASO has a structure similar to that of a mixmer disclosed herein (e.g., one having interspaced modifications), except that the second modification in the gap is changed to a third modification (e.g., deoxyribonucleotide). In certain embodiments, the ASO has a structure similar to that of a gapmer disclosed herein, except that in the gap the nucleotides are modified in a mixmer pattern.

In certain embodiments, the ASO further comprises a ligand moiety, e.g., a ligand moiety that specifically targets a tissue or organ in a subject. For example, N-acetylgalactosamine (GalNAc) specifically targets liver. In certain embodiments, the ligand moiety comprises GalNAc. In certain embodiments, the ligand moiety comprises a three-cluster GalNAc moiety, commonly denoted GAlNAc3. Other types of GalNAc moieties are one-cluster, two cluster or four cluster GalNAc, denoted as GalNAc1, GalNAc2, or GalNAc4. In certain embodiments, the ligand moiety comprises GalNAc1, GalNAc2, GalNAc3, or GalNAc4.

In certain embodiments, the ligand moiety comprises biotin. In certain embodiments, the ligand moiety comprises palmitic acid. In certain embodiments, the ligand moiety comprises a Spacer 18 moiety (C18).

### III. Pharmaceutical Compositions

In certain embodiments, an ASOs disclosed herein can be present in pharmaceutical compositions. The pharmaceutical composition can be formulated for use in a variety of drug delivery systems. One or more pharmaceutically acceptable excipients or carriers can also be included in the composition for proper formulation. In some embodiments, the pharmaceutical acceptable carrier comprises sterile saline, sterile water, or phosphate buffered saline (PBS). Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

Exemplary carriers and pharmaceutical formulations suitable for delivering nucleic acids are described in Durymanov and Reineke (2018) Front. Pharmacol. 9:971; Barba et al. (2019) Pharmaceutics 11(8): 360; Ni et al. (2019) Life (Basel) 9(3): 59, each of which is incorporated herein by reference. It is understood that the presence of a ligand moiety conjugated to the ASO may circumvent the need for a carrier for delivery to a tissue or organ targeted by the ligand moiety.

The delivery of an oligonucleotide of the disclosure to a cell e.g., a cell within a subject, such as a human subject e.g., a subject in need thereof, such as a subject having or at risk of developing a SYNGAP1 related disorder can be achieved in a number of different ways. For example, delivery may be performed by contacting a cell with an oligonucleotide of the disclosure either in vitro or in vivo. In vivo delivery may also be performed directly by administering a composition comprising an oligonucleotide to a subject. These alternatives are discussed further below.

In general, any method of delivering a nucleic acid molecule (in vitro or in vivo) can be adapted for use with an oligonucleotide of the disclosure (see e.g., Akhtar S. and Julian R L., (1992) Trends Cell. Biol. 2(5):139-144 and WO 94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleotide linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the disclosure. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the disclosure are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

In some embodiments, the compounds described herein may be administered in combination with additional therapeutics (e.g., using a simultaneous or alternating regimen). Examples of additional therapeutics include an anti-epileptic agent such as quinidine and/or sodium channel blockers, an anti-convulsant, a cholinesterase inhibitor, a dopamine agonist, levodopa, a dopamine reuptake inhibitor (SSRI), a selective serotonin reuptake inhibitor (NRI), a norepinephrine-dopamine reuptake inhibitor (NDRI), a serotonin-norepinephrine-dopamine reuptake inhibitor (SNDRI). Additionally, the compounds described herein may be administered in combination with recommended lifestyle changes.

### Membranous Molecular Assembly Delivery Methods

Oligonucleotides of the disclosure can also be delivered using a variety of membranous molecular assembly delivery methods including polymeric, biodegradable microparticle, or microcapsule delivery devices known in the art. For example, a colloidal dispersion system may be used for targeted delivery of an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169). These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes may also be sterically stabilized liposomes, comprising one or more specialized lipids that result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotide (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Ind.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAMINE, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotide into the skin. In some implementations, liposomes are used for delivering oligonucleotide to epidermal cells and also to enhance the penetration of oligonucleotide into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising NOVASOME I (glyceryl dilaurate/cholesterol/polyoxyethylene-10-stearyl ether) and NOVASOME II (glyceryl distearate/cholesterol/polyoxyethylene-10-stearyl ether) were used to deliver a drug into the dermis of mouse skin. Such formulations with oligonucleotides are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present disclosure are described in PCT Publication Nos. WO 2009/088891, WO 2009/132131, and WO 2008/042973, which are hereby incorporated by reference in their entirety.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotides for use in the methods of the disclosure can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

### Lipid Nanoparticle-Based Delivery Methods

Oligonucleotides in the disclosure may be fully encapsulated in a lipid formulation, e.g., a lipid nanoparticle (LNP), or other nucleic acid-lipid particle. LNPs are useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

Non-limiting examples of cationic lipids include N,N-dioleyl-N,N-dimethylammonium chloride (DODAC), N,N-distearyl-N,N-dimethylammonium bromide (DDAB), N--(I-(2,3-dioleoyloxy)propyl)-N,N,N-trimethylammonium chloride (DOTAP), N--(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyetetrahydro- 3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can comprise, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 60 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (C₁₂), a PEG-dimyristyloxypropyl (C₁₄), a PEG-dipalmityloxypropyl (C₁₆), or a PEG-distearyloxypropyl (Cis). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

The ASO may also be delivered in a lipidoid. The synthesis of lipidoids has been extensively described and formulations containing these compounds are particularly suited for delivery of modified nucleic acid molecules or ASOs (see Mahon et al, Bioconjug Chem. 2010 21: 1448-1454; Schroeder et al, J Intern Med. 2010 267:9-21; Akinc et al, Nat Biotechnol. 2008 26:561-569; Love et al, Proc Natl Acad Sci USA. 2010 107: 1864-1869; Siegwart et al, Proc Natl Acad Sci USA. 2011 108: 12996-3001; all of which are incorporated herein in their entireties).

Lipid compositions for RNA delivery are disclosed in WO2012170930A1, WO2013149141A1, and WO2014152211A1, each of which are hereby incorporated by reference.

### IV. Therapeutic Applications

The present disclosure provides methods for treating or preventing diseases and disorders of the central nervous system (CNS) and peripheral nervous system (PNS) in a subject in need thereof, including SYNGAP1-related disorders (e.g., associated with SYNGAP1 mutations), such as SYNGAP1-related intellectual disability (ID), mental retardation, autosomal dominant 5 (MRDS), or SYNGAP1-related non-syndromic intellectual disability (NSID)), affective disorders (e.g., depression), schizophrenia, Alzheimer's disease, Parkinson's disease, Huntington's disease, an autism spectrum disorder (ASD) (e.g., Asperger's syndrome, autistic disorder, and Pervasive Developmental Disorder-Not Otherwise Specified (PDD-NOS)). Subjects having a CNS or PNS trauma (e.g., brain or spinal cord ischemia or trauma, stroke, or a neurological deficit associated with surgery or anesthesia) may also be treated in accordance with the methods provided herein. The methods include administering an ASO provided herein or a pharmaceutical composition comprising the ASO to the subject. While not wishing to be bound by theory, the ASOs provided herein are believed to exert their desirable effects through their ability to modulate (e.g., increase or decrease) the levels of SYNGAP1 protein, SYNGAP1 mRNA, and/or SYNGAP1 activity within a cell of a subject, e.g., by increasing the level of the SYNGAP1 protein in a cell of the subject (e.g., a human, a mouse, a hamster, a non-human primate (e.g., a monkey)).

Another aspect of the present disclosure includes methods of modulating (e.g., increasing or decreasing) expression of SYNGAP1 in a cell of a subject, comprising contacting the cell with an ASO of the disclosure (or a pharmaceutical composition including the ASO), thereby treating a disease or disorder in the subject (e.g., a disease or disorder provided herein).

Another aspect of the disclosure includes methods of modulating (e.g., increasing or reducing) the level of SYNGAP1 mRNA or protein in a cell of a subject identified as having a disease or disorder provided herein (e.g., a SYNGAP1-related disorder).

Still another aspect includes methods of modulating (e.g., increasing or reducing) expression of a SYNGAP1 gene in a cell (e.g., in vivo, ex vivo, or in vitro) including contacting the cell with an ASO of the disclosure (or a pharmaceutical composition including the ASO), thereby increasing the expression of a SYNGAP1 gene in the cell. In some embodiments, the cell is a mammalian cell (e.g., a human cell such as a human neuron). The methods may include contacting a cell with an ASO of the disclosure (or a pharmaceutical composition including the ASO), in an amount effective to increase expression of a SYNGAP1 gene in the cell, thereby increasing expression of a SYNGAP1 gene in the cell. In some embodiments, contacting the cell with the ASO (or a pharmaceutical composition including the ASO) modulates (e.g., increases) the amount of SYNGAP1 mRNA in the cell. In some embodiments, contacting the cell with the ASO (or a pharmaceutical composition including the ASO) modulates (e.g., increases or decreases) the amount of SYNGAP1 protein in the cell. In some embodiments, contacting the cell with the ASO (or a pharmaceutical composition including the ASO) modulates (e.g., increases or decreases) the amount of SYNGAP1 activity in the cell.

In yet another aspect, the disclosure provides an ASO of the disclosure (or a pharmaceutical composition including the ASO) for use as a medicament. Further, the disclosure provides for an ASO of the disclosure (or a pharmaceutical composition including the ASO) for use in therapy.

Contacting of a cell with an ASO may be performed in vitro, ex vivo, or in vivo. Contacting a cell in vivo with the oligonucleotide includes contacting a cell or group of cells within a subject, e.g., a human subject, with the ASO. Combinations of in vitro, ex vivo, and in vivo methods of contacting a cell are also possible. Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc3 ligand, or any other ligand that directs the oligonucleotide to a site of interest. In some embodiments, the cell can be a neuron. For example, the neuron can be a neuron from the CNS, prefrontal cortex, motor cortex, or hippocampus. In some embodiments, the cell is a neuron. In some embodiments, the neuron is a glutamatergic neuron. In some embodiment, the ASO is administered with one or more agents capable of promoting penetration of the ASO across the blood-brain barrier. For example, in some embodiments, the ASO is coupled to a composition that promotes penetration or transportation of the ASO across the blood-brain barrier, e.g., a viral vector or an antibody to transferrin receptor.

Administration of an ASOs or pharmaceutical compositions disclosed herein to a subject can be intravenous, intraarterial, intraperitoneal, intramuscular, subcutaneous, transdermal, intrapleural, intrathecal, intracerebral, intraventricular, intracerebroventicular, intracisternal, intraspinal, peri-spinal, intracavitary, by perfusion through a catheter or by direct intralesional injection. In certain embodiments, the ASO or pharmaceutical composition is administered using an intracranial or intravertebral needle or catheter. In certain embodiments, the ASO or pharmaceutical composition is administered systemically. In certain embodiments, the ASO or pharmaceutical composition is administered by a parenteral route. For example, in certain embodiments, the ASO or pharmaceutical composition is administered by intravenously (e.g., by intravenous infusion), for example, with a prefilled bag, a prefilled pen, or a prefilled syringe. In other embodiments, the ASO or pharmaceutical composition is administered locally to an organ or tissue in which an increase in the target gene expression is desirable (e.g., neuron cells).

In some embodiments, the ASO is administered to a subject such that the ASO is delivered to a specific site within the subject. Such targeted delivery can be achieved by either systemic administration or local administration. The increase of expression of SYNGAP1 may be assessed by measuring the level or change in the level of SYNGAP1 mRNA or SYNGAP1 protein in a sample (e.g., blood, tissue (e.g., neurological tissue), a neuron cell sample (e.g., hippocampal cells, motor cortex cells, or prefrontal cortex cells), or neurological fluid (e.g., cerebrospinal fluid (CSF)) derived from a specific site within the subject. In certain embodiments, the methods include a clinically relevant increase of expression of SYNGAP1, e.g., as demonstrated by a clinically relevant outcome after treatment of a subject with an agent to reduce the expression of SYNGAP1.

In some embodiments, the methods provided herein may ameliorate or prevent the onset of one or more symptoms or conditions associated with a disease or disorder described herein (e.g. a SYNGAP1-related disorder), including epilepsy, cognitive impairment (e.g., moderate to severe cognitive impairment), hypotonia (e.g., mild hypotonia), global developmental delay, delayed language development, disordered sleep, oral dyspraxia, inattention, impulsivity, physical aggression, mood swings, sullenness, and rigidity. In some embodiments of the methods provided herein, an ASO provided herein (or a pharmaceutical composition including the ASO) is administered in an amount and for a time effective to result in reduction or improvement (e.g., by 10%, 20%, 30%, 40%, 50%, 60%, 70%, 80%, 90%, or 100%) of one or more symptoms associated with a disease or disorder described herein (e.g., a SYNGAP1-related disorder).

### Modulation of SYNGAP1 Expression Level

In some aspects, the therapeutic methods disclosed herein, using an ASO that targets a SYNGAP1 regRNA, result in modulated (e.g., increased or decreased) SYNGAP1 gene expression levels in a subject. Modulated expression of a SYNGAP1 gene includes any level of modulating of a SYNGAP1 gene, e.g., at least partial modulation of the expression of a SYNGAP1 gene. Modulated SYNGAP1 gene expression (e.g., increased or decreased) may be assessed by determining absolute or relative levels of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only (vehicle) control or inactive agent control). In certain embodiments, the methods provided herein result a clinically relevant modulation (e.g., an increase or decrease) of expression of SYNGAP1 gene, e.g. as demonstrated by a clinically relevant outcome after treatment of a subject with an agent to modulate (e.g., increase) the expression of SYNGAP1.

In certain embodiments, the methods disclosed herein result in increased SYNGAP1 gene expression in a cell, tissue (e.g., neurological tissue), a neuron cell sample (e.g., hippocampal cells, motor cortex cells, or prefrontal cortex cells), or sample (e.g., neurological fluid (e.g., cerebrospinal fluid (CSF)) of a subject by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose, pre-administration, or pre-exposure baseline level. In certain embodiments, the methods disclosed herein increases SYNGAP1 gene expression by at least 2-fold, at least 3-fold, at least 4-fold, at least 5-fold, at least 6-fold, at least-7 fold, at least 8-fold, at least 9-fold, or at least 10-fold relative to the pre-dose baseline level. In certain embodiments, the subject has a deficiency in SYNGAP1 expression, and the method disclosed herein restores the SYNGAP1 expression level (e.g., SYNGAP1 protein level or SYNGAP1 mRNA level) or SYNGAP1 protein activity to at least 20%, at least 30%, at least 40%, at least 50%, at least 60%, at least 70%, at least 80%, at least 90%, or at least 100% of the average SYNGAP1 expression level (e.g., SYNGAP1 protein level or SYNGAP1 mRNA level) or SYNGAP1 protein activity in similar cells, tissues or subjects (e.g., of the same species, of the like age and/or of the same sex) that do not have a deficiency in SYNGAP1 expression.

In some embodiments, an ASO of the disclosure may enhance the production of SYNGAP1 mRNA (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may enhance the production of SYNGAP1 mRNA (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

In certain embodiments, the methods disclosed herein result in decreased SYNGAP1 gene expression in a cell, tissue (e.g., neurological tissue), a neuron cell sample (e.g., hippocampal cells, motor cortex cells, or prefrontal cortex cells), or sample (e.g., neurological fluid (e.g., cerebrospinal fluid (CSF)) of a subject by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, relative to the pre-dose, pre-administration, or pre-exposure baseline level. In certain embodiments, the methods disclosed herein result in decreased SYNGAP1 gene expression by at least 2-fold, at least 3-fold, at least 4-fold, at least 5-fold, at least 6-fold, at least-7 fold, at least 8-fold, at least 9-fold, or at least 10-fold relative to the pre-dose baseline level.

In some embodiments, an ASO of the disclosure may reduce the production of SYNGAP1 mRNA (e.g., in a cell or in a cell, tissue, or sample of a subject) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, an ASO of the disclosure may reduce the production of SYNGAP1 mRNA (e.g., in a cell or in a cell, tissue, or sample of a subject) by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

In some embodiments, the expression of SYNGAP1 protein (e.g., one or more isoforms of SYNGAP1 protein, e.g., SYNGAP1 Aα1, SYNGAP1 Aα2, SYNGAP1 Aβ, SYNGAP1 Aγ, SYNGAP1 Bα1, SYNGAP1 Bα2, SYNGAP1 Bβ, SYNGAP1 Bγ, SYNGAP1 Cα1, SYNGAP1 Cα2, SYNGAP1 Cβ, SYNGAP1 Cγ, or any combination thereof) is modulated (e.g., increased or decreased) following treatment with, or administration of, an ASO of the disclosure. In some embodiments, the expression of SYNGAP1 protein (e.g., in a cell or in a cell, tissue, or sample of a subject (e.g., neurological tissue or neurological fluid (e.g., cerebrospinal fluid (CSF))) is modulated (e.g., increased or decreased) by at least about 1%, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%, at least about 100%, at least about 150%, at least about 200%, at least about 250%, at least about 300%, at least about 350%, at least about 400%, at least about 500%, at least about 600%, at least about 700%, at least about 800%, at least about 900% or more, relative to the pre-dose, pre-administration, or pre-exposure baseline level. In some embodiments, the expression of SYNGAP1 protein (e.g., in a cell or in a cell, tissue, or sample of a subject (e.g., neurological tissue or neurological fluid (e.g., cerebrospinal fluid (CSF))) is increased by at least 1-fold, at least 2-fold, at least 3-fold, at least 4-fold, at least 5-fold, at least 6-fold, at least 7-fold, at least 8-fold, at least 9-fold, or at least 10-fold or more, relative to the pre-dose, pre-administration, or pre-exposure baseline level.

The expression of a SYNGAP1 gene may be assessed based on the level of any variable associated with SYNGAP1 gene expression, e.g., SYNGAP1 mRNA level or SYNGAP1 protein level. In certain embodiments, the expression level or activity of SYNGAP1 herein refers to the average expression level or activity of SYNGAP1 in the brain (e.g., in neuronal cells of a mammal or human subject).

In certain embodiments, surrogate markers can be used to detect modulation (e.g., an increase or decrease) of SYNGAP1 expression level or SYNGAP1 activity. For example, effective treatment of a disease or disorder provided herein (e.g., a SYNGAP1-related disorder), as demonstrated by acceptable diagnostic and monitoring criteria with an agent to increase SYNGAP1 expression can be understood to demonstrate a clinically relevant increase in SYNGAP1.

Increased expression of SYNGAP1 gene may be manifested by an increase of the amount of mRNA expressed by a first cell or group of cells (such cells may be present, for example, in a sample derived from a subject) in which a SYNGAP1 gene is transcribed and which has or have been treated (e.g., by contacting the cell or cells with an oligonucleotide of the disclosure, or by administering an oligonucleotide of the disclosure to a subject in which the cells are or were present) such that the expression of a SYNGAP1 gene is increased, as compared to a second cell or group of cells substantially identical to the first cell or group of cells but which has not or have not been so treated (control cell(s) not treated with an oligonucleotide or not treated with an oligonucleotide targeted to the gene of interest). Decreased expression of SYNGAP1 gene may be manifested by a decrease of the amount of mRNA expressed by a first cell or group of cells (such cells may be present, for example, in a sample derived from a subject) in which a SYNGAP1 gene is transcribed and which has or have been treated (e.g., by contacting the cell or cells with an oligonucleotide of the disclosure, or by administering an oligonucleotide of the disclosure to a subject in which the cells are or were present) such that the expression of a SYNGAP1 gene is decreased, as compared to a second cell or group of cells substantially identical to the first cell or group of cells but which has not or have not been so treated (control cell(s) not treated with an oligonucleotide or not treated with an oligonucleotide targeted to the gene of interest).

In other embodiments, an increase or decrease in the expression of a SYNGAP1 gene may be assessed in terms of an increase of a parameter that is functionally linked to SYNGAP1 gene expression, e.g., SYNGAP1 protein expression or SYNGAP1 activity. An increase or decrease in SYNGAP1 protein levels, mRNA levels or activity may be determined in any cell expressing SYNGAP1, either endogenous or heterologous from an expression construct, and using any assay known in the art.

An increase or decrease of SYNGAP1 expression may be manifested by an increase or decrease in the level of the SYNGAP1 protein that is expressed by a cell or group of cells (e.g., the level of protein expressed in a sample derived from a subject), relative to a control cell or a control group of cells. An increase or decrease of SYNGAP1 expression may also be manifested by an increase in the level of the SYNGAP1 mRNA level in a treated cell or group of cells, relative to a control cell or a control group of cells.

A control cell or group of cells that may be used to assess the increase or decrease of the expression of a SYNGAP1 gene includes a cell or group of cells that has not yet been contacted with an oligonucleotide of the disclosure. For example, the control cell or group of cells may be derived from an individual subject (e.g., a human or animal subject) prior to treatment of the subject with an oligonucleotide.

The level of SYNGAP1 mRNA that is expressed by a cell or group of cells may be determined using any method known in the art for assessing mRNA expression. In one embodiment, the level of expression of SYNGAP1 in a sample is determined by detecting a transcribed polynucleotide, or portion thereof, e.g., mRNA of the SYNGAP1 gene. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating SYNGAP1 mRNA may be detected using methods the described in PCT Publication WO 2012/177906, the entire contents of which are hereby incorporated herein by reference. In some embodiments, the level of expression of SYNGAP1 is determined using a nucleic acid probe. The term “probe,” as used herein, refers to any molecule that is capable of selectively binding to a specific SYNGAP1 sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to SYNGAP1 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of SYNGAP1 mRNA.

An alternative method for determining the level of expression of SYNGAP1 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of SYNGAP1 is determined by quantitative fluorogenic RT-PCR (i.e., the TaqMan™ System) or the DUAL-GLO® Luciferase assay.

The expression levels of SYNGAP1 mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722; 5,874,219; 5,744,305; 5,677,195; and 5,445,934, which are incorporated herein by reference. The determination of SYNGAP1 expression level may also comprise using nucleic acid probes in solution.

In some embodiments, the level of mRNA expression is assessed using branched DNA (bDNA) assays, quantitative PCR (qPCR), real-time quantitative PCR (RT-qPCR), multiplex qPCR or RT-qPCR, RNA-seq, or microarray analysis. Such methods can also be used for the detection of SYNGAP1 nucleic acids.

The level of SYNGAP1 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, FACS, immunodiffusion (single or double), immunoelectrophoresis, western blotting, radioimmunoassay (RIA), enzyme-linked immunosorbent assays (ELISAs), immunofluorescent assays, electrochemiluminescence assays, Luminex, MSD, FISH, and the like. Such assays can also be used for the detection of proteins indicative of the presence or replication of SYNGAP1 proteins.

### Examples

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Modulation of SYNGAP1 Expression Using SYNGAP1 regRNA-Targeting ASOs

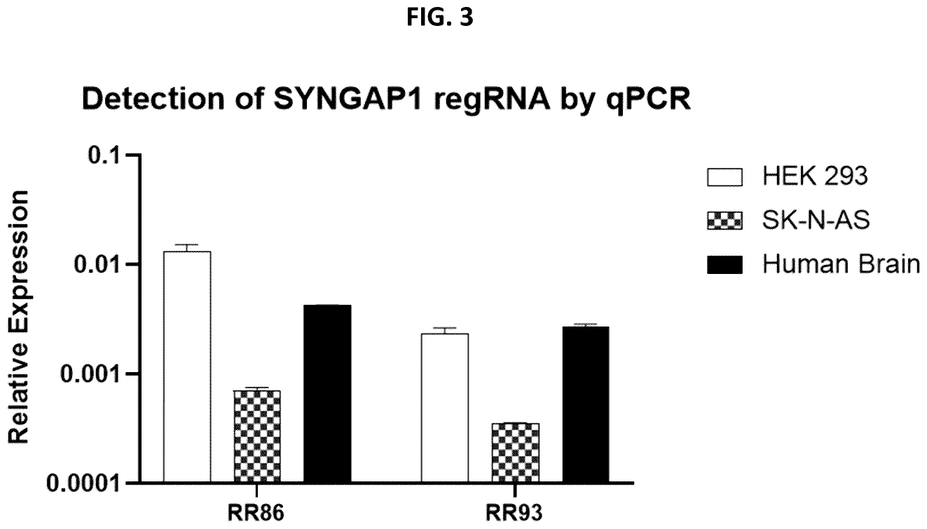

Four human SYNGAP1 regRNA targets were identified in the human genome (RR86, RR87, RR88, and RR93). To assess the expression of the human SYNGAP1 (hSYNGAP1) regRNAs RR86 and RR93 in HEK293 cells, SK-N-AS cells and human brain tissue, RNA capture seq and real time quantitative PCR (qPCR) were used. For this analysis, the qPCR reference gene was PPIA. As shown in FIG. 3, SYNGAP1 regRNAs RR86 and RR93 were detected in HEK293 and SK-N-AS cells, as well as human brain samples.

To assess the ability of ASOs targeting hSYGNAP1 regRNAs to modulate the expression of SYNGAP1, 210 ASOs targeting the human SYNGAP1 regRNAs were synthesized. 105 ASOs were steric oligonucleotides and 105 ASOs were gapmers. The ASOs were screened in SK-N-AS and HEK293 cells at 120 nM to determine their efficacy in increasing human SYNGAP1 mRNA levels. Briefly, SK-N-AS or HEK293 cells were reverse transfected with ASOs at 120 nM on Day 0 and cells were collected for mRNA quantification via qPCR on Day 2. Cells that were not treated with ASOs or were treated with either a gapmer non-targeting control (NTC) ASO (CO-1588) or a steric NTC ASO (CO-1589) were used as controls. mRNA was normalized to mRNA from cells treated with the gapmer NTC ASO (CO-1588).

From this initial screen, 11 ASOs were selected for chemistry fine tuning by altering the chemistry, type, and position of chemical modification of the selected ASOs. 39 additional ASOs were synthesized from the basewalking and tiling were further tested for dose dependent efficacy. 44 ASOs including additional chemical modifications were synthesized, including 4 extended gapmers, 24 LNA gapmers, 8 with P0/PS bonds, and 8 mixmers. ASOs that showed efficacy in increasing hSYNGAP1 mRNA at 120 nM in SK-N-AS and 7EK293 cells (as described above) were further tested for dose-dependent efficacy at 60 nM, 90 nM, or 120 nM in SK-N-AS cells or 12, 30, 60, 90, 120, or 150 nM in HEK293 cells.

ASO sequences and chemical modifications for the above screens are provided in FIG. 2 and Table 2. Table 5 provides the SYNGAP1 mRNA fold change for each of the ASOs tested at 120 or 150 nM in the CEK293 and SK-N-AS cells. Data in Table 5 is the highest fold change in either BTEK293 or SK-N-AS cells.

TABLE 5

| ASO | RegRNA | Fold | ASO | RegRNA | Fold | ASO | RegRNA | Fold |
|---|---|---|---|---|---|---|---|---|
| Name | ID | Change | Name | ID | Change | Name | ID | Change |
| CO-9338 | RR93_v1 | 1.321594 | CO-11485 | RR93_v1 | 1.098 | CO-7456 | RR86_v1 | 0.9449606 |
| CO-9339 | RR93_v1 | 1.3706311 | CO-11486 | RR93_v1 | 1.405 | CO-7457 | RR86_v1 | 1.0319805 |
| CO-9340 | RR93_v1 | 1.1504698 | CO-11487 | RR93_v1 | 1.023 | CO-7458 | RR86_v1 | 1.2095657 |
| CO-9341 | RR93_v1 | 1.2625709 | CO-11488 | RR93_v1 | 1.072 | CO-7459 | RR86_v1 | 0.985811 |
| CO-9342 | RR93_v1 | 1.1148151 | CO-11489 | RR93_v1 | 1.003 | CO-7460 | RR86_v1 | 1.0390162 |
| CO-9343 | RR93_v1 | 1.3888901 | CO-11490 | RR93_v1 | 1.315 | CO-7461 | RR86_v1 | 0.904346 |
| CO-9344 | RR93_v1 | 0.9657844 | CO-11491 | RR93_v1 | 1.259 | CO-7462 | RR86_v1 | 0.8664023 |
| CO-9345 | RR93_v1 | 1.2362275 | CO-11492 | RR93_v1 | 1.039 | CO-7463 | RR86_v1 | 1.0854104 |
| CO-9346 | RR93_v1 | 1.1890224 | CO-11493 | RR93_v1 | 0.967 | CO-7464 | RR86_v1 | 0.9973143 |
| CO-9347 | RR93_v1 | 1.2048252 | CO-11494 | RR93_v1 | 1.075 | CO-7465 | RR86_v1 | 0.9350017 |
| CO-9348 | RR93_v1 | 1.2810625 | CO-11495 | RR93_v1 | 0.918 | CO-7466 | RR86_v1 | 0.8167716 |
| CO-9349 | RR93_v1 | 1.2775086 | CO-11496 | RR93_v1 | 1.009 | CO-7467 | RR86_v1 | 1.1587845 |
| CO-9350 | RR93_v1 | 1.0835416 | CO-11497 | RR93_v1 | 1.041 | CO-7468 | RR86_v1 | 0.9602621 |
| CO-9351 | RR93_v1 | 1.3758323 | CO-11498 | RR93_v1 | 1.014 | CO-7469 | RR86_v1 | 0.9481977 |
| CO-9352 | RR93_v1 | 1.1376858 | CO-11499 | RR93_v1 | 1.01 | CO-7470 | RR86_v1 | 1.1200934 |
| CO-9353 | RR93_v1 | 1.2197741 | CO-11500 | RR93_v1 | 1.289 | CO-7471 | RR86_v1 | 1.0447312 |
| CO-9354 | RR93_v1 | 1.1876314 | CO-11501 | RR93_v1 | 1.085 | CO-7472 | RR86_v1 | 1.1077642 |
| CO-9355 | RR93_v1 | 1.2610805 | CO-11502 | RR93_v1 | 1.108 | CO-7473 | RR86_v1 | 1.04298 |
| CO-9356 | RR93_v1 | 1.2775573 | CO-11503 | RR93_v1 | 1.048 | CO-7474 | RR86_v1 | 1.0067661 |
| CO-9357 | RR93_v1 | 1.2064543 | CO-11504 | RR93_v1 | 1.009 | CO-7475 | RR86_v1 | 0.8484672 |
| CO-9358 | RR93_v1 | 1.1944626 | CO-11505 | RR93_v1 | 1.077 | CO-7476 | RR87_v1 | 0.9989343 |
| CO-9359 | RR93_v1 | 1.5521272 | CO-11506 | RR93_v1 | 1.116 | CO-7477 | RR87_v1 | 1.063341 |
| CO-9360 | RR93_v1 | 1.2819913 | CO-11507 | RR93_v1 | 1.192 | CO-7478 | RR87_v1 | 1.0571886 |
| CO-9361 | RR93_v1 | 1.2026502 | CO-11508 | RR93_v1 | 1.267 | CO-7479 | RR87_v1 | 0.9307392 |
| CO-9362 | RR93_v1 | 1.302456 | CO-11509 | RR93_v1 | 1.16 | CO-7480 | RR87_v1 | 1.1291831 |
| CO-9363 | RR93_v1 | 1.1767388 | CO-11510 | RR93_v1 | 1.284 | CO-7481 | RR87_v1 | 1.0592261 |
| CO-9364 | RR93_v1 | 1.2839077 | CO-11511 | RR93_v1 | 0.966 | CO-7482 | RR87_v1 | 1.6736641 |
| CO-9365 | RR93_v1 | 1.2768352 | CO-11512 | RR93_v1 | 1.024 | CO-7483 | RR87_v1 | 0.9840308 |
| CO-9366 | RR93_v1 | 2.0581875 | CO-11513 | RR93_v1 | 0.964 | CO-7484 | RR87_v1 | 1.0044173 |
| CO-9367 | RR93_v1 | 1.9645819 | CO-11514 | RR93_v1 | 1.893 | CO-7485 | RR87_v1 | 0.9125338 |
| CO-9368 | RR93_v1 | 1.2862002 | CO-11515 | RR93_v1 | 1.58 | CO-7486 | RR87_v1 | 0.9283744 |
| CO-9369 | RR93_v1 | 1.7420414 | CO-11516 | RR93_v1 | 1.471 | CO-7487 | RR87_v1 | 0.8363852 |
| CO-9370 | RR93_v1 | 1.790755 | CO-11517 | RR93_v1 | 1.687 | CO-7488 | RR87_v1 | 1.0833093 |
| CO-9371 | RR93_v1 | 1.3136199 | CO-11518 | RR93_v1 | 2.019 | CO-7489 | RR87_v1 | 1.1887636 |
| CO-9372 | RR93_v1 | 1.5815611 | CO-11519 | RR93_v1 | 1.982 | CO-7490 | RR88_v1 | 0.9341973 |
| CO-9373 | RR93_v1 | 2.4613097 | CO-11520 | RR93_v1 | 2.112 | CO-7491 | RR88_v1 | 1.0489649 |
| CO-9374 | RR93_v1 | 1.4548151 | CO-11521 | RR93_v1 | 1.889 | CO-7492 | RR88_v1 | 1.0391064 |
| CO-9375 | RR93_v1 | 1.6152445 | CO-11522 | RR93_v1 | 1.817 | CO-7493 | RR88_v1 | 1.0124646 |
| CO-9376 | RR93_v1 | 1.9087868 | CO-11523 | RR93_v1 | 1.067 | CO-7494 | RR88_v1 | 1.5231282 |
| CO-9377 | RR93_v1 | 1.1746632 | CO-11524 | RR93_v1 | 2.209 | CO-7495 | RR88_v1 | 1.1644063 |
| CO-9378 | RR93_v1 | 1.3066085 | CO-11525 | RR93_v1 | 2.002 | CO-7496 | RR88_v1 | 1.238685 |
| CO-9379 | RR93_v1 | 1.1252958 | CO-11526 | RR93_v1 | 2.078 | CO-7497 | RR88_v1 | 1.2139934 |
| CO-9380 | RR93_v1 | 1.3441474 | CO-11527 | RR93_v1 | 2.421 | CO-7498 | RR88_v1 | 1.3566668 |
| CO-9381 | RR93_v1 | 1.3566714 | CO-11528 | RR93_v1 | 2.557 | CO-7499 | RR88_v1 | 0.9723212 |
| CO-9382 | RR93_v1 | 1.1444834 | CO-11529 | RR93_v1 | 2.257 | CO-7500 | RR88_v1 | 1.1049107 |
| CO-9383 | RR93_v1 | 1.1938737 | CO-11530 | RR93_v1 | 1.908 | CO-7501 | RR88_v1 | 1.2699296 |
| CO-9384 | RR93_v1 | 1.1880869 | CO-11531 | RR93_v1 | 1.953 | CO-7502 | RR88_v1 | 0.9702517 |
| CO-9385 | RR93_v1 | 1.2090633 | CO-11532 | RR93_v1 | 1.947 | CO-7503 | RR88_v1 | 1.0589459 |
| CO-9386 | RR93_v1 | 1.0815572 | CO-11533 | RR93_v1 | 2.188 | CO-7504 | RR88_v1 | 0.9997099 |
| CO-9387 | RR93_v1 | 1.1823235 | CO-11534 | RR93_v1 | 2.207 | CO-7505 | RR88_v1 | 0.9774405 |
| CO-9388 | RR93_v1 | 1.2022095 | CO-11535 | RR93_v1 | 2.078 | CO-7506 | RR88_v1 | 0.9328241 |
| CO-9389 | RR93_v1 | 1.126392 | CO-11536 | RR93_v1 | 1.462 | CO-7507 | RR88_v1 | 1.0555954 |
| CO-9390 | RR93_v1 | 1.1360314 | CO-11537 | RR93_v1 | 1.519 | CO-7508 | RR88_v1 | 0.9813804 |
| CO-9391 | RR93_v1 | 1.1583284 | CO-11538 | RR93_v1 | 1.483 | CO-7509 | RR88_v1 | 0.8658039 |
| CO-9392 | RR93_v1 | 1.2230058 | CO-11539 | RR93_v1 | 1.443 | CO-7510 | RR88_v1 | 0.9170467 |
| CO-9393 | RR93_v1 | 1.2711363 | CO-11540 | RR93_v1 | 1.747 | CO-7511 | RR88_v1 | 0.9914842 |
| CO-9394 | RR93_v1 | 1.3902505 | CO-11541 | RR93_v1 | 1.62 | CO-7512 | RR88_v1 | 1.4082365 |
| CO-9395 | RR93_v1 | 1.012632 | CO-11542 | RR93_v1 | 1.787 | CO-7513 | RR88_v1 | 0.9855844 |
| CO-9396 | RR93_v1 | 1.0814878 | CO-11543 | RR93_v1 | 1.64 | CO-7514 | RR88_v1 | 1.028286 |
| CO-9397 | RR93_v1 | 1.2710926 | CO-11544 | RR93_v1 | 1.565 | CO-7515 | RR88_v1 | 0.9290347 |
| CO-9398 | RR93_v1 | 1.3041249 | CO-11545 | RR93_v1 | 1.129 | CO-7516 | RR88_v1 | 1.1425184 |
| CO-9399 | RR93_v1 | 0.707424 | CO-11546 | RR93_v1 | 1.516 | CO-7517 | RR88_v1 | 0.886103 |
| CO-9400 | RR93_v1 | 1.1224393 | CO-11547 | RR93_v1 | 1.525 | CO-7518 | RR88_v1 | 1.0418982 |
| CO-9401 | RR93_v1 | 1.0500868 | CO-11548 | RR93_v1 | 1.574 | CO-7519 | RR88_v1 | 1.0195556 |
| CO-9402 | RR93_v1 | 0.993377 | CO-11549 | RR93_v1 | 1.513 | CO-7520 | RR88_v1 | 1.0000126 |
| CO-9403 | RR93_v1 | 1.0394972 | CO-11550 | RR93_v1 | 1.438 | CO-7521 | RR88_v1 | 0.996495 |
| CO-9404 | RR93_v1 | 0.9099642 | CO-11551 | RR93_v1 | 1.544 | CO-7522 | RR88_v1 | 0.8529326 |
| CO-9405 | RR93_v1 | 1.1611975 | CO-11552 | RR93_v1 | 1.403 | | | |
| CO-9406 | RR93_v1 | 1.3640809 | CO-11553 | RR93_v1 | 1.264 | CO-7524 | RR88_v1 | 1.2802595 |
| CO-9407 | RR93_v1 | 1.0988679 | CO-11554 | RR93_v1 | 1.309 | CO-7525 | RR88_v1 | 0.9690307 |
| CO-9408 | RR93_v1 | 1.2155807 | CO-11555 | RR93_v1 | 1.513 | CO-10604 | RR86_v1 | 0.943379 |
| CO-9409 | RR93_v1 | 1.1399418 | CO-11556 | RR93_v1 | 1.606 | CO-10605 | RR86_v1 | 1.1428818 |
| CO-9410 | RR93_v1 | 1.0341938 | CO-11557 | RR93_v1 | 1.482 | CO-10606 | RR86_v1 | 0.9547887 |
| CO-9411 | RR93_v1 | 0.9782816 | CO-11558 | RR93_v1 | 1.013 | CO-10607 | RR86_v1 | 1.042436 |
| CO-9412 | RR93_v1 | 1.0786407 | CO-11559 | RR93_v1 | 1.226 | CO-10608 | RR86_v1 | 0.7367796 |
| CO-9413 | RR93_v1 | 0.8585804 | CO-11560 | RR93_v1 | 0.981 | CO-10609 | RR86_v1 | 0.9074972 |
| CO-9414 | RR93_v1 | 1.0130657 | CO-11561 | RR93_v1 | 1.224 | CO-10610 | RR86_v1 | 1.2663714 |
| CO-9415 | RR93_v1 | 1.1754598 | CO-11562 | RR93_v1 | 1.083 | CO-10611 | RR86_v1 | 1.0540569 |
| CO-9416 | RR93_v1 | 1.0307946 | CO-11563 | RR93_v1 | 1.164 | CO-10612 | RR86_v1 | 1.1257978 |
| CO-9417 | RR93_v1 | 1.2610847 | CO-11564 | RR93_v1 | 1.246 | CO-10613 | RR86_v1 | 1.000489 |
| CO-9418 | RR93_v1 | 1.0150563 | CO-11565 | RR93_v1 | 1.133 | CO-10614 | RR86_v1 | 1.470392 |
| CO-9419 | RR93_v1 | 1.1605032 | CO-11566 | RR93_v1 | 1.147 | CO-10615 | RR86_v1 | 1.6432752 |
| CO-9420 | RR93_v1 | 1.0259147 | CO-11567 | RR93_v1 | 1.024 | CO-10616 | RR86_v1 | 1.2661259 |
| CO-9421 | RR93_v1 | 1.0412051 | CO-11568 | RR93_v1 | 1.111 | CO-10617 | RR86_v1 | 1.2149476 |
| CO-9422 | RR93_v1 | 1.0320985 | CO-11569 | RR93_v1 | 1.219 | CO-10618 | RR86_v1 | 1.4933121 |
| CO-9423 | RR93_v1 | 0.9519308 | CO-11570 | RR93_v1 | 1.46 | CO-10619 | RR86_v1 | 1.42294 |
| CO-9424 | RR93_v1 | 1.0312607 | CO-11571 | RR93_v1 | 1.362 | CO-10620 | RR86_v1 | 1.4835377 |
| CO-9425 | RR93_v1 | 1.2115176 | CO-11572 | RR93_v1 | 1.223 | CO-10621 | RR86_v1 | 1.3489807 |
| CO-9426 | RR93_v1 | 1.0959396 | CO-11573 | RR93_v1 | 1.13 | CO-10622 | RR86_v1 | 1.283321 |
| CO-9427 | RR93_v1 | 1.3668122 | CO-11574 | RR93_v1 | 1.04 | CO-10623 | RR86_v1 | 1.3603462 |
| CO-9428 | RR93_v1 | 1.360219 | CO-11575 | RR93_v1 | 1.122 | CO-10624 | RR86_v1 | 1.0883013 |
| CO-9429 | RR93_v1 | 1.1775405 | CO-11576 | RR93_v1 | 1.253 | CO-10625 | RR86_v1 | 1.2365178 |
| CO-9430 | RR93_v1 | 1.2839459 | CO-11577 | RR93_v1 | 1.172 | CO-10626 | RR86_v1 | 1.2080423 |
| CO-9431 | RR93_v1 | 1.1963223 | CO-11578 | RR93_v1 | 1.361 | CO-10627 | RR86_v1 | 0.7432697 |
| CO-9432 | RR93_v1 | 0.9567216 | CO-11579 | RR93_v1 | 1.26 | CO-10628 | RR86_v1 | 0.9995231 |
| CO-9433 | RR93_v1 | 1.1487297 | CO-7426 | RR86_v1 | 1.0288763 | CO-10629 | RR86_v1 | 0.8889495 |
| CO-9434 | RR93_v1 | 0.8242675 | CO-7427 | RR86_v1 | 1.1754702 | CO-10630 | RR86_v1 | 1.3443304 |
| CO-9435 | RR93_v1 | 0.9650148 | CO-7428 | RR86_v1 | 1.0154496 | CO-10631 | RR86_v1 | 1.4537813 |
| CO-9436 | RR93_v1 | 1.0985332 | CO-7429 | RR86_v1 | 1.0081403 | CO-10632 | RR86_v1 | 1.3538474 |
| CO-9437 | RR93_v1 | 1.4831135 | CO-7430 | RR86_v1 | 0.9908796 | CO-10633 | RR86_v1 | 1.3639591 |
| CO-9438 | RR93_v1 | 1.5335774 | CO-7431 | RR86_v1 | 1.2733722 | CO-10634 | RR88_v1 | 1.3080051 |
| CO-9439 | RR93_v1 | 0.66379 | CO-7432 | RR86_v1 | 1.7380756 | CO-10635 | RR88_v1 | 1.3032588 |
| CO-9440 | RR93_v1 | 0.968923 | CO-7433 | RR86_v1 | 1.7766038 | CO-10636 | RR88_v1 | 1.5718849 |
| CO-9441 | RR93_v1 | 1.0276241 | CO-7434 | RR86_v1 | 1.068472 | CO-10637 | RR88_v1 | 1.5657107 |
| CO-9442 | RR93_v1 | 0.9190491 | CO-7435 | RR86_v1 | 1.8628839 | CO-10638 | RR88_v1 | 1.6248555 |
| CO-9443 | RR93_v1 | 1.0212564 | CO-7436 | RR86_v1 | 1.3504051 | CO-10639 | RR88_v1 | 1.3942842 |
| CO-9444 | RR93_v1 | 1.068958 | CO-7437 | RR86_v1 | 1.1638969 | CO-10640 | RR88_v1 | 1.4714194 |
| CO-9445 | RR93_v1 | 1.0122161 | CO-7438 | RR86_v1 | 1.0901726 | CO-10641 | RR88_v1 | 1.3662879 |
| CO-9446 | RR93_v1 | 1.27272 | CO-7439 | RR86_v1 | 1.04526 | CO-10642 | RR88_v1 | 1.207777 |
| CO-9447 | RR93_v1 | 1.0944535 | CO-7440 | RR86_v1 | 0.9528664 | CO-10643 | RR88_v1 | 0.7155983 |
| CO-11470 | RR93_v1 | 1.104 | CO-7441 | RR86_v1 | 1.4767496 | CO-10644 | RR86_v1 | 0.7624614 |
| CO-11471 | RR93_v1 | 1.222 | CO-7442 | RR86_v1 | 1.2390386 | CO-10645 | RR86_v1 | 1.1312052 |
| CO-11472 | RR93_v1 | 1.523 | CO-7443 | RR86_v1 | 1.0569675 | CO-10646 | RR86_v1 | 1.1087194 |
| CO-11473 | RR93_v1 | 1.156 | CO-7444 | RR86_v1 | 1.0135355 | CO-10647 | RR86_v1 | 1.2833403 |
| CO-11474 | RR93_v1 | 1.02 | CO-7445 | RR86_v1 | 0.8286293 | CO-10648 | RR86_v1 | 1.2953941 |
| CO-11475 | RR93_v1 | 1.368 | CO-7446 | RR86_v1 | 1.0410719 | CO-10649 | RR86_v1 | 1.0075258 |
| CO-11476 | RR93_v1 | 1.404 | CO-7447 | RR86_v1 | 1.5447964 | CO-10650 | RR86_v1 | 1.1894634 |
| CO-11477 | RR93_v1 | 1.522 | CO-7448 | RR86_v1 | 1.04163 | CO-10651 | RR88_v1 | 1.3664382 |
| CO-11478 | RR93_v1 | 1.349 | CO-7449 | RR86_v1 | 0.965321 | CO-10652 | RR88_v1 | 1.2517985 |
| CO-11479 | RR93_v1 | 1.378 | CO-7450 | RR86_v1 | 0.9393038 | CO-10653 | RR88_v1 | 1.2148849 |
| CO-11480 | RR93_v1 | 1.382 | CO-7451 | RR86_v1 | 0.9720582 | CO-10654 | RR88_v1 | 1.2079456 |
| CO-11481 | RR93_v1 | 1.426 | CO-7452 | RR86_v1 | 1.0010828 | CO-10655 | RR86_v1 | 0.9305552 |
| CO-11482 | RR93_v1 | 1.355 | CO-7453 | RR86_v1 | 0.9373946 | CO-10656 | RR86_v1 | 1.4071696 |
| CO-11483 | RR93_v1 | 1.423 | CO-7454 | RR86_v1 | 0.9346072 | CO-10657 | RR86_v1 | 1.0344922 |
| CO-11484 | RR93_v1 | 1.362 | CO-7455 | RR86_v1 | 0.9085137 | CO-10658 | RR86_v1 | 0.7174548 |

Gapmer ASOs CO-7432, CO-7433, CO-7435, CO-7441, CO-7447, CO-7482, and CO-7494, which target the regRNAs RR86, RR87, and RR88, upregulated SYNGAP1 mRNA levels more than 1.4 fold in 1TEK293 cells as compared to the control ASOs CO-1588 and CO-1589.

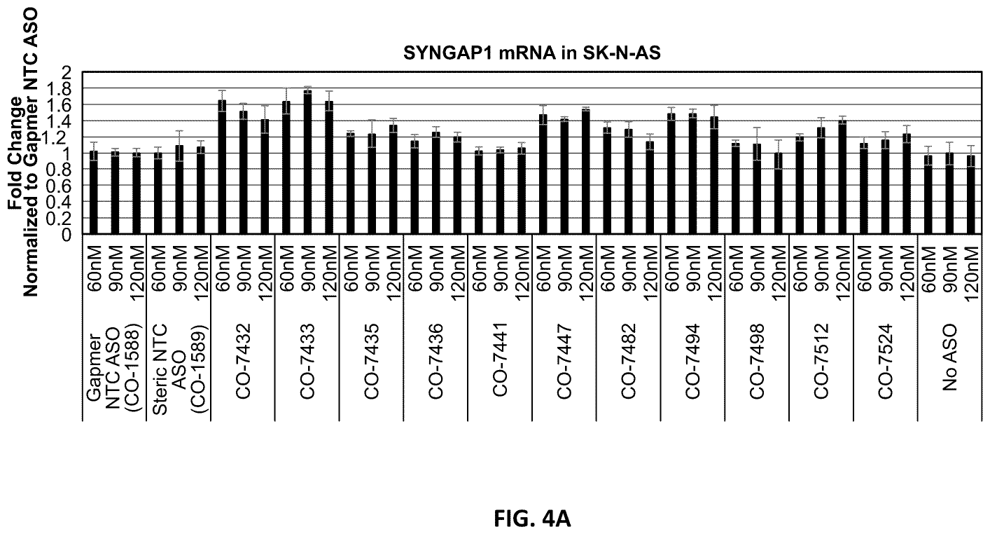

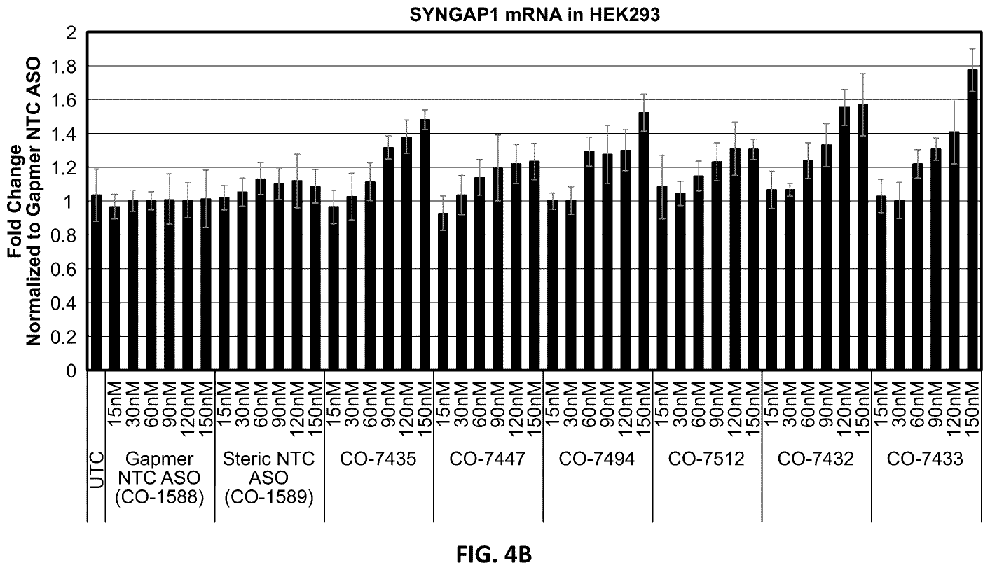

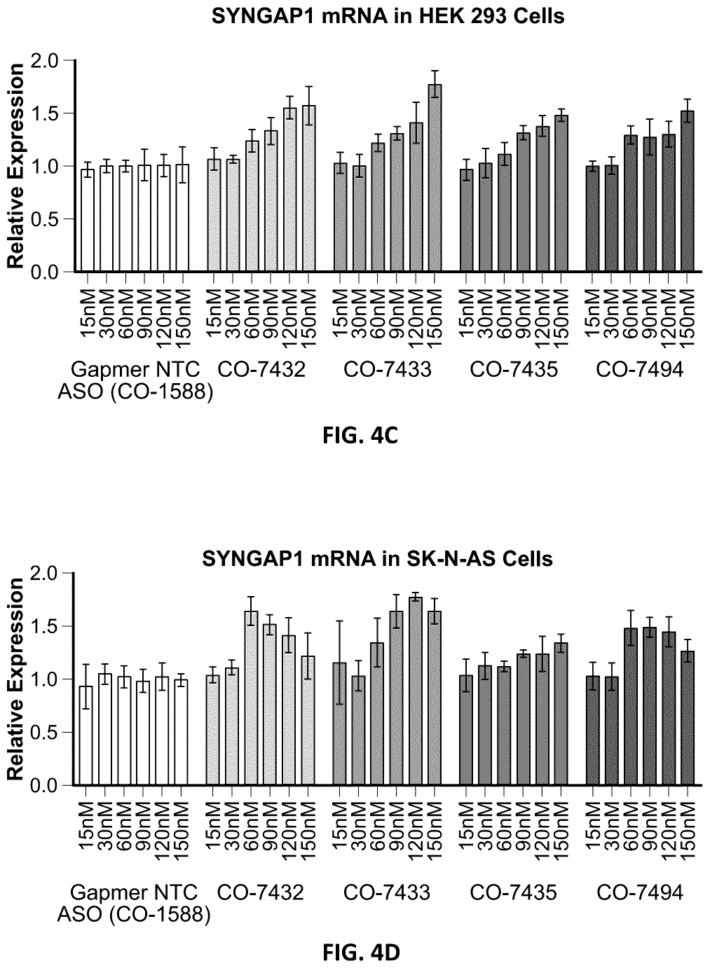

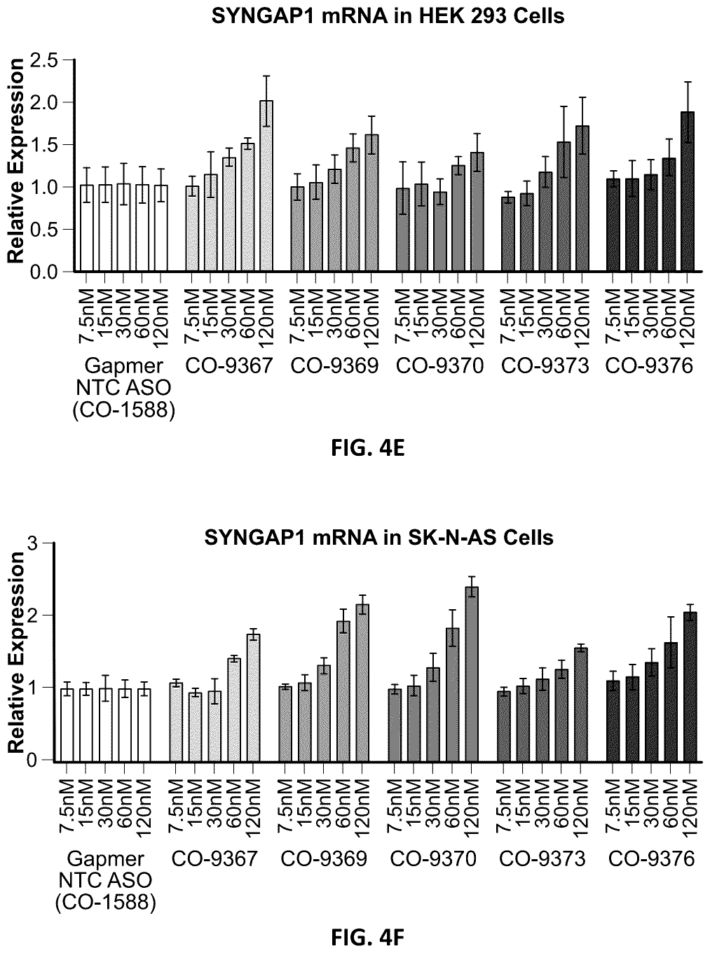

As shown in FIGS. 4A, 4B, 4C and FIG. 41D, a dose-dependent increase of SYNGAP1 mRNA in HTEK293 cells was observed after treatment with selected ASOs CO-7435, CO-7447, CO-7494, CO-7512, CO-7432, and CO-7433. In addition, ASOs CO-7432, CO-7433, CO-7435, CO-7436, CO-7447, CO-7482, CO-7494, CO-7498, CO-7512, and CO-7524 increased SYNGAP1 mRNA in SK-N-AS cells. As shown in FIGS. 4E and 4F, a dose-dependent increase of SYNGAP1 mRNA in 1TEK293 cells and SK-N-AS was observed after treatment with selected ASOs CO-9367, CO-9369, CO-9370, CO-9373, and CO-9376.

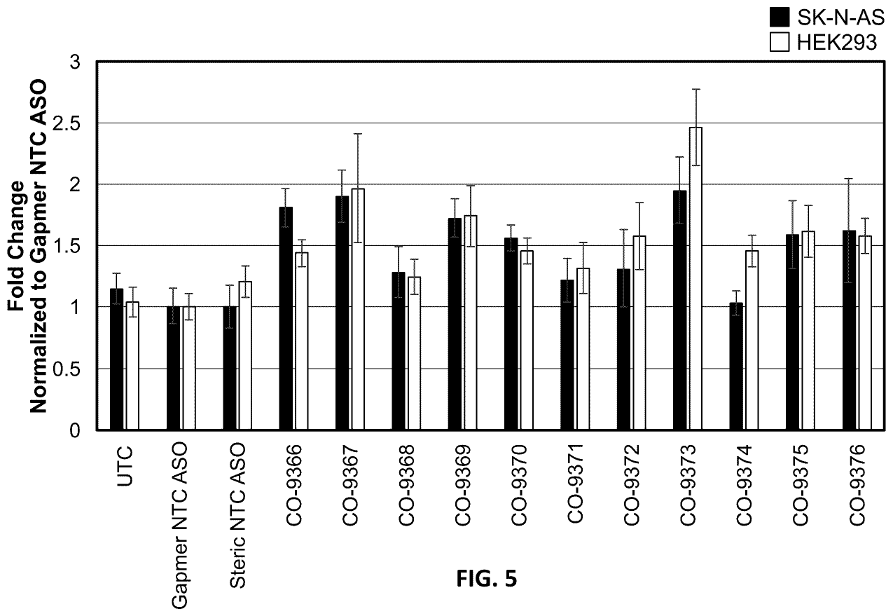

A gapmer hotspot at SYNGAP1 chr6:33419695-33419939 was identified between CO-9366 and CO-9376 (FIG. 5). SK-N-AS or HEK293 cells were reverse transfected on Day 0 with 120 nM of selected ASOs. Cells were collected on Day 2 for SYNGAP1 mRNA quantification using qPCR (as described above). Tiled ASOs CO-9366 to CO-9376 covering regRNA RR93 at this hotspot increased SYNGAP1 mRNA in both SK-N-AS and HEK239 cells 1.2- to 2.5-fold as compared to control ASOs CO-1588 and CO-1589 (FIG. 5).

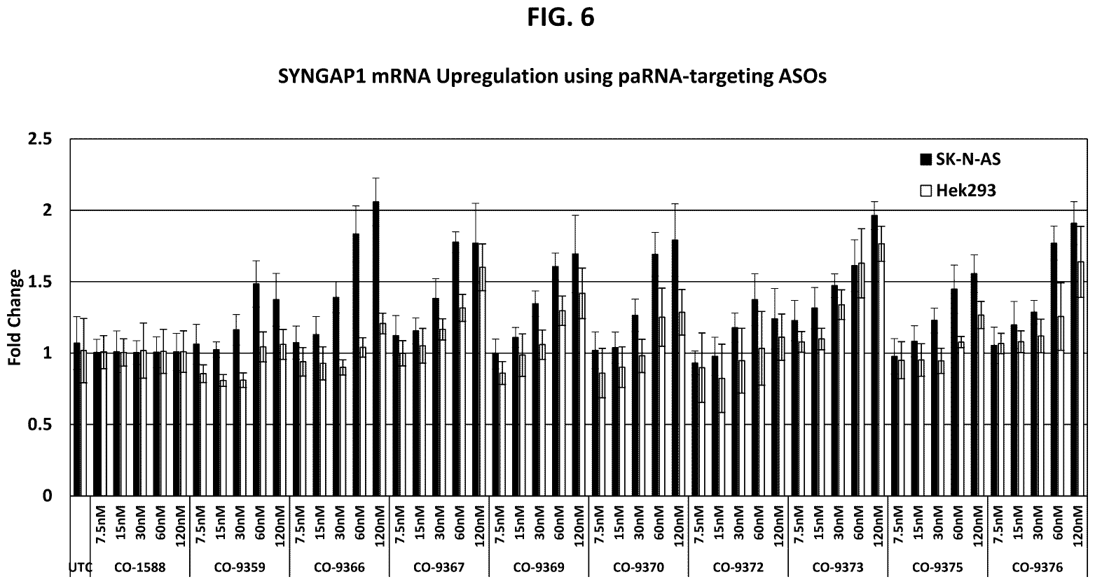

Additional dose response characterization of this regRNA hotspot in SK-N-AS and HEK293 cells was also performed by reverse transfecting cells with 7.5, 15, 30, 60, and 120 nM of ASOs as previously described. As shown in FIG. 6, the ASOs targeting the regRNA hotspot induced a dose-dependent upregulation of SYNGAP1 mRNA in both SK-N-AS and HEK293 cells.

### Example 2: ASOs Targeting SYNGAP1 regRNA RR86 and RR96 Induce Increased Expression of SYNGAP1 mRNA in iPSC-Differentiated Neurons

To assess the ability of ASOs targeting hSYGNAP1 regRNAs to modulate the expression of SYNGAP1 in a CNS translational model, the following experiment was performed using human iPSC differentiated neurons. Briefly, human induced pluripotent stem cells (iPSCs) were differentiated into neurons by overexpression of transcription factor Neurogenin-2 through viral transduction as described in Zhang et al. (2013) Neuron 78(5): 785-98, incorporated herein by reference. The differentiated neurons were allowed to mature in culture for 7-10 days, and transfected using Lipofectamine™ 2000 (INVITROGEN) with either 12.5 nM, 25 nM, 50 nM, 100 nM of ASOs targeting the SYNGAP1 regRNAs RR86_v2 and RR93: CO-10645, CO-11528, CO-7432, CO-7435, CO-9367, CO-9369, or CO-9370. SYNGAP1 mRNA was detected using qPCR as described above.

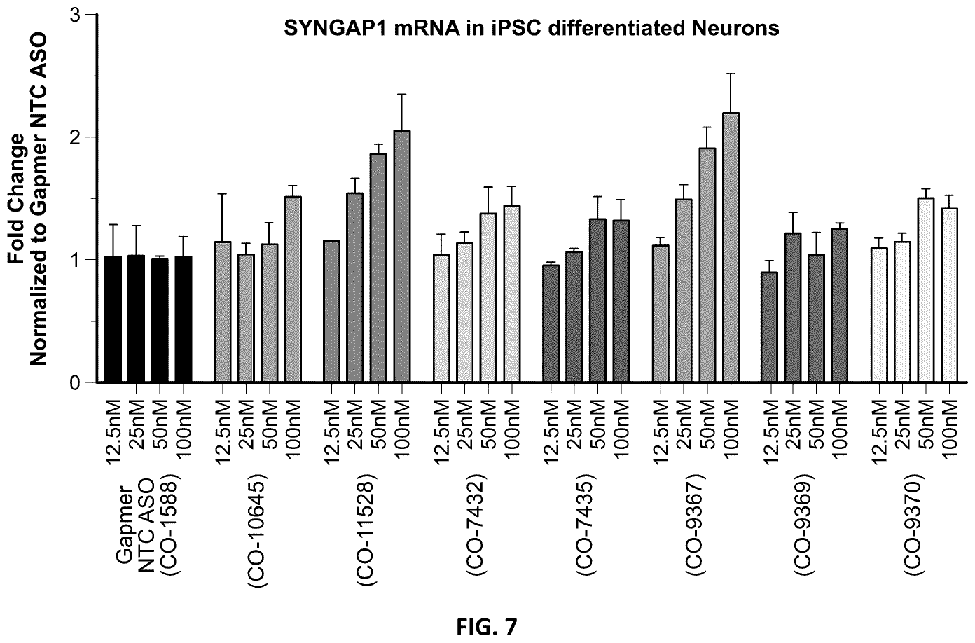

As shown in FIG. 7, the ASOs targeting RR86v2 and RR93 upregulated SYNGAP1 mRNA by about 1.5 to 2.2-fold as compared to control ASO CO-1588, indicating that targeting these regRNAs can be used to increase the expression of SYNGAP1 in a clinically relevant cell model.

### INCORPORATION BY REFERENCE

Unless stated to the contrary, the entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Scope of the disclosure is thus indicated by the appended claims rather than by the foregoing description, and all changes that come within the meaning and range of equivalency of the claims are intended to be embraced therein.

What is claimed is:

1. An antisense oligonucleotide (ASO) complementary to at least 8 contiguous nucleotides of a regulatory RNA of human SYNGAP1, wherein the regulatory RNA has a nucleotide sequence selected from the group consisting of SEQ ID NOs: 4, 5, or 6.

2. The ASO of claim 1, wherein the ASO is complementary to a sequence in the regRNA that is no more than 200 nucleotides from the 3′ end of the regRNA.

3. The ASO of claim 1, wherein the ASO is complementary to a sequence in the regRNA that is no more than 200 nucleotides from the 5′ end of the regRNA.

4. The ASO of claims 1-3, wherein the regRNA is not a polyadenylated RNA.

5. The ASO of any one of claims 1-4, wherein the regulatory RNA has a nucleotide sequence of SEQ ID NO: 5, and the ASO comprises a nucleotide sequence selected from the group consisting of SEQ ID NOs: 14-59, 220-250, 261-267, 272-278, 526-528, 542-591, 702-728, 729-735-741, 988-990, and 1007-2961.

6. The ASO of any one of claims 1-4, wherein the regulatory RNA has a nucleotide sequence of SEQ ID NO: 4 or 6, and the ASO comprises a nucleotide sequence selected from the group consisting of SEQ ID NOs: 110-219, 280-525, 529-541, 592-701, 742-987, 991-1003, and 2962-4852.

7. The ASO of any one of claims 1-4 or 6, wherein the ASO comprises the nucleotide sequence of at least 8 contiguous nucleotides of chr6:33419695-33419939.

8. The ASO of any one of claims 1-5, wherein the ASO comprises the nucleotide sequence of at least 8 contiguous nucleotides of chr6:33453987-33454269.

9. The ASO of any one of claims 1-4 or 6, wherein the ASO comprises the nucleotide sequence of at least 8 contiguous nucleotides of chr6:33419674-33419940.

10. The ASO of any one of claims 1-9, wherein the ASO is no more than 50, 40, 30, 25, 20, 18, or 16 nucleotides in length.

11. The ASO of any one of claims 1-10, wherein the ASO comprises a RNA polynucleotide comprising one or more chemical modifications.

12. The ASO of claim 11, wherein at least 3, 4, or 5 nucleotides at the 5′ end and at least 3, 4, or 5 nucleotides at the 3′ end of the ASO comprise ribonucleotides with one or more chemical modifications.

13. The ASO of claim 11 or 12, wherein the one or more chemical modifications comprise a nucleotide sugar modification comprising one or more of 2′-O-C1-4alkyl such as 2′-O-methyl (2′-OMe), 2′-deoxy (2′-H), 2′-OC1-3alkyl-O-C1-3alkyl such as 2′-methoxyethyl (“2′-MOE”), 2′-fluoro (“2′-F”), 2′-amino (“2′-NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-amido bridge nucleic acid (AmNA), 2′-unlocked nucleic acid (“ULNA”) nucleotide, a sugar in L form (“L-sugar”), 4′-thioribosyl nucleotide, constrained ethyl (cET), 2′-fluoro-arabino (FANA), or thiomorpholino.

14. The ASO of any one of claims 11-13, wherein the one or more chemical modifications comprise an internucleotide linkage modification comprising one or more of phosphorothioate (“PS” or (P(S))), phosphoramidate (P(NR1R2) such as dimethylaminophosphoramidate (P(N(CH3)2)), phosphonocarboxylate (P(CH2)nCOOR) such as phosphonoacetate “PACE” (P(CH2COO—)), thiophosphonocarboxylate ((S)P(CH2)nCOOR) such as thiophosphonoacetate “thioPACE” ((S)P(CH2COO—)), alkylphosphonate (P(C1-3alkyl) such as methylphosphonate —P(CH3), boranophosphonate (P(BH3)), or phosphorodithioate (P(S)2).

15. The ASO of any one of claims 11-14, wherein the one or more chemical modifications comprise a nucleobase modification comprising one or more of 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytosine (“5-allylC”), 5-aminoallyluracil (“5-aminoallylU”), 5-aminoallyl-cytosine (“5-aminoallylC”), an abasic nucleotide, Z base, P base, Unstructured Nucleic Acid (“UNA”), isoguanine (“isoG”), isocytosine (“isoC”) a glycerol nucleic acid (GNA), glycerol nucleic acid (GNA), or thiophosphoramidate morpholinos (TMOs).

16. The ASO of any one of claims 11-15, wherein the one or more chemical modifications comprise 2′-O-methoxyethyl, 5-methyl on cytidine, locked nucleic acid (LNA), phosphodiester (PO) internucleotide bond, or phosphorothioate (PS) internucleotide bond.

17. The ASO of any one of claims 11-16, wherein the ASO does not comprise 10 or more contiguous nucleotides of unmodified DNA.

18. The ASO of claim 17, wherein the ASO does not comprise a deoxyribonucleotide.

19. The ASO of any one of claims 11-18, wherein the ASO does not comprise an unmodified ribonucleotide.

20. The ASO of any one of claims 11-19, wherein the length of the ASO is 5×n+5 nucleotides (n is an integer of 3 or greater), wherein the nucleotides at positions 5×m are ribonucleotides modified by LNA (m is an integer from 1 to n) and the nucleotides at the remaining positions are ribonucleotides modified by 2′-O-methoxyethyl.

21. The ASO of any one of claims 11-19, wherein the length of the ASO is 3×n+2 nucleotides (n is an integer of 6 or greater), wherein the nucleotides at positions 3×m are ribonucleotides modified by LNA (m is an integer from 1 to n) and the nucleotides at the remaining positions are ribonucleotides modified by 2′-O-methoxyethyl.

22. The ASO of any one of claims 11-19, wherein each ribonucleotide of the ASO is modified by 2′-O-methoxyethyl.

23. The ASO of any one of claims 11-19, wherein each nucleotide of the ASO is a ribonucleotide modified by 2′-O-methoxyethyl.

24. The ASO of any one of claims 11-23, wherein the ASO comprises 10 or more contiguous nucleotides of unmodified DNA flanked by at least 3 nucleotides of modified ribonucleotides at each ofthe 5′ end and the 3′ end.

25. The ASO of any one of claims 11-24, wherein each cytidine in the ASO is modified by 5-methyl.

26. The ASO of any one of claims 1-25, wherein the regRNA is an paRNA.

27. A pharmaceutical composition comprising the ASO of any one of claims 1-26 and a pharmaceutically acceptable carrier or excipient carrier.

28. A method of increasing transcription of SYNGAP1 in a human cell, the method comprising contacting the cell with the ASO of any one of claims 1-26 or the pharmaceutical composition of claim 27.

29. The method of claim 28, wherein the cell is a neuron.

30. The method of claim 28 or 29, wherein the ASO increases the amount of the regulatory RNA in the cell.

31. The method of any one of claims 28-30, wherein the ASO increases the stability of the regulatory RNA in the cell.

32. The method of any one of claims 28-31, wherein the method results in increased SYNGAP1 mRNA in the cell.

33. The method of any one of claims 28-32, wherein the method results in increased SYNGAP1 protein in the cell.

34. A method of treating disease or disorder, the method comprising administering to a subject in need thereof an effective amount of the ASO of any one of claims 1-26 or the pharmaceutical composition of claim 27.

35. The method of claim 34, wherein the disease or disorder is a SYNGAP1-related disease or disorder.

36. The method of claim 34 or 37, wherein the SYNGAP1-related disorder is SYNGAPi-related intellectual disability (ID), mental retardation, autosomal dominant 5 (MHRD5), or SYNGAP1-related non-syndromic intellectual disability (NSID).

37. The method of claim 34, wherein the disease or disorder is a central nervous system (CNS) disorder or a peripheral nervous system (PNS) disorder.

38. The method of claim 38, wherein the disease or disorder is an affective disorder (e.g., depression), schizophrenia, Alzheimer's disease, Parkinson's disease, Huntington's disease, an autism spectrum disorder (ASD), (e.g., Asperger's syndrome, autistic disorder, Pervasive Developmental Disorder-Not Otherwise Specified (PDD-NOS)), or a CNS or PNS trauma (e.g., brain or spinal cord ischemia or trauma, stroke, or a neurological deficit associated with surgery or anesthesia)

39. The method of any one of claims 34-38, wherein administration of the ASO increases SYNGAP1 gene expression in the subject relative to a pre-administration baseline level.

40. The method of any one of claims 34-39, wherein the ASO increases the amount of the regulatory RNA in a cell of the subject.

41. The method of any one of claims 34-40, wherein the ASO increases the stability of the regulatory RNA in a cell of the subject.

42. The method of any one of claims 34-41, wherein administration of the ASO increases SYNGAP1 gene expression in a cell of the subject relative to a pre-administration baseline level.

43. The method of claim 40-42, wherein the cell is a neuron.
